(19) United States
(12) Patent Application Publication
(10) Pub. No.: US 2020/0185620 A1
(43) Pub. Date: Jun. 11, 2020

(54) COMPOUND, ORGANIC ELECTROLUMINESCENT ELEMENT, AND ELECTRONIC DEVICE

(71) Applicant: IDEMITSU KOSAN CO., LTD., Tokyo (JP)

(72) Inventors: Yuki NAKANO, Sodegaura-shi (JP); Ryota TAKAHASHI, Sodegaura-shi (JP); Kazuma MASE, Sodegaura-shi (JP); Tomoki KATO, Sodegaura-shi (JP)

(73) Assignee: IDEMITSU KOSAN CO., LTD., Tokyo (JP)

(21) Appl. No.: 16/699,442

(22) Filed: Nov. 29, 2019

(30) Foreign Application Priority Data
Nov. 30, 2018 (JP) 2018-226107

(51) Int. Cl. H01L 51/00; C07D 487/22; C09K 11/06

(52) U.S. Cl. CPC H01L 51/0072; C07D 487/22; H01L 51/5012; H01L 51/0061; H01L 51/0073; C09K 11/06

(57) ABSTRACT

A compound is represented by a formula (2) below,

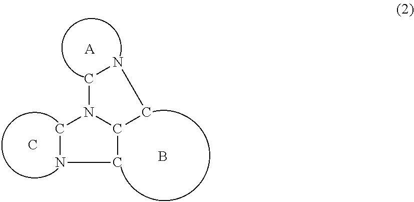

where rings A and C each independently represent a substituted or unsubstituted aromatic heterocycle having 5 to 50 ring atoms, a ring B represents a substituted or unsubstituted aromatic heterocycle having 5 to 50 ring atoms or a substituted or unsubstituted aromatic hydrocarbon ring having 6 to 50 ring carbon atoms.

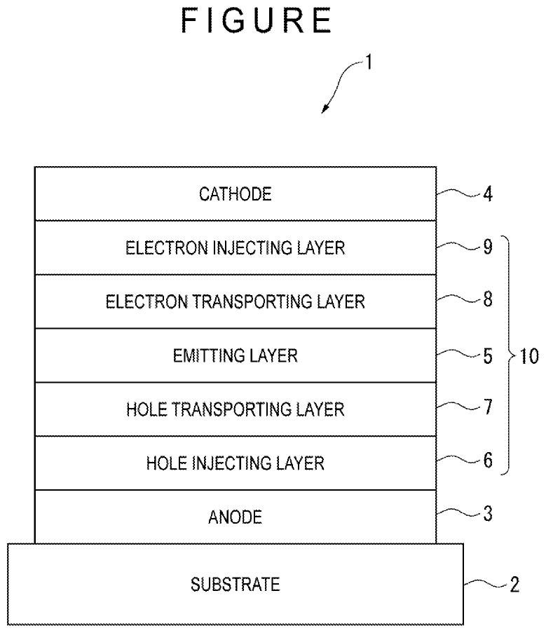

### TECHNICAL FIELD

The present invention relates to a compound, an organic electroluminescence device, and an electronic device.

### BACKGROUND ART

When a voltage is applied to an organic electroluminescence device (hereinafter, occasionally referred to as “organic EL device”), holes are injected from an anode and electrons are injected from a cathode into an emitting layer. The injected electrons and holes are recombined in the emitting layer to form excitons. Specifically, according to the electron spin statistics theory, singlet excitons and triplet excitons are generated at a ratio of 25%:75%.

Organic EL device finds its application in full-color displays of cellular phones, televisions, and the like. In order to enhance the performance of the organic EL device, various studies have been made for compounds used in the organic EL device (see, for instance, Patent Literature 1: WO 2013/077344). The performance of the organic EL device is evaluatable in terms of, for instance, luminance, emission wavelength, chromaticity, emission efficiency, drive voltage, and lifetime.

### SUMMARY OF THE INVENTION

An object of the invention is to provide a compound capable of enhancing performance of an organic electroluminescence device. Another object of the invention is to provide an organic electroluminescence device containing the compound capable of enhancing performance of the organic electroluminescence device and an electronic device including the organic electroluminescence device.

According to an aspect of the invention, a compound represented by a formula (2) below is provided.

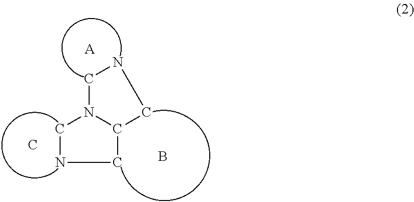

In the formula (2):

the rings A and C each independently represent a substituted or unsubstituted aromatic heterocycle having 5 to 50 ring atoms;

a ring B represents a substituted or unsubstituted aromatic heterocycle having 5 to 50 ring atoms, or a substituted or unsubstituted aromatic hydrocarbon ring having 6 to 50 ring carbon atoms;

when the ring A, the ring B, and the ring C have respective substituents, the substituents are each independently a substituted or unsubstituted alkyl group having 1 to 50 carbon atoms, a substituted or unsubstituted fluoroalkyl group having 1 to 50 carbon atoms, a substituted or unsubstituted cycloalkyl group having 3 to 50 ring carbon atoms, a cyano group, a halogen atom, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms;

R₉₀₁, R₉₀₂, R₉₀₃, R₉₀₆, and R₉₀₇ are each independently a substituted or unsubstituted aryl group having 6 to 50 ring carbon atoms, a substituted or unsubstituted heterocyclic group having 5 to 50 ring atoms, a substituted or unsubstituted alkyl group having 1 to 50 carbon atoms, or a substituted or unsubstituted cycloalkyl group having 3 to 50 ring carbon atoms; and

R₉₀₄ and R₉₀₅ are each independently a substituted or unsubstituted aryl group having 6 to 50 ring carbon atoms, a substituted or unsubstituted heterocyclic group having 5 to 50 ring atoms, a substituted or unsubstituted alkyl group having 1 to 50 carbon atoms, a substituted or unsubstituted cycloalkyl group having 3 to 50 ring carbon atoms, or a substituted or unsubstituted fluoroalkyl group having 1 to 50 carbon atoms.

According to another aspect of the invention, an organic electroluminescence device including an anode, a cathode, and a first organic layer interposed between the anode and the cathode is provided, the first organic layer containing the compound according to the above-described aspect of the invention.

According to still another aspect of the invention, an electronic device including the organic electroluminescence device according to the above aspect of the invention is provided.

According to the invention, a compound capable of enhancing performance of an organic electroluminescence device can be provided. Further, according to the invention, an organic electroluminescence device containing the compound capable of enhancing performance of the organic electroluminescence device and an electronic device including the organic electroluminescence device can be provided.

### BRIEF EXPLANATION OF DRAWING(S)

THE FIGURE schematically illustrates an arrangement of an organic electroluminescence device according to an exemplary embodiment of the invention.

### DESCRIPTION OF EMBODIMENT(S)

### Definitions

Herein, a hydrogen atom includes isotope having different numbers of neutrons, specifically, protium, deuterium and tritium.

In chemical formulae herein, it is assumed that a hydrogen atom (i.e. protium, deuterium and tritium) is bonded to each of bondable positions that are not annexed with signs “R” or the like or “D” representing a protium.

Herein, the ring carbon atoms refer to the number of carbon atoms among atoms forming a ring of a compound (e.g., a monocyclic compound, fused-ring compound, crosslinking compound, carbon ring compound, and heterocyclic compound) in which the atoms are bonded with each other to form the ring. When the ring is substituted by a substituent(s), carbon atom(s) contained in the substituent(s) is not counted in the ring carbon atoms. Unless otherwise specified, the same applies to the “ring carbon atoms” described later. For instance, a benzene ring has 6 ring carbon atoms, a naphthalene ring has 10 ring carbon atoms, a pyridine ring has 5 ring carbon atoms, and a furan ring has 4 ring carbon atoms. Further, for instance, 9,9-diphenylfluorenyl group has 13 ring carbon atoms and 9,9′-spirobifluorenyl group has 25 ring carbon atoms.

When a benzene ring is substituted by a substituent in a form of, for instance, an alkyl group, the number of carbon atoms of the alkyl group is not counted in the number of the ring carbon atoms of the benzene ring. Accordingly, the benzene ring substituted by an alkyl group has 6 ring carbon atoms. When a naphthalene ring is substituted by a substituent in a form of, for instance, an alkyl group, the number of carbon atoms of the alkyl group is not counted in the number of the ring carbon atoms of the naphthalene ring. Accordingly, the naphthalene ring substituted by an alkyl group has 10 ring carbon atoms.

Herein, the ring atoms refer to the number of atoms forming a ring of a compound (e.g., a monocyclic compound, fused-ring compound, crosslinking compound, carbon ring compound, and heterocyclic compound) in which the atoms are bonded to each other to form the ring (e.g., monocyclic ring, fused ring, and ring assembly). Atom(s) not forming the ring (e.g., hydrogen atom(s) for saturating the valence of the atom which forms the ring) and atom(s) in a substituent by which the ring is substituted are not counted as the ring atoms. Unless otherwise specified, the same applies to the “ring atoms” described later. For instance, a pyridine ring has 6 ring atoms, a quinazoline ring has 10 ring atoms, and a furan ring has 5 ring atoms. For instance, the number of hydrogen atom(s) bonded to a pyridine ring or the number of atoms forming a substituent are not counted as the pyridine ring atoms. Accordingly, a pyridine ring bonded with a hydrogen atom(s) or a substituent(s) has 6 ring atoms. For instance, the hydrogen atom(s) bonded to a quinazoline ring or the atoms forming a substituent are not counted as the quinazoline ring atoms. Accordingly, a quinazoline ring bonded with hydrogen atom(s) or a substituent(s) has 10 ring atoms.

Herein, “XX to YY carbon atoms” in the description of “substituted or unsubstituted ZZ group having XX to YY carbon atoms” represent carbon atoms of an unsubstituted ZZ group and do not include carbon atoms of a substituent(s) of the substituted ZZ group. Herein, “YY” is larger than “XX,” “XX” representing an integer of 1 or more and “YY” representing an integer of 2 or more.

Herein, “XX to YY atoms” in the description of “substituted or unsubstituted ZZ group having XX to YY atoms” represent atoms of an unsubstituted ZZ group and does not include atoms of a substituent(s) of the substituted ZZ group. Herein, “YY” is larger than “XX,” “XX” representing an integer of 1 or more and “YY” representing an integer of 2 or more.

Herein, an unsubstituted ZZ group refers to an “unsubstituted ZZ group” in a “substituted or unsubstituted ZZ group,” and a substituted ZZ group refers to a “substituted ZZ group” in a “substituted or unsubstituted ZZ group.”

Herein, the term “unsubstituted” used in a “substituted or unsubstituted ZZ group” means that a hydrogen atom(s) in the ZZ group is not substituted with a substituent(s). The hydrogen atom(s) in the “unsubstituted ZZ group” is protium, deuterium, or tritium.

Herein, the term “substituted” used in a “substituted or unsubstituted ZZ group” means that at least one hydrogen atom in the ZZ group is substituted with a substituent. Similarly, the term “substituted” used in a “BB group substituted by AA group” means that at least one hydrogen atom in the BB group is substituted with the AA group.

### Substituent Mentioned Herein

Substituents mentioned herein will be described below.

An “unsubstituted aryl group” mentioned herein has, unless otherwise specified herein, 6 to 50, preferably 6 to 30, more preferably 6 to 18 ring carbon atoms.

An “unsubstituted heterocyclic group” mentioned herein has, unless otherwise specified herein, 5 to 50, preferably 5 to 30, more preferably 5 to 18 ring atoms.

An “unsubstituted alkyl group” mentioned herein has, unless otherwise specified herein, 1 to 50, preferably 1 to 20, more preferably 1 to 6 carbon atoms.

An “unsubstituted alkenyl group” mentioned herein has, unless otherwise specified herein, 2 to 50, preferably 2 to 20, more preferably 2 to 6 carbon atoms.

An “unsubstituted alkynyl group” mentioned herein has, unless otherwise specified herein, 2 to 50, preferably 2 to 20, more preferably 2 to 6 carbon atoms.

An “unsubstituted cycloalkyl group” mentioned herein has, unless otherwise specified herein, 3 to 50, preferably 3 to 20, more preferably 3 to 6 ring carbon atoms.

An “unsubstituted arylene group” mentioned herein has, unless otherwise specified herein, 6 to 50, preferably 6 to 30, more preferably 6 to 18 ring carbon atoms.

An “unsubstituted divalent heterocyclic group” mentioned herein has, unless otherwise specified herein, 5 to 50, preferably 5 to 30, more preferably 5 to 18 ring atoms.

An “unsubstituted alkylene group” mentioned herein has, unless otherwise specified herein, 1 to 50, preferably 1 to 20, more preferably 1 to 6 carbon atoms.

### Substituted or Unsubstituted Aryl Group

Specific examples (specific example group G1) of the “substituted or unsubstituted aryl group” mentioned herein include unsubstituted aryl groups (specific example group G1A) below and substituted aryl groups (specific example group G1B) (herein, an unsubstituted aryl group refers to an “unsubstituted aryl group” in a “substituted or unsubstituted aryl group,” and a substituted aryl group refers to a “substituted aryl group” in a “substituted or unsubstituted aryl group.”) A simply termed “aryl group” herein includes both of “unsubstituted aryl group” and “substituted aryl group.”

The “substituted aryl group” refers to a group derived by substituting at least one hydrogen atom in an “unsubstituted aryl group” with a substituent. Examples of the “substituted aryl group” include a group derived by substituting at least one hydrogen atom in the “unsubstituted aryl group” in the specific example group G1A below with a substituent, and examples of the substituted aryl group in the specific example group G1B below. It should be noted that the examples of the “unsubstituted aryl group” and the “substituted aryl group” mentioned herein are merely exemplary, and the “substituted aryl group” mentioned herein includes a group derived by substituting a hydrogen atom bonded to a carbon atom of a skeleton of a “substituted aryl group” in the specific example group G1B below, and a group derived by substituting a hydrogen atom of a substituent of the “substituted aryl group” in the specific example group G1B below.

Unsubstituted Aryl Group (Specific Example Group G1A):

a phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, benzanthryl group, phenanthryl group, benzophenanthryl group, phenalenyl group, pyrenyl group, chrysenyl group, benzochrysenyl group, triphenylenyl group, benzotriphenylenyl group, tetracenyl group, pentacenyl group, fluorenyl group, 9,9′-spirobifluorenyl group, benzofluorenyl group, dibenzofluorenyl group, fluoranthenyl group, benzofluoranthenyl group, a perylenyl group, and a monovalent aryl group derived by removing one hydrogen atom from cyclic structures represented by formulae (TEMP-1) to (TEMP-15) below.

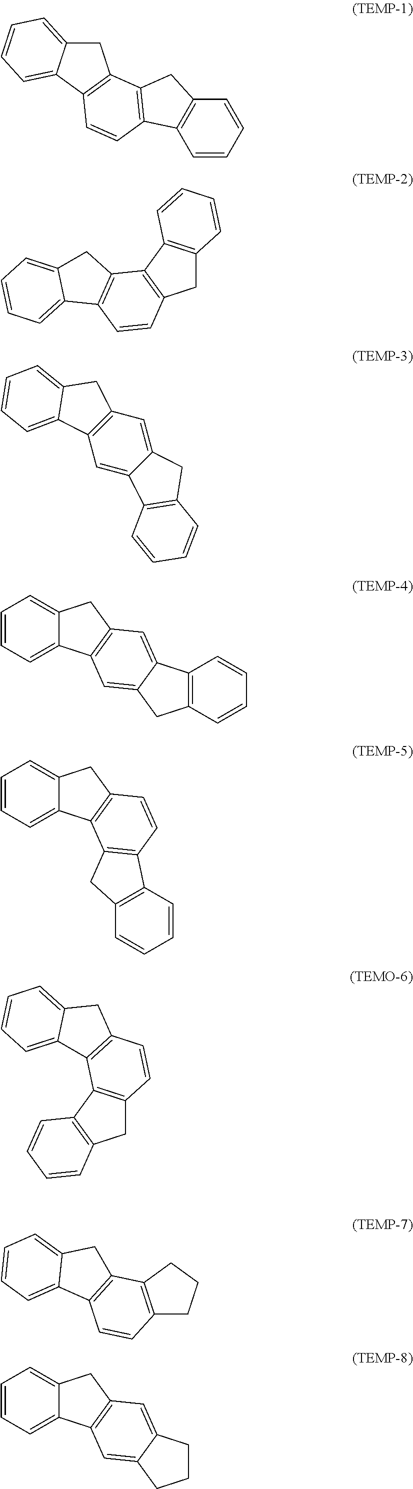

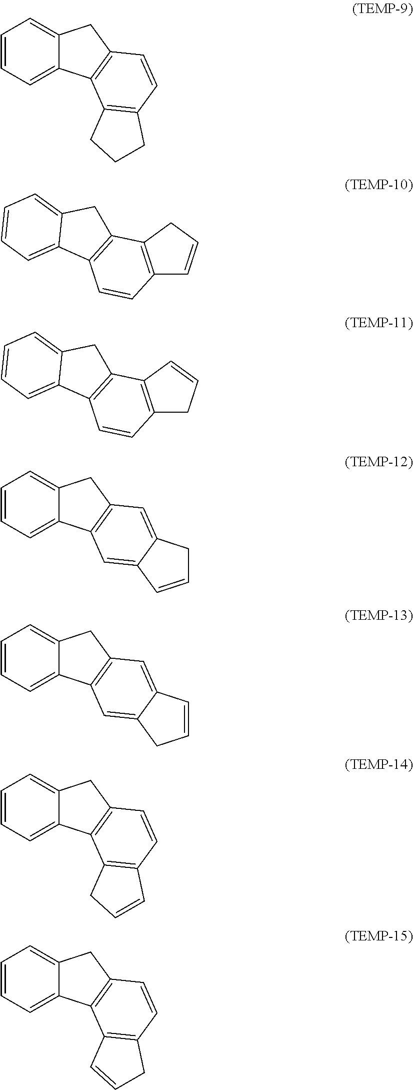

Substituted Aryl Group (Specific Example Group G1B):

o-tolyl group, m-tolyl group, p-tolyl group, para-xylyl group, meta-xylyl group, ortho-xylyl group, para-isopropylphenyl group, meta-isopropylphenyl group, ortho-isopropylphenyl group, para-t-butylphenyl group, meta-t-butylphenyl group, ortho-t-butylphenyl group, 3,4,5-trimethylphenyl group, 9,9-dimethylfluorenyl group, 9,9-diphenylfluorenyl group, 9,9-bis(4-methylphenyl)fluorenyl group, 9,9-bis(4-isopropylphenyl)fluorenyl group, 9,9-bis(4-t-butylphenyl)fluorenyl group, cyanophenyl group, triphenylsilylphenyl group, trimethylsilylphenyl group, phenylnaphthyl group, naphthylphenyl group, and a group derived by substituting at least one hydrogen atom of a monovalent group derived from one of the cyclic structures represented by the formulae (TEMP-1) to (TEMP-15) with a substituent.

### Substituted or Unsubstituted Heterocyclic Group

The “heterocyclic group” mentioned herein refers to a cyclic group having at least one hetero atom in the ring atoms. Specific examples of the hetero atom include a nitrogen atom, oxygen atom, sulfur atom, silicon atom, phosphorus atom, and boron atom.

The “heterocyclic group” mentioned herein is a monocyclic group or a fused-ring group.

The “heterocyclic group” mentioned herein is an aromatic heterocyclic group or a non-aromatic heterocyclic group.

Specific examples (specific example group G2) of the “substituted or unsubstituted heterocyclic group” mentioned herein include unsubstituted heterocyclic groups (specific example group G2A) and substituted heterocyclic groups (specific example group G2B) (herein, an unsubstituted heterocyclic group refers to an “unsubstituted heterocyclic group” in a “substituted or unsubstituted heterocyclic group,” and a substituted heterocyclic group refers to a “substituted heterocyclic group” in a “substituted or unsubstituted heterocyclic group.”) A simply termed “heterocyclic group” herein includes both of “unsubstituted heterocyclic group” and “substituted heterocyclic group.”

The “substituted heterocyclic group” refers to a group derived by substituting at least one hydrogen atom in an “unsubstituted heterocyclic group” with a substituent. Specific examples of the “substituted heterocyclic group” include a group derived by substituting at least one hydrogen atom in the “unsubstituted heterocyclic group” in the specific example group G2A below with a substituent, and examples of the substituted heterocyclic group in the specific example group G2B below. It should be noted that the examples of the “unsubstituted heterocyclic group” and the “substituted heterocyclic group” mentioned herein are merely exemplary, and the “substituted heterocyclic group” mentioned herein includes a group derived by substituting a hydrogen atom bonded to a carbon atom of a skeleton of a “substituted heterocyclic group” in the specific example group G2B below, and a group derived by substituting a hydrogen atom of a substituent of the “substituted heterocyclic group” in the specific example group G2B below.

The specific example group G2A includes, for instance, unsubstituted heterocyclic groups including a nitrogen atom (specific example group G2A1) below, unsubstituted heterocyclic groups including an oxygen atom (specific example group G2A2) below, unsubstituted heterocyclic groups including a sulfur atom (specific example group G2A3) below, and monovalent heterocyclic groups (specific example group G2A4) derived by removing a hydrogen atom from cyclic structures represented by formulae (TEMP-16) to (TEMP-33) below.

The specific example group G2B includes, for instance, substituted heterocyclic groups including a nitrogen atom (specific example group G2B1) below, substituted heterocyclic groups including an oxygen atom (specific example group G2B2) below, substituted heterocyclic groups including a sulfur atom (specific example group G2B3) below, and groups derived by substituting at least one hydrogen atom of the monovalent heterocyclic groups (specific example group G2B4) derived from the cyclic structures represented by formulae (TEMP-16) to (TEMP-33) below.

Unsubstituted Heterocyclic Groups Including Nitrogen Atom (Specific Example Group G2A1):

pyrrolyl group, imidazolyl group, pyrazolyl group, triazolyl group, tetrazolyl group, oxazolyl group, isoxazolyl group, oxadiazolyl group, thiazolyl group, isothiazolyl group, thiadiazolyl group, a pyridyl group, pyridazynyl group, a pyrimidinyl group, pyrazinyl group, a triazinyl group, indolyl group, isoindolyl group, indolizinyl group, quinolizinyl group, quinolyl group, isoquinolyl group, cinnolyl group, phthalazinyl group, quinazolinyl group, quinoxalinyl group, benzimidazolyl group, indazolyl group, phenanthrolinyl group, phenanthridinyl group, acridinyl group, phenazinyl group, carbazolyl group, benzocarbazolyl group, morpholino group, phenoxazinyl group, phenothiazinyl group, azacarbazolyl group, and diazacarbazolyl group.

Unsubstituted Heterocyclic Groups Including Oxygen Atom (Specific Example Group G2A2):

furyl group, oxazolyl group, isoxazolyl group, oxadiazolyl group, xanthenyl group, benzofuranyl group, isobenzofuranyl group, dibenzofuranyl group, naphthobenzofuranyl group, benzoxazolyl group, benzisoxazolyl group, phenoxazinyl group, morpholino group, dinaphthofuranyl group, azadibenzofuranyl group, diazadibenzofuranyl group, azanaphthobenzofuranyl group, and diazanaphthobenzofuranyl group.

Unsubstituted Heterocyclic Groups Including Sulfur Atom (Specific Example Group G2A3):

thienyl group, thiazolyl group, isothiazolyl group, thiadiazolyl group, benzothiophenyl group (benzothienyl group), isobenzothiophenyl group (isobenzothienyl group), dibenzothiophenyl group (dibenzothienyl group), naphthobenzothiophenyl group (nahthobenzothienyl group), benzothiazolyl group, benzisothiazolyl group, phenothiazinyl group, dinaphthothiophenyl group (dinaphthothienyl group), azadibenzothiophenyl group (azadibenzothienyl group), diazadibenzothiophenyl group (diazadibenzothienyl group), azanaphthobenzothiophenyl group (azanaphthobenzothienyl group), and diazanaphthobenzothiophenyl group (diazanaphthobenzothienyl group).

Monovalent Heterocyclic Groups Derived by Removing a Hydrogen Atom from Cyclic Structures Represented by Formulae (TEMP-16) to (TEMP-33) below (Specific Example Group G2A4):

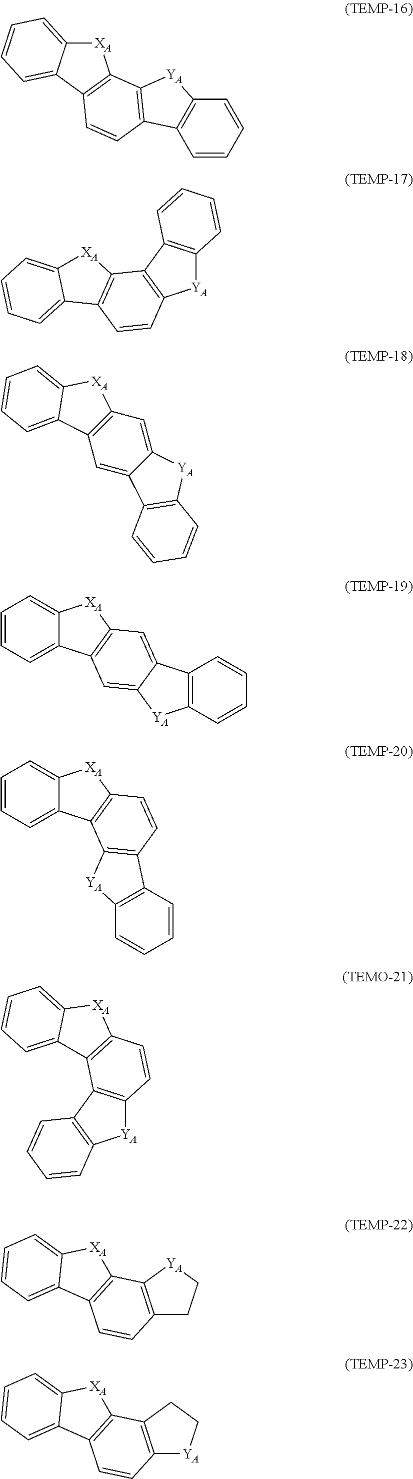

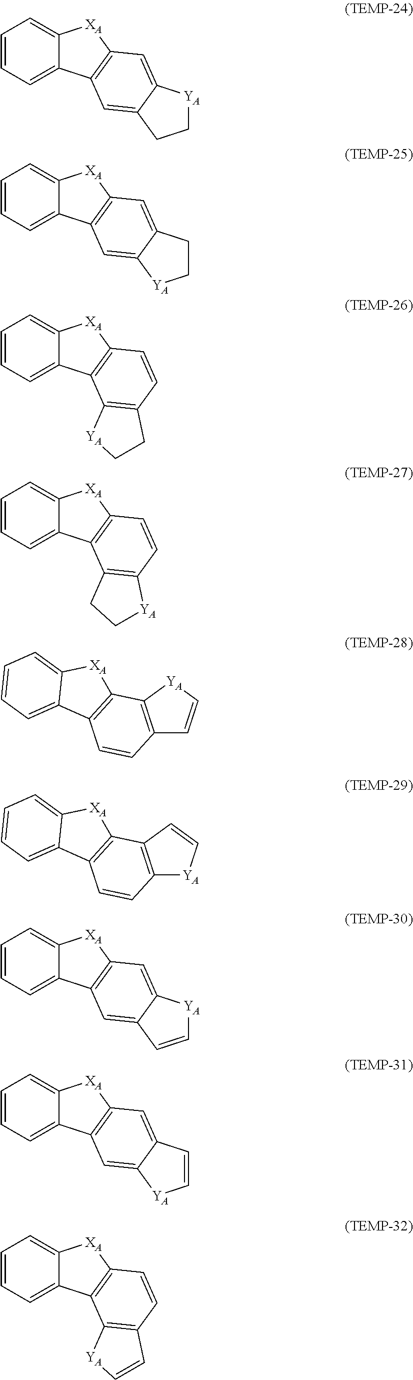

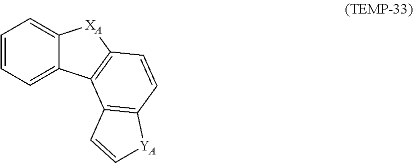

In the formulae (TEMP-16) to (TEMP-33), X_{A} and Y_{A} are each independently an oxygen atom, a sulfur atom, NH or CH₂, with a proviso that at least one of X_{A} and Y_{A} is an oxygen atom, a sulfur atom, or NH.

The monovalent heterocyclic groups (specific example group G2A4) derived from the cyclic structures represented by the formulae (TEMP-16) to (TEMP-33) represent groups derived by removing one hydrogen atom from the cyclic structures represented by the formulae (TEMP-16) to (TEMP-33). When at least one of X_{A} and Y_{A} in the formulae (TEMP-16) to (TEMP-33) is NH or CH₂, the monovalent heterocyclic groups derived from the cyclic structures represented by the formulae (TEMP-16) to (TEMP-33) include a monovalent group derived by removing one hydrogen atom from NH or CH₂.

Substituted Heterocyclic Groups Including Nitrogen Atom (Specific Example Group G2B1):

(9-phenyl)carbazolyl group, (9-biphenylyl)carbazolyl group, (9-phenyl)phenylcarbazolyl group, (9-naphthyl)carbazolyl group, diphenylcarbazole-9-yl group, phenylcarbazole-9-yl group, methylbenzimidazolyl group, ethylbenzimidazolyl group, phenyltriazinyl group, biphenylyltriazinyl group, diphenyltriazinyl group, phenylquinazolinyl group, and biphenylylquinazolinyl group.

Substituted Heterocyclic Groups Including Oxygen Atom (Specific Example Group G2B2):

phenyldibenzofuranyl group, methyldibenzofuranyl group, t-butyldibenzofuranyl group, and monovalent residue of spiro[9H-xanthene-9,9′-[9H]fluorene].

Substituted Heterocyclic Groups Including Sulfur Atom (Specific Example Group G2B3):

phenyldibenzothiophenyl group, methyldibenzothiophenyl group, t-butyldibenzothiophenyl group, and monovalent residue of spiro[9H-thioxanthene-9,9′-[9H]fluorene].

Groups Derived by Substituting at Least One Hydrogen Atom of Monovalent Heterocyclic Group Derived from Cyclic Structures Represented by Formulae (TEMP-16) to (TEMP-33) with Substituent (Specific Example Group G2B4):

The groups derived by substituting at least one hydrogen atom of the monovalent heterocyclic group derived from the cyclic structures represented by the formulae (TEMP-16) to (TEMP-33) are groups derived by substituting at least one hydrogen atom bonded to a carbon atom of a skeleton of the cyclic structures represented by the formulae (TEMP-16) to (TEMP-33), or a group derived by substituting at least one hydrogen atom of at least one of X_{A} or Y_{A} in a form of NH or CH₂ with a substituent.

The “at least one hydrogen atom of a monovalent heterocyclic group” means at least one hydrogen atom selected from a hydrogen atom bonded to a ring carbon atom of the monovalent heterocyclic group, a hydrogen atom bonded to a nitrogen atom of at least one of X_{A} or Y_{A} in a form of NH, and a hydrogen atom of one of X_{A} and Y_{A} in a form of a methylene group (CH₂).

### Substituted or Unsubstituted Alkyl Group

Specific examples (specific example group G3) of the “substituted or unsubstituted alkyl group” mentioned herein include unsubstituted alkyl groups (specific example group G3A) and substituted alkyl groups (specific example group G3B below) (herein, an unsubstituted alkyl group refers to an “unsubstituted alkyl group” in a “substituted or unsubstituted alkyl group,” and a substituted alkyl group refers to a “substituted alkyl group” in a “substituted or unsubstituted alkyl group.”) A simply termed “alkyl group” herein includes both of “unsubstituted alkyl group” and “substituted alkyl group.”

The “substituted alkyl group” refers to a group derived by substituting at least one hydrogen atom in an “unsubstituted alkyl group” with a substituent. Specific examples of the “substituted alkyl group” include a group derived by substituting at least one hydrogen atom of an “unsubstituted alkyl group” (specific example group G3A) below with a substituent, and examples of the substituted alkyl group (specific example group G3B) below. Herein, the alkyl group for the “unsubstituted alkyl group” refers to a chain alkyl group. Accordingly, the “unsubstituted alkyl group” include linear “unsubstituted alkyl group” and branched “unsubstituted alkyl group.” It should be noted that the examples of the “unsubstituted alkyl group” and the “substituted alkyl group” mentioned herein are merely exemplary, and the “substituted alkyl group” mentioned herein includes a group derived by substituting a hydrogen atom bonded to a carbon atom of a skeleton of the “substituted alkyl group” in the specific example group G3B, and a group derived by substituting a hydrogen atom of a substituent of the “substituted alkyl group” in the specific example group G3B.

Unsubstituted Alkyl Group (Specific Example Group G3A):

methyl group, ethyl group, n-propyl group, isopropyl group, n-butyl group, isobutyl group, s-butyl group, and t-butyl group.

Substituted Alkyl Group (Specific Example Group G3B):

heptafluoropropyl group (including isomer thereof), pentafluoroethyl group, 2,2,2-trifluoroethyl group, and trifluoromethyl group.

### Substituted or Unsubstituted Alkenyl Group

Specific examples (specific example group G4) of the “substituted or unsubstituted alkenyl group” mentioned herein include unsubstituted alkenyl groups (specific example group G4A) and substituted alkenyl groups (specific example group G4B) (herein, an unsubstituted alkenyl group refers to an “unsubstituted alkenyl group” in a “substituted or unsubstituted alkenyl group,” and a substituted alkenyl group refers to a “substituted alkenyl group” in a “substituted or unsubstituted alkenyl group.”) A simply termed “alkenyl group” herein includes both of “unsubstituted alkenyl group” and “substituted alkenyl group.”

The “substituted alkenyl group” refers to a group derived by substituting at least one hydrogen atom in an “unsubstituted alkenyl group” with a substituent. Specific examples of the “substituted alkenyl group” include an “unsubstituted alkenyl group” (specific example group G4A) substituted by a substituent, and examples of the substituted alkenyl group (specific example group G4B) below. It should be noted that the examples of the “unsubstituted alkenyl group” and the “substituted alkenyl group” mentioned herein are merely exemplary, and the “substituted alkenyl group” mentioned herein includes a group derived by substituting a hydrogen atom of a skeleton of the “substituted alkenyl group” in the specific example group G4B with a substituent, and a group derived by substituting a hydrogen atom of a substituent of the “substituted alkenyl group” in the specific example group G4B with a substituent.

Unsubstituted Alkenyl Group (Specific Example Group G4A):

vinyl group, allyl group, 1-butenyl group, 2-butenyl group, and 3-butenyl group.

Substituted Alkenyl Group (Specific Example Group G4B):

1,3-butanedienyl group, 1-methylvinyl group, 1-methylallyl group, 1,1-dimethylallyl group, 2-methylallyl group, and 1,2-dimethylallyl group.

### Substituted or Unsubstituted Alkynyl Group

Specific examples (specific example group G5) of the “substituted or unsubstituted alkynyl group” mentioned herein include unsubstituted alkynyl groups (specific example group G5A) below (herein, an unsubstituted alkynyl group refers to an “unsubstituted alkynyl group” in the “substituted or unsubstituted alkynyl group.”) A simply termed “alkynyl group” herein includes both of “unsubstituted alkynyl group” and “substituted alkynyl group.”

The “substituted alkynyl group” refers to a group derived by substituting at least one hydrogen atom in an “unsubstituted alkynyl group” with a substituent. Specific examples of the “substituted alkynyl group” include a group derived by substituting at least one hydrogen atom of the “unsubstituted alkynyl group” (specific example group G5A) below with a substituent.

Unsubstituted Alkynyl Group (Specific Example Group G5A):

ethynyl group

### Substituted or Unsubstituted Cycloalkyl Group

Specific examples (specific example group G6) of the “substituted or unsubstituted cycloalkyl group” mentioned herein include unsubstituted cycloalkyl groups (specific example group G6A) and substituted cycloalkyl groups (specific example group G6B) (herein, an unsubstituted cycloalkyl group refers to an “unsubstituted cycloalkyl group” in the “substituted or unsubstituted cycloalkyl group,” and a substituted cycloalkyl group refers to the “substituted cycloalkyl group” in a “substituted or unsubstituted cycloalkyl group.”) A simply termed “cycloalkyl group” herein includes both of “unsubstituted cycloalkyl group” and “substituted cycloalkyl group.”

The “substituted cycloalkyl group” refers to a group derived by substituting at least one hydrogen atom of an “unsubstituted cycloalkyl group” with a substituent. Specific examples of the “substituted cycloalkyl group” include a group derived by substituting at least one hydrogen atom of the “unsubstituted cycloalkyl group” (specific example group G6A) below with a substituent, and examples of the substituted cycloalkyl group (specific example group G6B) below. It should be noted that the examples of the “unsubstituted cycloalkyl group” and the “substituted cycloalkyl group” mentioned herein are merely exemplary, and the “substituted cycloalkyl group” mentioned herein includes a group derived by substituting at least one hydrogen atom bonded to a carbon atom of a skeleton of the “substituted cycloalkyl group” in the specific example group G6B with a substituent, and a group derived by substituting a hydrogen atom of a substituent of the “substituted cycloalkyl group” in the specific example group G6B with a substituent.

Unsubstituted Cycloalkyl Group (Specific Example Group G6A):

cyclopropyl group, cyclobutyl group, cyclopentyl group, cyclohexyl group, 1-adamantyl group, 2-adamantyl group, 1-norbornyl group, and 2-norbornyl group.

Substituted Cycloalkyl Group (Specific Example Group G6B): 4-methylcyclohexyl group.

Group Represented by “—Si(R₉₀₁)(R₉₀₂)(R₉₀₃)”

Specific examples (specific example group G7) of the group represented herein by —Si(R₉₀₁)(R₉₀₂)(R₉₀₃) include:

### —Si(G1)(G1)(G1);

### —Si(G1)(G2)(G2);

### —Si(G1)(G1)(G2);

### —Si(G2)(G2)(G2);

### —Si(G3)(G3)(G3); and

### —Si(G6)(G6)(G6)

where:

G1 represents a “substituted or unsubstituted aryl group” in the specific example group G1;

G2 represents a “substituted or unsubstituted heterocyclic group” in the specific example group G2;

G3 represents a “substituted or unsubstituted alkyl group” in the specific example group G3;

G6 represents a “substituted or unsubstituted cycloalkyl group” in the specific example group G6;

the plurality of G1 in —Si(G1)(G1)(G1) are mutually the same or different;

the plurality of G2 in —Si(G1)(G2)(G2) are mutually the same or different;

the plurality of G1 in —Si(G1)(G1)(G2) are mutually the same or different;

the plurality of G2 in —Si(G2)(G2)(G2) are mutually the same or different; The plurality of G3 in —Si(G3)(G3)(G3) are mutually the same or different; and

the plurality of G6 in —Si(G6)(G6)(G6) are mutually the same or different.

### Group Represented by “—O—(R₉₀₄)”

Specific examples (specific example group G8) of a group represented by —O—(R₉₀₄) herein include

### —O(G1);

### —O(G2);

### —O(G3); and

### —O(G6)

where:

G1 represents a “substituted or unsubstituted aryl group” in the specific example group G1;

G2 represents a “substituted or unsubstituted heterocyclic group” in the specific example group G2;

G3 represents a “substituted or unsubstituted alkyl group” in the specific example group G3; and

G6 represents a “substituted or unsubstituted cycloalkyl group” in the specific example group G6.

### Group Represented by “—S—(R₉₀₅)”

Specific examples (specific example group G9) of a group represented herein by —S—(R₉₀₅) include:

### —S(G1);

### —S(G2);

### —S(G3); and

### —S(G6)

where:

G1 represents a “substituted or unsubstituted aryl group” in the specific example group G1;

G2 represents a “substituted or unsubstituted heterocyclic group” in the specific example group G2;

G3 represents a “substituted or unsubstituted alkyl group” in the specific example group G3; and

G6 represents a “substituted or unsubstituted cycloalkyl group” in the specific example group G6.

Group Represented by “—N(R₉₀₆)(R₉₀₇)”

Specific examples (specific example group G10) of a group represented herein by —N(R₉₀₆)(R₉₀₇) include:

### —N(G1)(G1);

### —N(G2)(G2);

### —N(G1)(G2);

### —N(G3)(G3); and

### —N(G6)(G6)

where:

G1 represents a “substituted or unsubstituted aryl group” in the specific example group G1;

G2 represents a “substituted or unsubstituted heterocyclic group” in the specific example group G2;

G3 represents a “substituted or unsubstituted alkyl group” in the specific example group G3;

G6 represents a “substituted or unsubstituted cycloalkyl group” in the specific example group G6;

the plurality of G1 in —N(G1)(G1) are mutually the same or different;

the plurality of G2 in —N(G2)(G2) are mutually the same or different;

the plurality of G3 in —N(G3)(G3) are mutually the same or different; and

the plurality of G6 in —N(G6)(G6) are mutually the same or different.

### Halogen Atom

Specific examples (specific example group G11) of “halogen atom” mentioned herein include a fluorine atom, chlorine atom, bromine atom, and iodine atom.

### Substituted or Unsubstituted Fluoroalkyl Group

The “substituted or unsubstituted fluoroalkyl group” mentioned herein refers to a group derived by substituting at least one hydrogen atom of the “substituted or unsubstituted alkyl group” with a fluorine atom. More specifically, it refers to a group derived by substituting at least one hydrogen atom bonded to a carbon atom of an alkyl group in the “substituted or unsubstituted alkyl group” with a fluorine atom. The “substituted or unsubstituted fluoroalkyl group” mentioned herein also includes a group (perfluoro group) derived by substituting all of hydrogen atoms bonded to carbon atom(s) of an alkyl group(s) in the “substituted or unsubstituted alkyl group” with fluorine atoms. An “unsubstituted fluoroalkyl group” has, unless otherwise specified herein, 1 to 50, preferably 1 to 30, more preferably 1 to 18 carbon atoms. The “substituted fluoroalkyl group” refers to a group derived by substituting at least one hydrogen atom in a “fluoroalkyl group” with a substituent. It should be noted that the examples of the “substituted fluoroalkyl group” mentioned herein includes a group derived by substituting at least one hydrogen atom bonded to a carbon atom of an alkyl chain of a “substituted fluoroalkyl group” with a substituent, and a group derived by substituting at least one hydrogen atom of a substituent of the “substituted fluoroalkyl group” with a substituent. Specific examples of the “substituted fluoroalkyl group” include a group derived by substituting at least one hydrogen atom of the “alkyl group” (specific example group G3) with a fluorine atom.

### Substituted or Unsubstituted Haloalkyl Group

The “substituted or unsubstituted haloalkyl group” mentioned herein refers to a group derived by substituting at least one hydrogen atom of the “substituted or unsubstituted alkyl group” with a halogen atom, and also includes a group derived by substituting all of the hydrogen atoms bonded to a carbon atom(s) of the alkyl group in the “substituted or unsubstituted alkyl group” with halogen atoms. An “unsubstituted haloalkyl group” has, unless otherwise specified herein, 1 to 50, preferably 1 to 30, more preferably 1 to 18 carbon atoms. The “substituted haloalkyl group” refers to a group derived by substituting at least one hydrogen atom in a “haloalkyl group” with a substituent. It should be noted that the examples of the “substituted haloalkyl group” mentioned herein includes a group derived by substituting at least one hydrogen atom bonded to a carbon atom of an alkyl chain of a “substituted haloalkyl group” with a substituent, and a group derived by substituting at least one hydrogen atom of a substituent of the “substituted haloalkyl group” with a substituent. Specific examples of the “substituted haloalkyl group” include a group derived by substituting at least one hydrogen atom of the “alkyl group” (specific example group G3) with a halogen atom. The haloalkyl group is sometimes referred to as a halogenated alkyl group.

### Substituted or Unsubstituted Alkoxy Group

Specific examples of a “substituted or unsubstituted alkoxy group” mentioned herein include a group represented by —O(G3), G3 being the “substituted or unsubstituted alkyl group” in the specific example group G3. An “unsubstituted alkoxy group” has, unless otherwise specified herein, 1 to 50, preferably 1 to 30, more preferably 1 to 18 carbon atoms.

### Substituted or Unsubstituted Alkylthio Group

Specific examples of a “substituted or unsubstituted alkylthio group” mentioned herein include a group represented by —S(G3), G3 being the “substituted or unsubstituted alkyl group” in the specific example group G3. An “unsubstituted alkylthio group” has, unless otherwise specified herein, 1 to 50, preferably 1 to 30, more preferably 1 to 18 carbon atoms.

### Substituted or Unsubstituted Aryloxy Group

Specific examples of a “substituted or unsubstituted aryloxy group” mentioned herein include a group represented by —O(G1), G1 being the “substituted or unsubstituted aryl group” in the specific example group G1. An “unsubstituted aryloxy group” has, unless otherwise specified herein, 6 to 50, preferably 6 to 30, more preferably 6 to 18 ring carbon atoms.

### Substituted or Unsubstituted Arylthio Group

Specific examples of a “substituted or unsubstituted arylthio group” mentioned herein include a group represented by —S(G1), G1 being the “substituted or unsubstituted aryl group” in the specific example group G1. An “unsubstituted arylthio group” has, unless otherwise specified herein, 6 to 50, preferably 6 to 30, more preferably 6 to 18 ring carbon atoms.

### Substituted or Unsubstituted Trialkylsilyl Group

Specific examples of a “substituted or unsubstituted trialkylsilyl group” mentioned herein include a group represented by —Si(G3)(G3)(G3), G3 being the “substituted or unsubstituted alkyl group” in the specific example group G3. The plurality of G3 in —Si(G3)(G3)(G3) are mutually the same or different. Each of the alkyl groups in the “trialkylsilyl group” has, unless otherwise specified herein, 1 to 50, preferably 1 to 20, more preferably 1 to 6 carbon atoms.

### Substituted or Unsubstituted Aralkyl Group

Specific examples of a “substituted or unsubstituted aralkyl group” mentioned herein include a group represented by (G3)-(G1), G3 being the “substituted or unsubstituted alkyl group” in the specific example group G3, G1 being the “substituted or unsubstituted aryl group” in the specific example group G1. Accordingly, the “aralkyl group” is a group derived by substituting a hydrogen atom of the “alkyl group” with a substituent in a form of the “aryl group,” which is an example of the “substituted alkyl group.” An “unsubstituted aralkyl group,” which is an “unsubstituted alkyl group” substituted by an “unsubstitued aryl group,” has, unless otherwise specified herein, 7 to 50 carbon atoms, preferably 7 to 30 carbon atoms, more preferably 7 to 18 carbon atoms.

Specific examples of the “substituted or unsubstituted aralkyl group” include a benzyl group, 1-phenylethyl group, 2-phenylethyl group, 1-phenylisopropyl group, 2-phenylisopropyl group, phenyl-t-butyl group, α-naphthylmethyl group, 1-α-naphthylethyl group, 2-α-naphthylethyl group, 1-α-naphthylisopropyl group, 2-α-naphthylisopropyl group, β-naphthylmethyl group, 1-β-naphthylethyl group, 2-β-naphthylethyl group, 1-β-naphthylisopropyl group, and 2-β-naphthylisopropyl group.

Preferable examples of the substituted or unsubstituted aryl group mentioned herein include, unless otherwise specified herein, a phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, phenanthryl group, pyrenyl group, chrysenyl group, triphenylenyl group, fluorenyl group, 9,9′-spirobifluorenyl group, 9,9-dimethylfluorenyl group, and 9,9-diphenylfluorenyl group.

Preferable examples of the substituted or unsubstituted heterocyclic group mentioned herein include, unless otherwise specified herein, a pyridyl group, pyrimidinyl group, triazinyl group, quinolyl group, isoquinolyl group, quinazolinyl group, benzimidazolyl group, phenanthrolinyl group, carbazolyl group (1-carbazolyl group, 2-carbazolyl group, 3-carbazolyl group, 4-carbazolyl group, or 9-carbazolyl group), benzocarbazolyl group, azacarbazolyl group, diazacarbazolyl group, dibenzofuranyl group, naphthobenzofuranyl group, azadibenzofuranyl group, diazadibenzofuranyl group, dibenzothiophenyl group, naphthobenzothiophenyl group, azadibenzothiophenyl group, diazadibenzothiophenyl group, (9-phenyl)carbazolyl group ((9-phenyl)carbazole-1-yl group, (9-phenyl)carbazole-2-yl group, (9-phenyl)carbazole-3-yl group, or (9-phenyl)carbazole-4-yl group), (9-biphenylyl)carbazolyl group, (9-phenyl)phenylcarbazolyl group, diphenylcarbazole-9-yl group, phenylcarbazole-9-yl group, phenyltriazinyl group, biphenylyltriazinyl group, diphenyltriazinyl group, phenyldibenzofuranyl group, and phenyldibenzothiophenyl group.

The carbazolyl group mentioned herein is, unless otherwise specified herein, specifically a group represented by one of formulae below.

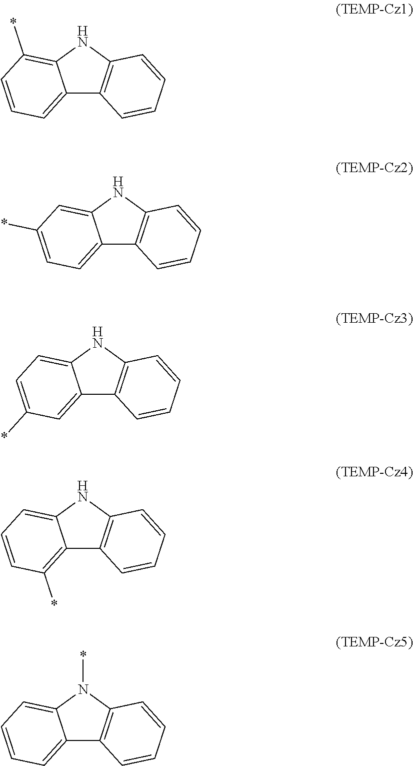 [SMILES: *c1cccc2c1[nH]c1ccccc12 ; *c1ccc2c(c1)[nH]c1ccccc12 ; *c1ccc2[nH]c3ccccc3c2c1 ; *c1cccc2[nH]c3ccccc3c12 ; *n1c2ccccc2c2ccccc21]

The (9-phenyl)carbazolyl group mentioned herein is, unless otherwise specified herein, specifically a group represented by one of formulae below.

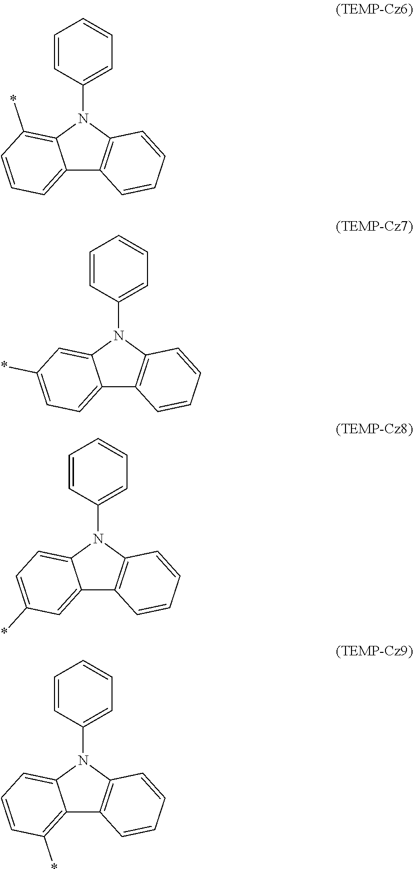

In the formulae (TEMP-Cz1) to (TEMP-Cz9), * represents a bonding position.

The dibenzofuranyl group and dibenzothiophenyl group mentioned herein are, unless otherwise specified herein, each specifically represented by one of formulae below.

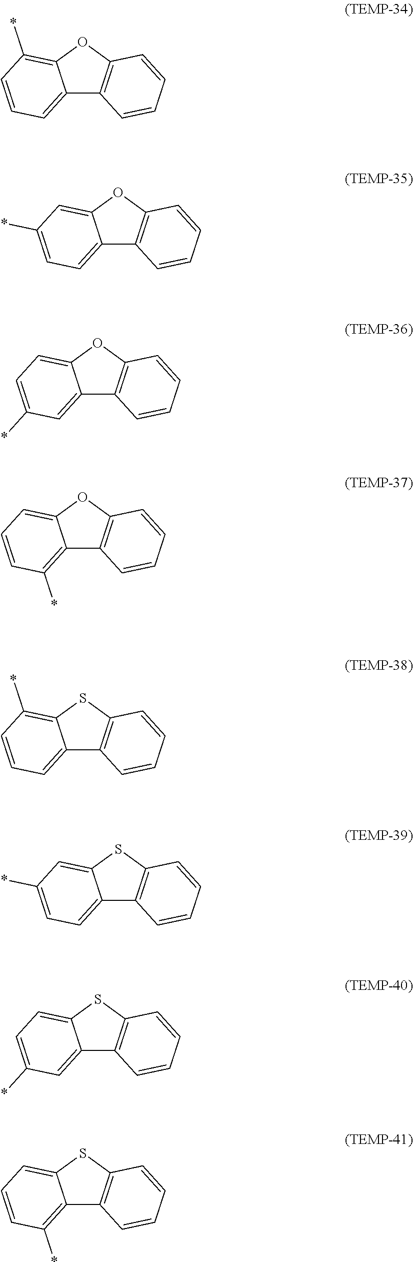

In the formulae (TEMP-34) to (TEMP-41), * represents a bonding position.

Preferable examples of the substituted or unsubstituted alkyl group mentioned herein include, unless otherwise specified herein, a methyl group, ethyl group, propyl group, isopropyl group, n-butyl group, isobutyl group, and t-butyl group.

### Substituted or Unsubstituted Arylene Group

The “substituted or unsubstituted arylene group” mentioned herein is, unless otherwise specified herein, a divalent group derived from the “substituted or unsubstituted aryl group,” and more specifically is a divalent group derived by removing one hydrogen atom on an aryl ring of the “substituted or unsubstituted aryl group.” Specific examples of the “substituted or unsubstituted arylene group” (specific example group G12) include a divalent group derived from the “substituted or unsubstituted aryl group” in the specific example group G1, and more specifically include a divalent group derived by removing one hydrogen atom on an aryl ring of the “substituted or unsubstituted aryl group” in the specific example group G1.

### Substituted or Unsubstituted Divalent Heterocyclic Group

The “substituted or unsubstituted divalent heterocyclic group” mentioned herein is, unless otherwise specified herein, a divalent group derived from the “substituted or unsubstituted heterocyclic group,” and more specifically is a divalent group derived by removing one hydrogen atom on a heterocyclic ring of the “substituted or unsubstituted heterocyclic group.” Specific examples of the “substituted or unsubstituted divalent heterocyclic group” (specific example group G13) include a divalent group derived from the “substituted or unsubstituted heterocyclic group” in the specific example group G2, and more specifically include a divalent group derived by removing one hydrogen atom on a heterocyclic ring of the “substituted or unsubstituted heterocyclic group” in the specific example group G2.

### Substituted or Unsubstituted Alkylene Group

The “substituted or unsubstituted alkylene group” mentioned herein is, unless otherwise specified herein, a divalent group derived from the “substituted or unsubstituted alkyl group,” and more specifically is a divalent group derived by removing one hydrogen atom on an alkyl chain of the “substituted or unsubstituted alkyl group.” Specific examples of the “substituted or unsubstituted alkylene group” (specific example group G14) include a divalent group derived from the “substituted or unsubstituted alkyl group” in the specific example group G3, and more specifically include a divalent group derived by removing one hydrogen atom on an alkyl chain of the “substituted or unsubstituted alkyl group” in the specific example group G3.

The substituted or unsubstituted arylene group mentioned herein is, unless otherwise specified herein, preferably any one of groups represented by formulae (TEMP-42) to (TEMP-68) below.

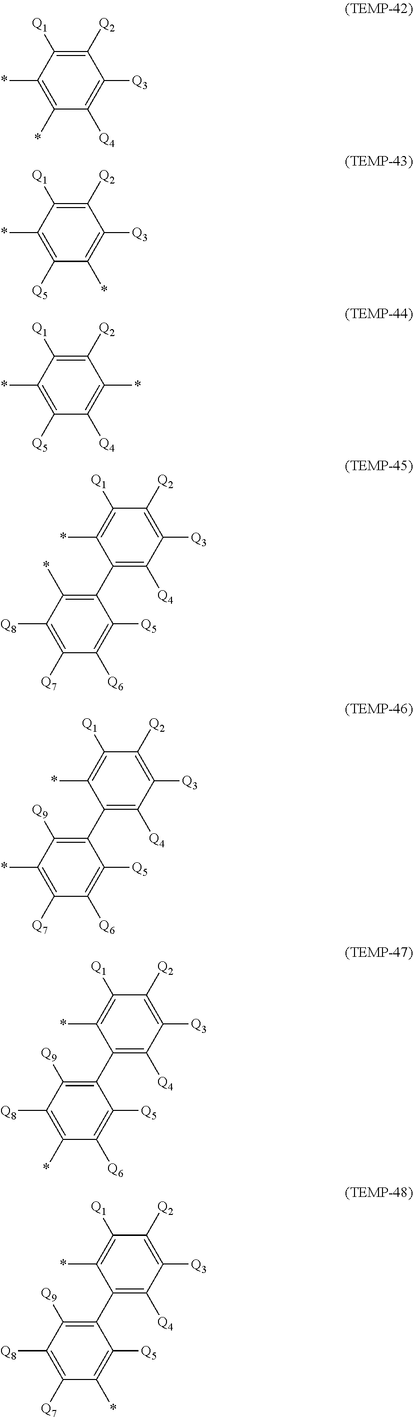

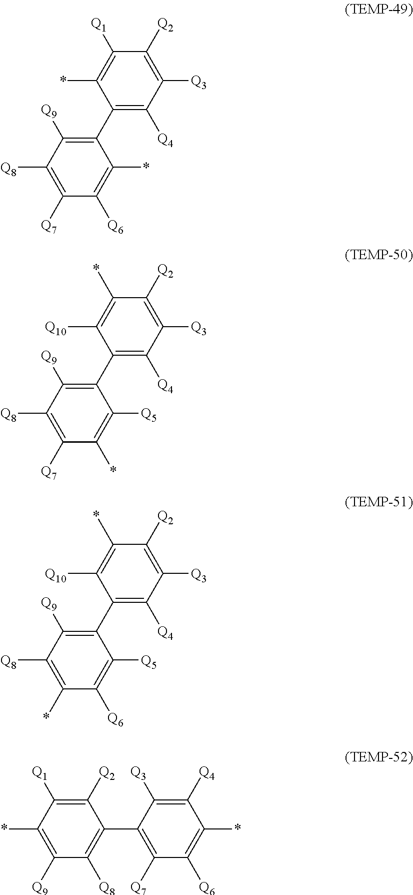

In the formulae (TEMP-42) to (TEMP-52), Q₁ to Q₁₀ each independently represent a hydrogen atom or a substituent.

In the formulae (TEMP-42) to (TEMP-52), * represents a bonding position.

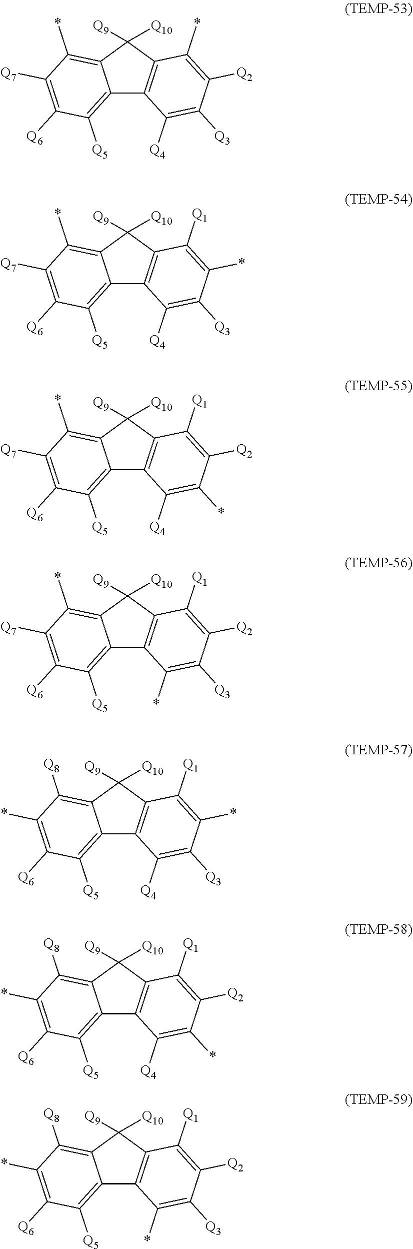

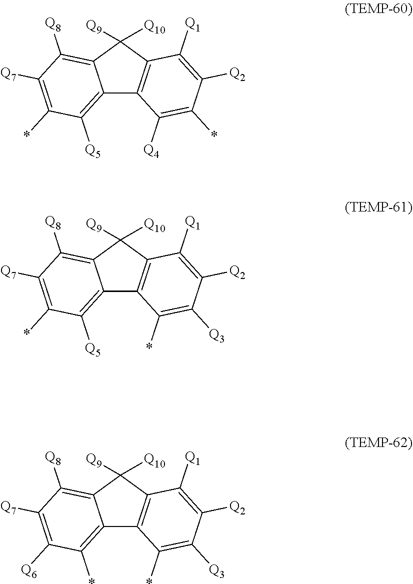

In the formulae (TEMP-53) to (TEMP-62), Q₁ to Q₁₀ each independently represent a hydrogen atom or a substituent.

In the formulae, Q₉ and Q₁₀ may be mutually bonded through a single bond to form a ring.

In the formulae (TEMP-53) to (TEMP-62), * represents a bonding position.

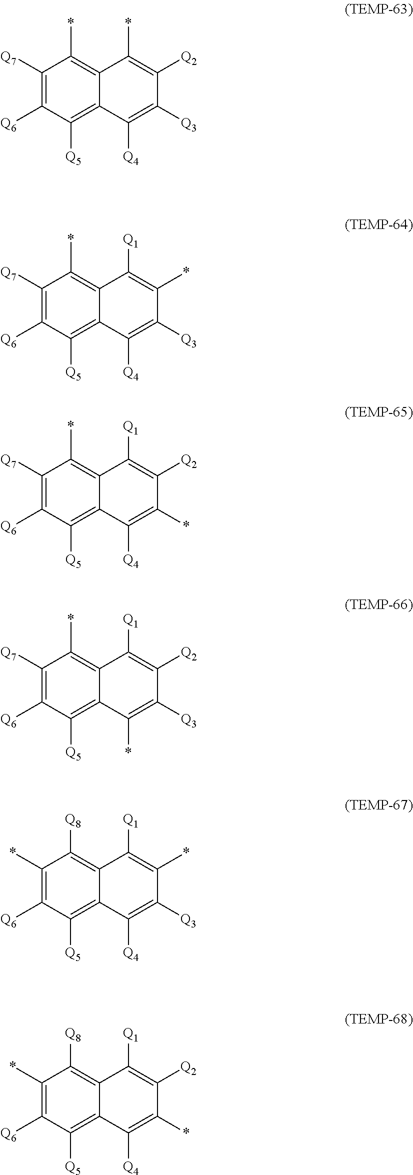

In the formulae (TEMP-63) to (TEMP-68), Q₁ to Q₈ each independently represent a hydrogen atom or a substituent.

In the formulae (TEMP-63) to (TEMP-68), * represents a bonding position.

The substituted or unsubstituted divalent heterocyclic group mentioned herein is, unless otherwise specified herein, preferably a group represented by any one of formulae (TEMP-69) to (TEMP-102) below.

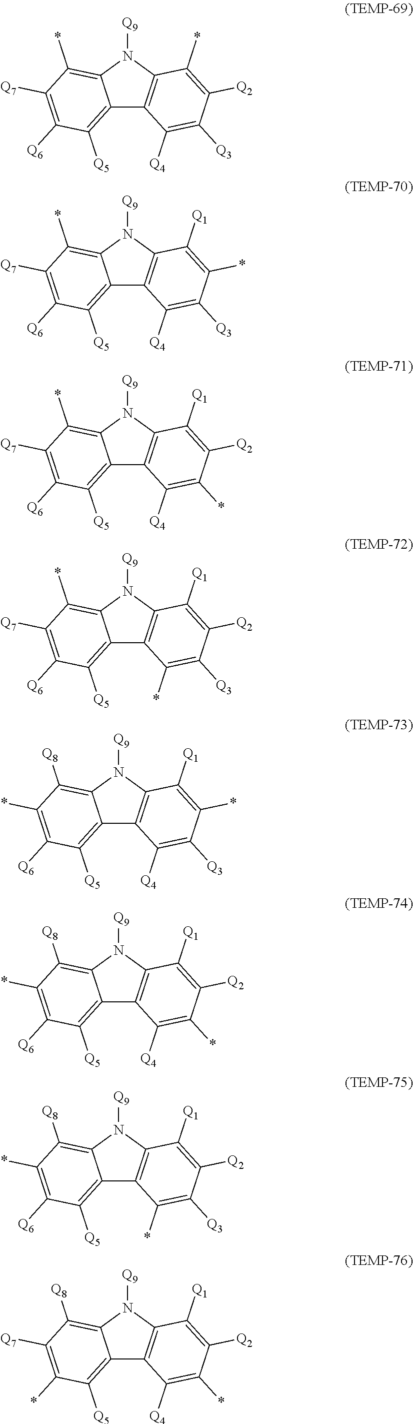

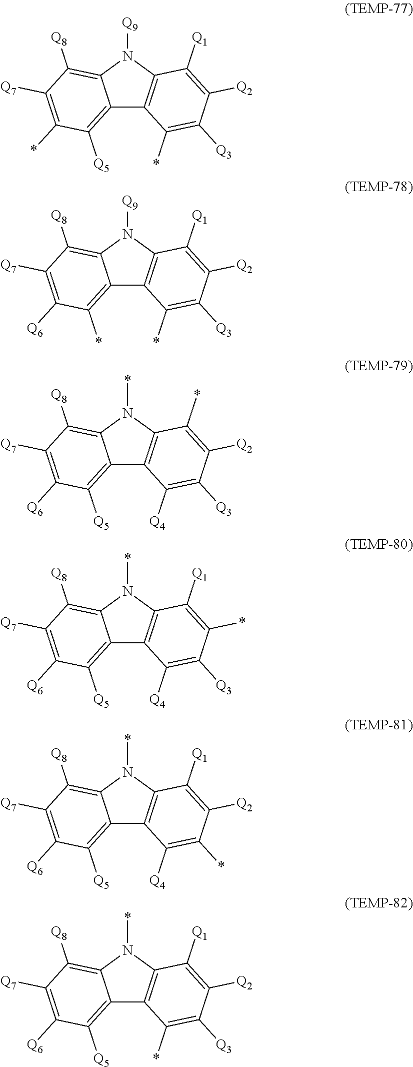

In the formulae (TEMP-69) to (TEMP-82), Q₁ to Q₉ each independently represent a hydrogen atom or a substituent.

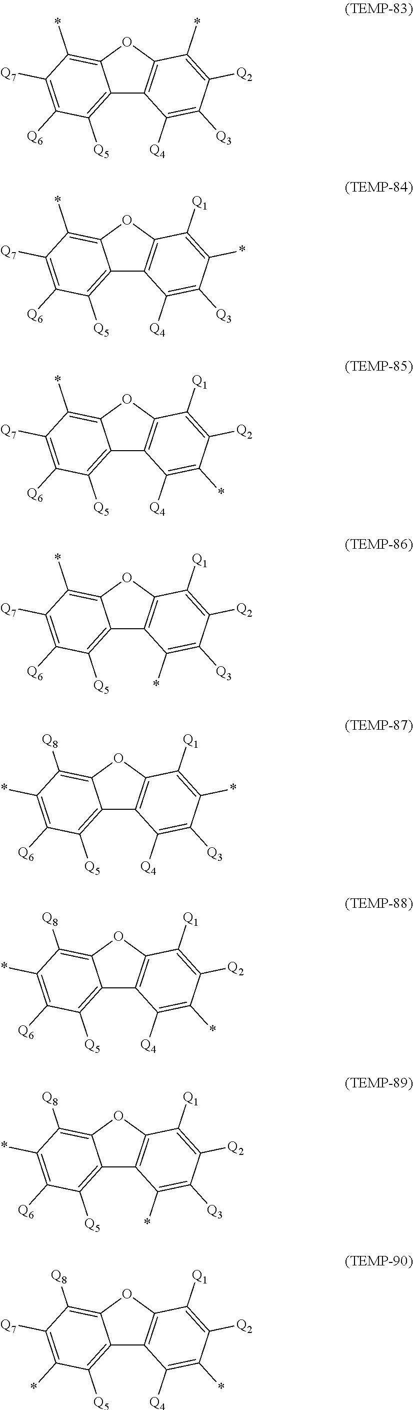

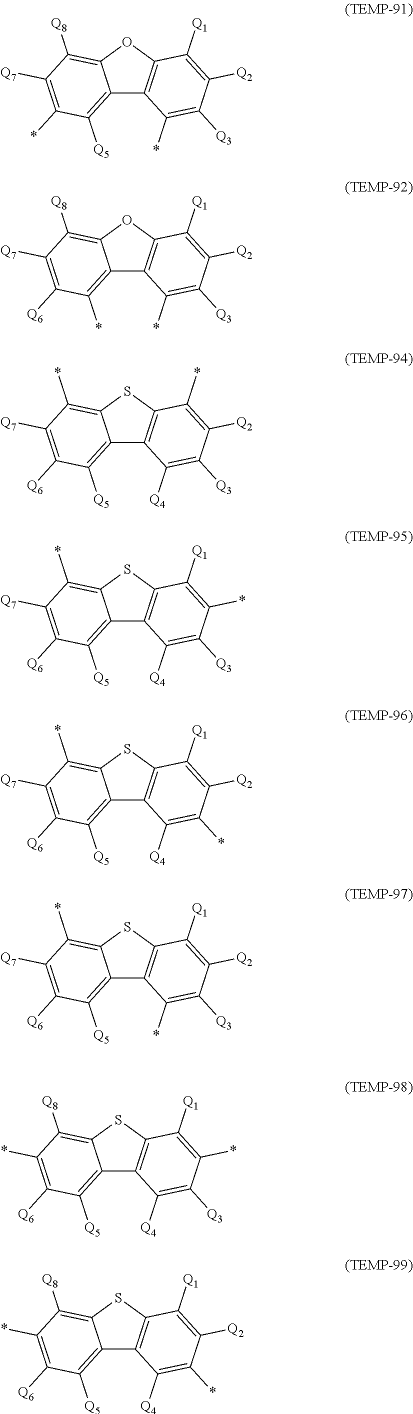

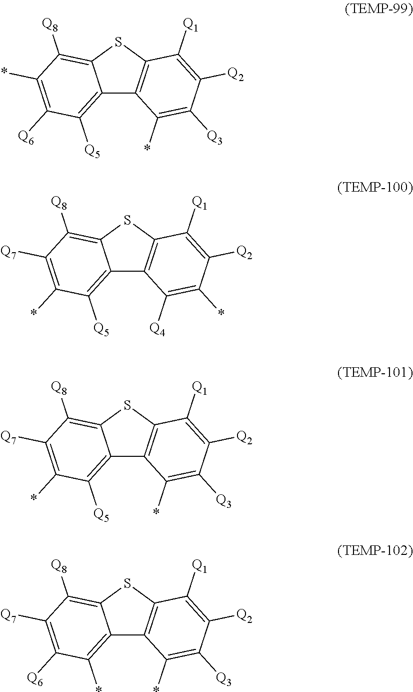

In the formulae (TEMP-83) to (TEMP-102), Q₁ to Q₈ each independently represent a hydrogen atom or a substituent.

The substituent mentioned herein has been described above.

### Instance “Bonded to Form a Ring”

Instances where “at least one combination of adjacent two or more (of . . . ) are mutually bonded to form a substituted or unsubstituted saturated ring” and “at least one combination of adjacent two or more (of . . . ) are mutually bonded to form a substituted or unsubstituted unsaturated ring” mentioned herein (these instances will be sometimes collectively referred to as an instance “bonded to form a ring” hereinafter) will be described below. An anthracene compound having a basic skeleton in a form of an anthracene ring and represented by a formula (TEMP-103) below will be used as an example for the description.

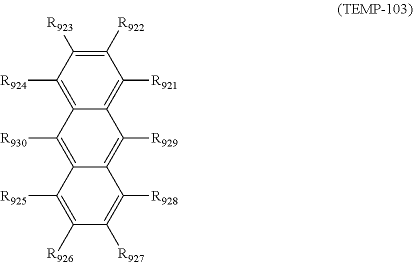

For instance, when “at least one combination of adjacent two or more of” R₉₂₁ to R₉₃₀ “are mutually bonded to form a ring,” the pair of adjacent ones of R₉₂₁ to R₉₃₀ (i.e. the combination at issue) is a pair of R₉₂₁ and a pair of R₉₂₂, R₉₂₂ and R₉₂₃, a pair of R₉₂₃ and R₉₂₄, a pair of R₉₂₄ and R₉₃₀, a pair of R₉₃₀ and R₉₂₅, a pair of R₉₂₅ and R₉₂₆, a pair of R₉₂₆ and R₉₂₇, a pair of R₉₂₇ and R₉₂₈, a pair of R₉₂₈ and R₉₂₉, or a pair of R₉₂₉ and R₉₂₁.

The term “at least one combination” means that two or more of the above combinations of adjacent two or more of R₉₂₁ to R₉₃₀ may simultaneously form rings. For instance, when R₉₂₁ and R₉₂₂ are mutually bonded to form a ring Q_{A} and R₉₂₅ and R₉₂₆ are simultaneously mutually bonded to form a ring Q_{B}, the anthracene compound represented by the formula (TEMP-103) is represented by a formula (TEMP-104) below.

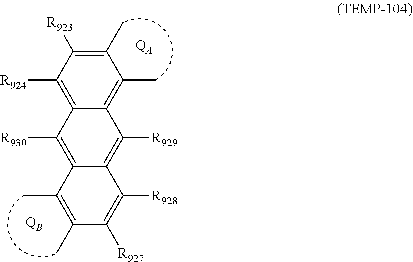

The instance where the “combination of adjacent two or more” form a ring means not only an instance where the “two” adjacent components are bonded but also an instance where adjacent “three or more” are bonded. For instance, R₉₂₁ and R₉₂₂ are mutually bonded to form a ring Q_{A} and R₉₂₂, R₉₂₃ are mutually bonded to form a ring Q_{C}, and mutually adjacent three components (R₉₂₁, R₉₂₂ and R₉₂₃) are mutually bonded to form a ring fused to the anthracene basic skeleton. In this case, the anthracene compound represented by the formula (TEMP-103) is represented by a formula (TEMP-105) below. In the formula (TEMP-105) below, the ring Q_{A} and the ring Q_{C} share R₉₂₂.

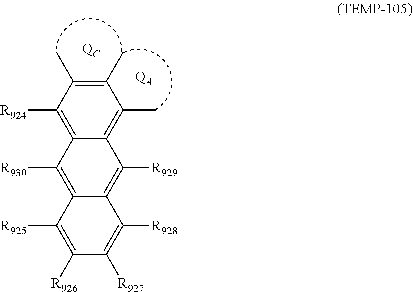

The formed “saturated ring” or “unsaturated ring” may be, in terms of the formed ring in itself, a monocyclic ring or a fused ring. When the “combination of adjacent two” form a “saturated ring” or an “unsaturated ring,” the “saturated ring” or “unsaturated ring” may be a monocyclic ring or a fused ring. For instance, the ring Q_{A}, the ring Q_{B}, and the ring Q_{C} formed in the formulae (TEMP-104) and (TEMP-105) are each independently a “saturated ring” or an “unsaturated ring.” The ring Q_{A} and the ring Q_{C} in the formula (TEMP-105) are fused to form a fused ring. The ring Q_{A} in the formula (TEMP-104), which is not fused with the ring Q_{B}, may be a monocyclic ring or a fused ring. When the ring Q_{A} in the formula (TEMP-104) is a benzene ring, the ring Q_{A} is a monocyclic ring. When the ring Q_{A} in the formula (TEMP-104) is a naphthalene ring, the ring Q_{A} is a fused ring.

The “unsaturated ring” represents an aromatic hydrocarbon ring or an aromatic heterocycle. The “saturated ring” represents an aliphatic hydrocarbon ring or a non-aromatic heterocycle.

Specific examples of the aromatic hydrocarbon ring include a ring formed by bonding a hydrogen atom to an end of a bond of a group in the specific example of the specific example group G1.

Specific examples of the aromatic heterocycle include a ring formed by bonding a hydrogen atom to an end of a bond of an aromatic heterocyclic group in the specific example of the specific example group G2.

Specific examples of the aliphatic hydrocarbon ring include a ring formed by bonding a hydrogen atom to an end of a bond of a group in the specific example of the specific example group G6.

The phrase “to form a ring” herein means that a ring is formed only by a plurality of atoms of a basic skeleton, or by a combination of a plurality of atoms of the basic skeleton and one or more optional atoms. For instance, the ring Q_{A} formed by mutually bonding R₉₂₁ and R₉₂₂ shown in the formula (TEMP-104) is a ring formed by a carbon atom of the anthracene skeleton bonded with R₉₂₁, a carbon atom of the anthracene skeleton bonded with R₉₂₂, and one or more optional atoms. Specifically, when the ring Q_{A} is an unsaturated ring formed by R₉₂₁ and R₉₂₂, the ring formed by a carbon atom of the anthracene skeleton bonded with R₉₂₁, a carbon atom of the anthracene skeleton bonded with R₉₂₂, and four carbon atoms is a benzene ring. Alternatively, when the ring Q_{A} is a saturated ring formed by R₉₂₁ and R₉₂₂, the ring formed by a carbon atom of the anthracene skeleton bonded with R₉₂₁, a carbon atom of the anthracene skeleton bonded with R₉₂₂, and four carbon atoms is a cyclohexane ring.

The “optional atom” is, unless otherwise specified herein, preferably at least one atom selected from the group consisting of a carbon atom, nitrogen atom, oxygen atom, and sulfur atom. A bond of the optional atom (e.g. a carbon atom and a nitrogen atom) not forming a ring may be terminated by a hydrogen atom or the like or may be substituted by an “optional substituent” described later. When the ring includes an optional element other than carbon atom, the resultant ring is a heterocycle.

The number of “one or more optional atoms” forming the saturated ring or unsaturated ring is, unless otherwise specified herein, preferably in a range from 2 to 15, more preferably in a range from 3 to 12, further preferably in a range from 3 to 5.

Unless otherwise specified herein, the ring, which may be a “saturated ring” or “unsaturated ring,” is preferably an “unsaturated ring.”

Unless otherwise specified herein, the “unsaturated ring” is preferably a benzene ring.

When “at least one combination of adjacent two or more” (of . . . ) are “mutually bonded to form a substituted or unsubstituted saturated ring” or “mutually bonded to form a substituted or unsubstituted unsaturated ring,” unless otherwise specified herein, at least one combination of adjacent two or more of components are preferably mutually bonded to form a substituted or unsubstituted “unsaturated ring” formed of a plurality of atoms of the basic skeleton, and 1 to 15 atoms of at least one element selected from the group consisting of carbon, nitrogen, oxygen and sulfur.

When the “saturated ring” or the “unsaturated ring” has a substituent, the substituent is the substituent described in the above under the subtitle “Substituent Mentioned Herein.”

In an exemplary embodiment herein, the substituent meant by the phrase “substituted or unsubstituted” (sometimes referred to as an “optional substituent” hereinafter) is, for instance, a group selected from the group consisting of an unsubstituted alkyl group having 1 to 50 carbon atoms, an unsubstituted alkenyl group having 2 to 50 carbon atoms, an unsubstituted alkynyl group having 2 to 50 carbon atoms, an unsubstituted cycloalkyl group having 3 to 50 ring carbon atoms, —Si(R₉₀₁)(R₉₀₂)(R₉₀₃), —O—(R₉₀₄), —S—(R₉₀₅), —N(R₉₀₆)(R₉₀₇), a halogen atom, a cyano group, a nitro group, an unsubstituted aryl group having 6 to 50 ring carbon atoms, and an unsubstituted heterocyclic group having 5 to 50 ring atoms, where: R₉₀₁ to R₉₀₇ each independently represent a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;

when two or more R₉₀₁ are present, the two or more R₉₀₁ are mutually the same or different;
when two or more R₉₀₂ are present, the two or more R₉₀₂ are mutually the same or different;
when two or more R₉₀₃ are present, the two or more R₉₀₃ are mutually the same or different;
when two or more R₉₀₄ are present, the two or more R₉₀₄ are mutually the same or different;
when two or more R₉₀₅ are present, the two or more R₉₀₅ are mutually the same or different;
when two or more R₉₀₆ are present, the two or more R₉₀₆ are mutually the same or different; and
when two or more R₉₀₇ are present, the two or more R₉₀₇ are mutually the same or different.

In an exemplary embodiment, the substituent meant by “substituted or unsubstituted” is selected from the group consisting of an alkyl group having 1 to 50 carbon atoms, an aryl group having 6 to 50 ring carbon atoms, and a heterocyclic group having 5 to 50 ring atoms.

In an exemplary embodiment, the substituent meant by “substituted or unsubstituted” is selected from the group consisting of an alkyl group having 1 to 18 carbon atoms, an aryl group having 6 to 18 ring carbon atoms, and a heterocyclic group having 5 to 18 ring atoms.

Specific examples of the above optional substituent are the same as the specific examples of the substituent described in the above under the subtitle “Substituent Mentioned Herein.”

Unless otherwise specified herein, adjacent ones of the optional substituents may form a “saturated ring” or an “unsaturated ring,” preferably a substituted or unsubstituted saturated five-membered ring, a substituted or unsubstituted saturated six-membered ring, a substituted or unsubstituted saturated five-membered ring, or a substituted or unsubstituted unsaturated six-membered ring, more preferably a benzene ring.

Unless otherwise specified herein, the optional substituent may further include a substituent. Examples of the substituent for the optional substituent are the same as the examples of the optional substituent.

The above is the description for the instances where “at least one combination of adjacent two or more (of . . . ) are mutually bonded to form a substituted or unsubstituted saturated ring” and “at least one combination of adjacent two or more (of . . . ) are mutually bonded to form a substituted or unsubstituted unsaturated ring” mentioned herein (sometimes referred to as an instance “bonded to form a ring”.

Herein, numerical ranges represented by “x to y” represents a range whose lower limit is the value (x) recited before “to” and whose upper limit is the value (y) recited after “to.” Herein, numerical ranges represented by “AA to BB” represents a range whose lower limit is the value (AA) recited before “to” and whose upper limit is the value (BB) recited after “to.”

### First Exemplary Embodiment

### Compound

A compound according to the first exemplary embodiment is a compound represented by a formula (2) below.

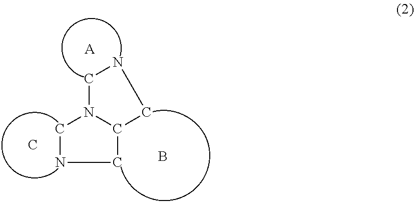

In the formula (2): the rings A and C each independently represent a substituted or unsubstituted aromatic heterocycle having 5 to 50 ring atoms;

a ring B represents a substituted or unsubstituted aromatic heterocycle having 5 to 50 ring atoms, or a substituted or unsubstituted aromatic hydrocarbon ring having 6 to 50 ring carbon atoms;

substituents for the ring A having a substituent, the ring B having a substituent, and the ring C having a substituent are each independently a substituted or unsubstituted alkyl group having 1 to 50 carbon atoms, a substituted or unsubstituted fluoroalkyl group having 1 to 50 carbon atoms, a substituted or unsubstituted cycloalkyl group having 3 to 50 ring carbon atoms, a cyano group, a halogen atom, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms;

R₉₀₁, R₉₀₂, R₉₀₃, R₉₀₆, and R₉₀₇ are each independently a substituted or unsubstituted aryl group having 6 to 50 ring carbon atoms, a substituted or unsubstituted heterocyclic group having 5 to 50 ring atoms, a substituted or unsubstituted alkyl group having 1 to 50 carbon atoms, or a substituted or unsubstituted cycloalkyl group having 3 to 50 ring carbon atoms; and

R₉₀₄ and R₉₀₅ are each independently a substituted or unsubstituted aryl group having 6 to 50 ring carbon atoms, a substituted or unsubstituted heterocyclic group having 5 to 50 ring atoms, a substituted or unsubstituted alkyl group having 1 to 50 carbon atoms, a substituted or unsubstituted cycloalkyl group having 3 to 50 ring carbon atoms, or a substituted or unsubstituted fluoroalkyl group having 1 to 50 carbon atoms.

A compound whose moiety corresponding to the ring A and the ring C of the structure represented by the formula (2) is a benzene ring has a fluorescent peak wavelength outside a visible light region. In contrast, it is believed that the compound according to the first exemplary embodiment, which is represented by the formula (2) and is provided with the ring A and the ring C in a form of a substituted or unsubstituted aromatic heterocycle having 5 to 50 ring atoms, exhibits high PLQY (PhotoLuminescence Quantum Yield) and has a fluorescent peak wavelength within the visible light region. It is believed that the compound according to the first exemplary embodiment has the fluorescent peak wavelength in the visible light region (more specifically, blue wavelength region).

As represented by the formula (2), the ring A and the ring C are each a heterocycle having at least one nitrogen atom and at least one carbon atom in a ring skeleton thereof. In the compound according to the first exemplary embodiment, the ring skeletons of the ring A and the ring C may each include, in addition to a nitrogen atom and carbon atom, at least one atom selected from the group consisting of an oxygen atom, sulfur atom, and silicon atom, or may consist of a nitrogen atom(s) and a carbon atom(s).

As represented by the formula (2), the ring B is a ring having at least three carbon atoms in a ring skeleton thereof. In the compound according to the first exemplary embodiment, when the ring B is an aromatic heterocycle, the ring skeleton of the ring B preferably includes, in addition to the carbon atoms, at least one atom selected from the group consisting of a nitrogen atom, an oxygen atom, sulfur atom, and silicon atom.

In the first exemplary embodiment, the rings A, B, and C are each independently a monocyclic ring or a fused ring.

In the first exemplary embodiment, the ring A and the ring C may also preferably be the same. Herein, when the rings are the same, ring skeletons of the rings are mutually the same and the substituents bonded to the ring skeletons are also mutually the same.

In the first exemplary embodiment, the ring A and the ring C may also preferably be different from each other. Herein, when the rings are different, unsubstituted ring skeletons of the rings are different from each other; while the ring skeletons are mutually the same, the substituents bonded to the ring skeletons are different from each other; or, while the substituents bonded to the ring skeletons are the same, the ring skeletons are different from each other.

The compound according to the first exemplary embodiment is also preferably represented by a formula (21) below.

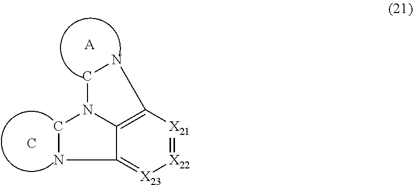

In the formula (21):

the ring A and the ring C represent the same as the ring A and the ring C in the formula (2);

X₂₁ is CR₂₁ or a nitrogen atom;

X₂₂ is CR₂₂ or a nitrogen atom;

X₂₃ is CR₂₃ or a nitrogen atom;

a combination of adjacent two or more of R₂₁, R₂₂ and R₂₃ are mutually bonded to form a substituted or unsubstituted saturated ring, mutually bonded to form a substituted or unsubstituted unsaturated ring, or not mutually bonded;

one or more of R₂₁, R₂₂ and R₂₃ not forming the saturated ring and not forming the unsaturated ring each independently represent a hydrogen atom, a substituted or unsubstituted alkyl group having 1 to 50 carbon atoms, a substituted or unsubstituted fluoroalkyl group having 1 to 50 carbon atoms, a substituted or unsubstituted cycloalkyl group having 3 to 50 ring carbon atoms, a cyano group, a halogen atom, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms; and

R₉₀₁, R₉₀₂, R₉₀₃, R₉₀₄, R₉₀₅, R₉₀₆ and R₉₀₇ represent the same as R₉₀₁, R₉₀₂, R₉₀₃, R₉₀₄, R₉₀₅, R₉₀₆ and R₉₀₇ in the formula (2).

The combination of adjacent two or more of R₂₁, R₂₂ and R₂₃ includes a pair of R₂₁ and R₂₂, a pair of R₂₂ and R₂₃, and a combination of R₂₁, R₂₂ and R₂₃.

The compound according to the first exemplary embodiment is also preferably represented by a formula (22) below.

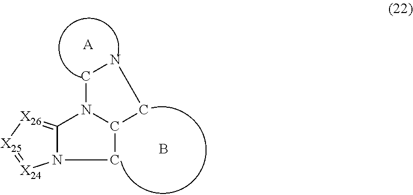

In the formula (22):

the ring A and the ring B represent the same as the ring A and the ring B in the formula (2);

X₂₄ is CR₂₄ or a nitrogen atom;

X₂₅ is CR₂₅ or a nitrogen atom;

X₂₆ is CR₂₆ or a nitrogen atom;

a combination of adjacent two or more of R₂₄, R₂₅ and R₂₆ are mutually bonded to form a substituted or unsubstituted saturated ring, mutually bonded to form a substituted or unsubstituted unsaturated ring, or not mutually bonded;

one or more of R₂₄, R₂₅ and R₂₆ not forming the saturated ring and not forming the unsaturated ring each independently represent a hydrogen atom, a substituted or unsubstituted alkyl group having 1 to 50 carbon atoms, a substituted or unsubstituted fluoroalkyl group having 1 to 50 carbon atoms, a substituted or unsubstituted cycloalkyl group having 3 to 50 ring carbon atoms, a cyano group, a halogen atom, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms; and

R₉₀₁, R₉₀₂, R₉₀₃, R₉₀₄, R₉₀₅, R₉₀₆ and R₉₀₇ represent the same as R₉₀₁, R₉₀₂, R₉₀₃, R₉₀₄, R₉₀₅, R₉₀₆ and R₉₀₇ in the formula (2).

The combination of adjacent two or more of R₂₄, R₂₅ and R₂₆ includes a pair of R₂₄ and R₂₅, a pair of R₂₅ and R₂₆, and a combination of R₂₄, R₂₅ and R₂₆.

The compound according to the first exemplary embodiment is also preferably represented by a formula (23) below.

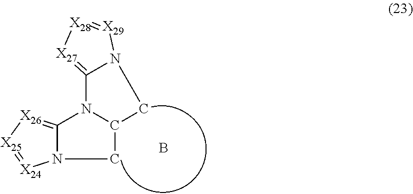

In the formula (23):

the ring B is the same as the ring B in the formula (2);

X₂₄ is CR₂₄ or a nitrogen atom;

X₂₅ is CR₂₅ or a nitrogen atom;

X₂₆ is CR₂₆ or a nitrogen atom;

X₂₇ is CR₂₇ or a nitrogen atom;

X₂₈ is CR₂₈ or a nitrogen atom;

X₂₉ is CR₂₉ or a nitrogen atom;

a combination of adjacent two or more of R₂₄, R₂₅ and R₂₆ are mutually bonded to form a substituted or unsubstituted saturated ring, mutually bonded to form a substituted or unsubstituted unsaturated ring, or not mutually bonded;

a combination of adjacent two or more of R₂₇, R₂₈ and R₂₉ are mutually bonded to form a substituted or unsubstituted saturated ring, mutually bonded to form a substituted or unsubstituted unsaturated ring, or not mutually bonded;

one or more of R₂₄, R₂₅, R₂₆, R₂₇, R₂₈, and R₂₉ not forming the saturated ring and not forming the unsaturated ring each independently represent a hydrogen atom, a substituted or unsubstituted alkyl group having 1 to 50 carbon atoms, a substituted or unsubstituted fluoroalkyl group having 1 to 50 carbon atoms, a substituted or unsubstituted cycloalkyl group having 3 to 50 ring carbon atoms, a cyano group, a halogen atom, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms; and

R₉₀₁, R₉₀₂, R₉₀₃, R₉₀₄, R₉₀₅, R₉₀₆ and R₉₀₇ represent the same as R₉₀₁, R₉₀₂, R₉₀₃, R₉₀₄, R₉₀₅, R₉₀₆ and R₉₀₇ in the formula (2).

The combination of adjacent two or more of R₂₇, R₂₈ and R₂₉ includes a pair of R₂₇ and R₂₈, a pair of R₂₈ and R₂₉, and a combination of R₂₇, R₂₈ and R₂₉.

The compound according to the first exemplary embodiment is also preferably represented by a formula (23A) below.

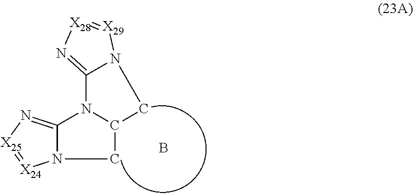

In the formula (23A):

the ring B is the same as the ring B in the formula (2);

X₂₄ is CR₂₄ or a nitrogen atom;

X₂₅ is CR₂₅ or a nitrogen atom;

X₂₈ is CR₂₈ or a nitrogen atom;

X₂₉ is CR₂₉ or a nitrogen atom;

R₂₄ and R₂₅ are mutually bonded to form a substituted or unsubstituted saturated ring, mutually bonded to form a substituted or unsubstituted unsaturated ring, or not mutually bonded;

R₂₈ and R₂₉ are mutually bonded to form a substituted or unsubstituted saturated ring, mutually bonded to form a substituted or unsubstituted unsaturated ring, or not mutually bonded;

one or more of R₂₄, R₂₅, R₂₈ and R₂₉ not forming the saturated ring and not forming the unsaturated ring each independently represent a hydrogen atom, a substituted or unsubstituted alkyl group having 1 to 50 carbon atoms, a substituted or unsubstituted fluoroalkyl group having 1 to 50 carbon atoms, a substituted or unsubstituted cycloalkyl group having 3 to 50 ring carbon atoms, a cyano group, a halogen atom, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms; and

R₉₀₁, R₉₀₂, R₉₀₃, R₉₀₄, R₉₀₅, R₉₀₆ and R₉₀₇ represent the same as R₉₀₁, R₉₀₂, R₉₀₃, R₉₀₄, R₉₀₅, R₉₀₆ and R₉₀₇ in the formula (2).

The compound according to the first exemplary embodiment is also preferably represented by a formula (24) below.

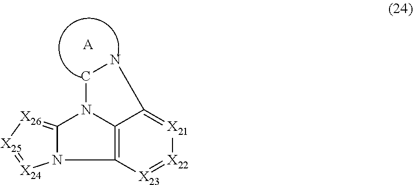

In the formula (24):

the ring A represents the same as the ring A in the formula (2);

X₂₁ is CR₂₁ or a nitrogen atom;

X₂₂ is CR₂₂ or a nitrogen atom;

X₂₃ is CR₂₃ or a nitrogen atom;

X₂₄ is CR₂₄ or a nitrogen atom;

X₂₅ is CR₂₅ or a nitrogen atom;

X₂₆ is CR₂₆ or a nitrogen atom;

a combination of adjacent two or more of R₂₁, R₂₂ and R₂₃ are mutually bonded to form a substituted or unsubstituted saturated ring, mutually bonded to form a substituted or unsubstituted unsaturated ring, or not mutually bonded;

a combination of adjacent two or more of R₂₄, R₂₅ and R₂₆ are mutually bonded to form a substituted or unsubstituted saturated ring, mutually bonded to form a substituted or unsubstituted unsaturated ring, or not mutually bonded;

one or more of R₂₁, R₂₂, R₂₃, R₂₄, R₂₅, and R₂₆ not forming the saturated ring and not forming the unsaturated ring each independently represent a hydrogen atom, a substituted or unsubstituted alkyl group having 1 to 50 carbon atoms, a substituted or unsubstituted fluoroalkyl group having 1 to 50 carbon atoms, a substituted or unsubstituted cycloalkyl group having 3 to 50 ring carbon atoms, a cyano group, a halogen atom, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms; and

R₉₀₁, R₉₀₂, R₉₀₃, R₉₀₄, R₉₀₅, R₉₀₆ and R₉₀₇ represent the same as R₉₀₁, R₉₀₂, R₉₀₃, R₉₀₄, R₉₀₅, R₉₀₆ and R₉₀₇ in the formula (2).

The compound according to the first exemplary embodiment is also preferably represented by a formula (25) below.

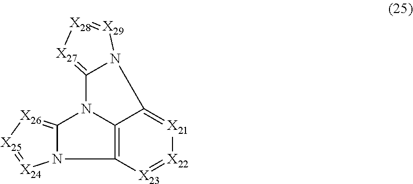

In the formula (25):

X₂₁ is CR₂₁ or a nitrogen atom;

X₂₂ is CR₂₂ or a nitrogen atom;

X₂₃ is CR₂₃ or a nitrogen atom;

X₂₄ is CR₂₄ or a nitrogen atom;

X₂₅ is CR₂₅ or a nitrogen atom;

X₂₆ is CR₂₆ or a nitrogen atom;

X₂₇ is CR₂₇ or a nitrogen atom;

X₂₈ is CR₂₈ or a nitrogen atom;

X₂₉ is CR₂₉ or a nitrogen atom;

a combination of adjacent two or more of R₂₁, R₂₂ and R₂₃ are mutually bonded to form a substituted or unsubstituted saturated ring, mutually bonded to form a substituted or unsubstituted unsaturated ring, or not mutually bonded;

a combination of adjacent two or more of R₂₄, R₂₅ and R₂₆ are mutually bonded to form a substituted or unsubstituted saturated ring, mutually bonded to form a substituted or unsubstituted unsaturated ring, or not mutually bonded;

a combination of adjacent two or more of R₂₇, R₂₈ and R₂₉ are mutually bonded to form a substituted or unsubstituted saturated ring, mutually bonded to form a substituted or unsubstituted unsaturated ring, or not mutually bonded;

one or more of R₂₁, R₂₂, R₂₃, R₂₄, R₂₅, R₂₆, R₂₇, R₂₈, and R₂₉ not forming the saturated ring and not forming the unsaturated ring each independently represent a hydrogen atom, a substituted or unsubstituted alkyl group having 1 to 50 carbon atoms, a substituted or unsubstituted fluoroalkyl group having 1 to 50 carbon atoms, a substituted or unsubstituted cycloalkyl group having 3 to 50 ring carbon atoms, a cyano group, a halogen atom, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms; and

R₉₀₁, R₉₀₂, R₉₀₃, R₉₀₄, R₉₀₅, R₉₀₆ and R₉₀₇ represent the same as R₉₀₁, R₉₀₂, R₉₀₃, R₉₀₄, R₉₀₅, R₉₀₆ and R₉₀₇ in the formula (2).

The compound according to the first exemplary embodiment is also preferably represented by a formula (25A) below.

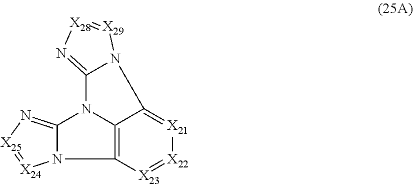

In the formula (25A):

X₂₁ is CR₂₁ or a nitrogen atom;

X₂₂ is CR₂₂ or a nitrogen atom;

X₂₃ is CR₂₃ or a nitrogen atom;

X₂₄ is CR₂₄ or a nitrogen atom;

X₂₅ is CR₂₅ or a nitrogen atom;

X₂₈ is CR₂₈ or a nitrogen atom;

X₂₉ is CR₂₉ or a nitrogen atom;

a combination of adjacent two or more of R₂₁, R₂₂ and R₂₃ are mutually bonded to form a substituted or unsubstituted saturated ring, mutually bonded to form a substituted or unsubstituted unsaturated ring, or not mutually bonded;

R₂₄ and R₂₅ are mutually bonded to form a substituted or unsubstituted saturated ring, mutually bonded to form a substituted or unsubstituted unsaturated ring, or not mutually bonded;

R₂₈ and R₂₉ are mutually bonded to form a substituted or unsubstituted saturated ring, mutually bonded to form a substituted or unsubstituted unsaturated ring, or not mutually bonded;

one or more of R₂₁, R₂₂, R₂₃, R₂₄, R₂₅, R₂₈ and R₂₉ not forming the saturated ring and not forming the unsaturated ring each independently represent a hydrogen atom, a substituted or unsubstituted alkyl group having 1 to 50 carbon atoms, a substituted or unsubstituted fluoroalkyl group having 1 to 50 carbon atoms, a substituted or unsubstituted cycloalkyl group having 3 to 50 ring carbon atoms, a cyano group, a halogen atom, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms; and

R₉₀₁, R₉₀₂, R₉₀₃, R₉₀₄, R₉₀₅, R₉₀₆ and R₉₀₇ represent the same as R₉₀₁, R₉₀₂, R₉₀₃, R₉₀₄, R₉₀₅, R₉₀₆ and R₉₀₇ in the formula (2).

The compound according to the first exemplary embodiment is also preferably represented by a formula (25B) below.

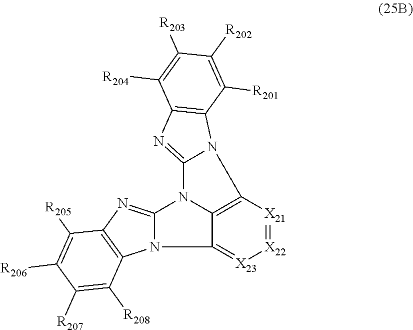

In the formula (25B):

X₂₁ is CR₂₁ or a nitrogen atom;

X₂₂ is CR₂₂ or a nitrogen atom;

X₂₃ is CR₂₃ or a nitrogen atom;

a combination of adjacent two or more of R₂₁, R₂₂ and R₂₃ are mutually bonded to form a substituted or unsubstituted saturated ring, mutually bonded to form a substituted or unsubstituted unsaturated ring, or not mutually bonded;

a combination of adjacent two or more of R₂₀₁, R₂₀₂, R₂₀₃ and R₂₀₄ are mutually bonded to form a substituted or unsubstituted saturated ring, mutually bonded to form a substituted or unsubstituted unsaturated ring, or not mutually bonded;

a combination of adjacent two or more of R₂₀₅, R₂₀₆, R₂₀₇ and R₂₀₈ are mutually bonded to form a substituted or unsubstituted saturated ring, mutually bonded to form a substituted or unsubstituted unsaturated ring, or not mutually bonded;

one or more of R₂₁, R₂₂, R₂₃, R₂₀₁, R₂₀₂, R₂₀₃, R₂₀₄, R₂₀₅, R₂₀₆, R₂₀₇, and R₂₀₈ not forming the saturated ring and not forming the unsaturated ring each independently represent a hydrogen atom, a substituted or unsubstituted alkyl group having 1 to 50 carbon atoms, a substituted or unsubstituted fluoroalkyl group having 1 to 50 carbon atoms, a substituted or unsubstituted cycloalkyl group having 3 to 50 ring carbon atoms, a cyano group, a halogen atom, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms; and

R₉₀₁, R₉₀₂, R₉₀₃, R₉₀₄, R₉₀₅, R₉₀₆ and R₉₀₇ represent the same as R₉₀₁, R₉₀₂, R₉₀₃, R₉₀₄, R₉₀₅, R₉₀₆ and R₉₀₇ in the formula (2).

The combination of adjacent two or more of R₂₀₁, R₂₀₂, R₂₀₃ and R₂₀₄ includes a pair of R₂₀₁ and R₂₀₂, a pair of R₂₀₂ and R₂₀₃, a pair of R₂₀₃ and R₂₀₄, a combination of R₂₀₁, R₂₀₂, and R₂₀₃ and a combination of R₂₀₂, R₂₀₃ and R₂₀₄.

The combination of adjacent two or more of R₂₀₅, R₂₀₆, R₂₀₇ and R₂₀₈ includes a pair of R₂₀₅ and R₂₀₆, a pair of R₂₀₆ and R₂₀₇, a pair of R₂₀₇ and R₂₀₈, a combination of R₂₀₅, R₂₀₆, and R₂₀₇ and a combination of R₂₀₆, R₂₀₇ and R₂₀₈.

The compound according to the first exemplary embodiment is also preferably represented by a formula (25C) below.

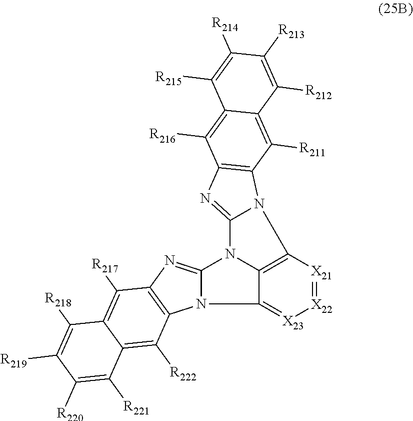

In the formula (25C):

X₂₁ is CR₂₁ or a nitrogen atom;

X₂₂ is CR₂₂ or a nitrogen atom;

X₂₃ is CR₂₃ or a nitrogen atom;

a combination of adjacent two or more of R₂₁, R₂₂ and R₂₃ are mutually bonded to form a substituted or unsubstituted saturated ring, mutually bonded to form a substituted or unsubstituted unsaturated ring, or not mutually bonded;

a combination of adjacent two or more of R₂₁₁, R₂₁₂, R₂₁₃, R₂₁₄, R₂₁₅ and R₂₁₆ are mutually bonded to form a substituted or unsubstituted saturated ring, mutually bonded to form a substituted or unsubstituted unsaturated ring, or not mutually bonded;

a combination of adjacent two or more of R₂₁₇, R₂₁₈, R₂₁₉, R₂₂₀, R₂₂₁ and R₂₂₂ are mutually bonded to form a substituted or unsubstituted saturated ring, mutually bonded to form a substituted or unsubstituted unsaturated ring, or not mutually bonded;

one or more of R₂₁, R₂₂, R₂₃, R₂₁₁, R₂₁₂, R₂₁₃, R₂₁₄, R₂₁₅, R₂₁₆, R₂₁₇, R₂₁₈, R₂₁₉, R₂₂₀, R₂₂₁ and R₂₂₂ not forming the saturated ring and not forming the unsaturated ring each independently represent a hydrogen atom, a substituted or unsubstituted alkyl group having 1 to 50 carbon atoms, a substituted or unsubstituted fluoroalkyl group having 1 to 50 carbon atoms, a substituted or unsubstituted cycloalkyl group having 3 to 50 ring carbon atoms, a cyano group, a halogen atom, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms; and

R₉₀₁, R₉₀₂, R₉₀₃, R₉₀₄, R₉₀₅, R₉₀₆ and R₉₀₇ represent the same as R₉₀₁, R₉₀₂, R₉₀₃, R₉₀₄, R₉₀₅, R₉₀₆ and R₉₀₇ in the formula (2).

The combination of adjacent two or more of R₂₁₁, R₂₁₂, R₂₁₃, R₂₁₄, R₂₁₅ and R₂₁₆ include a pair of R₂₁₁ and R₂₁₂, a pair of R₂₁₂ and R₂₁₃, a pair of R₂₁₃ and R₂₁₄, a pair of R₂₁₄ and R₂₁₅, a pair of R₂₁₅ and R₂₁₆, a combination of R₂₁₁, R₂₁₂, and R₂₁₃, a combination of R₂₁₂, R₂₁₃, and R₂₁₄, a combination of R₂₁₃, R₂₁₄, and R₂₁₅ and a combination of R₂₁₄, R₂₁₅ and R₂₁₆.

The combination of adjacent two or more of R₂₁₇, R₂₁₈, R₂₁₉, R₂₂₀, R₂₂₁ and R₂₂₂ include a pair of R₂₁₇ and R₂₁₈, a pair of R₂₁₈ and R₂₁₉, a pair of R₂₁₉ and R₂₂₀, a pair of R₂₂₀ and R₂₂₁, a pair of R₂₂₁ and R₂₂₂, a combination of R₂₁₇, R₂₁₈, and R₂₁₉, a combination of R₂₁₈, R₂₁₉, and R₂₂₀, a combination of R₂₁₉, R₂₂₀, and R₂₂₁ and a combination of R₂₂₀, R₂₂₁ and R₂₂₂.

The compound according to the first exemplary embodiment is also preferably represented by a formula (251B), formula (252B), formula (253B) or formula (254B) below.

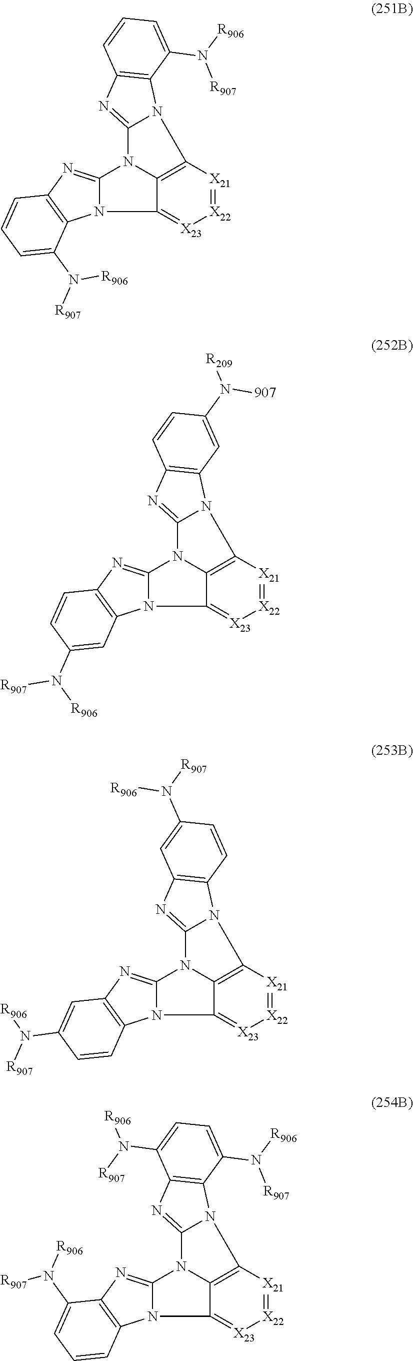

In the formula (251B), (252B), (253B) or (254B):

X₂₁ is CR₂₁ or a nitrogen atom;

X₂₂ is CR₂₂ or a nitrogen atom;

X₂₃ is CR₂₃ or a nitrogen atom;

a combination of adjacent two or more of R₂₁, R₂₂ and R₂₃ are mutually bonded to form a substituted or unsubstituted saturated ring, mutually bonded to form a substituted or unsubstituted unsaturated ring, or not mutually bonded;

one or more of R₂₁, R₂₂ and R₂₃ not forming the saturated ring and not forming the unsaturated ring each independently represent a hydrogen atom, a substituted or unsubstituted alkyl group having 1 to 50 carbon atoms, a substituted or unsubstituted fluoroalkyl group having 1 to 50 carbon atoms, a substituted or unsubstituted cycloalkyl group having 3 to 50 ring carbon atoms, a cyano group, a halogen atom, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms; and

R₉₀₁, R₉₀₂, R₉₀₃, R₉₀₄, R₉₀₅, R₉₀₆ and R₉₀₇ represent the same as R₉₀₁, R₉₀₂, R₉₀₃, R₉₀₄, R₉₀₅, R₉₀₆ and R₉₀₇ in the formula (2).

In the compound according to the first exemplary embodiment, R₉₀₆ and R₉₀₇ are also preferably each independently a substituted or unsubstituted aryl group having 6 to 50 ring carbon atoms.

In the compound represented by the formula (251B), formula (252B), formula (253B) or formula (254B), R₉₀₆ and R₉₀₇ are preferably each independently a substituted or unsubstituted aryl group having 6 to 50 ring carbon atoms.

In the compound according to the first exemplary embodiment, it is preferable that X₂₁ is CR₂₁, X₂₂ is CR₂₂, X₂₃ is CR₂₃, and R₂₁ and R₂₃ are hydrogen atoms. For instance, the compound according to the first exemplary embodiment is represented by a formula (25C-1) below when X₂₁ is CR₂₁, X₂₂ is CR₂₂, X₂₃ is CR₂₃, and R₂₁ and R₂₃ are hydrogen atoms in the formula (25C).

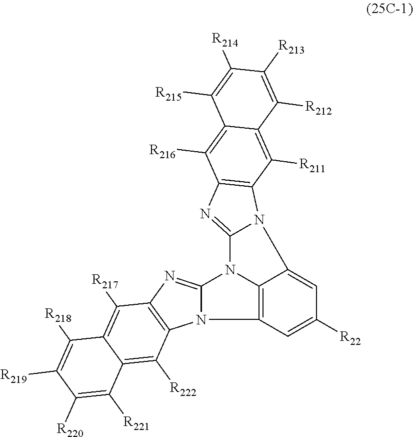

In the formula (25C-1), R₂₂ and R₂₁₁ to R₂₂₂ represent the same as R₂₂ and R₂₁₁ to R₂₂₂ in the formula (25C).

In the compound according to the first exemplary embodiment, it is preferable that R₂₂ is a substituted or unsubstituted alkyl group having 1 to 50 carbon atoms, a substituted or unsubstituted fluoroalkyl group having 1 to 50 carbon atoms, a substituted or unsubstituted cycloalkyl group having 3 to 50 ring carbon atoms, a cyano group, a halogen atom, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms. In this case, R₉₀₁, R₉₀₂, R₉₀₃, R₉₀₄, R₉₀₅, R₉₀₆ and R₉₀₇ represent the same as R₉₀₁, R₉₀₂, R₉₀₃, R₉₀₄, R₉₀₅, R₉₀₆ and R₉₀₇ in the formula (2).

In the compound according to the first exemplary embodiment, it is more preferable that R₂₂ is a substituted or unsubstituted alkyl group having 1 to 6 carbon atoms, a substituted or unsubstituted fluoroalkyl group having 1 to 6 carbon atoms, a substituted or unsubstituted cycloalkyl group having 3 to 6 ring carbon atoms, a cyano group, a halogen atom, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 18 ring carbon atoms, or a substituted or unsubstituted heterocyclic group having 5 to 18 ring atoms; R₉₀₁, R₉₀₂, R₉₀₃, R₉₀₆, and R₉₀₇ included in the group R₂₂ are each independently a substituted or unsubstituted aryl group having 6 to 18 ring carbon atoms, a substituted or unsubstituted heterocyclic group having 5 to 18 ring atoms, a substituted or unsubstituted alkyl group having 1 to 6 carbon atoms, or a substituted or unsubstituted cycloalkyl group having 3 to 6 ring carbon atoms; and R₉₀₄ and R₉₀₅ included in the group R₂₂ are each independently a substituted or unsubstituted aryl group having 6 to 18 ring carbon atoms, a substituted or unsubstituted heterocyclic group having 5 to 18 ring atoms, a substituted or unsubstituted alkyl group having 1 to 6 carbon atoms, a substituted or unsubstituted cycloalkyl group having 3 to 6 ring carbon atoms, or a substituted or unsubstituted fluoroalkyl group having 1 to 6 carbon atoms.

In the compound according to the first exemplary embodiment, it is also preferable that X₂₁ is CR₂₁, X₂₂ is CR₂₂, X₂₃ is CR₂₃, and R₂₁, R₂₂ and R₂₃ are hydrogen atoms.

### Method of Preparing Compound According to First Exemplary Embodiment

The compound according to the first exemplary embodiment can be prepared through, for instance, a process described later in Examples. The compound according to the first exemplary embodiment can be prepared, for instance, by application of known substitution reactions and/or materials depending on a target compound according to reactions described later in Examples.

Specific examples of the compound according to the first exemplary embodiment is exemplified by compounds below. It should however be noted that the invention is not limited to the specific examples of the compound.

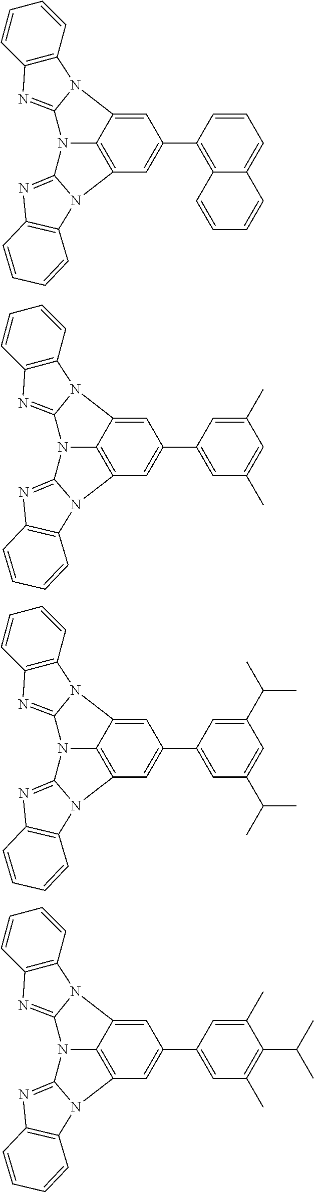

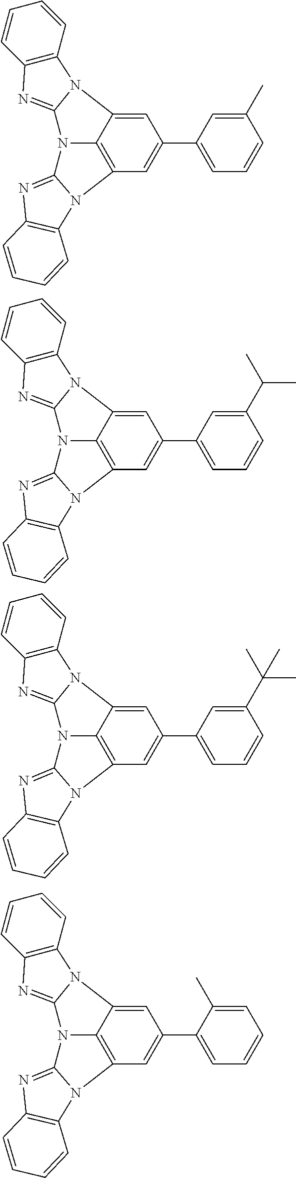

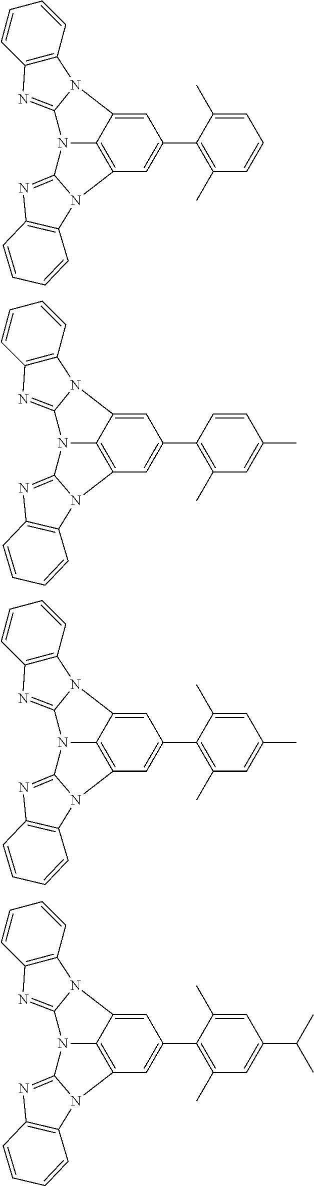

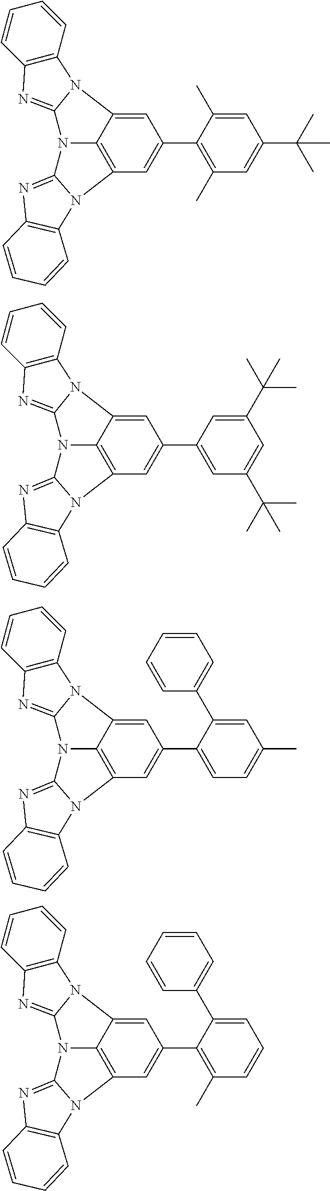

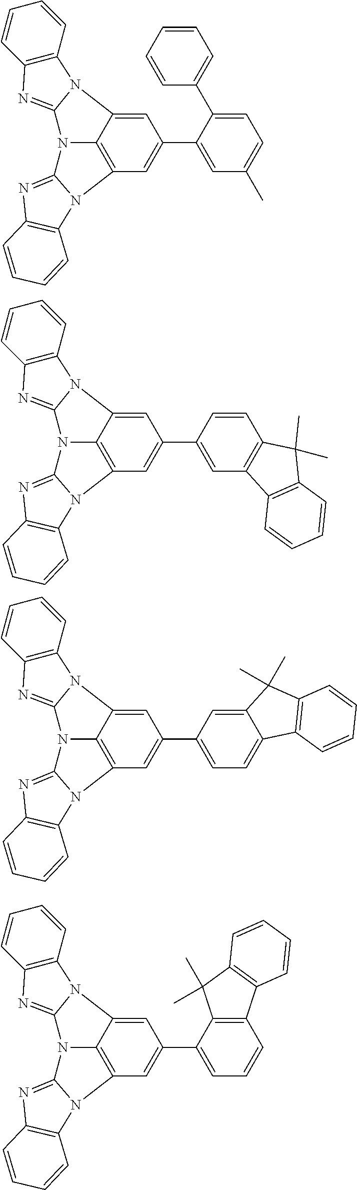

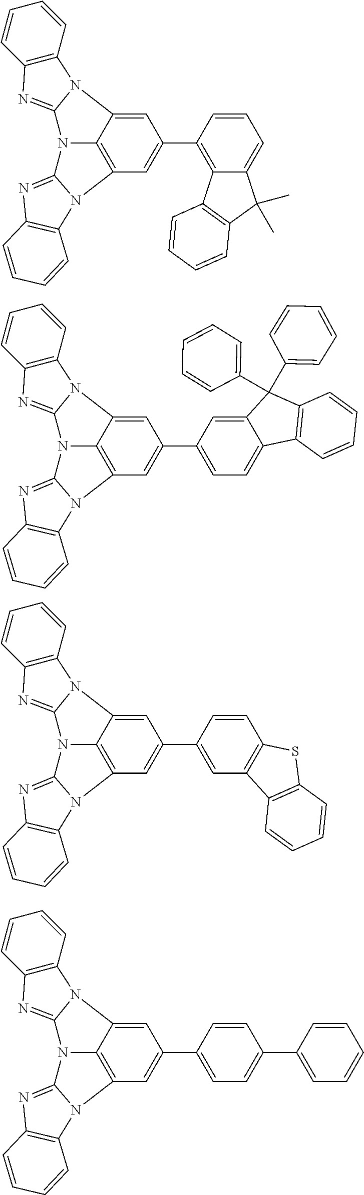

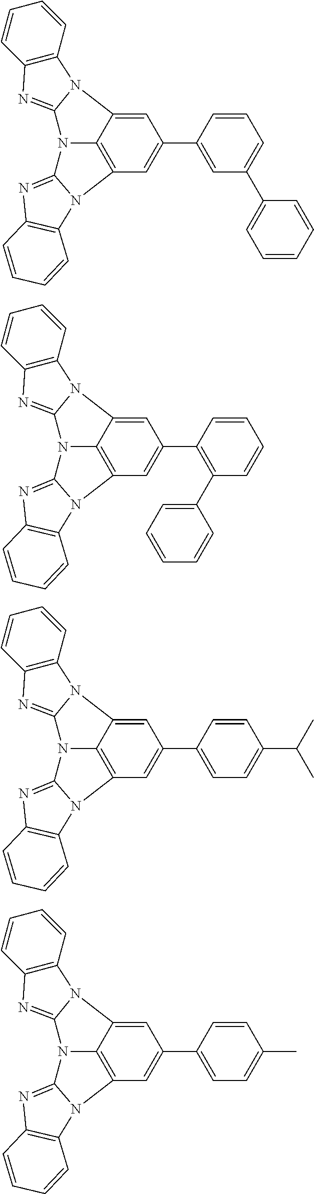

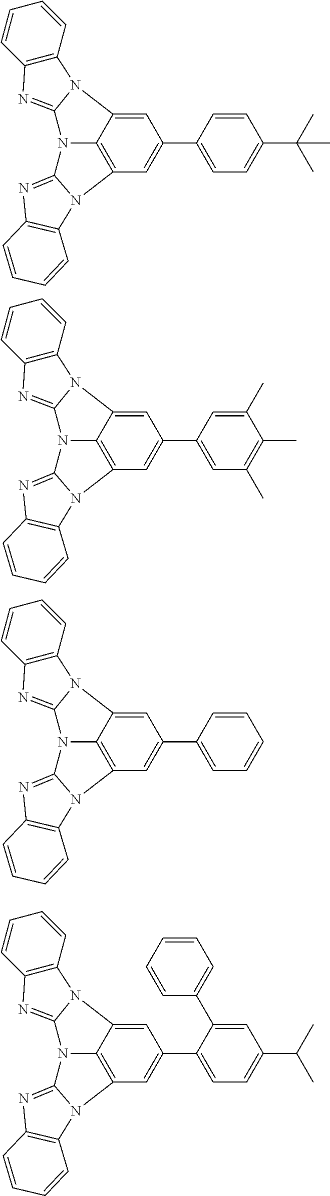

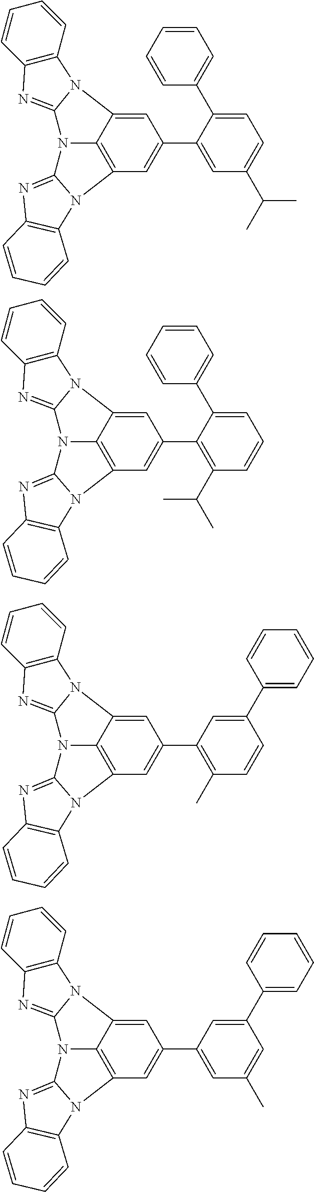

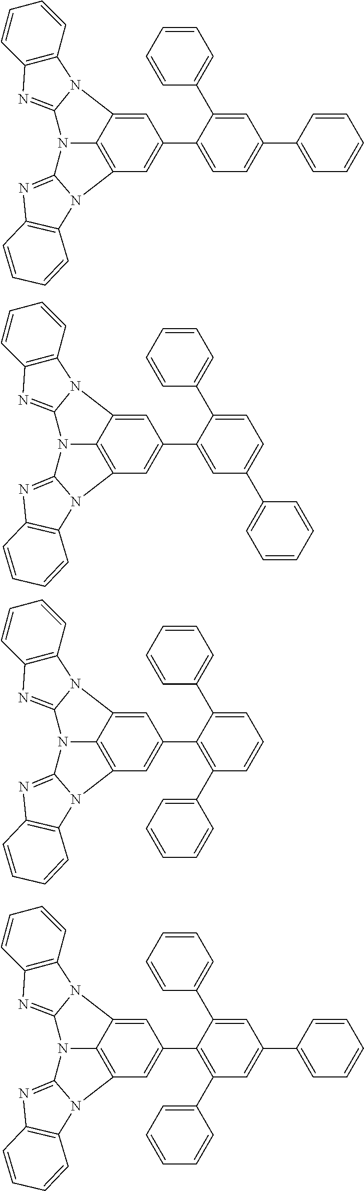

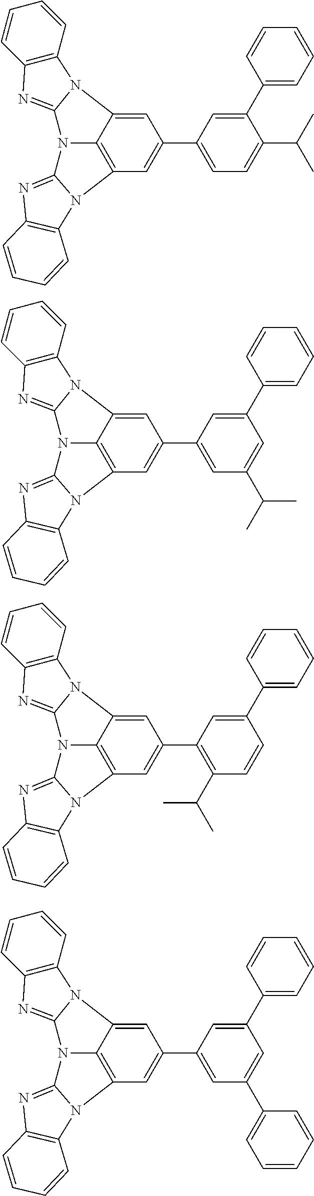

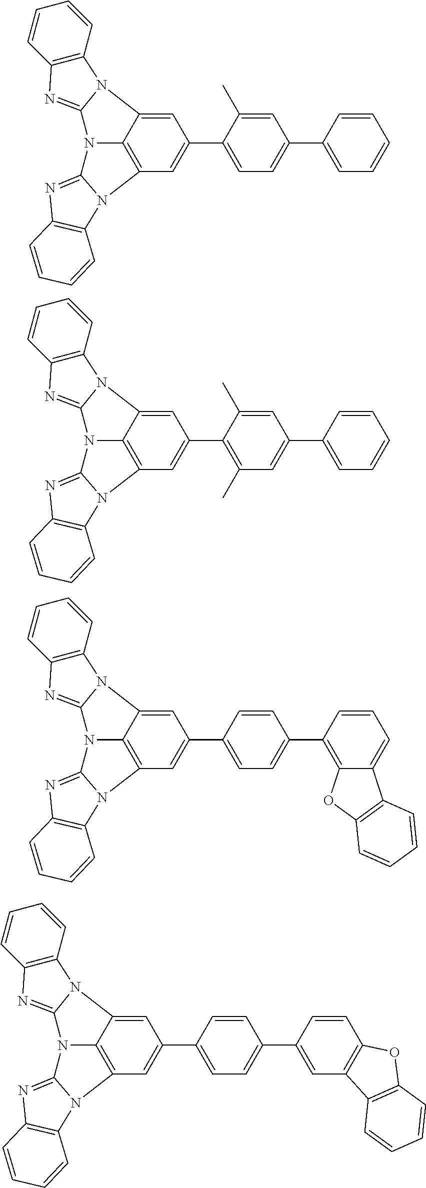

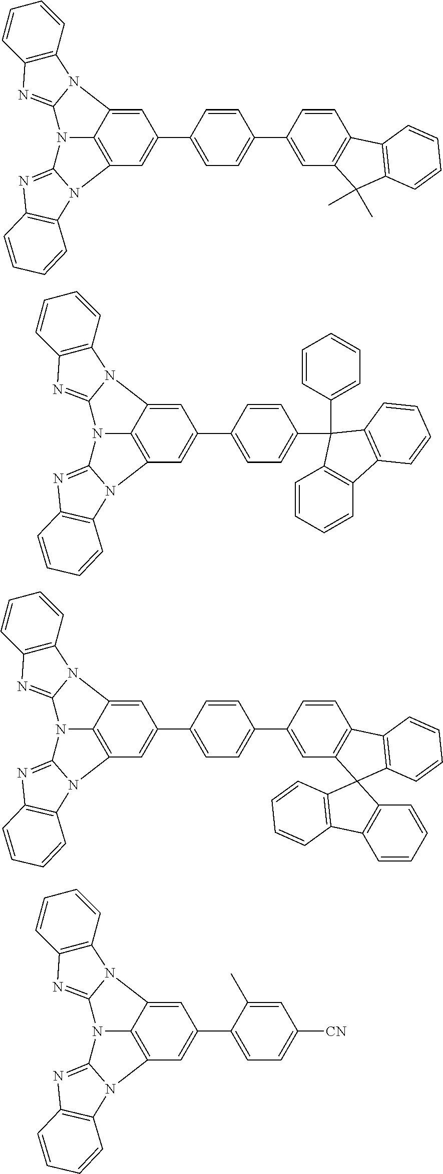

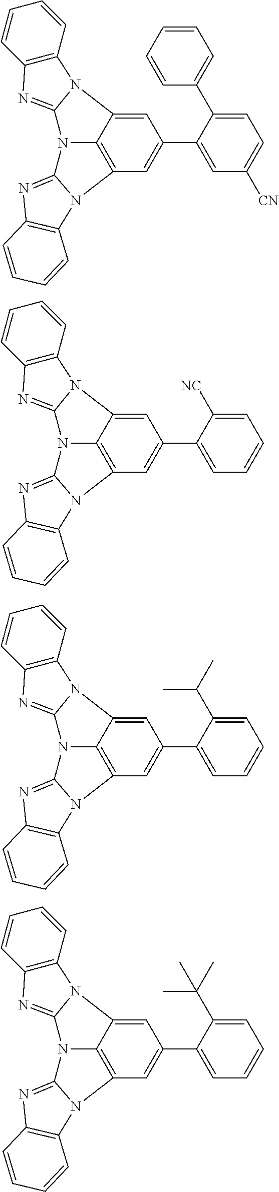

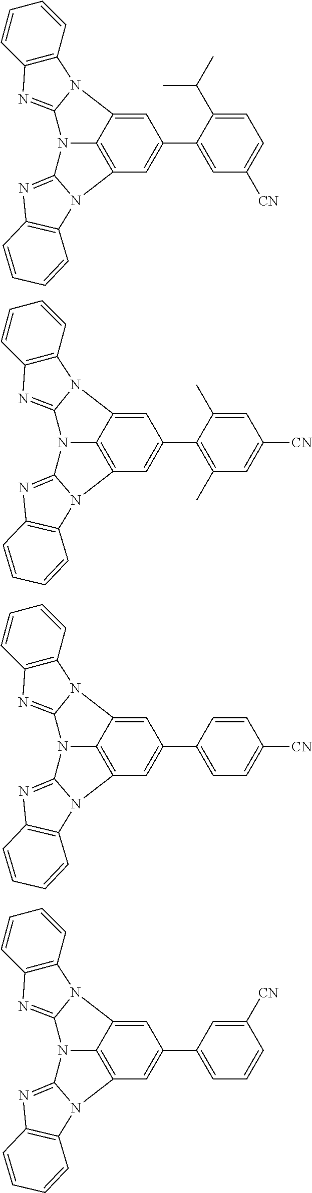

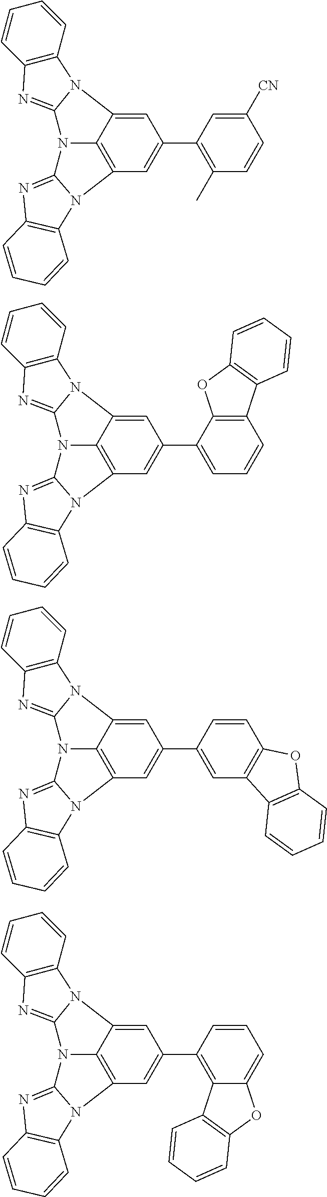

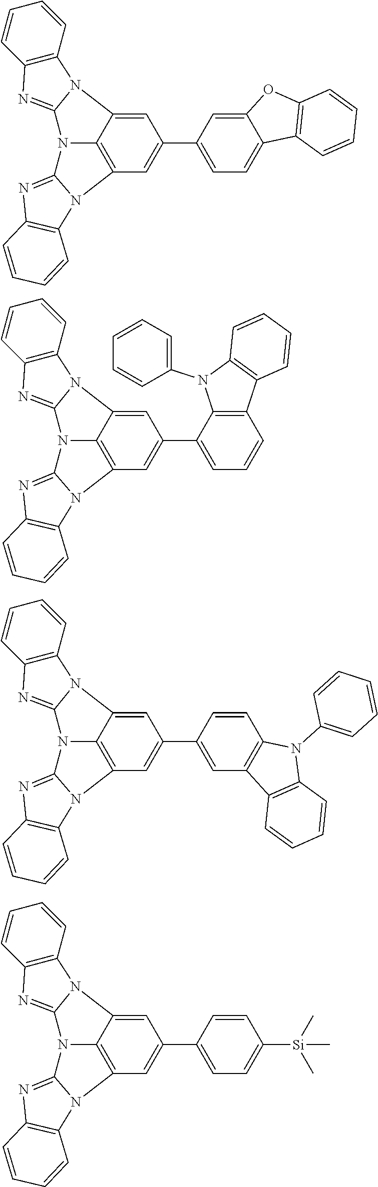

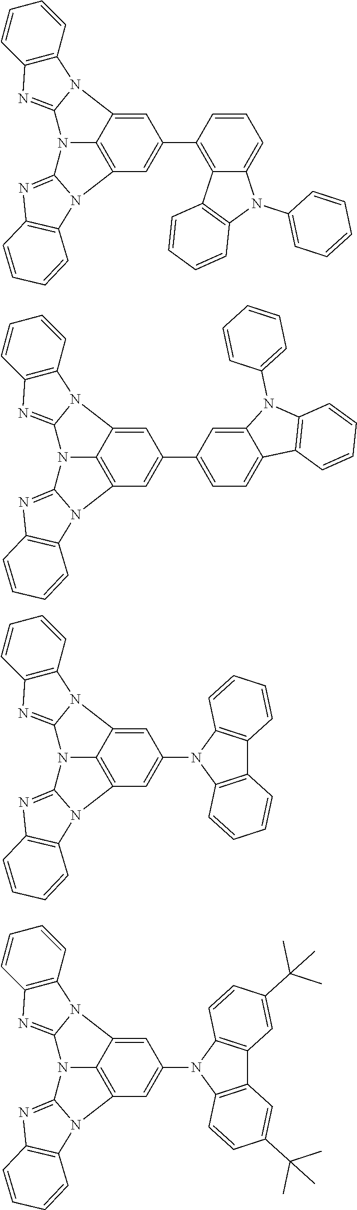

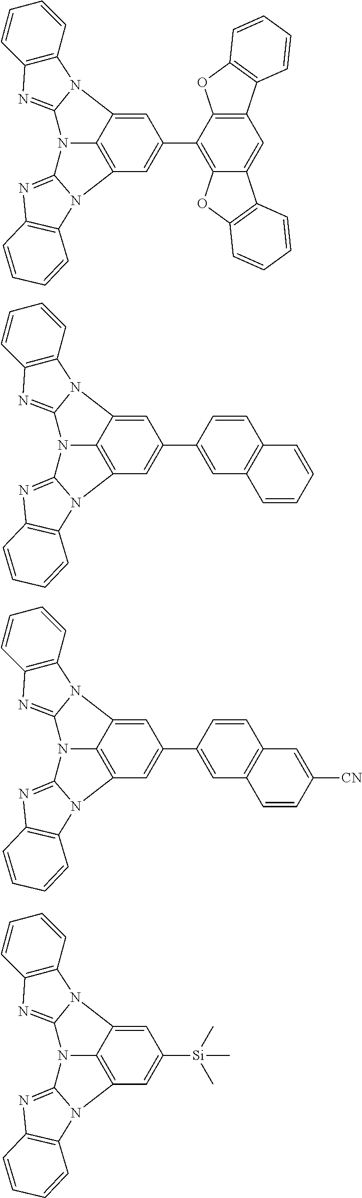

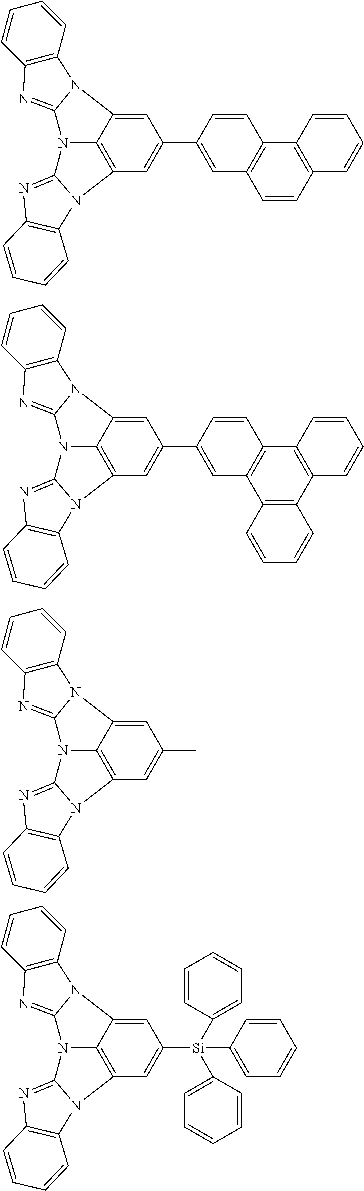

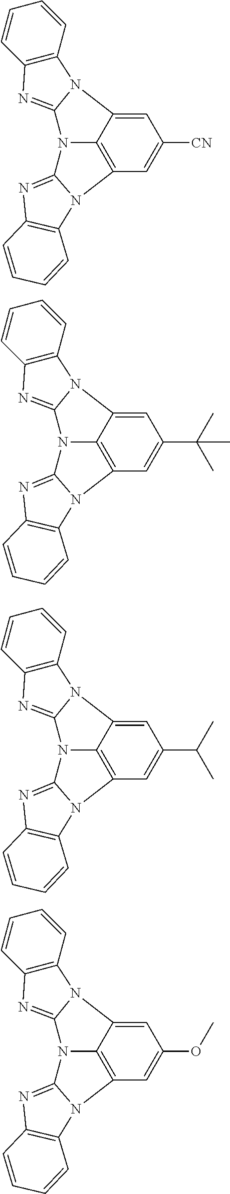

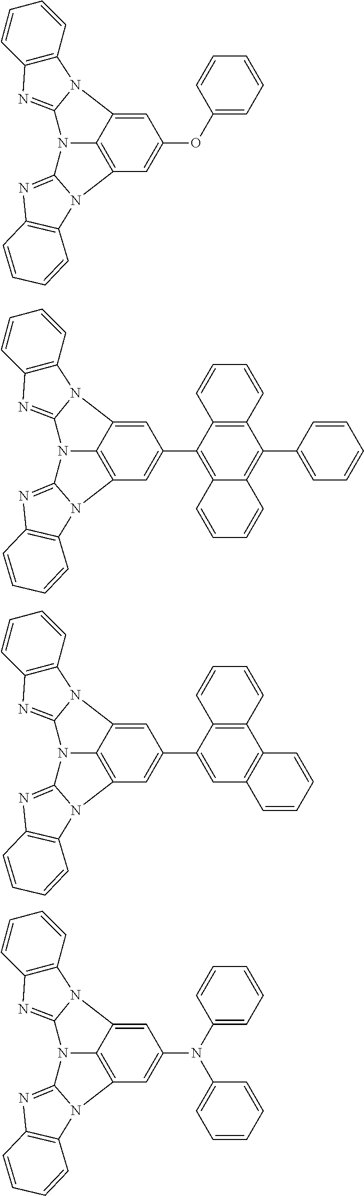

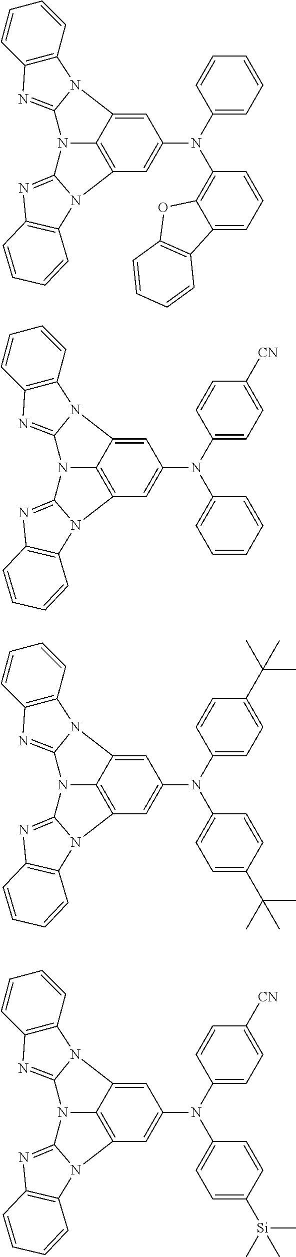

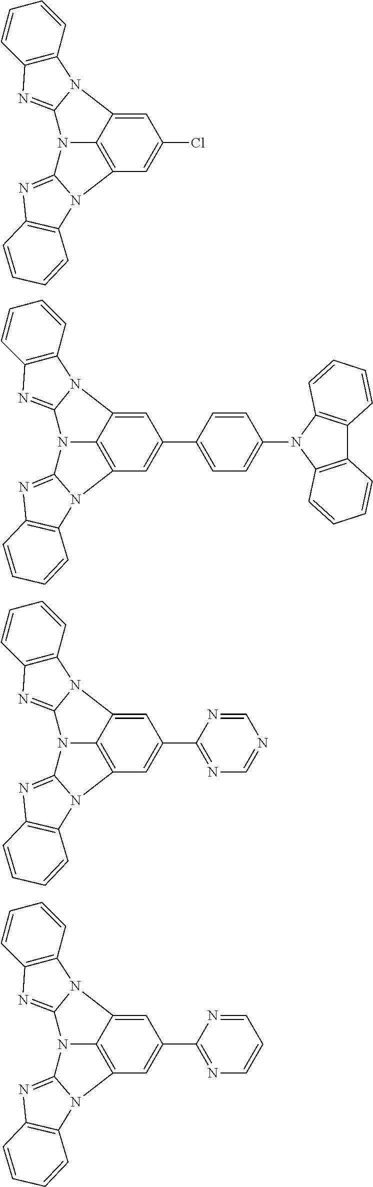

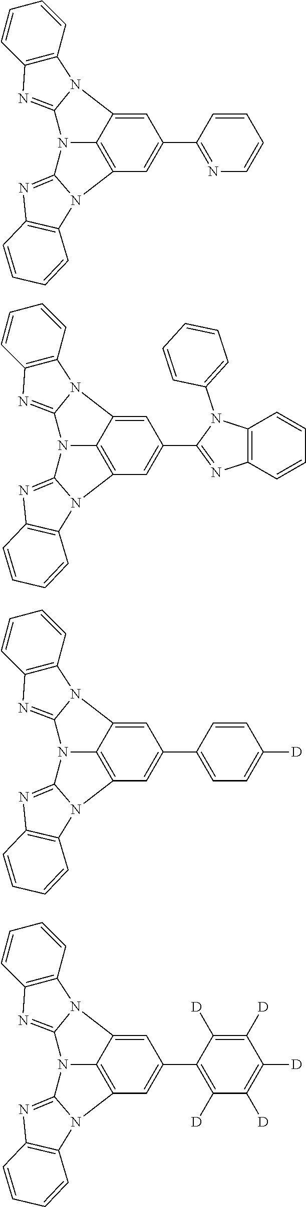

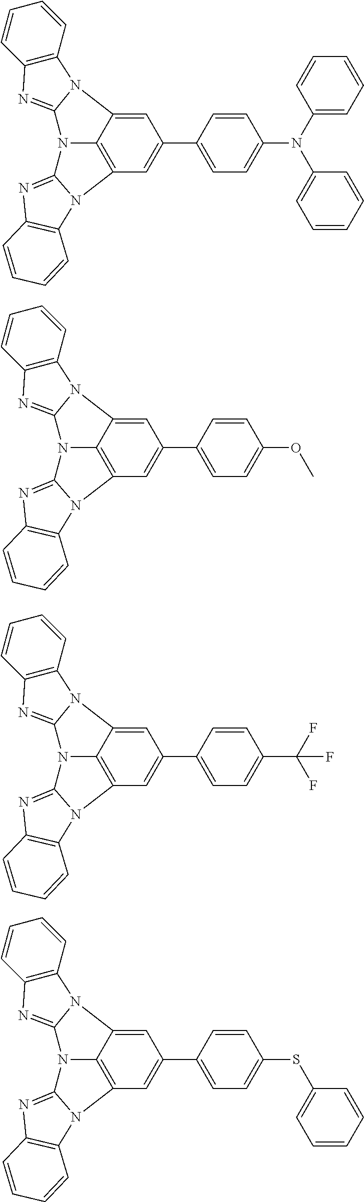

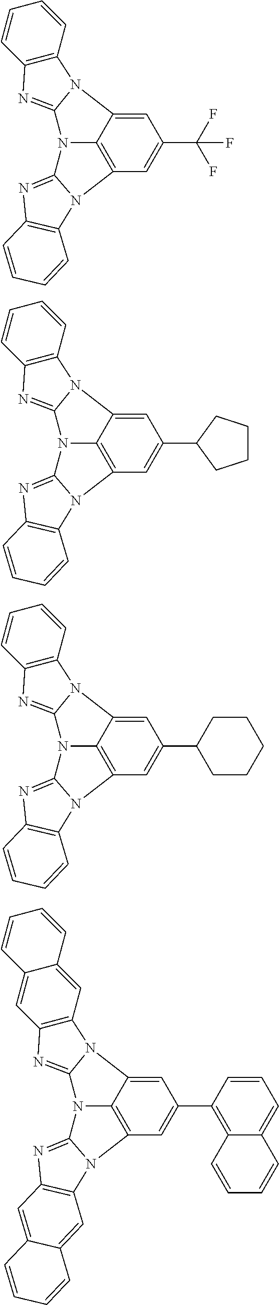

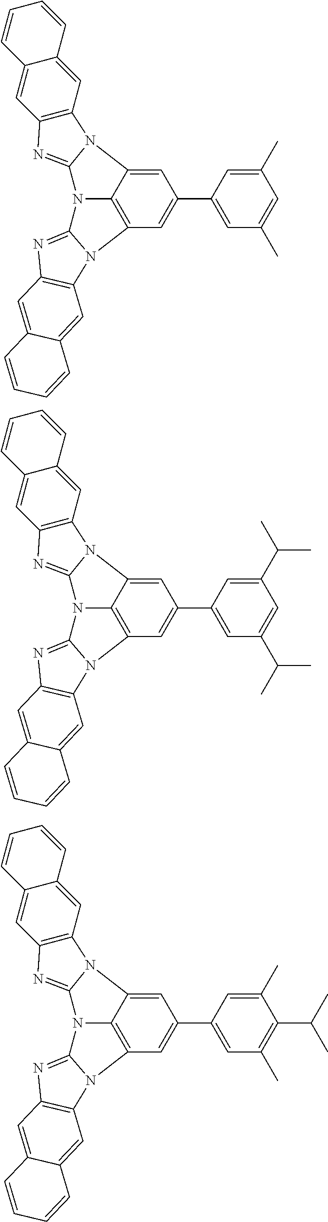

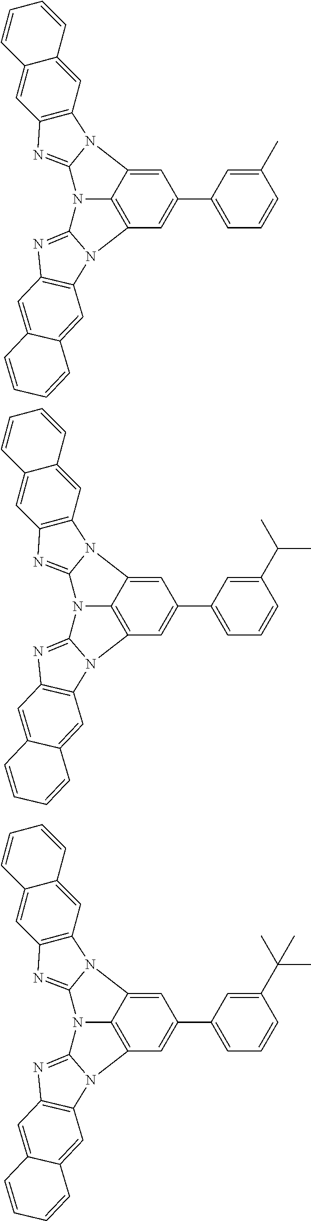

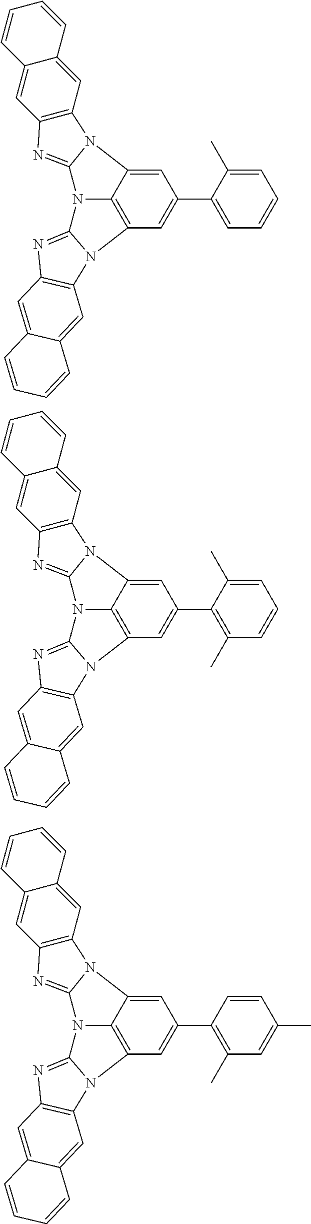

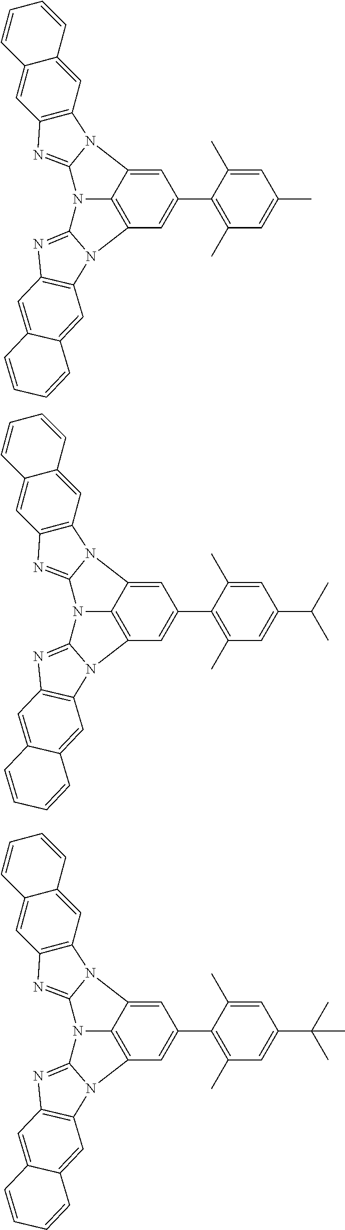

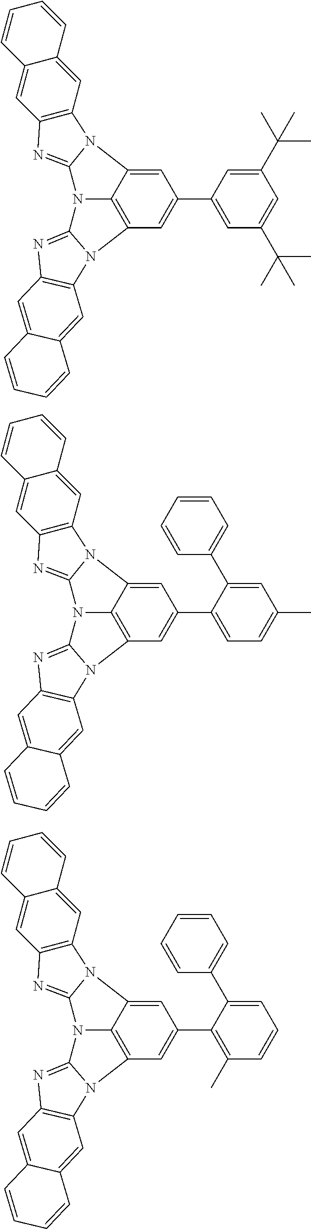

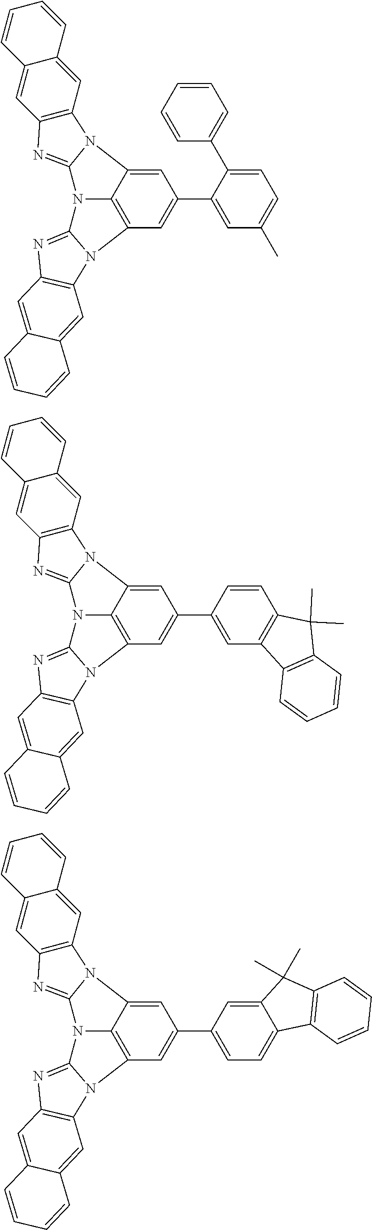

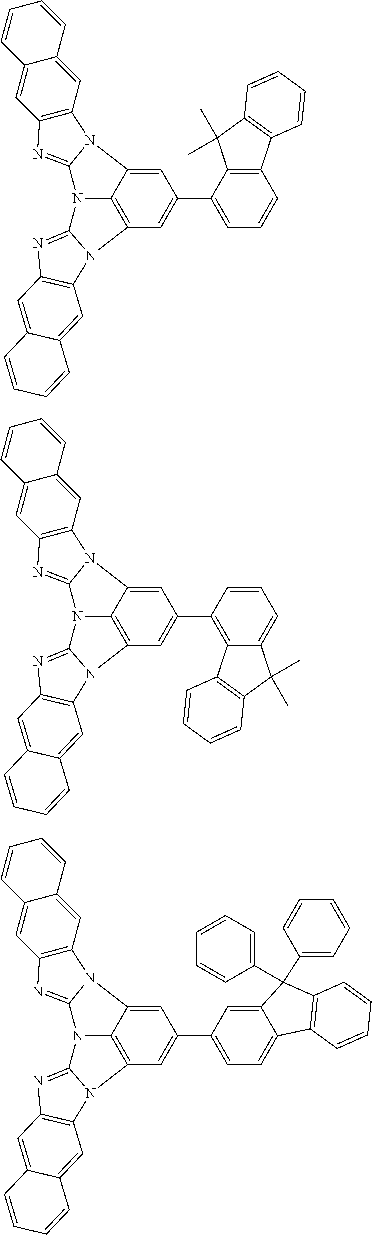

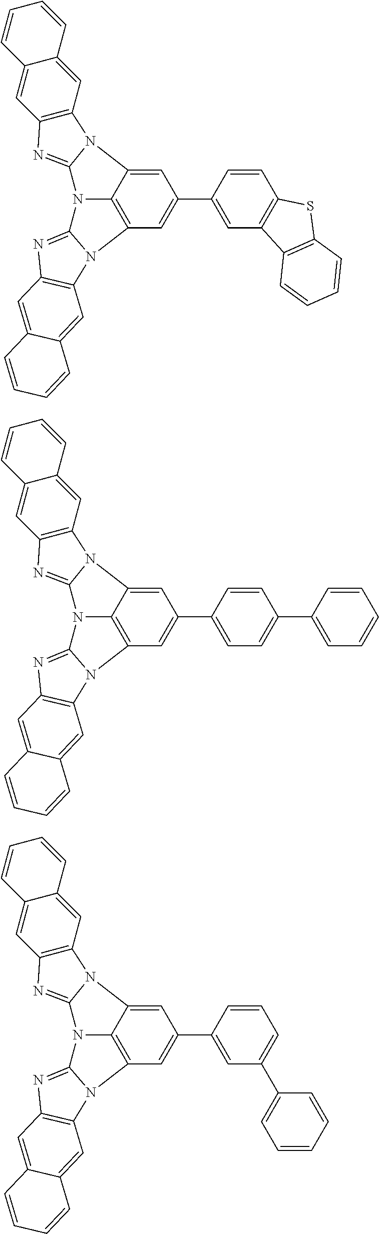

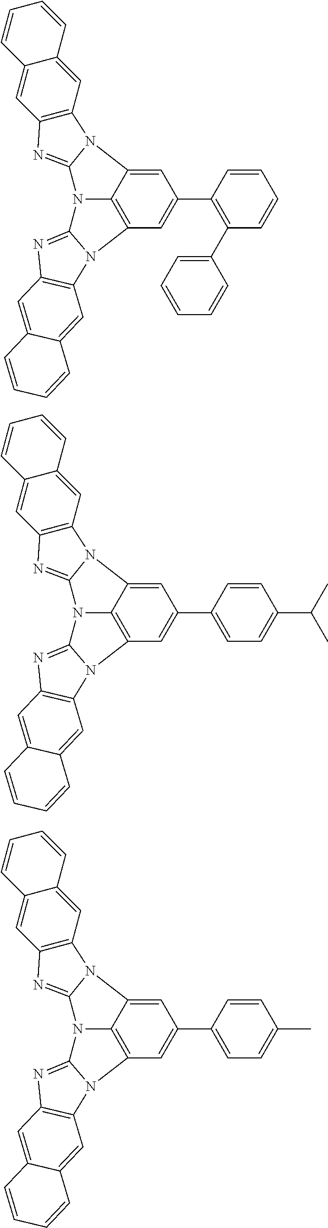

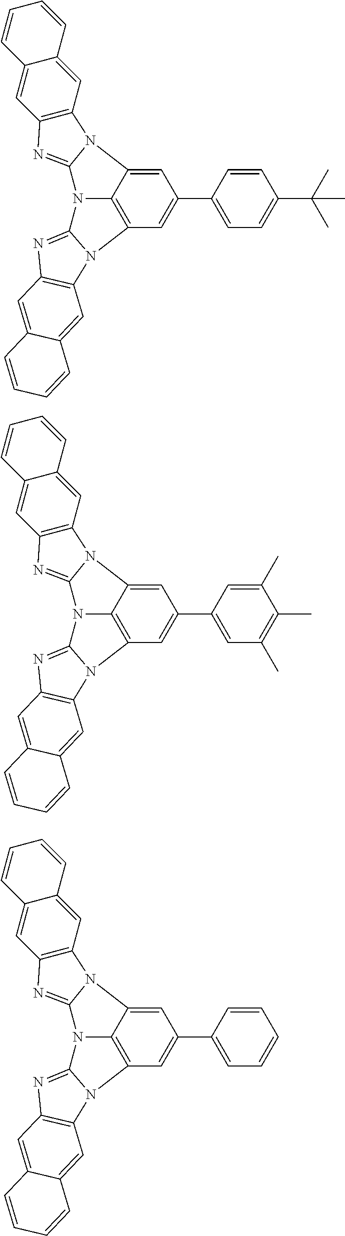

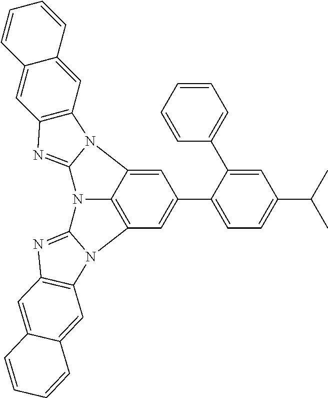 [SMILES: CC(C)c1ccc(-c2cc3c4c(c2)n2c5cc6ccccc6cc5nc2n4c2nc4cc5ccccc5cc4n32)c(-c2ccccc2)c1]

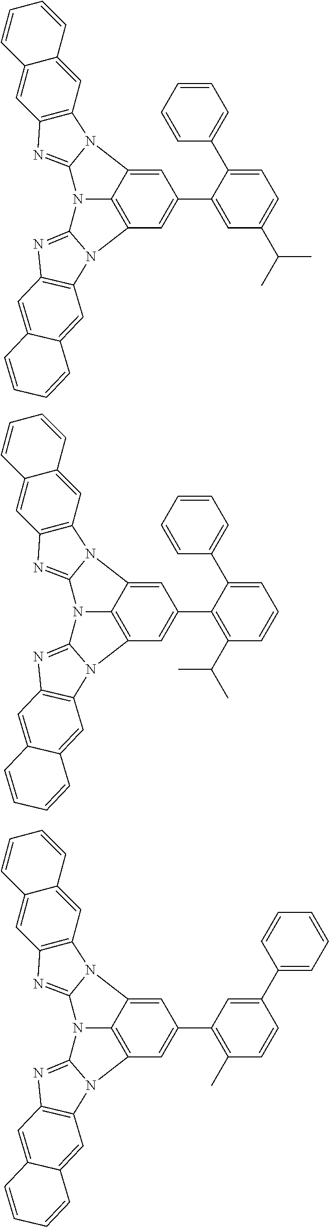

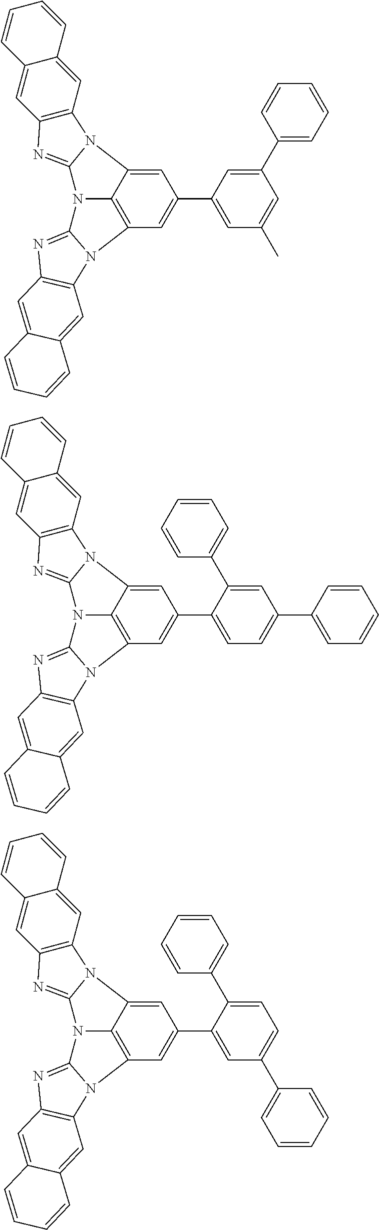

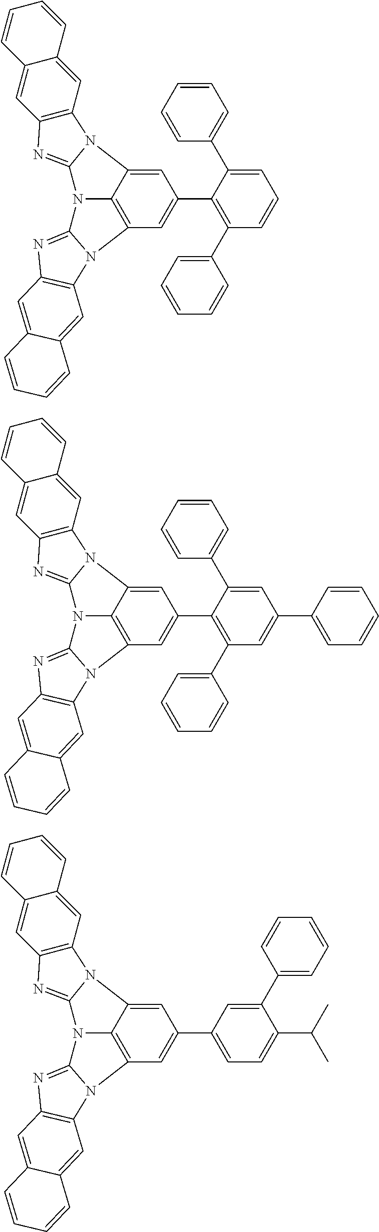

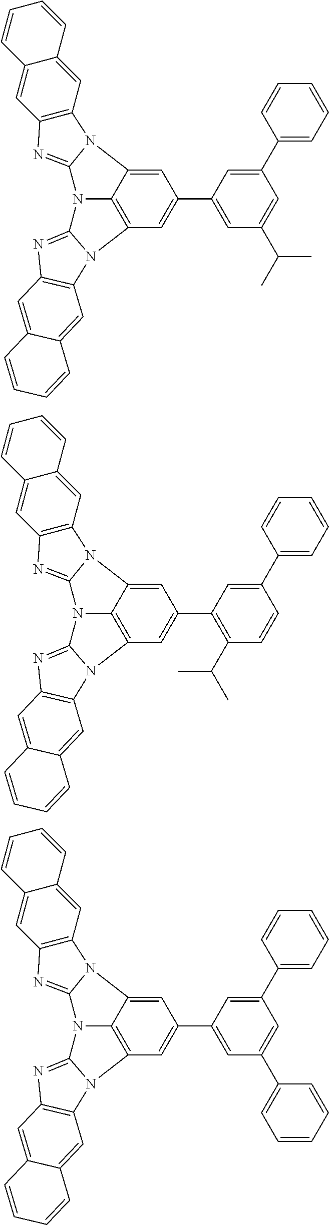

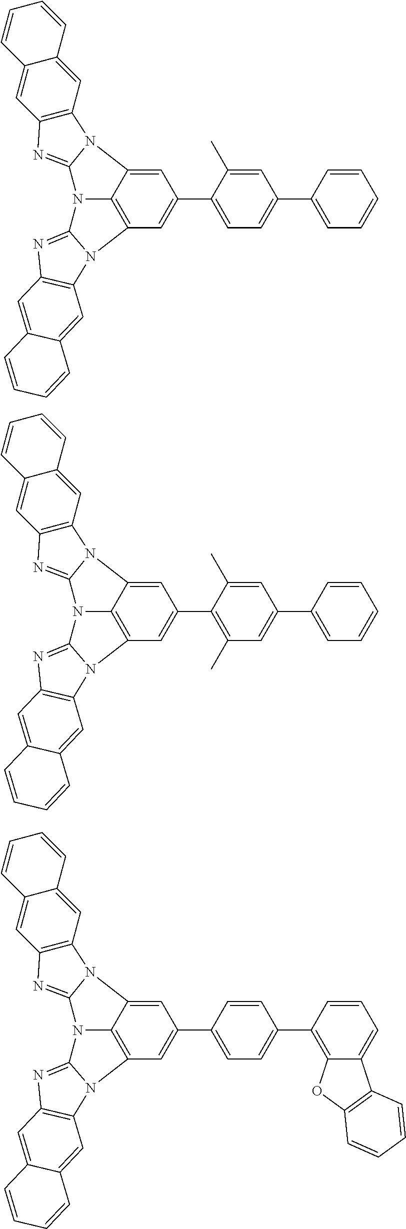

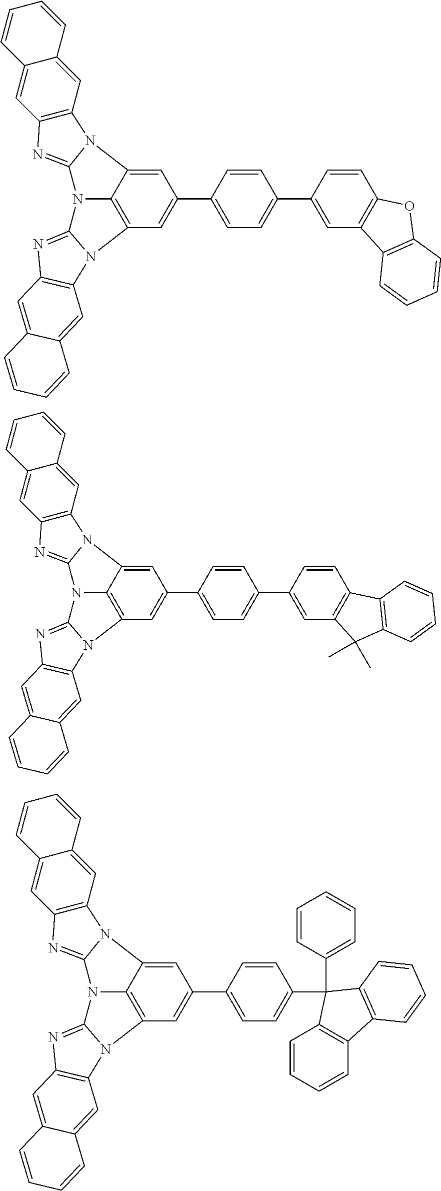

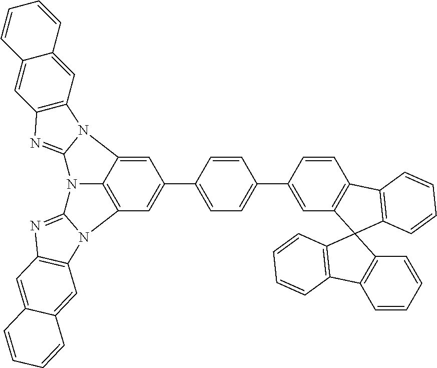 [SMILES: c1ccc2c(c1)-c1ccccc1C21c2ccccc2-c2ccc(-c3ccc(-c4cc5c6c(c4)n4c7cc8ccccc8cc7nc4n6c4nc6cc7ccccc7cc6n54)cc3)cc21]

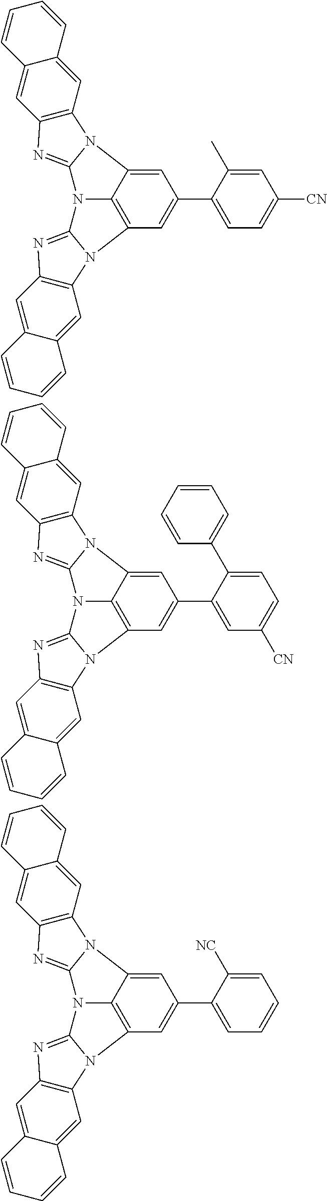

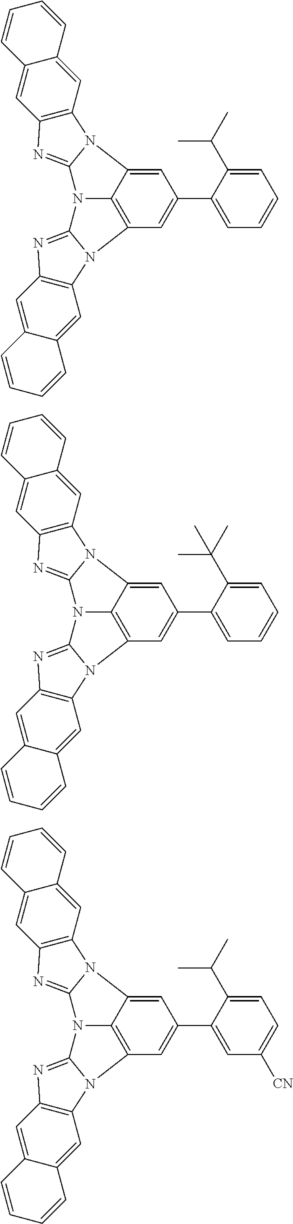

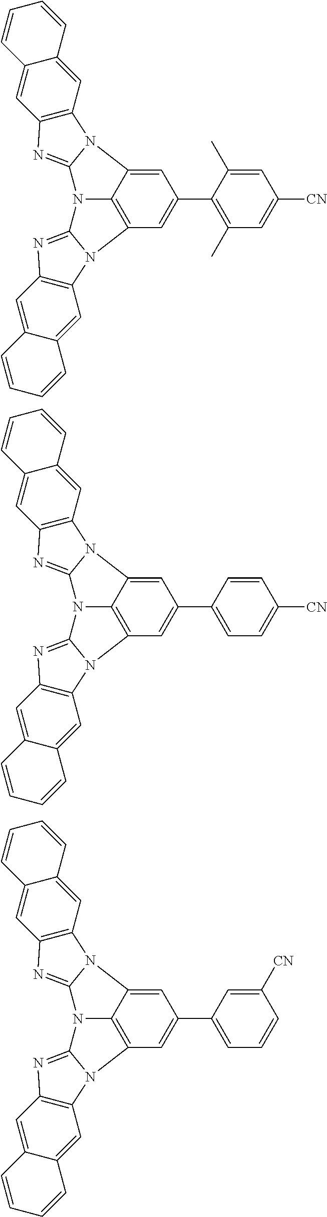

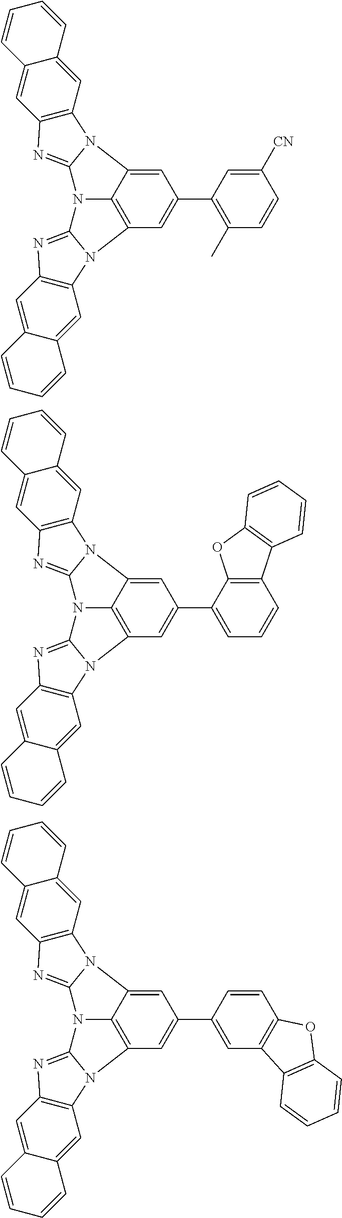

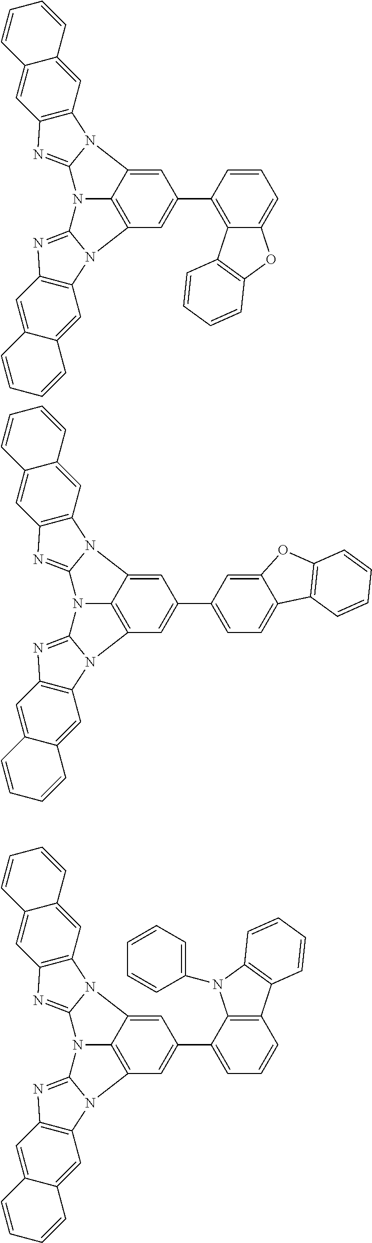

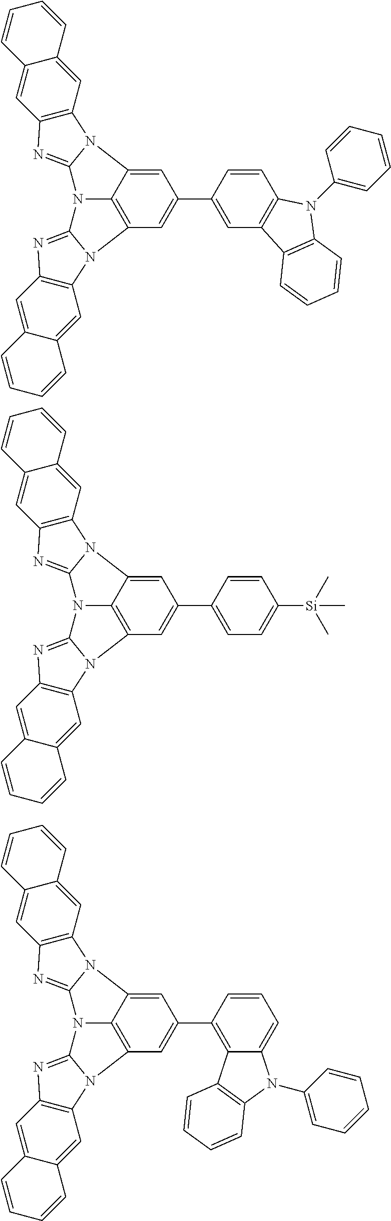

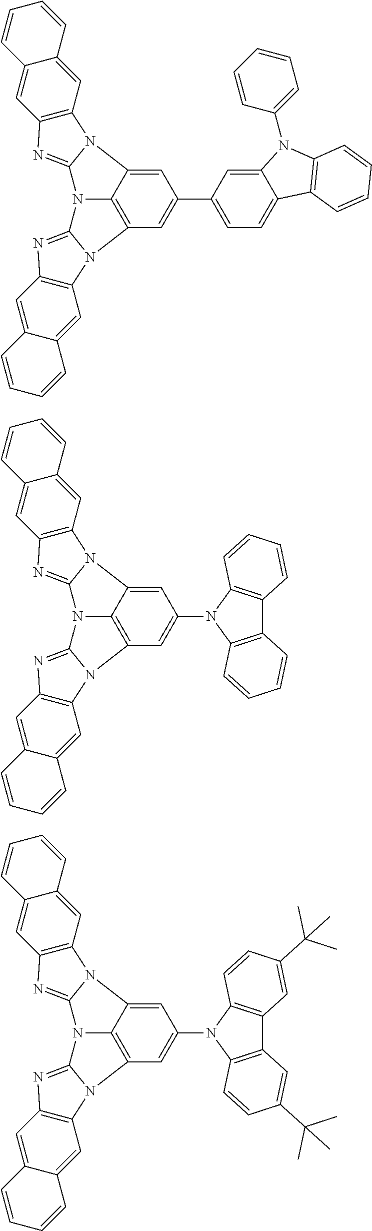

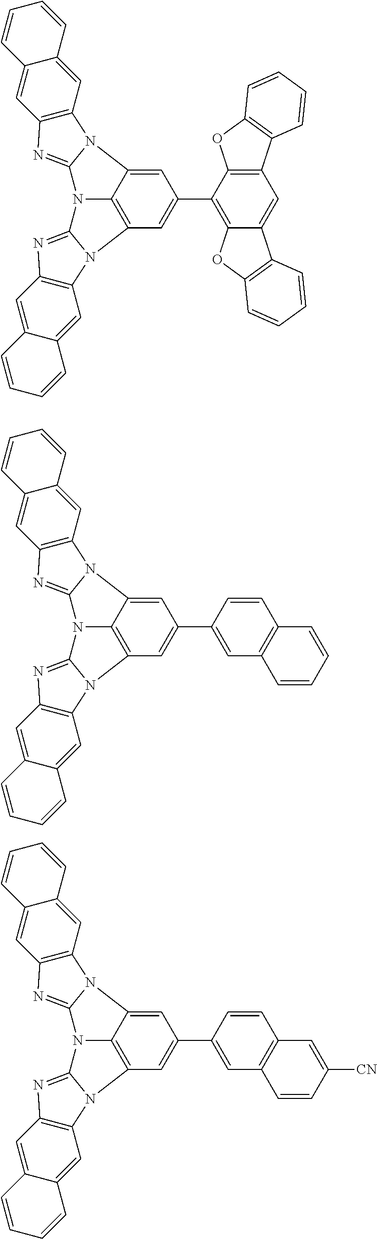

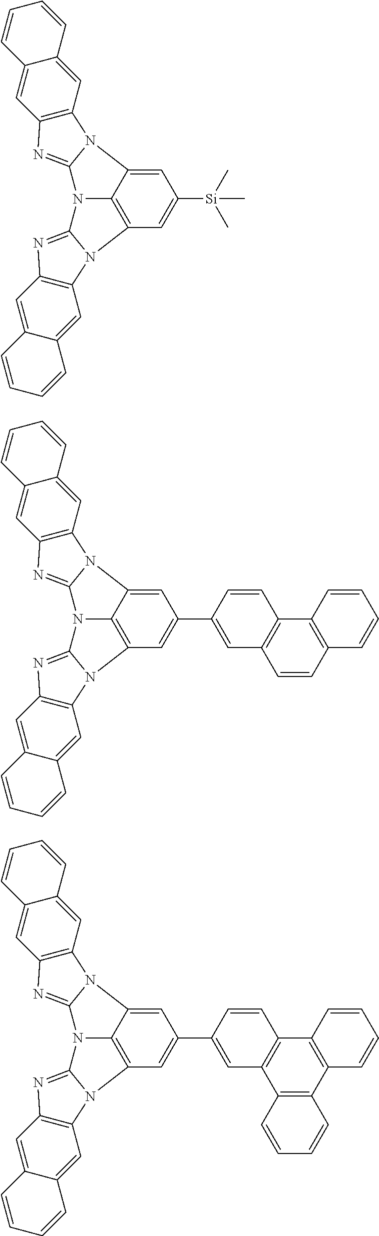

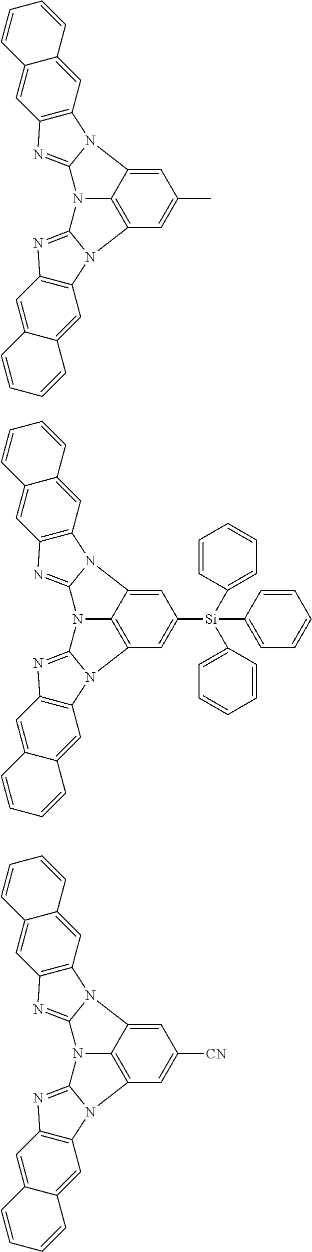

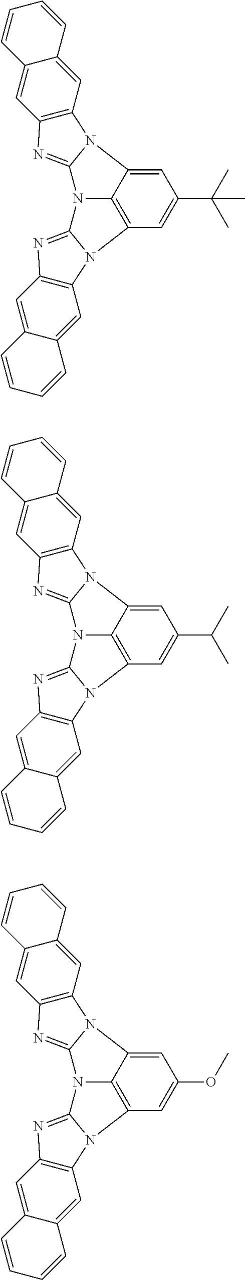

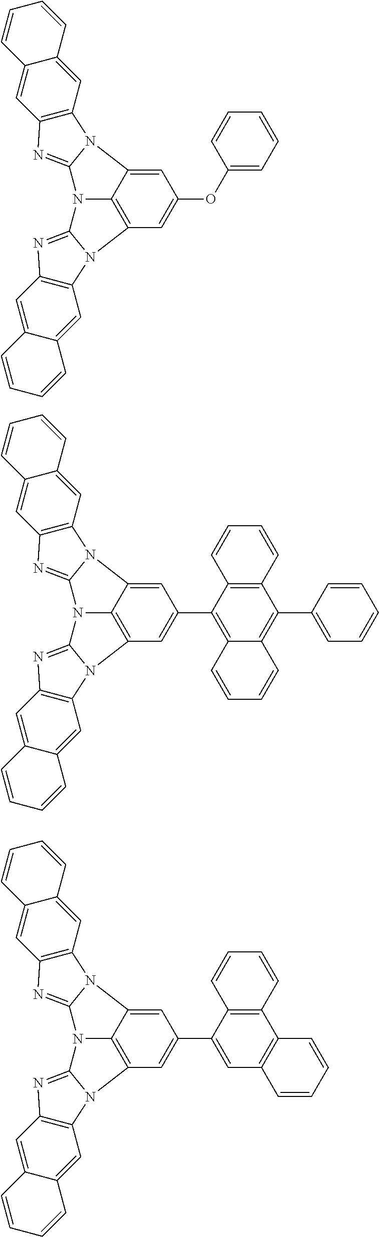

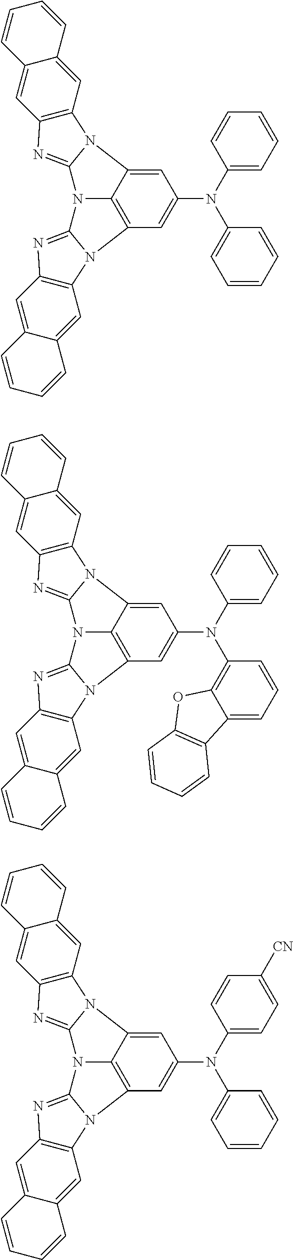

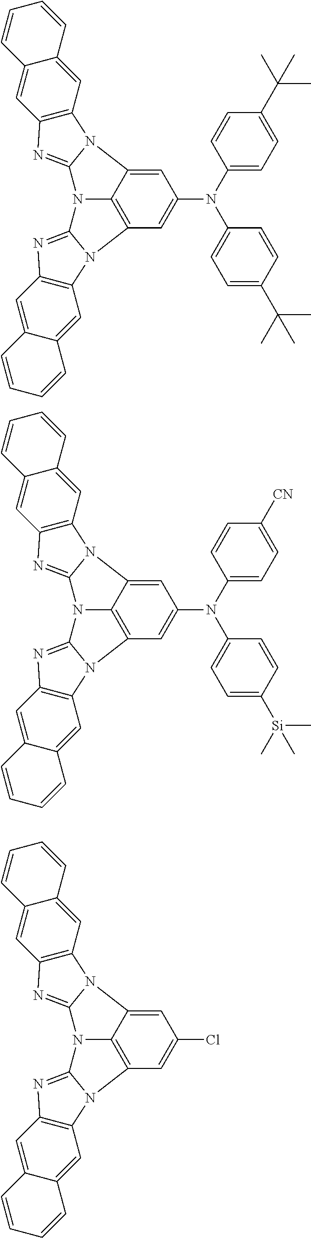

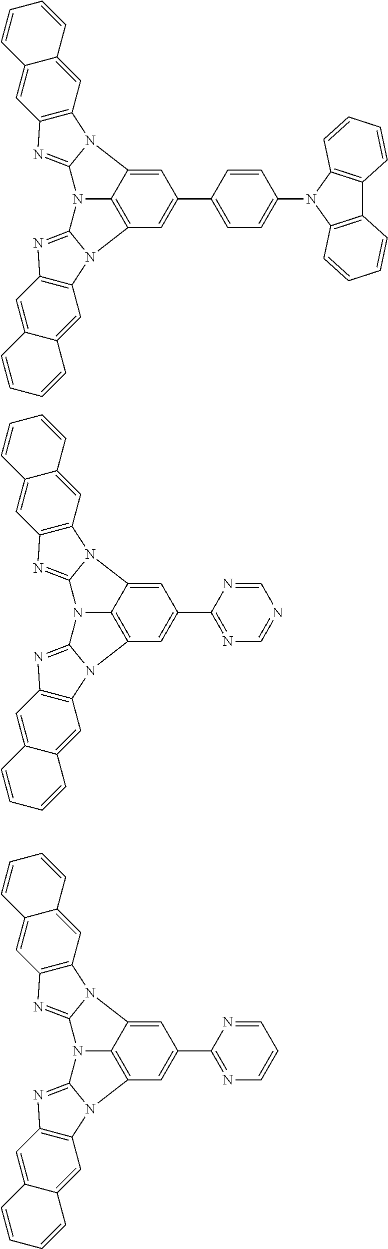

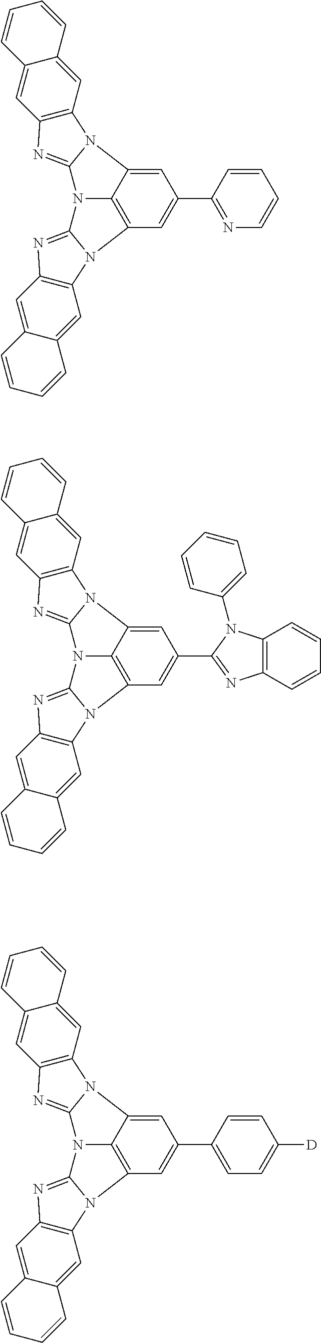 [SMILES: c1ccc(-c2cc3c4c(c2)n2c5cc6ccccc6cc5nc2n4c2nc4cc5ccccc5cc4n32)nc1 ; c1ccc(-n2c(-c3cc4c5c(c3)n3c6cc7ccccc7cc6nc3n5c3nc5cc6ccccc6cc5n43)nc3ccccc32)cc1 ; [2H]c1ccc(-c2cc3c4c(c2)n2c5cc6ccccc6cc5nc2n4c2nc4cc5ccccc5cc4n32)cc1]

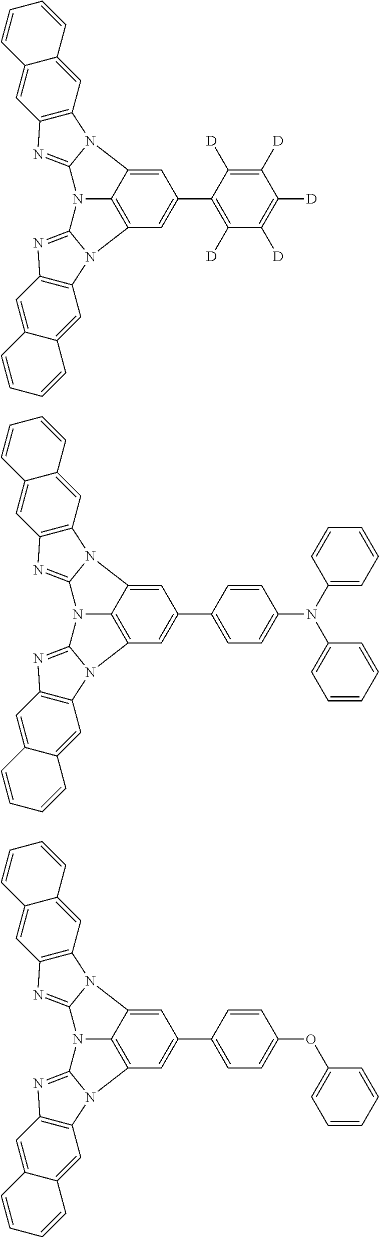

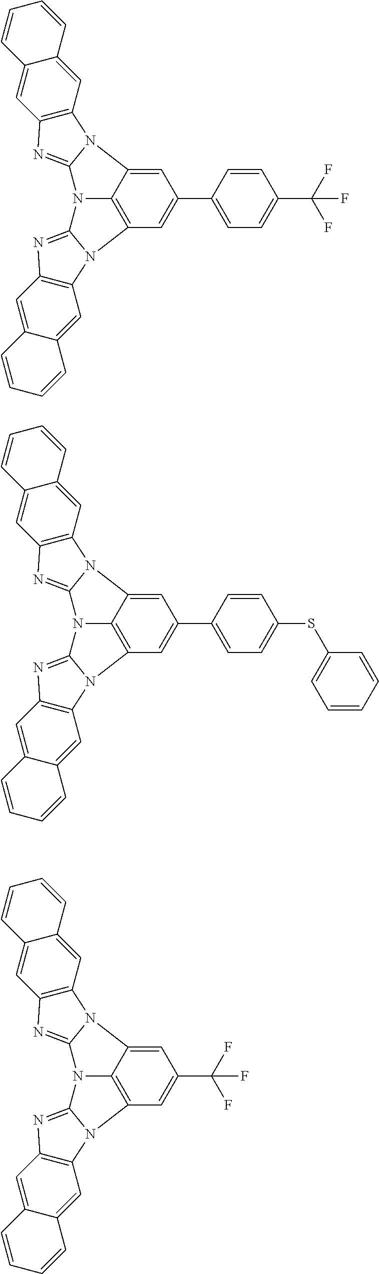

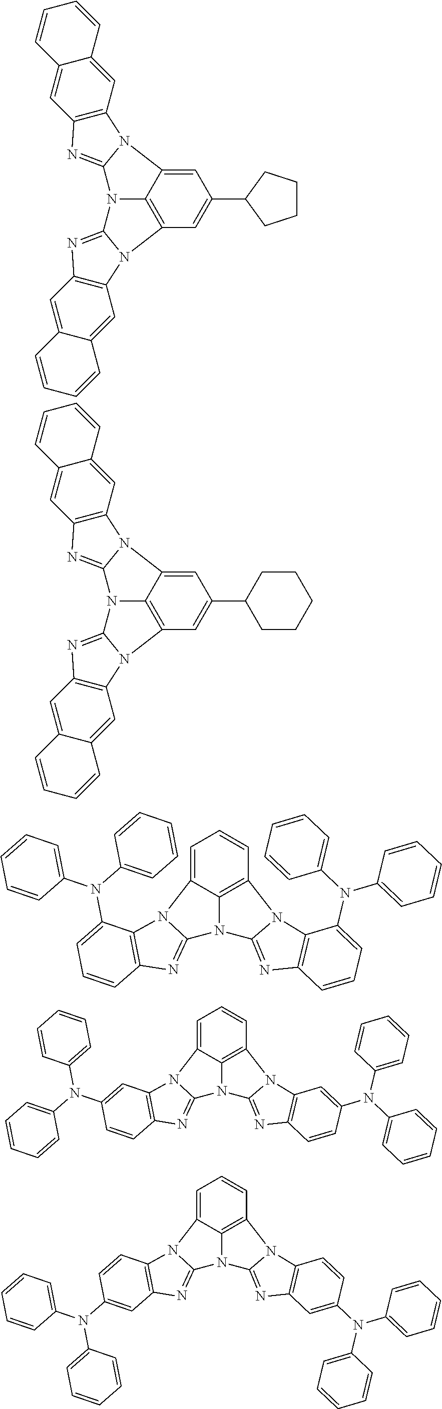

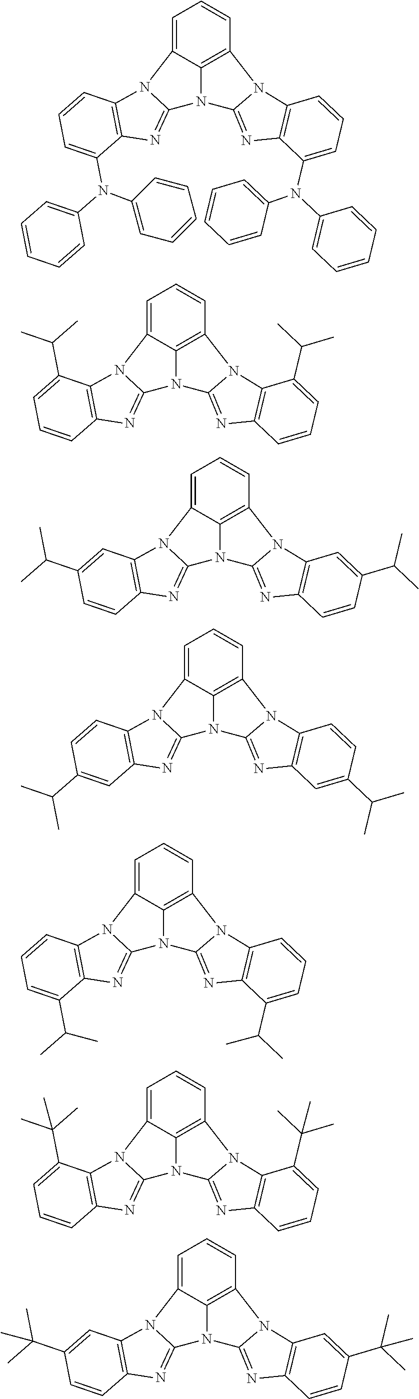

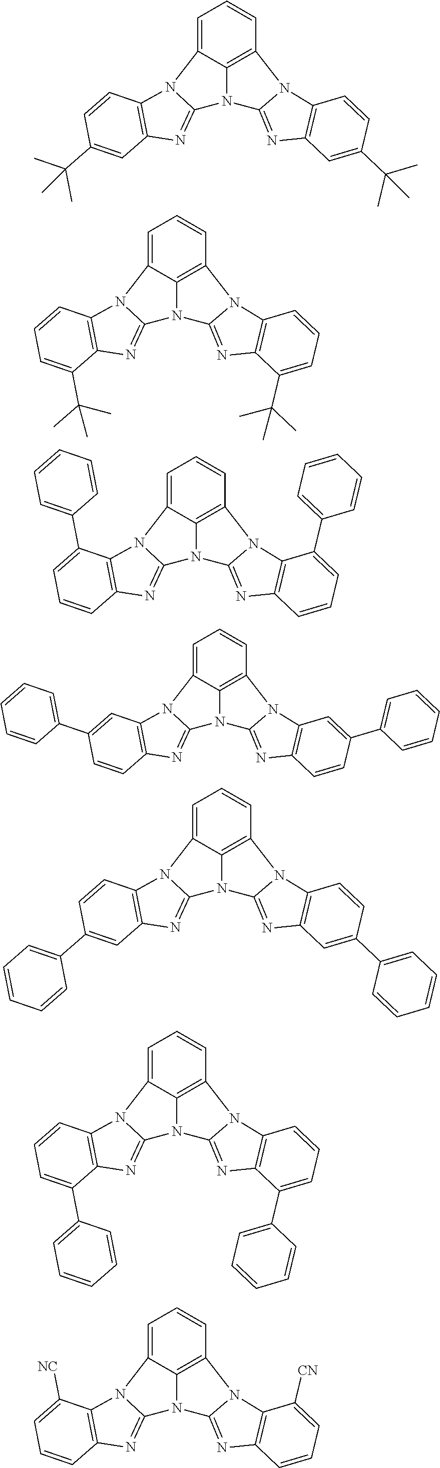

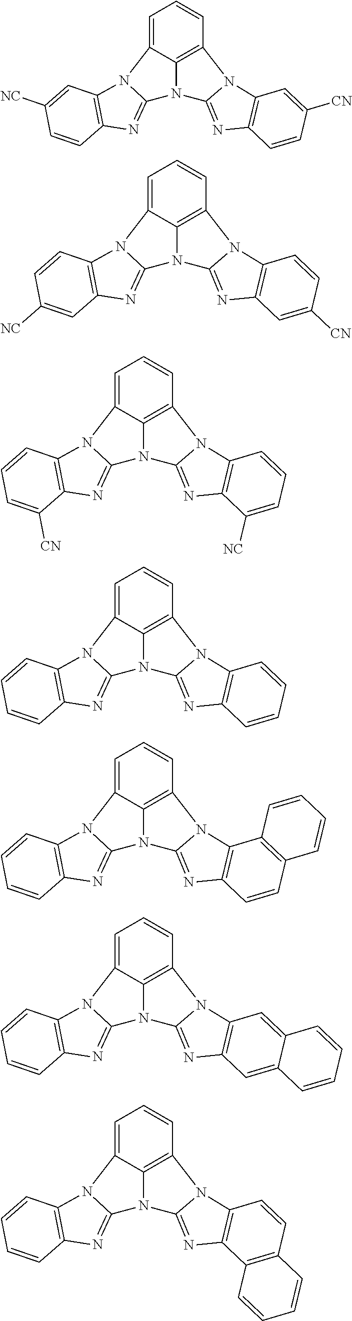

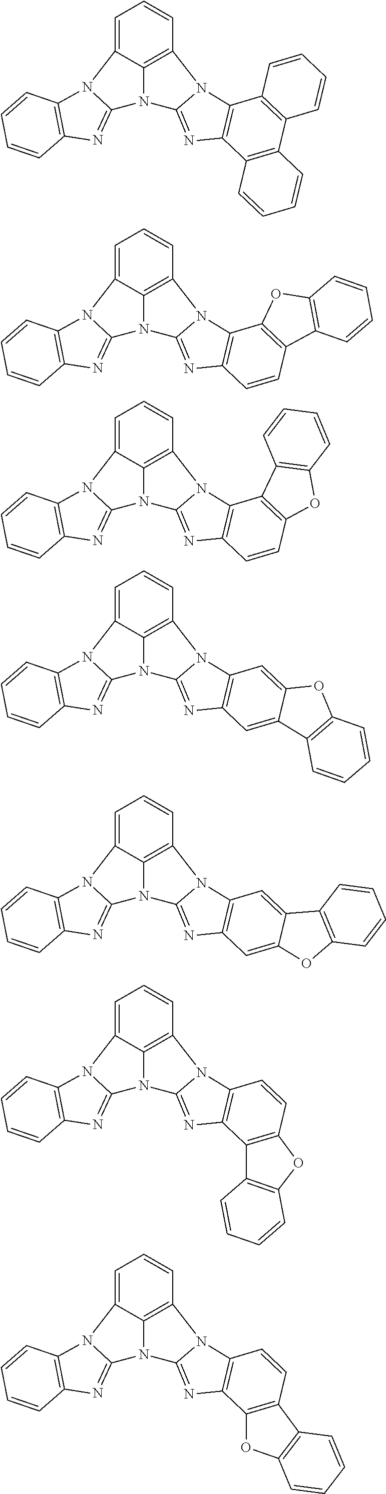

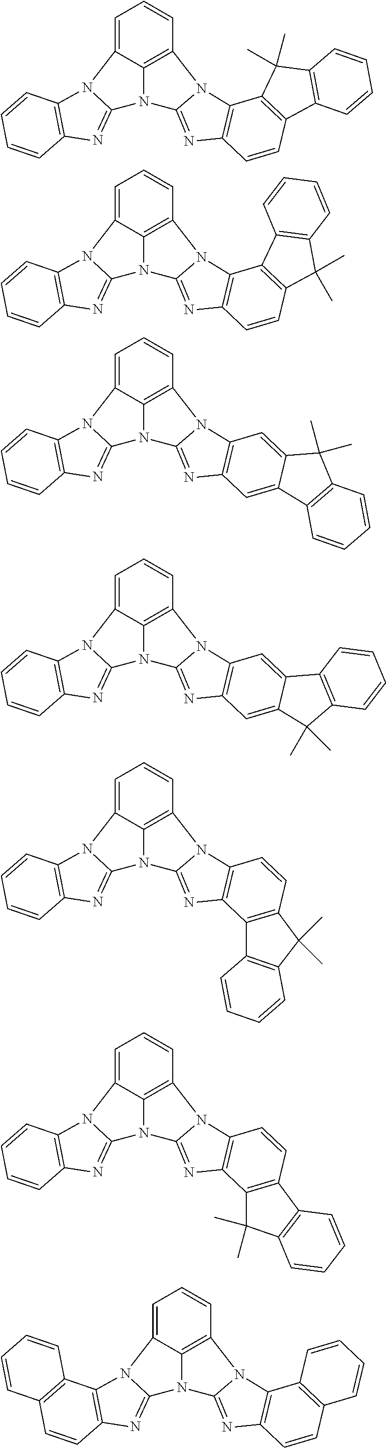

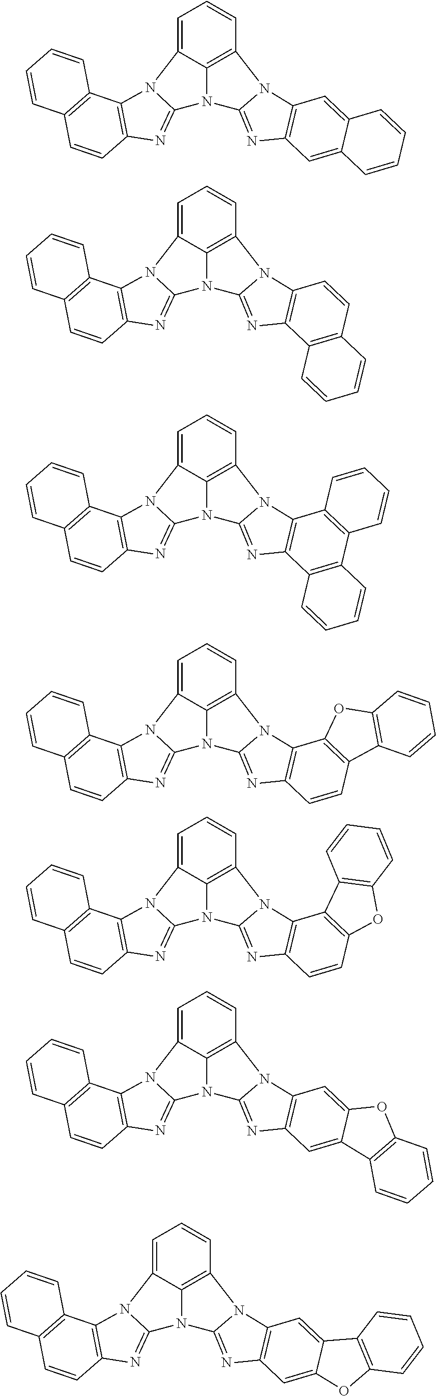

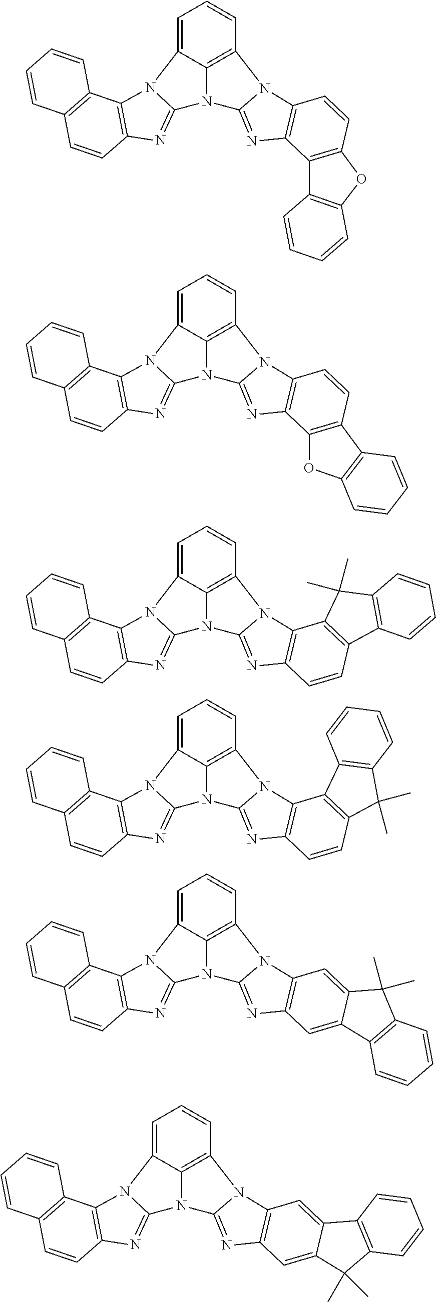

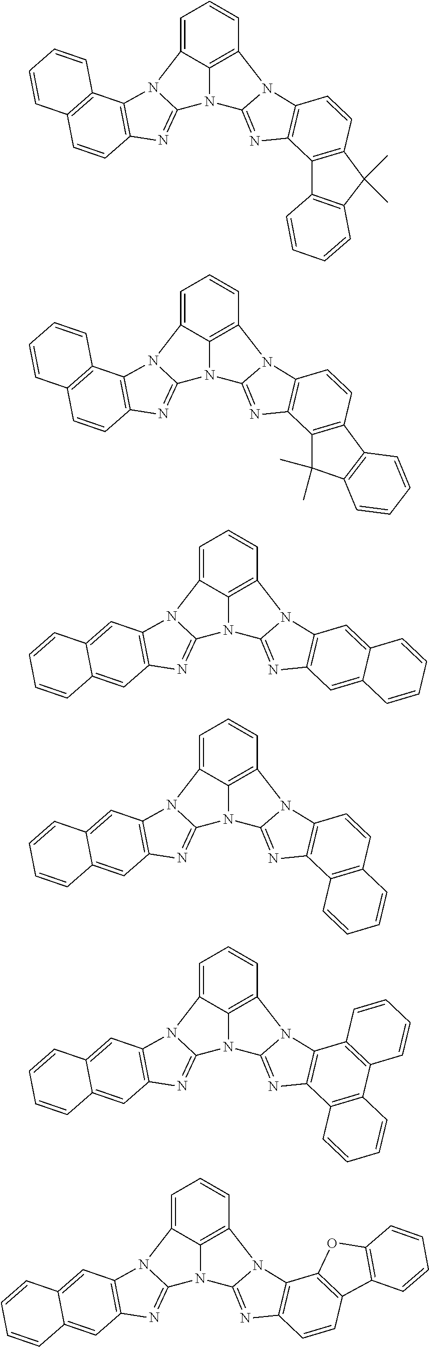 [SMILES: CC1(C)c2ccccc2-c2c1ccc1c2nc2n1c1cccc3c1n2c1nc2ccc4ccccc4c2n31 ; CC1(C)c2ccccc2-c2ccc3c(nc4n3c3cccc5c3n4c3nc4ccc6ccccc6c4n53)c21 ; c1ccc2cc3c(cc2c1)nc1n3c2cccc3c2n1c1nc2cc4ccccc4cc2n31 ; c1ccc2cc3c(cc2c1)nc1n3c2cccc3c2n1c1nc2c4ccccc4ccc2n31 ; c1ccc2cc3c(cc2c1)nc1n3c2cccc3c2n1c1nc2c4ccccc4c4ccccc4c2n31 ; c1ccc2cc3c(cc2c1)nc1n3c2cccc3c2n1c1nc2ccc4c5ccccc5oc4c2n31]

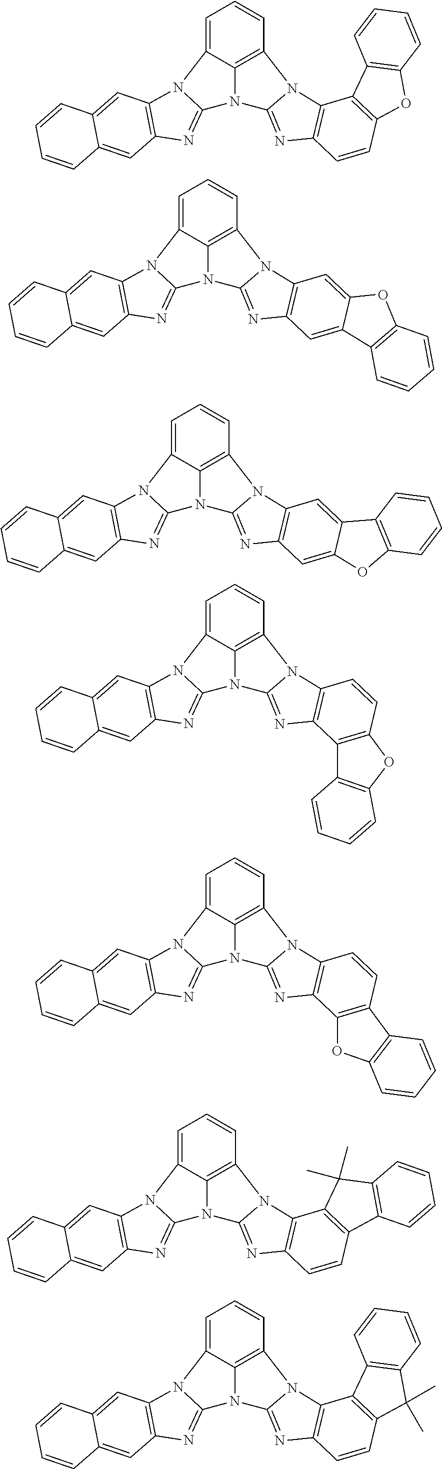

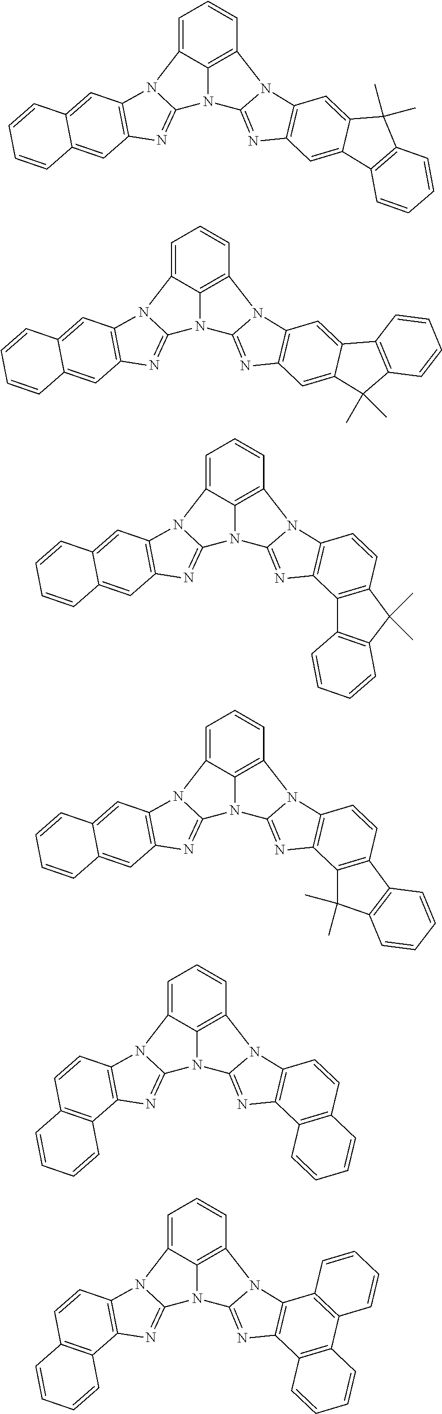 [SMILES: CC1(C)c2ccccc2-c2cc3nc4n(c3cc21)c1cccc2c1n4c1nc3cc4ccccc4cc3n21 ; CC1(C)c2ccccc2-c2cc3c(cc21)nc1n3c2cccc3c2n1c1nc2cc4ccccc4cc2n31 ; CC1(C)c2ccccc2-c2c1ccc1c2nc2n1c1cccc3c1n2c1nc2cc4ccccc4cc2n31 ; CC1(C)c2ccccc2-c2ccc3c(nc4n3c3cccc5c3n4c3nc4cc6ccccc6cc4n53)c21 ; c1ccc2c(c1)ccc1c2nc2n1c1cccc3c1n2c1nc2c4ccccc4ccc2n31 ; c1ccc2c(c1)ccc1c2nc2n1c1cccc3c1n2c1nc2c4ccccc4c4ccccc4c2n31]

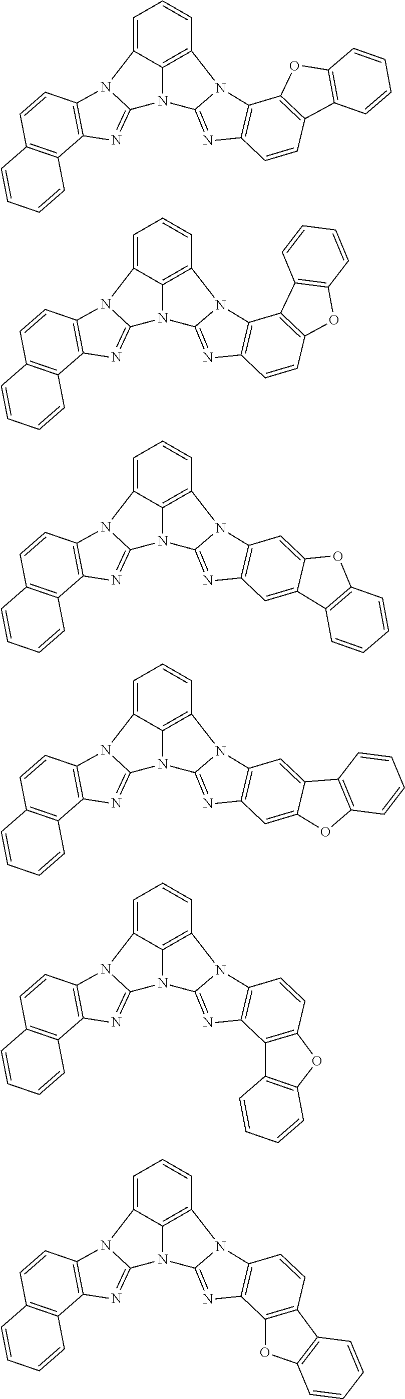 [SMILES: c1ccc2c(c1)ccc1c2nc2n1c1cccc3c1n2c1nc2ccc4c5ccccc5oc4c2n31 ; c1ccc2c(c1)ccc1c2nc2n1c1cccc3c1n2c1nc2ccc4oc5ccccc5c4c2n31 ; c1ccc2c(c1)ccc1c2nc2n1c1cccc3c1n2c1nc2cc4c(cc2n31)oc1ccccc14 ; c1ccc2c(c1)ccc1c2nc2n1c1cccc3c1n2c1nc2cc4oc5ccccc5c4cc2n31 ; c1ccc2c(c1)ccc1c2nc2n1c1cccc3c1n2c1nc2c4c(ccc2n31)oc1ccccc14 ; c1ccc2c(c1)ccc1c2nc2n1c1cccc3c1n2c1nc2c4oc5ccccc5c4ccc2n31]

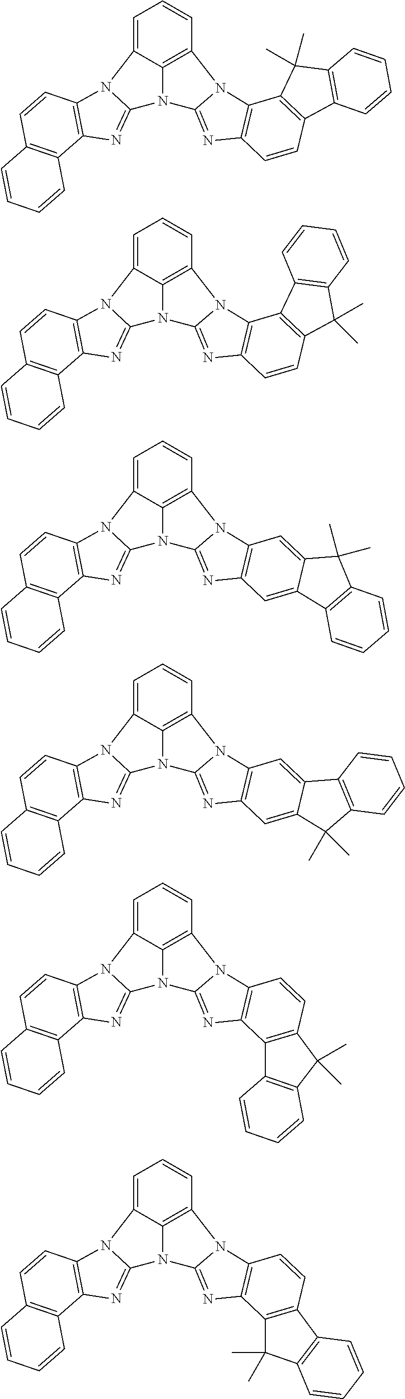 [SMILES: CC1(C)c2ccccc2-c2ccc3nc4n(c5cccc6c5n4c4nc5c7ccccc7ccc5n64)c3c21 ; CC1(C)c2ccccc2-c2c1ccc1nc3n(c4cccc5c4n3c3nc4c6ccccc6ccc4n53)c21 ; CC1(C)c2ccccc2-c2cc3nc4n(c3cc21)c1cccc2c1n4c1nc3c4ccccc4ccc3n21 ; CC1(C)c2ccccc2-c2cc3c(cc21)nc1n3c2cccc3c2n1c1nc2c4ccccc4ccc2n31 ; CC1(C)c2ccccc2-c2c1ccc1c2nc2n1c1cccc3c1n2c1nc2c4ccccc4ccc2n31 ; CC1(C)c2ccccc2-c2ccc3c(nc4n3c3cccc5c3n4c3nc4c6ccccc6ccc4n53)c21]

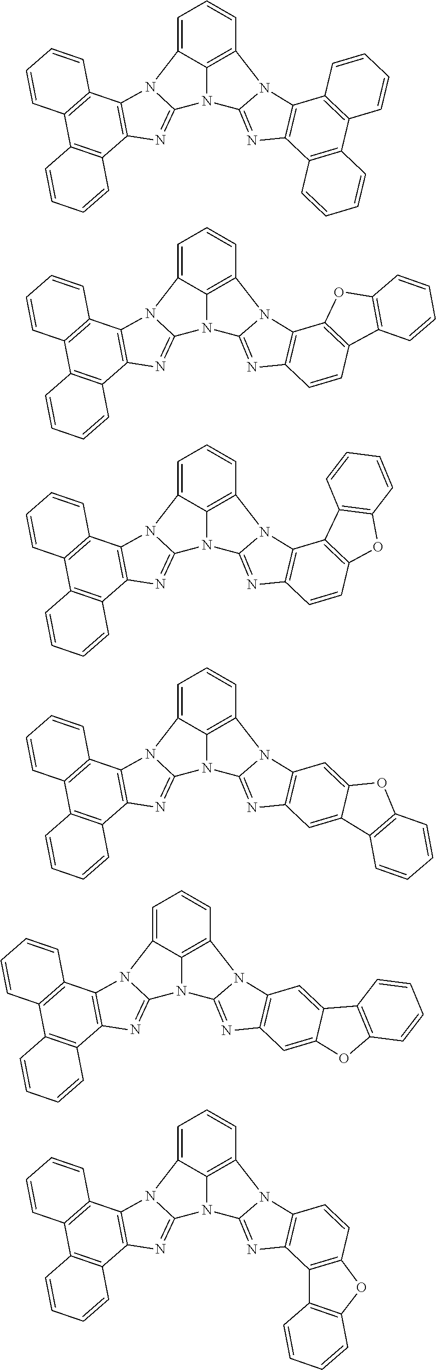 [SMILES: c1ccc2c(c1)c1ccccc1c1c2nc2n1c1cccc3c1n2c1nc2c4ccccc4c4ccccc4c2n31 ; c1ccc2c(c1)oc1c2ccc2nc3n(c4cccc5c4n3c3nc4c6ccccc6c6ccccc6c4n53)c21 ; c1ccc2c(c1)oc1ccc3nc4n(c5cccc6c5n4c4nc5c7ccccc7c7ccccc7c5n64)c3c12 ; c1ccc2c(c1)oc1cc3c(cc12)nc1n3c2cccc3c2n1c1nc2c4ccccc4c4ccccc4c2n31 ; c1ccc2c(c1)oc1cc3nc4n(c3cc12)c1cccc2c1n4c1nc3c4ccccc4c4ccccc4c3n21 ; c1ccc2c(c1)oc1ccc3c(nc4n3c3cccc5c3n4c3nc4c6ccccc6c6ccccc6c4n53)c12]

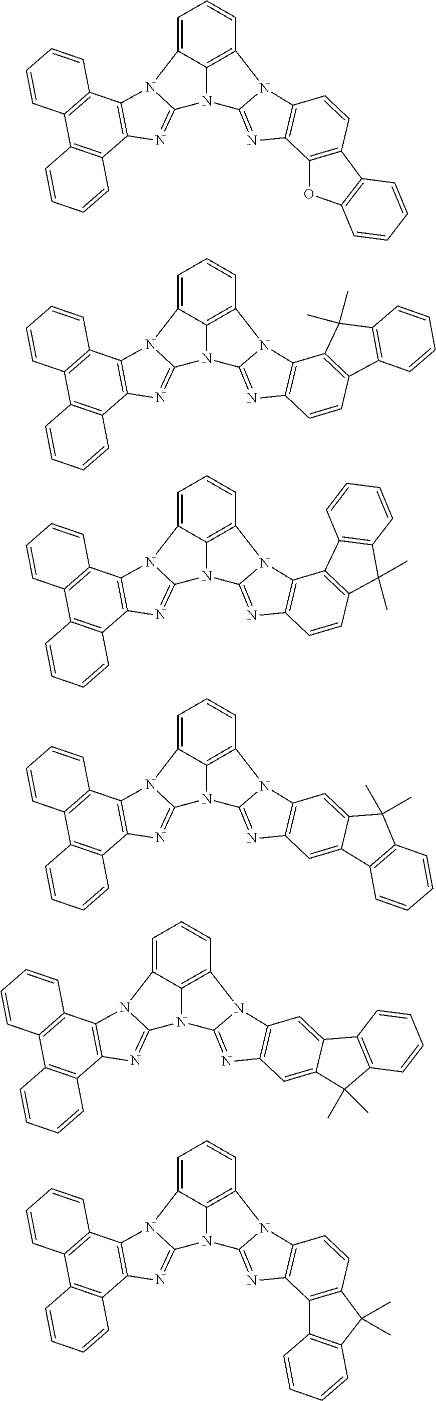 [SMILES: c1ccc2c(c1)oc1c2ccc2c1nc1n2c2cccc3c2n1c1nc2c4ccccc4c4ccccc4c2n31 ; CC1(C)c2ccccc2-c2ccc3nc4n(c5cccc6c5n4c4nc5c7ccccc7c7ccccc7c5n64)c3c21 ; CC1(C)c2ccccc2-c2c1ccc1nc3n(c4cccc5c4n3c3nc4c6ccccc6c6ccccc6c4n53)c21 ; CC1(C)c2ccccc2-c2cc3nc4n(c3cc21)c1cccc2c1n4c1nc3c4ccccc4c4ccccc4c3n21 ; CC1(C)c2ccccc2-c2cc3c(cc21)nc1n3c2cccc3c2n1c1nc2c4ccccc4c4ccccc4c2n31 ; CC1(C)c2ccccc2-c2c1ccc1c2nc2n1c1cccc3c1n2c1nc2c4ccccc4c4ccccc4c2n31]

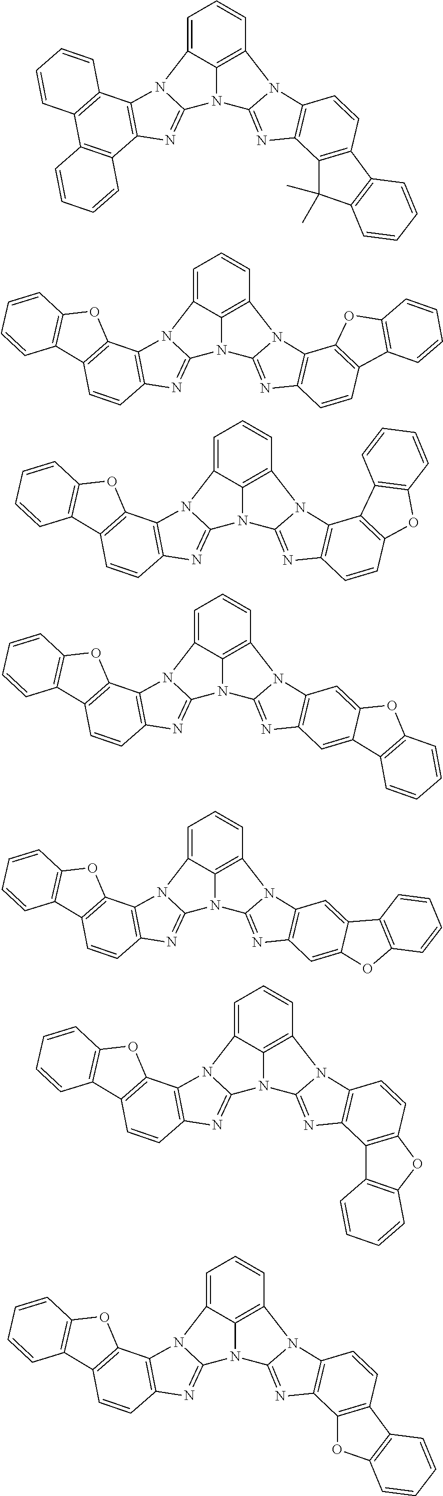

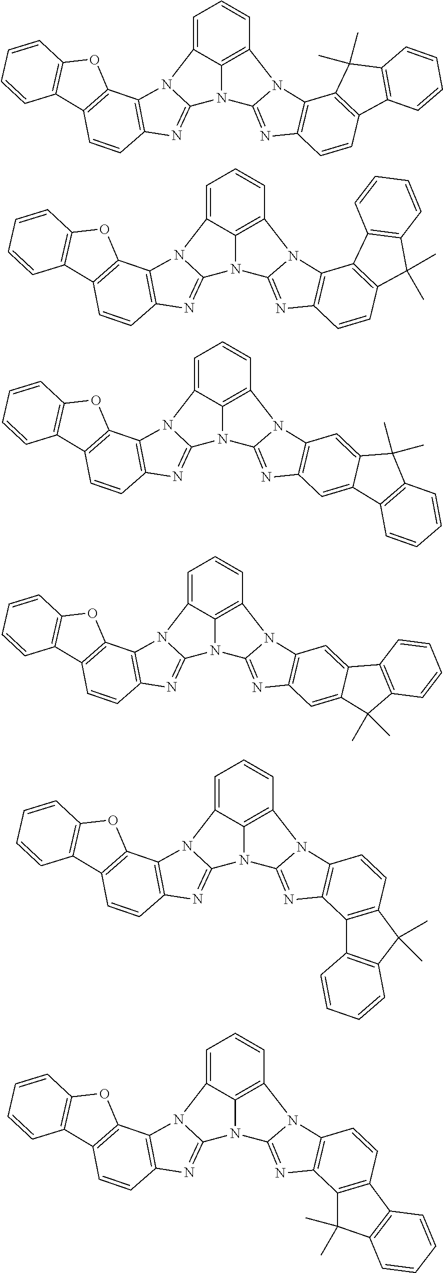

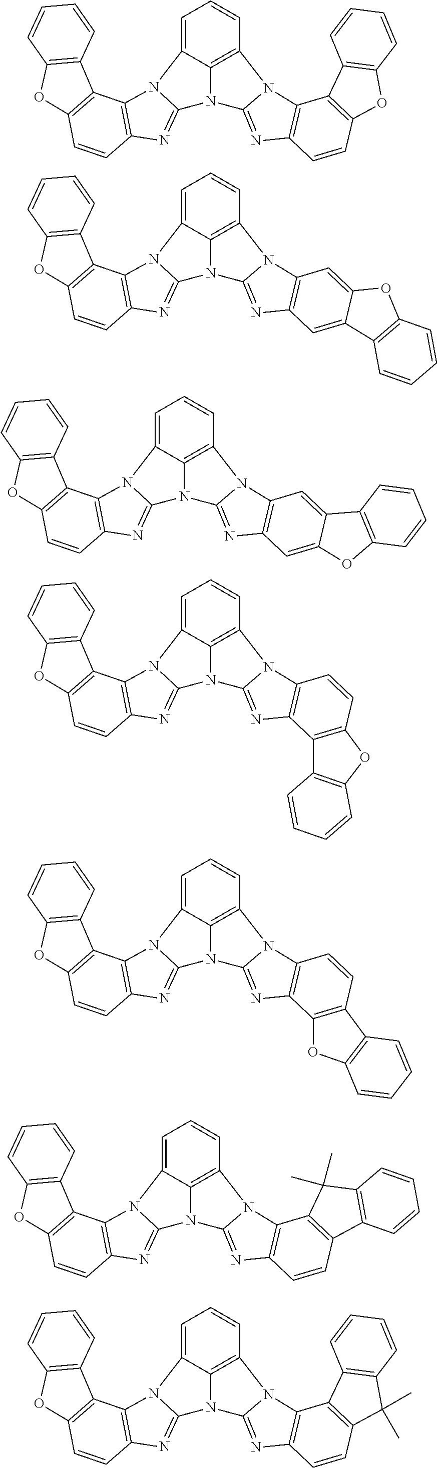

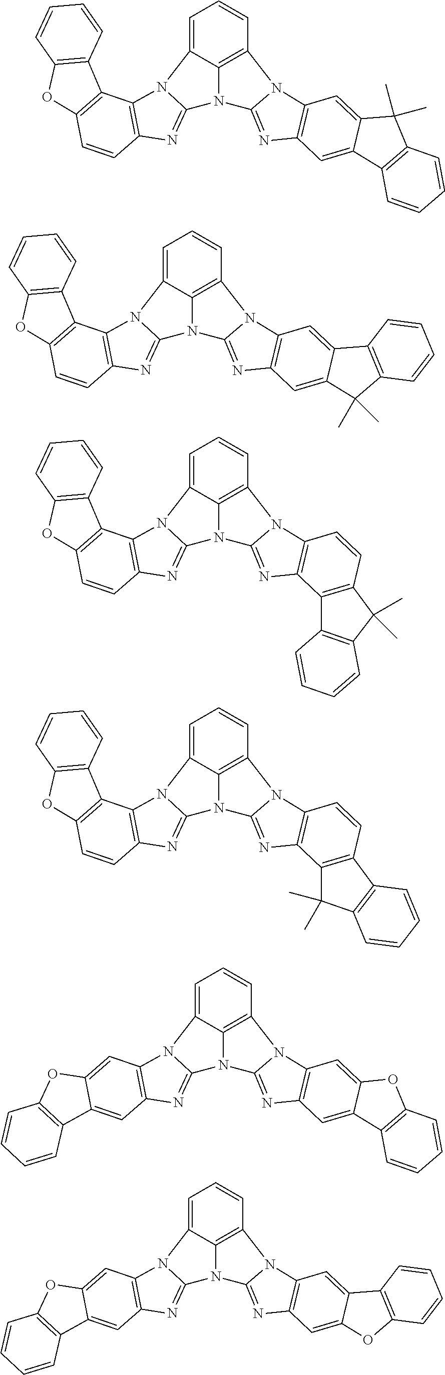 [SMILES: CC1(C)c2ccccc2-c2cc3nc4n(c3cc21)c1cccc2c1n4c1nc3ccc4oc5ccccc5c4c3n21 ; CC1(C)c2ccccc2-c2cc3c(cc21)nc1n3c2cccc3c2n1c1nc2ccc4oc5ccccc5c4c2n31 ; CC1(C)c2ccccc2-c2c1ccc1c2nc2n1c1cccc3c1n2c1nc2ccc4oc5ccccc5c4c2n31 ; CC1(C)c2ccccc2-c2ccc3c(nc4n3c3cccc5c3n4c3nc4ccc6oc7ccccc7c6c4n53)c21 ; c1ccc2c(c1)oc1cc3c(cc12)nc1n3c2cccc3c2n1c1nc2cc4c(cc2n31)oc1ccccc14 ; c1ccc2c(c1)oc1cc3c(cc12)nc1n3c2cccc3c2n1c1nc2cc4oc5ccccc5c4cc2n31]

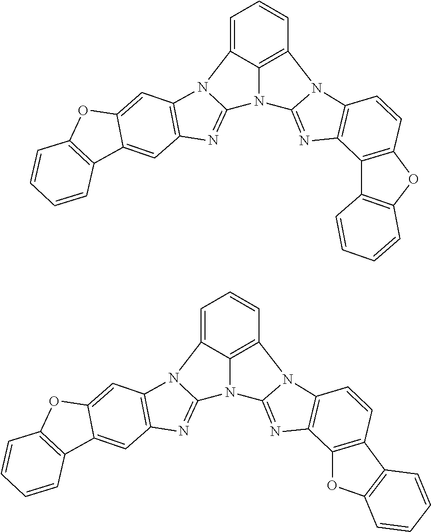 [SMILES: c1ccc2c(c1)oc1cc3c(cc12)nc1n3c2cccc3c2n1c1nc2c4c(ccc2n31)oc1ccccc14 ; c1ccc2c(c1)oc1cc3c(cc12)nc1n3c2cccc3c2n1c1nc2c4oc5ccccc5c4ccc2n31]

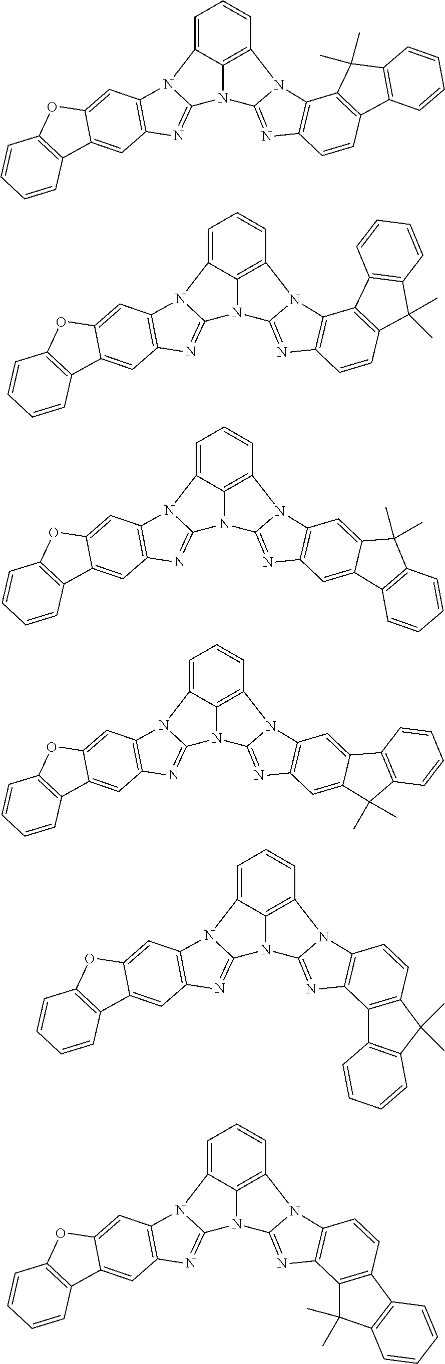 [SMILES: CC1(C)c2ccccc2-c2ccc3nc4n(c5cccc6c5n4c4nc5cc7c(cc5n64)oc4ccccc47)c3c21 ; CC1(C)c2ccccc2-c2c1ccc1nc3n(c4cccc5c4n3c3nc4cc6c(cc4n53)oc3ccccc36)c21 ; CC1(C)c2ccccc2-c2cc3nc4n(c3cc21)c1cccc2c1n4c1nc3cc4c(cc3n21)oc1ccccc14 ; CC1(C)c2ccccc2-c2cc3c(cc21)nc1n3c2cccc3c2n1c1nc2cc4c(cc2n31)oc1ccccc14 ; CC1(C)c2ccccc2-c2c1ccc1c2nc2n1c1cccc3c1n2c1nc2cc4c(cc2n31)oc1ccccc14 ; CC1(C)c2ccccc2-c2ccc3c(nc4n3c3cccc5c3n4c3nc4cc6c(cc4n53)oc3ccccc36)c21]

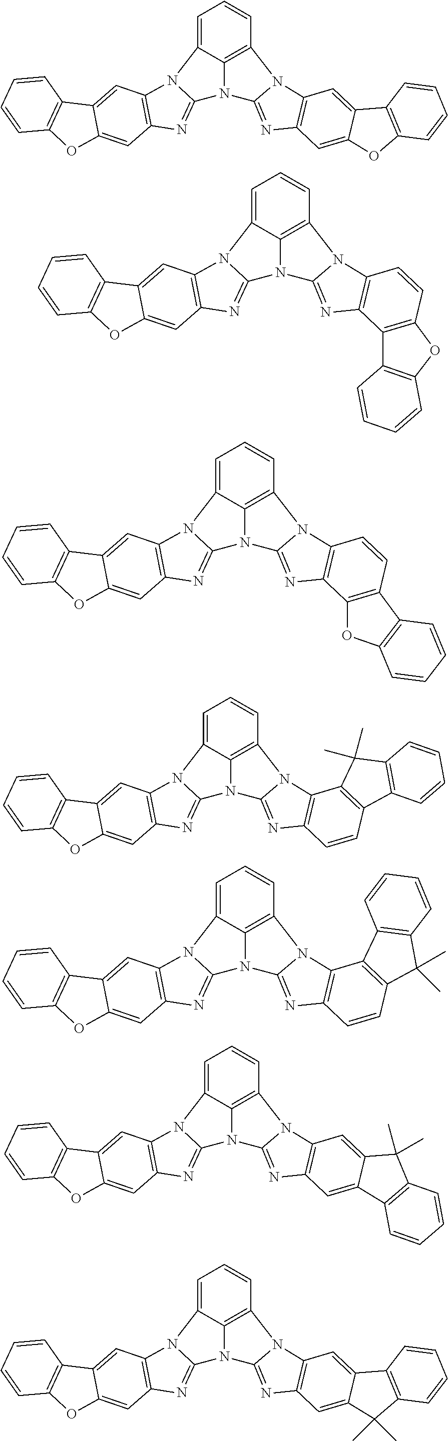

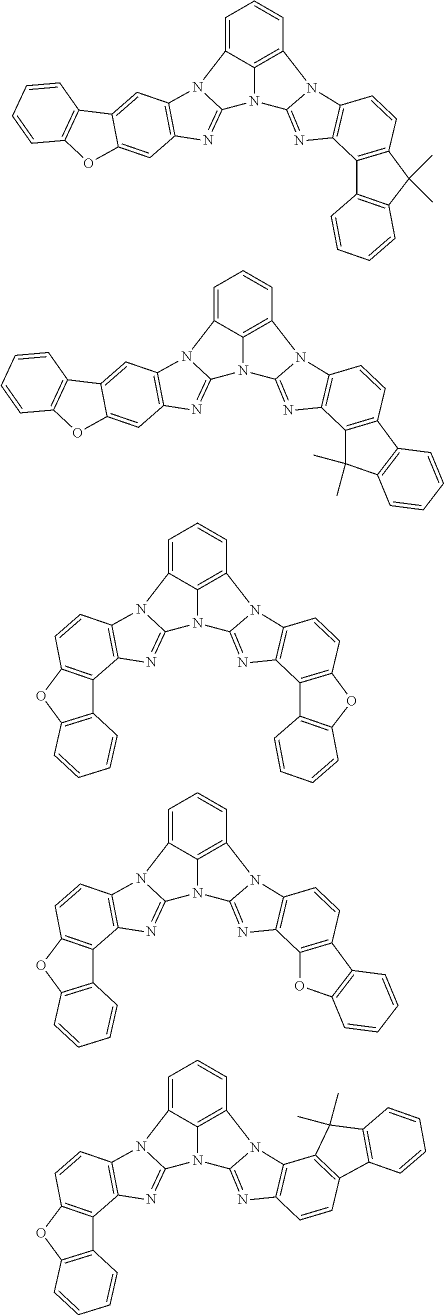 [SMILES: CC1(C)c2ccccc2-c2c1ccc1c2nc2n1c1cccc3c1n2c1nc2cc4oc5ccccc5c4cc2n31 ; CC1(C)c2ccccc2-c2ccc3c(nc4n3c3cccc5c3n4c3nc4cc6oc7ccccc7c6cc4n53)c21 ; c1ccc2c(c1)oc1ccc3c(nc4n3c3cccc5c3n4c3nc4c6c(ccc4n53)oc3ccccc36)c12 ; c1ccc2c(c1)oc1c2ccc2c1nc1n2c2cccc3c2n1c1nc2c4c(ccc2n31)oc1ccccc14 ; CC1(C)c2ccccc2-c2ccc3nc4n(c5cccc6c5n4c4nc5c7c(ccc5n64)oc4ccccc47)c3c21]

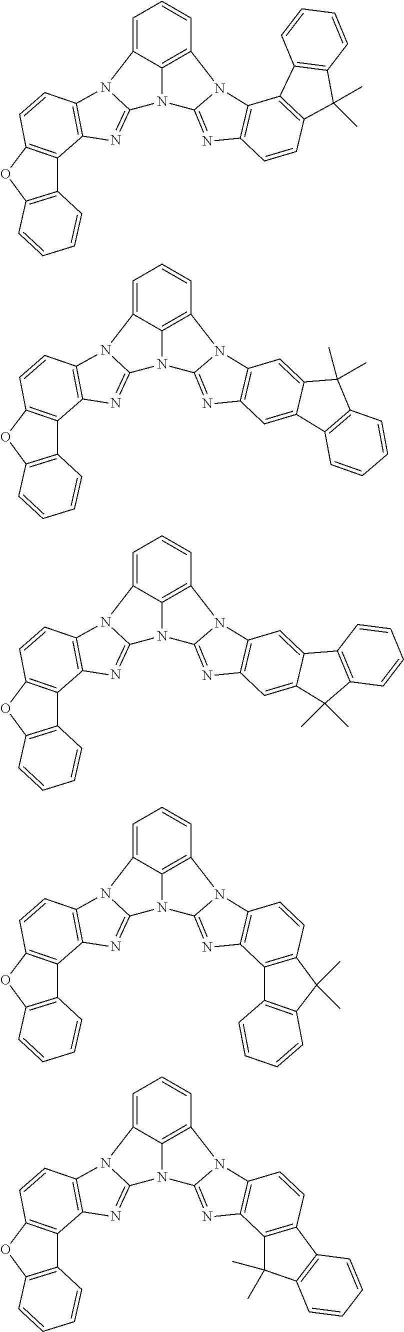 [SMILES: CC1(C)c2ccccc2-c2c1ccc1nc3n(c4cccc5c4n3c3nc4c6c(ccc4n53)oc3ccccc36)c21 ; CC1(C)c2ccccc2-c2cc3nc4n(c3cc21)c1cccc2c1n4c1nc3c4c(ccc3n21)oc1ccccc14 ; CC1(C)c2ccccc2-c2cc3c(cc21)nc1n3c2cccc3c2n1c1nc2c4c(ccc2n31)oc1ccccc14 ; CC1(C)c2ccccc2-c2c1ccc1c2nc2n1c1cccc3c1n2c1nc2c4c(ccc2n31)oc1ccccc14 ; CC1(C)c2ccccc2-c2ccc3c(nc4n3c3cccc5c3n4c3nc4c6c(ccc4n53)oc3ccccc36)c21]

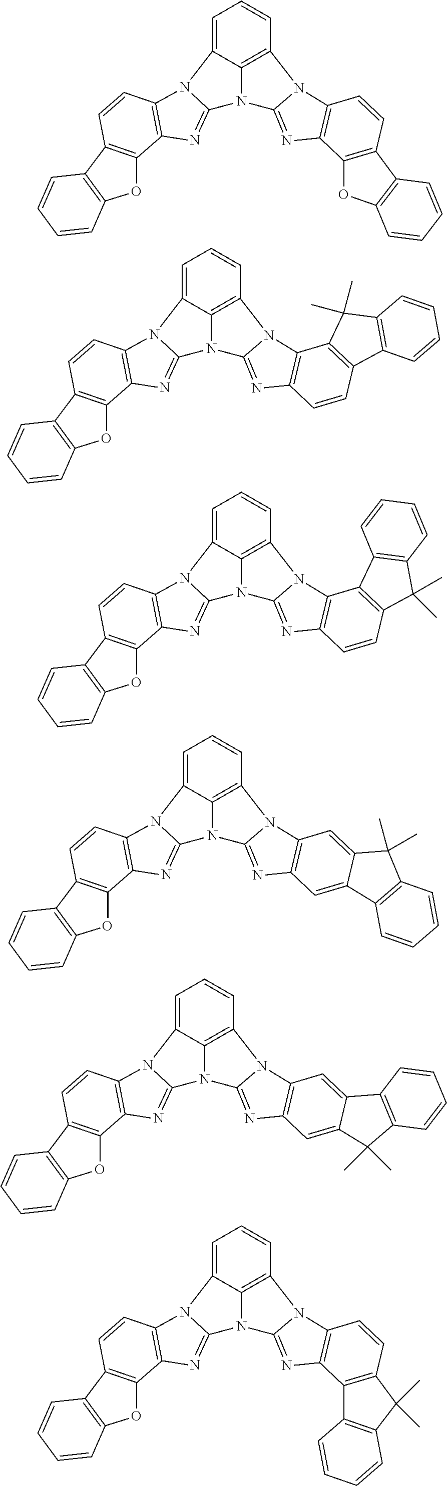 [SMILES: c1ccc2c(c1)oc1c2ccc2c1nc1n2c2cccc3c2n1c1nc2c4oc5ccccc5c4ccc2n31 ; CC1(C)c2ccccc2-c2ccc3nc4n(c5cccc6c5n4c4nc5c7oc8ccccc8c7ccc5n64)c3c21 ; CC1(C)c2ccccc2-c2c1ccc1nc3n(c4cccc5c4n3c3nc4c6oc7ccccc7c6ccc4n53)c21 ; CC1(C)c2ccccc2-c2cc3nc4n(c3cc21)c1cccc2c1n4c1nc3c4oc5ccccc5c4ccc3n21 ; CC1(C)c2ccccc2-c2cc3c(cc21)nc1n3c2cccc3c2n1c1nc2c4oc5ccccc5c4ccc2n31 ; CC1(C)c2ccccc2-c2c1ccc1c2nc2n1c1cccc3c1n2c1nc2c4oc5ccccc5c4ccc2n31]

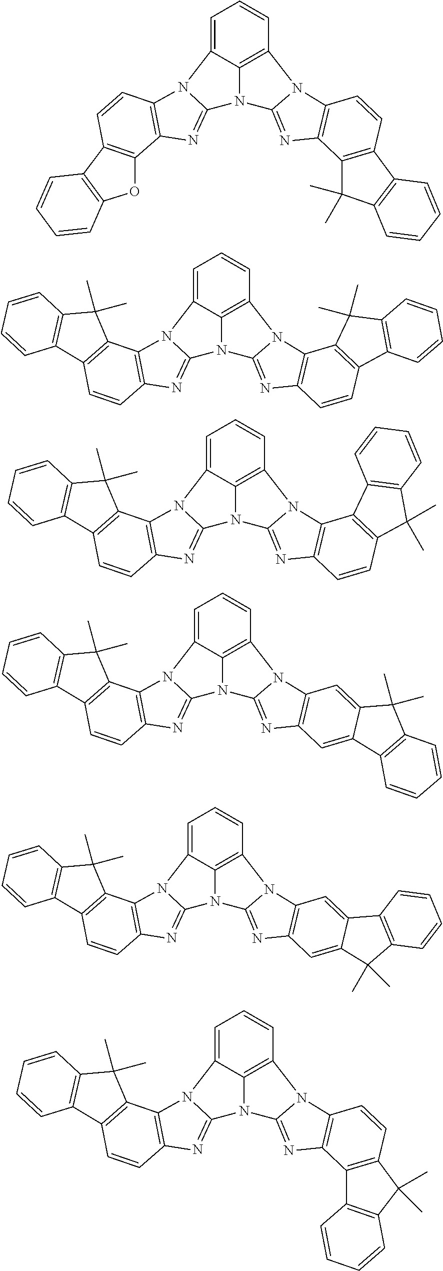

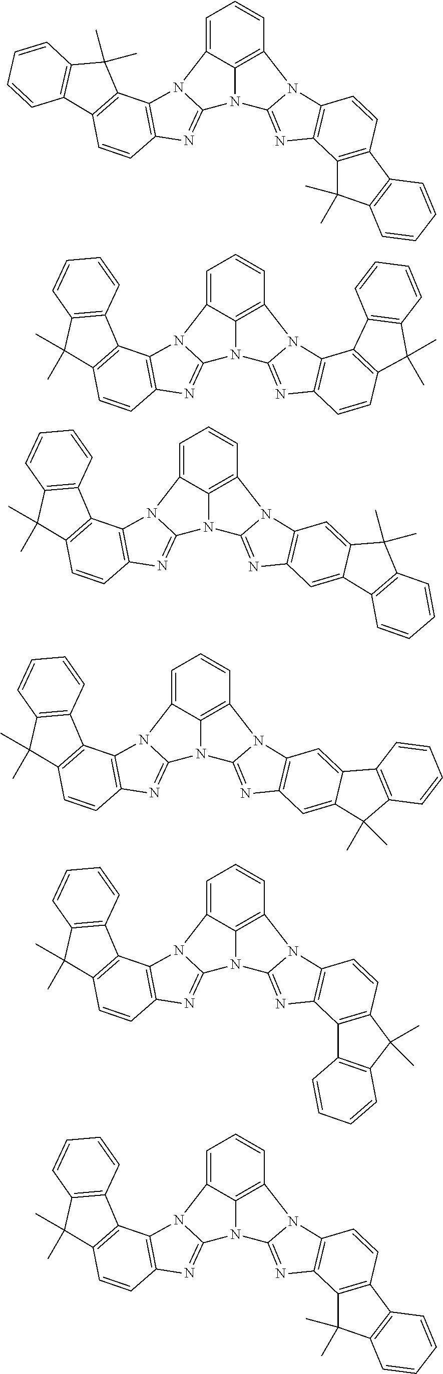

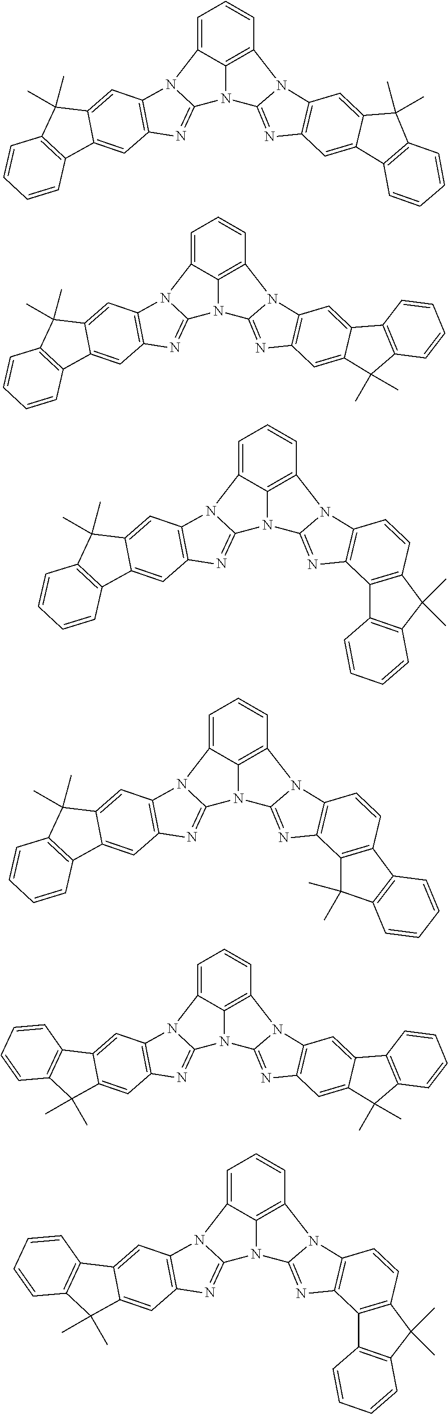 [SMILES: CC1(C)c2ccccc2-c2cc3nc4n(c3cc21)c1cccc2c1n4c1nc3cc4c(cc3n21)C(C)(C)c1ccccc1-4 ; CC1(C)c2ccccc2-c2cc3c(cc21)nc1n3c2cccc3c2n1c1nc2cc4c(cc2n31)C(C)(C)c1ccccc1-4 ; CC1(C)c2ccccc2-c2cc3nc4n(c3cc21)c1cccc2c1n4c1nc3c4c(ccc3n21)C(C)(C)c1ccccc1-4 ; CC1(C)c2ccccc2-c2cc3nc4n(c3cc21)c1cccc2c1n4c1nc3c4c(ccc3n21)-c1ccccc1C4(C)C ; CC1(C)c2ccccc2-c2cc3c(cc21)nc1n3c2cccc3c2n1c1nc2cc4c(cc2n31)-c1ccccc1C4(C)C ; CC1(C)c2ccccc2-c2cc3c(cc21)nc1n3c2cccc3c2n1c1nc2c4c(ccc2n31)C(C)(C)c1ccccc1-4]

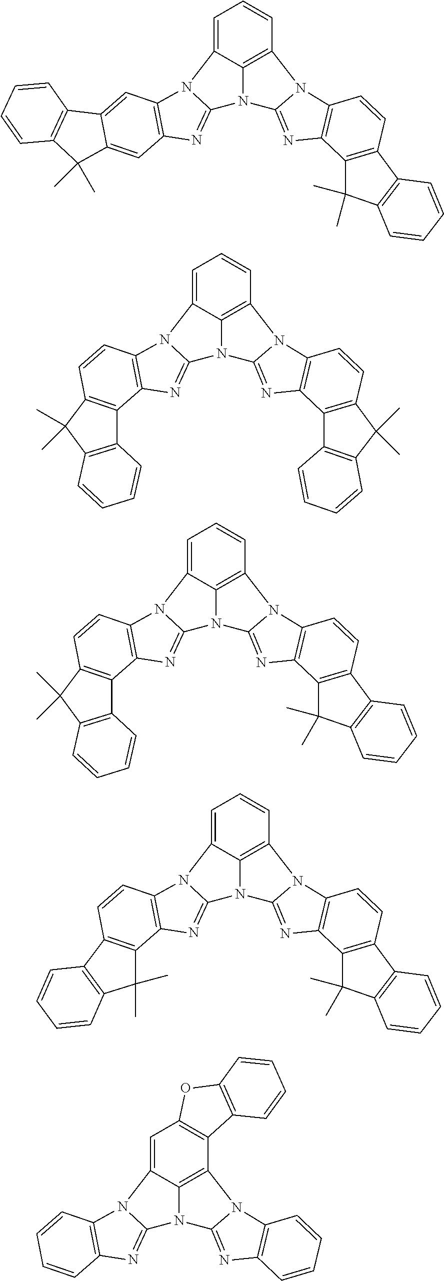

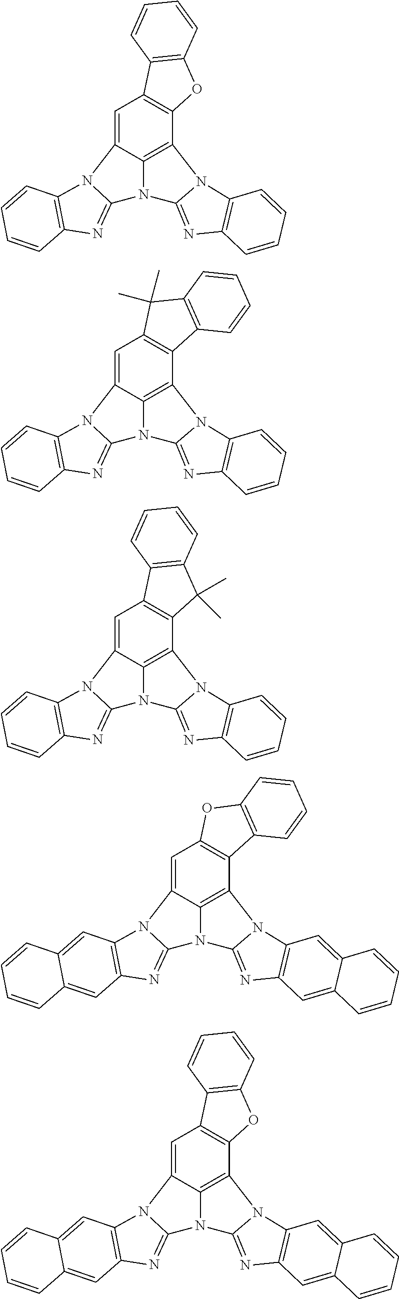

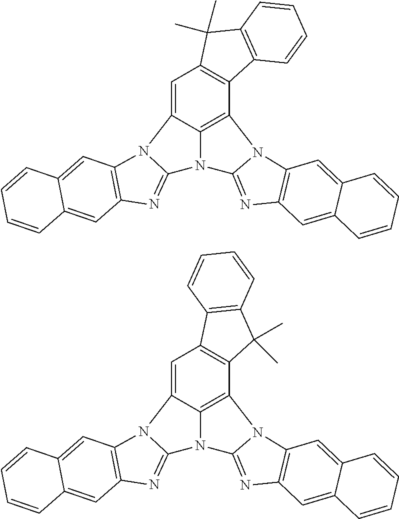 [SMILES: CC1(C)c2ccccc2-c2c1cc1c3c2n2c4cc5ccccc5cc4nc2n3c2nc3cc4ccccc4cc3n12 ; CC1(C)c2ccccc2-c2cc3c4c(c21)n1c2cc5ccccc5cc2nc1n4c1nc2cc4ccccc4cc2n31]

According to the compound of the first exemplary embodiment, at least one of the indices of performance of the organic EL device is improvable.

### Second Exemplary Embodiment

### Organic-EL-Device Material

An organic-EL-device material according to a second exemplary embodiment contains at least one of compounds according to the first exemplary embodiment.

The organic-EL-device material according to the second exemplary embodiment may further contain an additional compound. When the organic-EL-device material according to the second exemplary embodiment contains the additional compound, the additional compound may be a solid or a liquid.

### Third Exemplary Embodiment

### Organic Electroluminescence Device

An organic electroluminescence device (organic EL device) according to the third exemplary embodiment contains at least one of the compounds according to the first exemplary embodiment.

The organic EL device includes an anode, a cathode, and at least one organic layer between the anode and the cathode. The organic layer typically includes a plurality of layers formed of an organic compound(s). The organic layer may further include an inorganic compound.

The organic EL device according to the third exemplary embodiment includes the anode, the cathode and a first organic layer between the anode and the cathode. The first organic layer preferably contains at least one of the compounds according to the first exemplary embodiment. When the first organic layer contains at least one of the compounds according to the first exemplary embodiment, the first organic layer preferably contains the compound(s) according to the first exemplary embodiment in an amount of 45 mass % or less, more preferably 1 mass % or more and 20 mass % or less.

The first organic layer is, for instance, at least one layer selected from the group consisting of a hole injecting layer, a hole transporting layer, an emitting layer, an electron injecting layer, an electron transporting layer, a hole blocking layer and an electron blocking layer.

In the organic EL device of the third exemplary embodiment, the first layer is preferably an emitting layer.

In the organic EL device according to the third exemplary embodiment, the emitting layer preferably contains at least one of the compounds according to the first exemplary embodiment.

The organic layer other than the emitting layer may contain the compound according to the first exemplary embodiment.

In the organic EL device according to the third exemplary embodiment, the organic layer may consist of the emitting layer as the first organic layer. Alternatively, the organic layer may further include, for instance, at least one layer selected from the group consisting of a hole injecting layer, a hole transporting layer, an electron injecting layer, an electron transporting layer, a hole blocking layer, and an electron blocking layer.

### Hole Transporting Layer

The organic EL device according to the third exemplary embodiment preferably includes a second organic layer in a form of a hole transporting layer between the anode and the first organic layer.

### Electron Transporting Layer

The organic EL device according to the third exemplary embodiment preferably includes a third organic layer in a form of an electron transporting layer between the cathode and the first organic layer.

An exemplary structure of the organic EL device of the third exemplary embodiment is schematically shown in the FIGURE.

The organic EL device 1 includes a light-transmissive substrate 2, an anode 3, a cathode 4, and an organic layer 10 provided between the anode 3 and the cathode 4. The organic layer 10 includes a hole injecting layer 6, a hole transporting layer 7, an emitting layer 5 (the first organic layer), an electron transporting layer 8, and an electron injecting layer 9, which are sequentially layered on the anode 3.

It should be noted that the invention is not limited to the structure of the organic EL device shown in the FIGURE.

In the organic EL device according to the third exemplary embodiment, it is preferable that the first organic layer contains a first compound and a second compound, and it is more preferable that the first organic layer (the emitting layer) contains the first compound and the second compound.

The first compound is preferably a compound represented by a formula (10) below.

The second compound is preferably at least one of the compounds according to the first exemplary embodiment.

In the organic EL device according to the third exemplary embodiment, when the first organic layer (the emitting layer) contains the first compound and the second compound, the first compound is preferably a host material (sometimes referred to as a matrix material hereinafter), and the second compound is preferably a dopant material (sometimes referred to as a guest material, an emitter, or an emitting material hereinafter).

In the organic EL device of the third exemplary embodiment, when the first organic layer (the emitting layer) contains the first and second compounds, a singlet energy S₁(H) of the first compound and a singlet energy S₁(D) of the second compound preferably satisfy a relationship of a numerical formula (Numerical Formula 1) below.

S₁(H)>S₁(D) (Numerical Formula 1).

### Singlet Energy S1

A method of measuring the singlet energy S1 with use of a solution (occasionally referred to as a solution method) is exemplified by a method below.

A 10 μmol/L toluene solution of a measurement target compound is prepared and put in a quartz cell. An absorption spectrum (ordinate axis: luminous intensity, abscissa axis: wavelength) of the thus-obtained sample is measured at a normal temperature (300K). A tangent is drawn to the fall of the absorption spectrum on the long-wavelength side, and a wavelength value λledge (nm) at an intersection of the tangent and the abscissa axis is assigned to a conversion equation (F2) below to calculate singlet energy.

S₁[eV]=1239.85/λedge Conversion Equation (F2):

Any device for measuring absorption spectrum is usable. For instance, a spectrophotometer (U3310 manufactured by Hitachi, Ltd.) is usable.

The tangent to the fall of the absorption spectrum on the long-wavelength side is drawn as follows. While moving on a curve of the absorption spectrum from the maximum spectral value closest to the long-wavelength side in a long-wavelength direction, a tangent at each point on the curve is checked. An inclination of the tangent is decreased and increased in a repeated manner as the curve fell (i.e., a value of the ordinate axis is decreased). A tangent drawn at a point of the minimum inclination closest to the long-wavelength side (except when absorbance is 0.1 or less) is defined as the tangent to the fall of the absorption spectrum on the long-wavelength side.

The maximum absorbance of 0.2 or less is not included in the above-mentioned maximum absorbance on the long-wavelength side.

When the emitting layer contains at least one of the compounds according to the first exemplary embodiment, it is preferable that the emitting layer does not contain a phosphorescent material (dopant material).

When the emitting layer contains at least one of the compounds according to the first exemplary embodiment, it is preferable that the emitting layer does not contain a heavy metal complex and a phosphorescent rare-metal complex. Examples of the heavy metal complex herein include iridium complex, osmium complex, and platinum complex.

When the emitting layer contains at least one of the compounds according to the first exemplary embodiment, it is also preferable that the emitting layer does not contain any metal complex.

### Emission Wavelength of Organic EL Device

When the organic EL device of the third exemplary embodiment is powered, the main peak wavelength of the light emitted by the organic EL device is preferably in a range from 380 nm to 500 nm, more preferably in a range from 430 nm to 470 nm.

The main peak wavelength of the light emitted by the organic EL device is measured as follows. Voltage is applied on the organic EL device such that a current density becomes 10 mA/cm², where spectral radiance spectra are measured by a spectroradiometer CS-2000 (manufactured by Konica Minolta, Inc.). A peak wavelength of the emission spectrum of the measured spectral radiance spectra is measured and determined to be the main peak wavelength (unit: nm).

### Film Thickness of Emitting Layer

A film thickness of the emitting layer of the organic EL device in the third exemplary embodiment is preferably in a range of 5 nm to 50 nm, more preferably in a range of 7 nm to 50 nm, further preferably in a range of 10 nm to 50 nm. When the film thickness of the emitting layer is 5 nm or more, the formation of the emitting layer and adjustment of chromaticity are likely to be facilitated. When the film thickness of the emitting layer is 50 nm or less, an increase in the drive voltage is likely to be reducible.

### Content Ratio of Compound in Emitting Layer

When the emitting layer contains the first compound and the second compound, the content ratios of the first and second compounds in the emitting layer are, for instance, preferably determined as follows.

The content ratio of the first compound is preferably in a range from 80 mass % to 99 mass %, more preferably in a range from 90 mass % to 99 mass %, further preferably in a range from 95 mass % to 99 mass %.

The content ratio of the second compound is preferably in a range from 1 mass % to 10 mass %, more preferably in a range from 1 mass % to 7 mass %, further preferably in a range from 1 mass % to 5 mass %.

An upper limit of the total of the respective content ratios of the first and second compounds in the emitting layer is 100 mass %.

It should be noted that the emitting layer of the third exemplary embodiment may further contain material(s) other than the first and second compounds.

The emitting layer may include a single type of the first compound or may include two or more types of the first compound. The emitting layer may include a single type of the second compound or may include two or more types of the second compound.

### Second Compound

In the organic EL device according to the third exemplary embodiment, the compound represented by the formula (2) and the like described in the first exemplary embodiment is usable as the second compound.

### First Compound

In the organic EL device of the third exemplary embodiment, the first compound is preferably a compound represented by a formula (10) below.

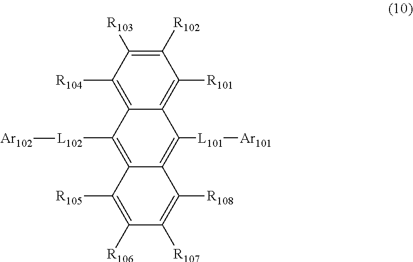

In the formula (10): R₁₀₁ to R₁₀₈ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₁₂₄, a group represented by —COOR₁₂₅, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by a formula (11) below,

-L₁₀₃-Ar₁₀₃ (11)

L₁₀₁, L₁₀₂, and L₁₀₃ are each independently a single bond, a substituted or unsubstituted arylene group having 6 to 50 ring carbon atoms, or a substituted or unsubstituted divalent heterocyclic group having 5 to 50 ring atoms;

Ar₁₀₂, and Ar₁₀₃ are each independently a substituted or unsubstituted aryl group having 6 to 50 ring carbon atoms, or a substituted or unsubstituted monovalent heterocyclic group having 5 to 50 ring atoms;

R₉₀₁, R₉₀₂, R₉₀₃, R₉₀₆, R₉₀₇, R₁₂₄ and R₁₂₅ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;

R₉₀₄ and R₉₀₅ are each independently a substituted or unsubstituted alkyl group having 1 to 50 carbon atoms, or a substituted or unsubstituted aryl group having 6 to 50 ring carbon atoms;

a plurality of R₉₀₁ are mutually the same or different;

a plurality of R₉₀₂ are mutually the same or different;

a plurality of R₉₀₃ are mutually the same or different;

a plurality of R₉₀₄ are mutually the same or different;

a plurality of R₉₀₅ are mutually the same or different;

a plurality of R₉₀₆ are mutually the same or different;

a plurality of R₉₀₇ are mutually the same or different;

a plurality of R₁₂₄ are mutually the same or different;

a plurality of R₁₂₅ are mutually the same or different; and

a plurality of groups represented by the formula (11) are mutually the same or different.

In the organic EL device of the third exemplary embodiment, it is preferable that R₁₀₁ to R₁₀₈ are not mutually bonded.

It is also preferable that the first compound is a compound represented by a formula (13) below.

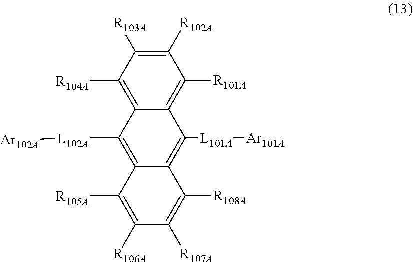

In the formula (13):

R_{101A} to R_{108A} each independently represent a hydrogen atom, or a substituted or unsubstituted aryl group having 6 to 50 ring carbon atoms;

L_{101A} and L_{102A} are each independently a single bond, or a substituted or unsubstituted arylene group having 6 to 30 ring carbon atoms; and

Ar_{101A} and Ar_{102A} are each independently a substituted or unsubstituted aryl group having 6 to 50 ring carbon atoms.

It is also preferable that the first compound is represented by a formula (14) below.

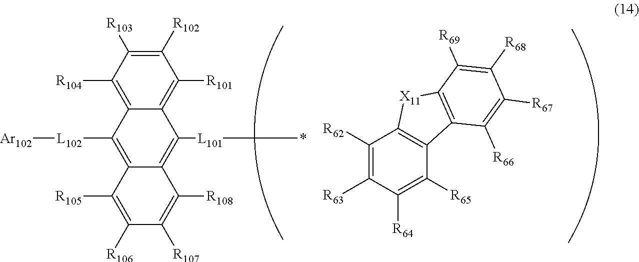

In the formula (14):

L₁₀₁, L₁₀₂ and Ar₁₀₂ represent the same as L₁₀₁, L₁₀₂ and Ar₁₀₂ in the formula (10);

R_{101A} to R_{108A} each independently represent a hydrogen atom, or a substituted or unsubstituted aryl group having 6 to 50 ring carbon atoms;

X₁₁ is an oxygen atom, sulfur atom, or N(R₆₁);

R₆₁ is a hydrogen atom, a substituted or unsubstituted alkyl group having 1 to 50 carbon atoms, or a substituted or unsubstituted aryl group having 6 to 50 ring carbon atoms;

one of R₆₂ to R₆₉ is a bond with L₁₀₁;

* represents a bonding position with L₁₀₁; and

at least one combination of adjacent two or more of R₆₂ to R₆₉ not bonded with L₁₀₁ are mutually bonded to form a substituted or unsubstituted saturated ring, mutually bonded to form a substituted or unsubstituted unsaturated ring, or not mutually bonded;

one or more of R₆₂ to R₆₉ not bonded with L₁₀₁, not forming the saturated ring and not forming the unsaturated ring each independently represent a hydrogen atom, a substituted or unsubstituted alkyl group having 1 to 50 carbon atoms, or a substituted or unsubstituted aryl group having 6 to 50 ring carbon atoms.

In the compound represented by the formula (14), it is also preferable that R₆₂ to R₆₉ not bonded with L₁₀₁ are not mutually boned.

The combination of adjacent two or more of R₆₂ to R₆₉ include a pair of R₆₂ and R₆₃, a pair of R₆₃ and R₆₄, a pair of R₆₄ and R₆₅, a combination of R₆₂, R₆₃ and R₆₄, a combination of R₆₃, R₆₄ and R₆₅, a pair of R₆₆ and R₆₇, a pair of R₆₇ and R₆₈, a pair of R₆₈ and R₆₉, a combination of R₆₆, R₆₇ and R₆₈, and a combination of R₆₇, R₆₈ and R₆₉.

It is also preferable that the first compound is represented by a formula (15) below.

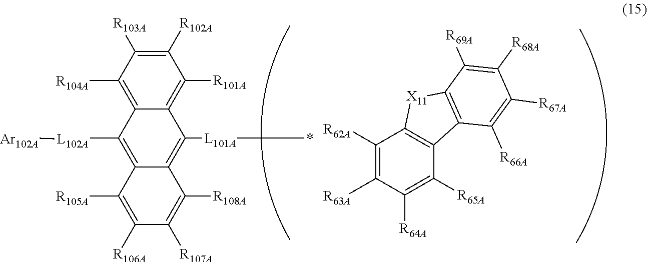

In the formula (15):

L₁₀₁, L₁₀₂ and Ar₁₀₂ represent the same as L₁₀₁, L₁₀₂ and Ar₁₀₂ in the formula (10);

R_{101A} to R_{108A} each independently represent a hydrogen atom, or a substituted or unsubstituted aryl group having 6 to 50 ring carbon atoms;

X₁₁ is an oxygen atom, sulfur atom, or N(R₆₁);

R₆₁ is a hydrogen atom, a substituted or unsubstituted alkyl group having 1 to 50 carbon atoms, or a substituted or unsubstituted aryl group having 6 to 50 ring carbon atoms;

at least one combination of adjacent two or more of R_{62A} to R_{69A} are mutually bonded to form a substituted or unsubstituted saturated ring, mutually bonded to form a substituted or unsubstituted unsaturated ring, or not mutually bonded;

the combination of adjacent two of R_{62A} to R_{69A} are mutually bonded to form a ring represented by a formula (15A) below;

one or more of R_{62A} to R_{69A} not forming the saturated ring and not forming the unsaturated ring each independently represent a hydrogen atom, a substituted or unsubstituted alkyl group having 1 to 50 carbon atoms, or a substituted or unsubstituted aryl group having 6 to 50 ring carbon atoms.

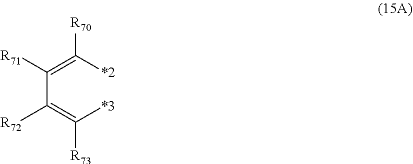

In the formula (15A):

*2 and *3 represent bonding portions with adjacent two of R_{62A} to R_{69A};

one of R_{62A} to R_{69A} and R₇₀ to R₇₃ is a bond with L₁₀₁;

* represents a bonding position with L₁₀₁; and

R₇₀ to R₇₃ not bonded with L₁₀₁ each independently represent a hydrogen atom, a substituted or unsubstituted alkyl group having 1 to 50 carbon atoms, or a substituted or unsubstituted aryl group having 6 to 50 ring carbon atoms.

The combination of adjacent two or more of R_{62A} to R_{69A} includes a pair of R_{62A} and R_{63A}, a pair of R_{63A} and R_{64A}, a pair of R_{64A} and R_{65A}, a combination of R_{62A}, R_{63A} and R_{64A}, a combination of R_{63A}, R_{64A} and R_{65A}, a pair of R_{66A} and R_{67A}, a pair of R_{67A} and R_{68A}, a pair of R_{68A} and R_{69A}, a combination of R_{66A}, R_{67A} and R_{68A}, and a combination of R_{67A}, R_{68A} and R_{69A}.

In the third exemplary embodiment, the first compound is preferably represented by a formula (16) below.

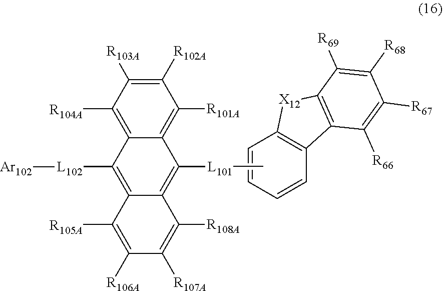

In the formula (16):

L₁₀₁, L₁₀₂ and Ar₁₀₂ represent the same as L₁₀₁, L₁₀₂ and Ar₁₀₂ in the formula (10);

R_{101A} to R_{108A} each independently represent a hydrogen atom, or a substituted or unsubstituted aryl group having 6 to 50 ring carbon atoms;

at least one combination of adjacent two or more of R₆₆ to R₆₉ are mutually bonded to form a substituted or unsubstituted saturated ring, mutually bonded to form a substituted or unsubstituted unsaturated ring, or not mutually bonded;

one or more of R₆₆ to R₆₉ not forming the saturated ring and not forming the unsaturated ring each independently represent a hydrogen atom, a substituted or unsubstituted alkyl group having 1 to 50 carbon atoms, or a substituted or unsubstituted aryl group having 6 to 50 ring carbon atoms; and

X₁₂ is an oxygen atom or a sulfur atom.

In the third exemplary embodiment, the first compound is also preferably represented by a formula (16A) below.

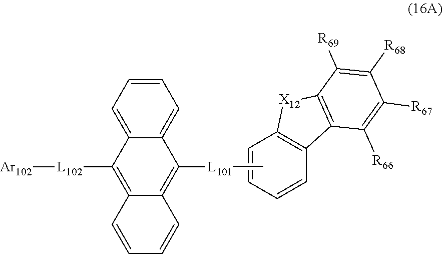

In the formula (16A):

L₁₀₁, L₁₀₂ and Ar₁₀₂ represent the same as L₁₀₁, L₁₀₂ and Ar₁₀₂ in the formula (10);

at least one combination of adjacent two or more of R₆₆ to R₆₉ are mutually bonded to form a substituted or unsubstituted saturated ring, mutually bonded to form a substituted or unsubstituted unsaturated ring, or not mutually bonded;

one or more of R₆₆ to R₆₉ not forming the saturated ring and not forming the unsaturated ring each independently represent a hydrogen atom, a substituted or unsubstituted alkyl group having 1 to 50 carbon atoms, or a substituted or unsubstituted aryl group having 6 to 50 ring carbon atoms; and

X₁₂ is an oxygen atom or a sulfur atom.

In the third exemplary embodiment, the first compound is also preferably represented by a formula (16B) below.

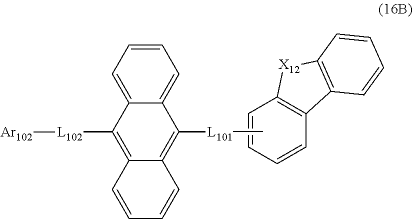

In the formula (16B):

L₁₀₁, L₁₀₂ and Ar₁₀₂ represent the same as L₁₀₁, L₁₀₂ and Ar₁₀₂ in the formula (10); and

X₁₂ is an oxygen atom or a sulfur atom.

In the third exemplary embodiment, the first compound is also preferably represented by a formula (16C), or a formula (16D) below.

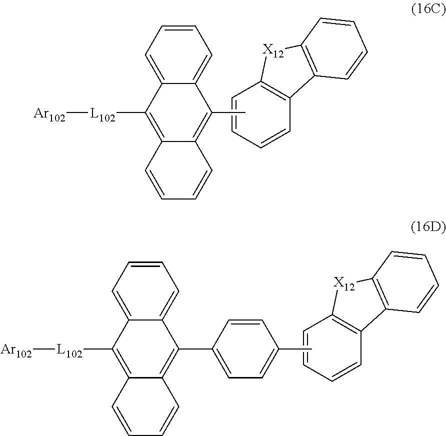

In the formulae (16C) and (16D):

L₁₀₂ and Ar₁₀₂ represent the same as L₁₀₂ and Ar₁₀₂ in the formula (10); and

X₁₂ is an oxygen atom or a sulfur atom.

In the third exemplary embodiment, the first compound is also preferably represented by a formula (17) below.

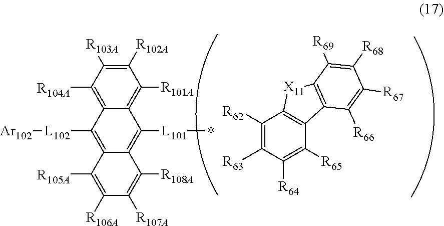

In the formula (17):

L₁₀₁, L₁₀₂ and Ar₁₀₂ represent the same as L₁₀₁, L₁₀₂ and Ar₁₀₂ in the formula (10);

R_{101A} to R_{108A} each independently represent a hydrogen atom, or a substituted or unsubstituted aryl group having 6 to 50 ring carbon atoms;

X₁₁ is an oxygen atom, sulfur atom, or N(R₆₁);

R₆₁ is a hydrogen atom, a substituted or unsubstituted alkyl group having 1 to 50 carbon atoms, or a substituted or unsubstituted aryl group having 6 to 50 ring carbon atoms;

one of R₆₂ to R₆₉ is a bond with L₁₀₁;

* represents a bonding position with L₁₀₁; and

at least one combination of adjacent two or more of R₆₂ to R₆₉ not bonded with L₁₀₁ are mutually bonded to form a substituted or unsubstituted saturated ring, mutually bonded to form a substituted or unsubstituted unsaturated ring, or not mutually bonded;

at least one of a pair of R₆₆ and R₆₇, a pair of R₆₇ and R₆₈, and a pair of R₆₈ and R₆₉ are mutually bonded to form a substituted or unsubstituted saturated ring or a substituted or unsubstituted unsaturated ring; and

one or more of R₆₂ to R₆₉ not bonded with L₁₀₁, not forming the saturated ring and not forming the unsaturated ring each independently represent a hydrogen atom, a substituted or unsubstituted alkyl group having 1 to 50 carbon atoms, or a substituted or unsubstituted aryl group having 6 to 50 ring carbon atoms.

In the third exemplary embodiment, the first compound is also preferably represented by a formula (17A) below.

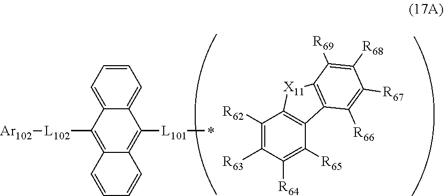

In the formula (17A):

L₁₀₁, L₁₀₂ and Ar₁₀₂ represent the same as L₁₀₁, L₁₀₂ and Ar₁₀₂ in the formula (10);

one of R₆₂ to R₆₉ is a bond with L₁₀₁;

* represents a bonding position with L₁₀₁;

X₁₁ represents the same as X₁₁ in the formula (14);

at least one combination of adjacent two or more of R₆₂ to R₆₉ not bonded with L₁₀₁ are mutually bonded to form a substituted or unsubstituted saturated ring, mutually bonded to form a substituted or unsubstituted unsaturated ring, or not mutually bonded;

at least one of a pair of R₆₆ and R₆₇, a pair of R₆₇ and R₆₈, and a pair of R₆₈ and R₆₉ are mutually bonded to form a substituted or unsubstituted saturated ring or a substituted or unsubstituted unsaturated ring; and

one or more of R₆₂ to R₆₉ not bonded with L₁₀₁, not forming the saturated ring and not forming the unsaturated ring each independently represent a hydrogen atom, a substituted or unsubstituted alkyl group having 1 to 50 carbon atoms, or a substituted or unsubstituted aryl group having 6 to 50 ring carbon atoms.

In the third exemplary embodiment, the first compound is also preferably represented by a formula (18) below.

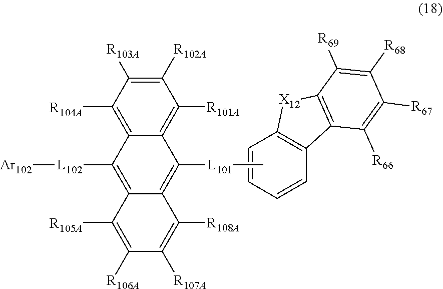

In the formula (18):

L₁₀₁, L₁₀₂ and Ar₁₀₂ represent the same as L₁₀₁, L₁₀₂ and Ar₁₀₂ in the formula (10);

R_{101A} to R_{108A} each independently represent a hydrogen atom, or a substituted or unsubstituted aryl group having 6 to 50 ring carbon atoms;

X₁₂ is an oxygen atom or a sulfur atom;

at least one of a pair of R₆₆ and R₆₇, a pair of R₆₇ and R₆₈, and a pair of R₆₈ and R₆₉ are mutually bonded to form a substituted or unsubstituted saturated ring or a substituted or unsubstituted unsaturated ring; and

one or more of R₆₆ to R₆₉ not forming the saturated ring and not forming the unsaturated ring each independently represent a hydrogen atom, a substituted or unsubstituted alkyl group having 1 to 50 carbon atoms, or a substituted or unsubstituted aryl group having 6 to 50 ring carbon atoms.

In the third exemplary embodiment, the first compound is also preferably represented by a formula (18A) below.

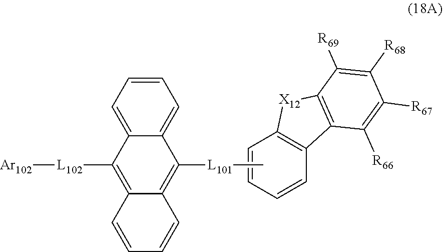

In the formula (18A):

L₁₀₁, L₁₀₂ and Ar₁₀₂ represent the same as L₁₀₁, L₁₀₂ and Ar₁₀₂ in the formula (10);

X₁₂ is an oxygen atom or a sulfur atom;

at least one of a pair of R₆₆ and R₆₇, a pair of R₆₇ and R₆₈, and a pair of R₆₈ and R₆₉ are mutually bonded to form a substituted or unsubstituted saturated ring or a substituted or unsubstituted unsaturated ring; and

one or more of R₆₆ to R₆₉ not forming the saturated ring and not forming the unsaturated ring each independently represent a hydrogen atom, a substituted or unsubstituted alkyl group having 1 to 50 carbon atoms, or a substituted or unsubstituted aryl group having 6 to 50 ring carbon atoms.

When the pair of R₆₆ and R₆₇ are mutually bonded to form a substituted or unsubstituted saturated ring, the pair of R₆₆ and R₆₇ also preferably form an unsubstituted benzene ring.

When the pair of R₆₇ and R₆₈ are mutually bonded to form a substituted or unsubstituted saturated ring, the pair of R₆₇ and R₆₈ also preferably form an unsubstituted benzene ring.

When the pair of R₆₈ and R₆₉ are mutually bonded to form a substituted or unsubstituted saturated ring, the pair of R₆₈ and R₆₉ also preferably form an unsubstituted benzene ring.

In the third exemplary embodiment, it is also preferable that at least one of the pair of R₆₆ and R₆₇, the pair of R₆₇ and R₆₈, and the pair of R₆₈ and R₆₉ in the compound represented by the formula (17), the formula (18) or the formula (18A) are mutually bonded to form a ring represented by a formula (18B) or (18C) below, and one or more of R₆₆ to R₆₉ not forming the ring represented by the formula (18B) below and not forming the ring represented by the formula (18C) below does not form a substituted or unsubstituted saturated ring or a substituted or unsubstituted unsaturated ring.

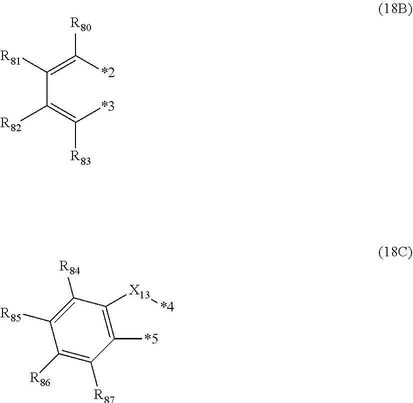

In the formulae (18B) and (18C):

*2 and *3 each represent a bonding portion with one of the pair of R₆₆ and R₆₇, the pair of R₆₇ and R₆₈, and the pair of R₆₈ and R₆₉;

*4 and *5 each represent a bonding portion with one of the pair of R₆₆ and R₆₇, the pair of R₆₇ and R₆₈, and the pair of R₆₈ and R₆₉;

R₈₀ to R₈₇ each independently represent a hydrogen atom, a substituted or unsubstituted alkyl group having 1 to 50 carbon atoms, or a substituted or unsubstituted aryl group having 6 to 50 ring carbon atoms; and

X₁₃ is an oxygen atom or a sulfur atom.

In the third exemplary embodiment, the first compound is also preferably represented by a formula (19) below.

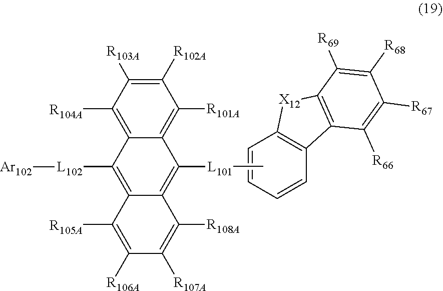

In the formula (19):

L₁₀₁, L₁₀₂ and Ar₁₀₂ represent the same as L₁₀₁, L₁₀₂ and Ar₁₀₂ in the formula (10);

R_{101A} to R_{108A} each independently represent a hydrogen atom, or a substituted or unsubstituted aryl group having 6 to 50 ring carbon atoms;

X₁₂ is an oxygen atom or a sulfur atom;

none of the pair of R₆₆ and R₆₇, the pair of R₆₇ and R₆₈, and the pair of R₆₈ and R₆₉ are mutually bonded; and

R₆₆ to R₆₉ each independently represent a hydrogen atom, a substituted or unsubstituted alkyl group having 1 to 50 carbon atoms, or a substituted or unsubstituted aryl group having 6 to 50 ring carbon atoms.

In the third exemplary embodiment, the first compound is also preferably represented by a formula (101), (102), (103) or (104) below.

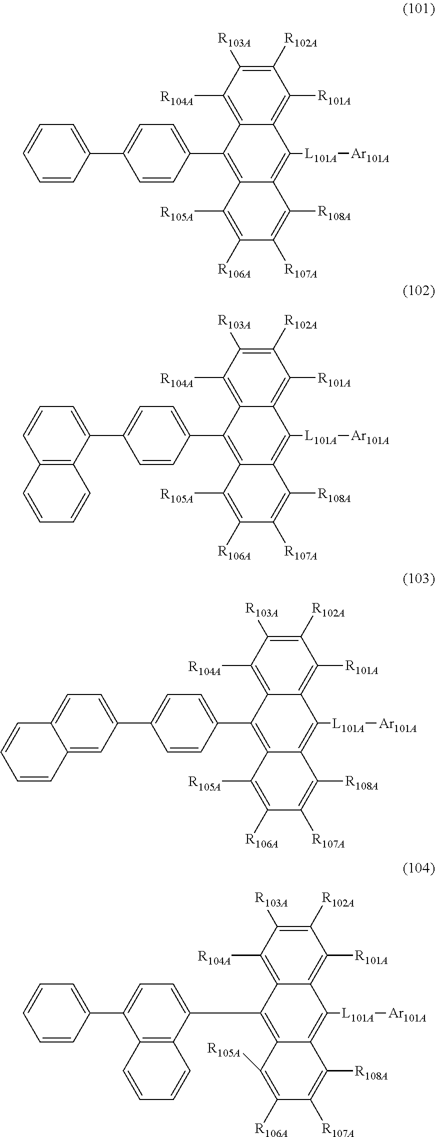

In the formulae (101) to (104):

L_{101A} and Ar_{101A} represent the same as L_{101A} and Ar_{101A} in the formula (13); and

R_{101A} to R_{108A} each independently represent the same as R_{101A} to R_{108A} of the formula (13).

In the third exemplary embodiment, the first compound is also preferably represented by a formula (101A), (102A), (103A) or (104A) below.

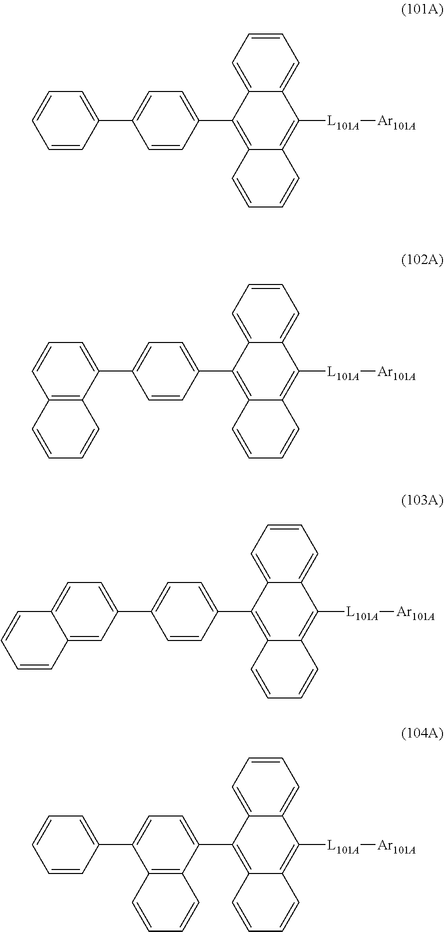

In the formulae (101A), (102A), (103A) and (104A), L_{101A} and Ar_{101A} are the same as L_{101A} and Ar_{101A} in the formula (13).

The details of the substituents, and substituents meant by “substituted or unsubstituted” in the formulae (10), (11), (13), (14), (15), (15A), (16), (16A), (16B), (16C), (16D), (17), (17A), (18), (18A), (18B), (18C), (19), (101), (102), (103), (104), (101A), (102A), (103A) and (104A) are the same as those described under the subtitle “Definitions.”

### Method of Preparing First Compound

The first compound can be prepared through any known method. The compound represented by the formula (10) also can be prepared through any known method.

Specific examples of the compound represented by the formula (10) include compounds shown below. It should however be noted that the invention is not limited to the specific examples of the compound.

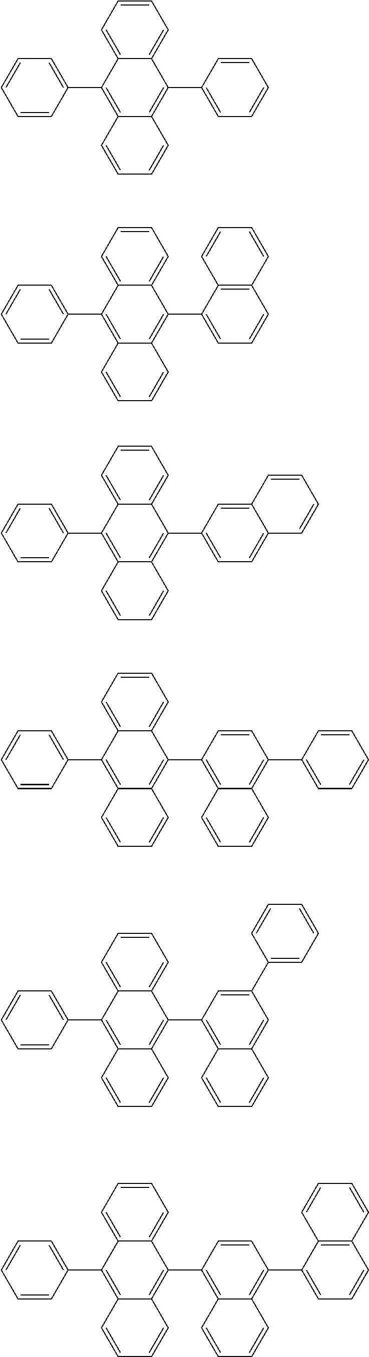

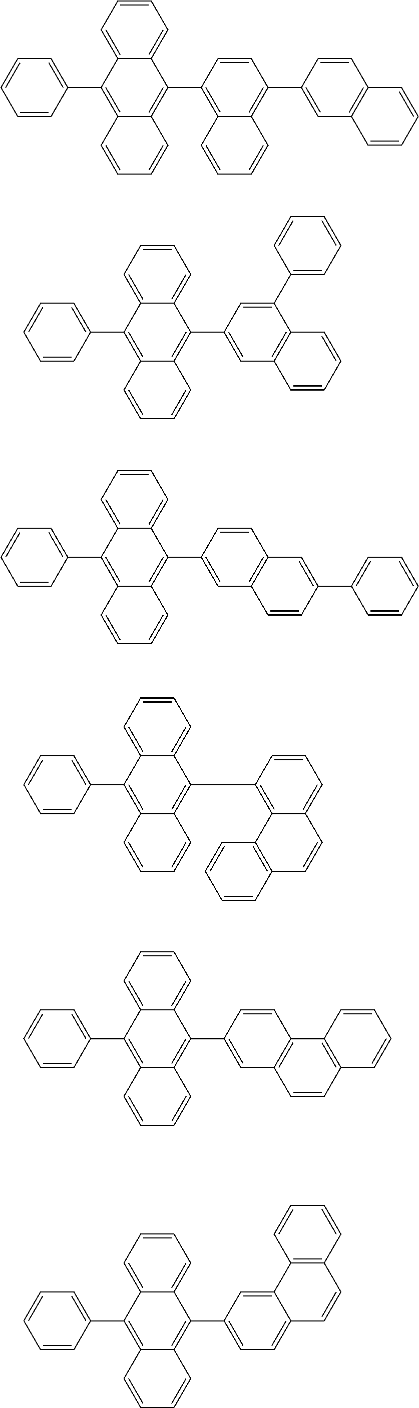

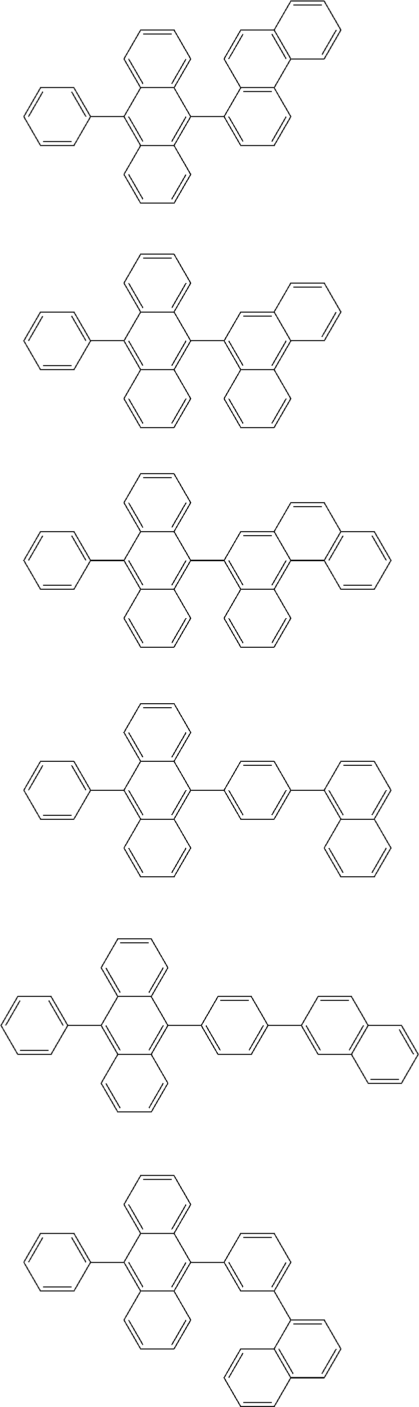

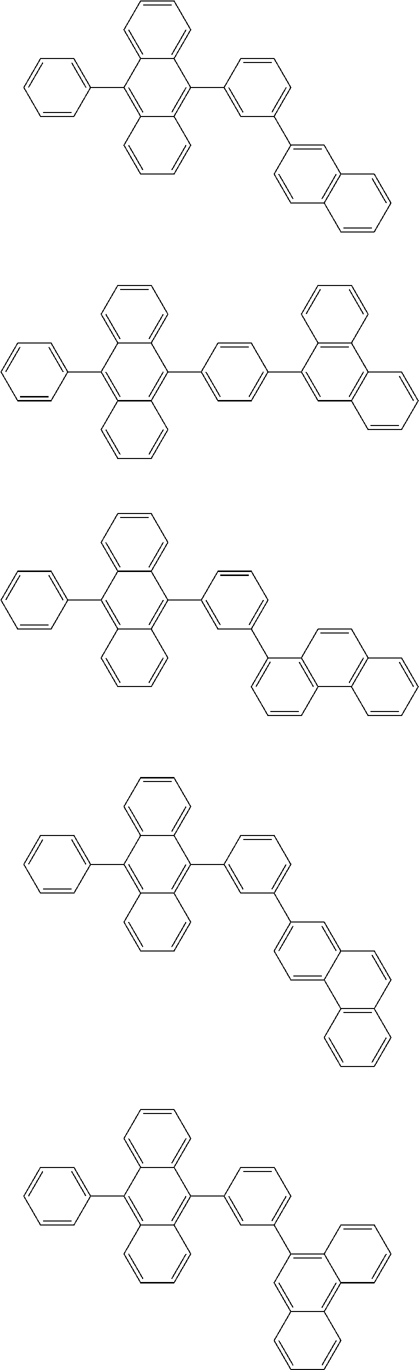

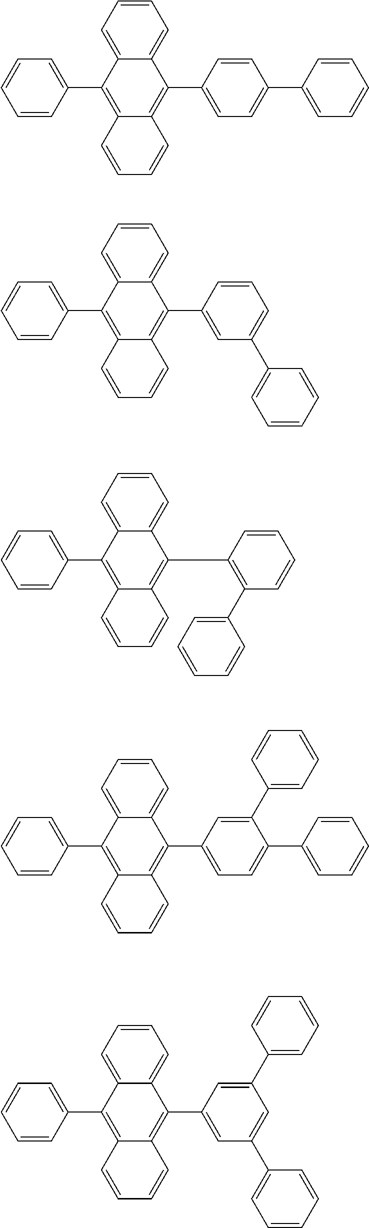

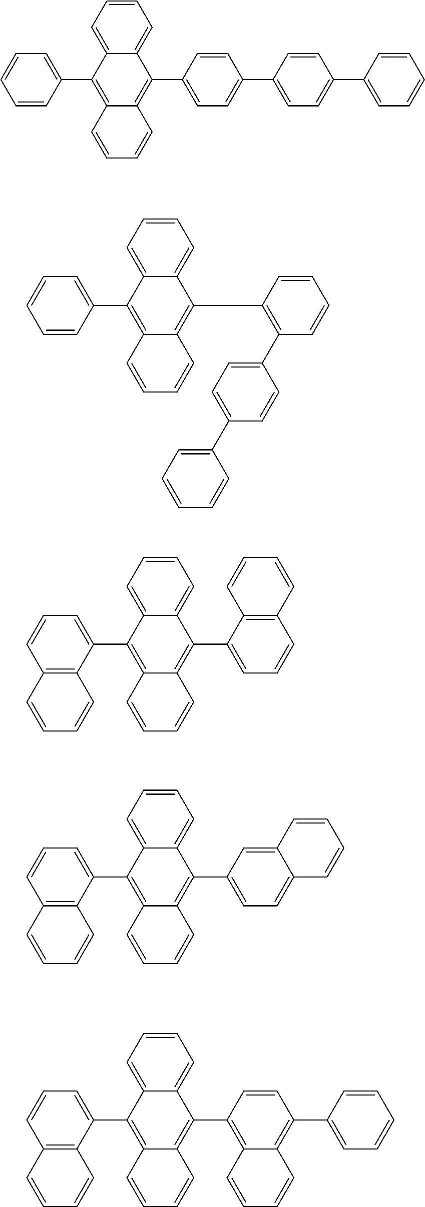

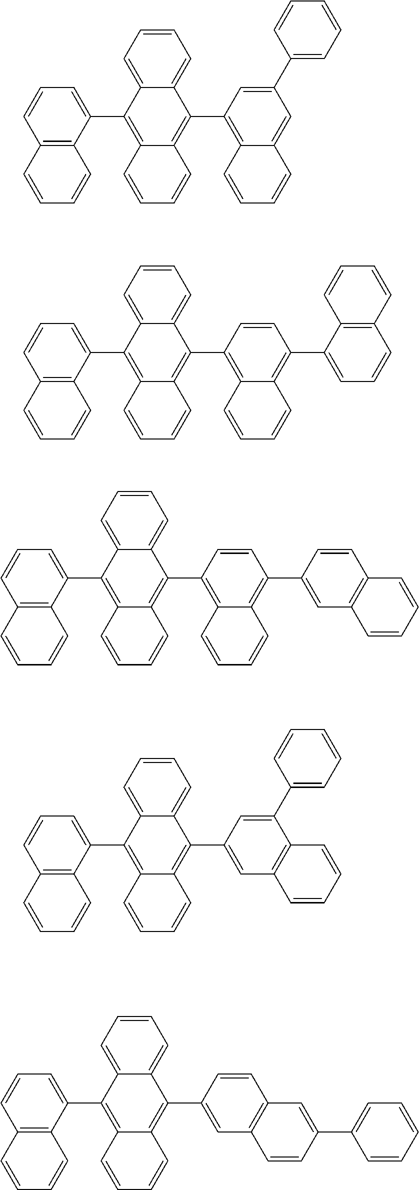

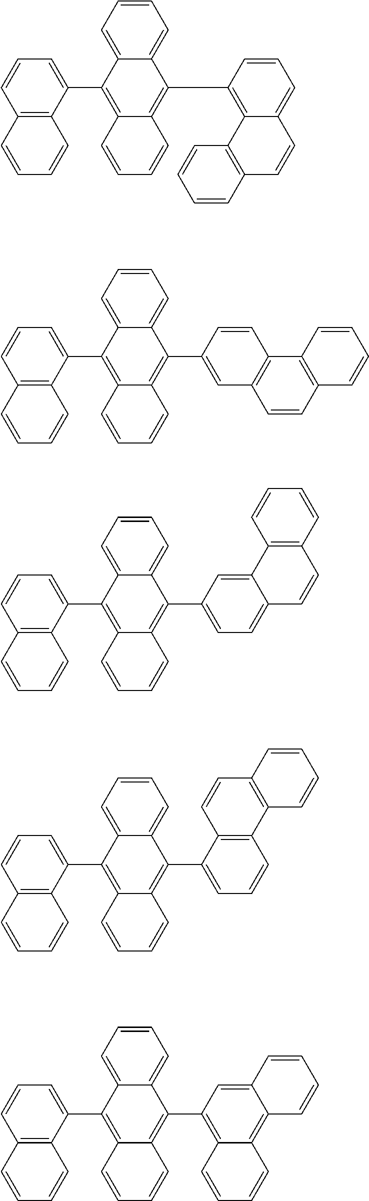

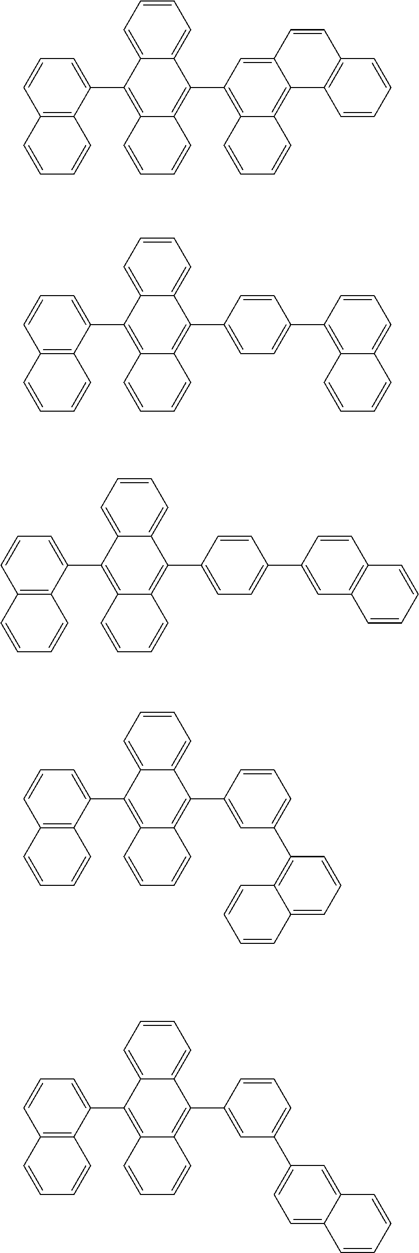

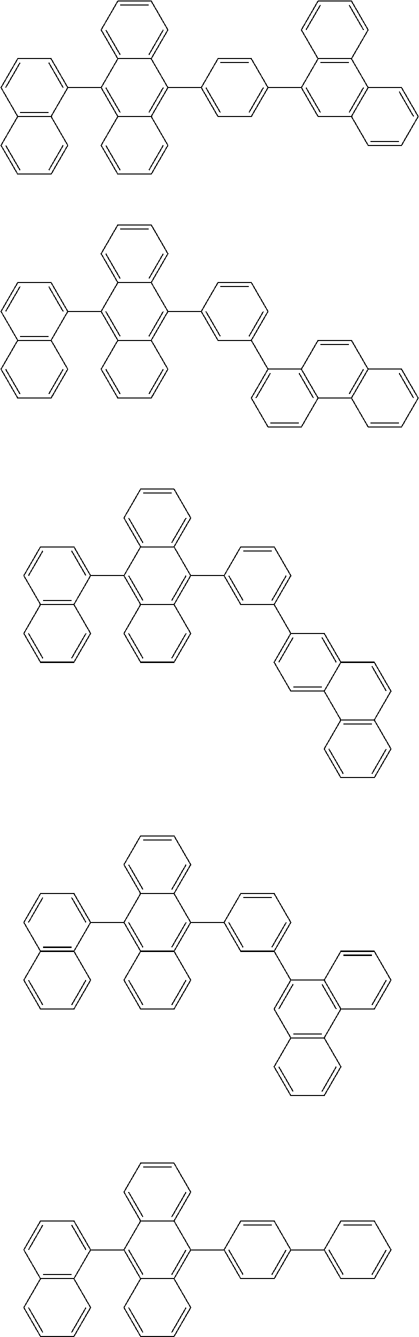

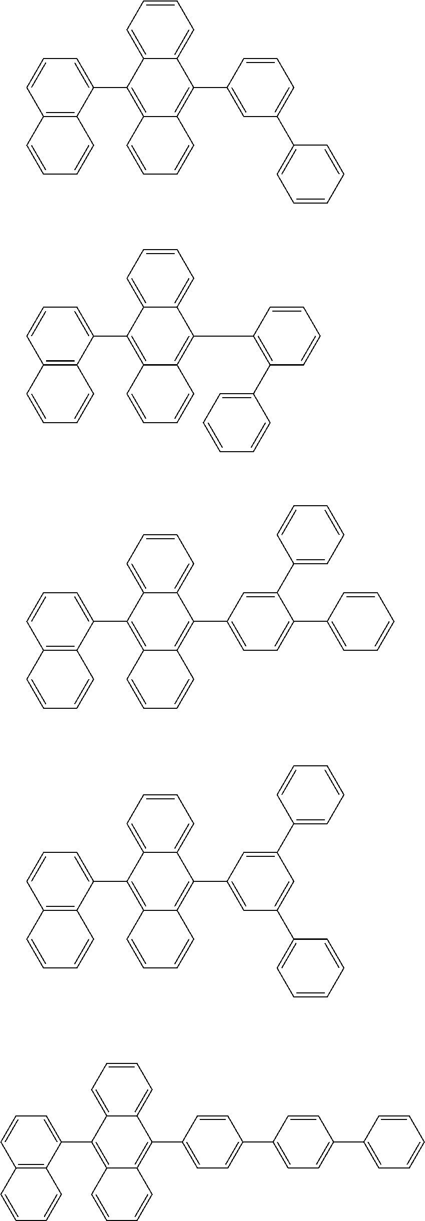

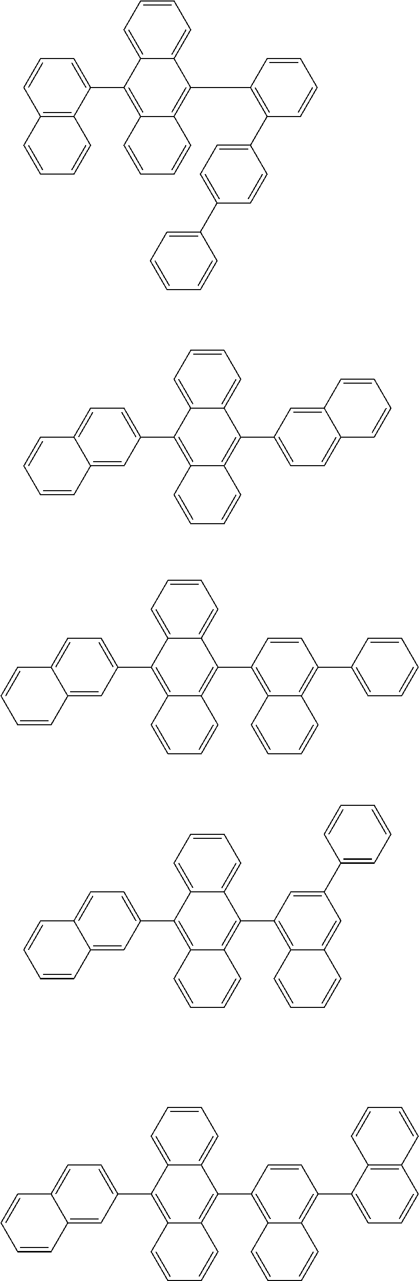

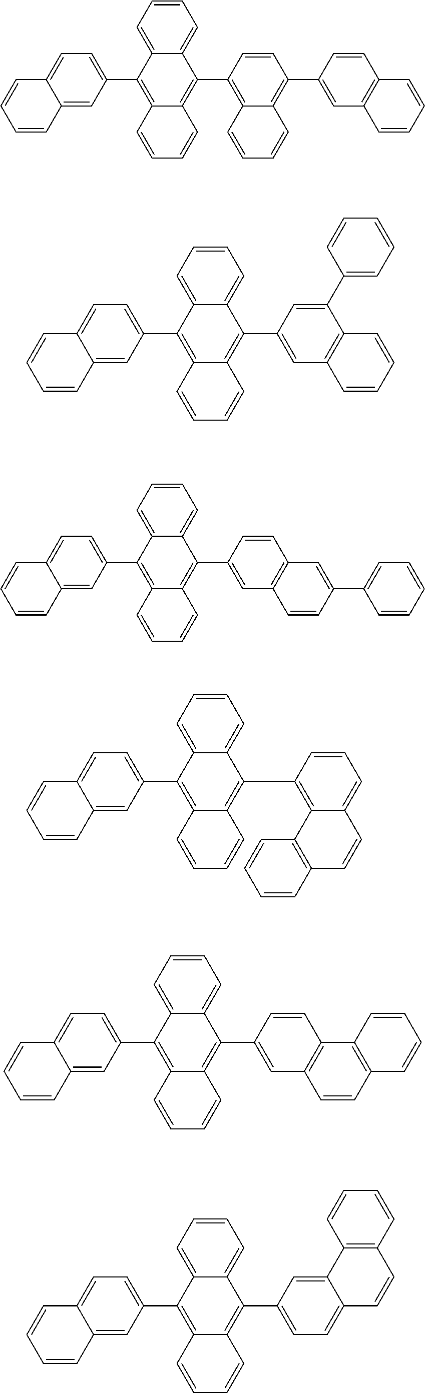

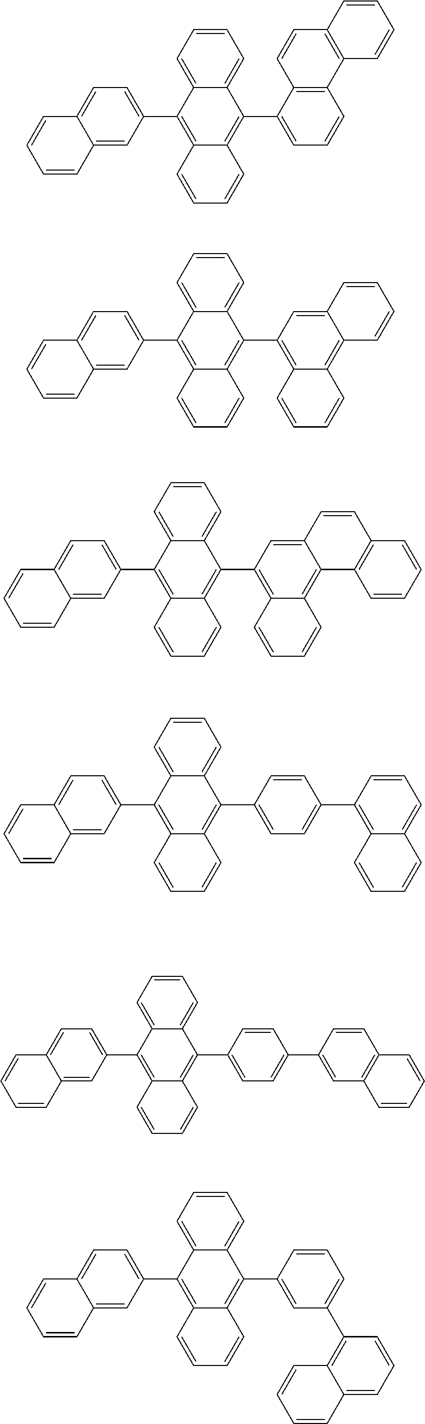

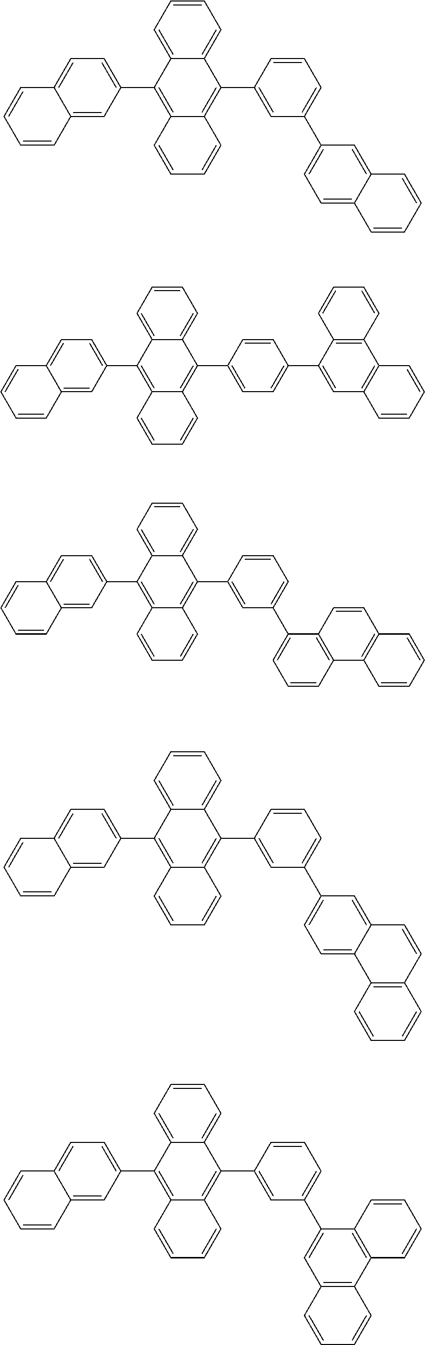

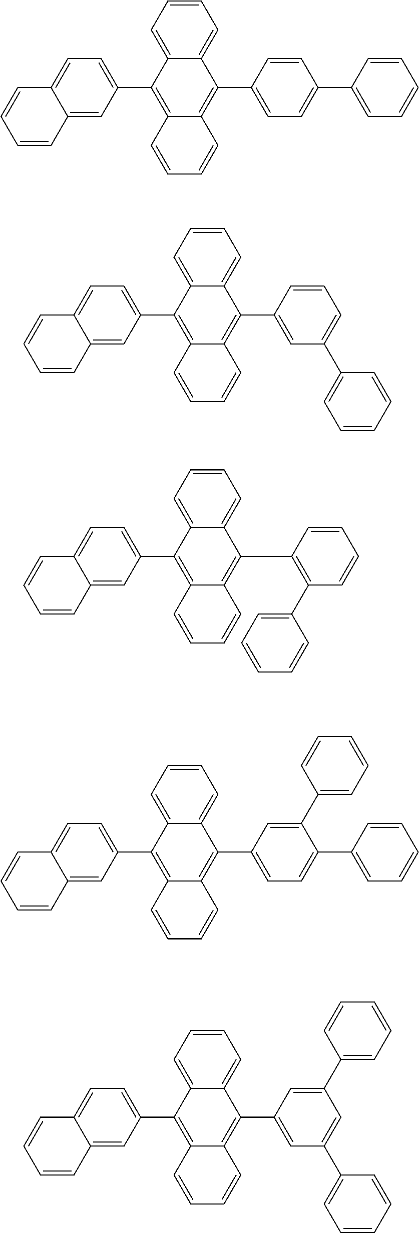

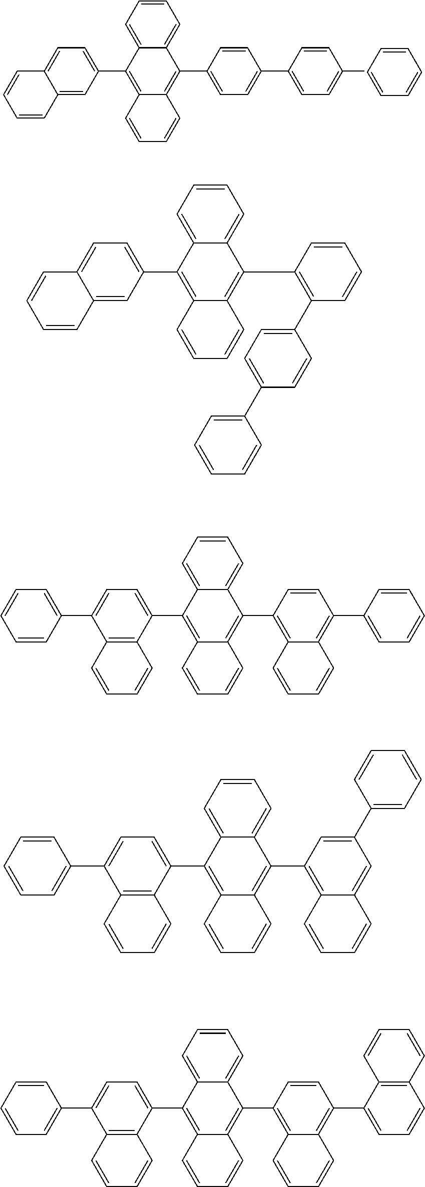

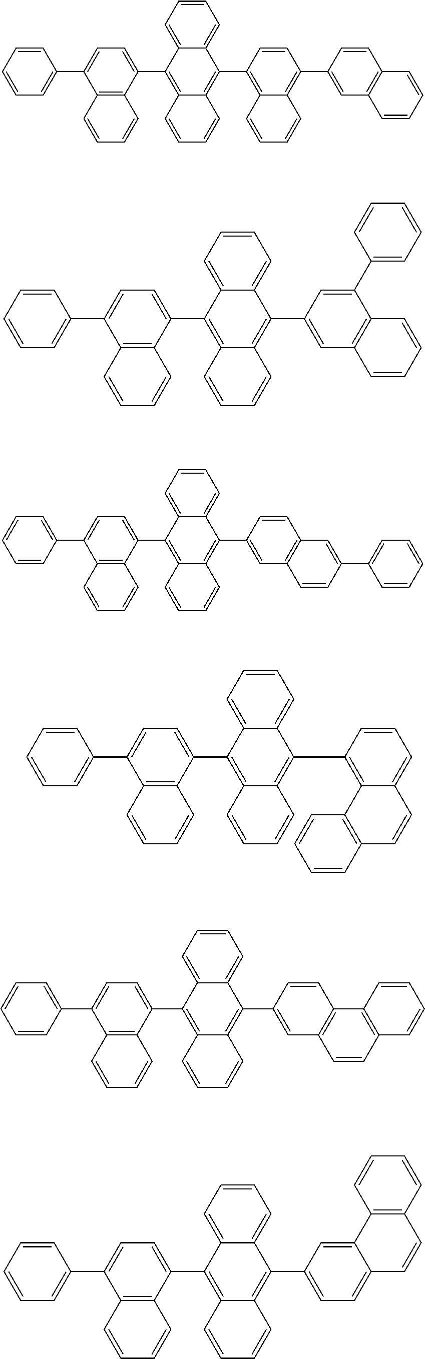

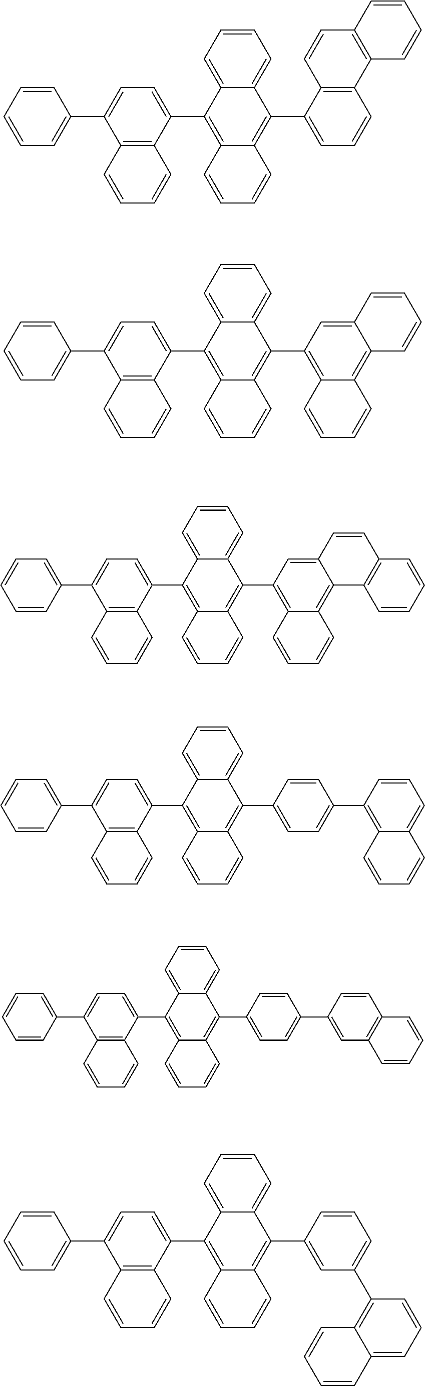

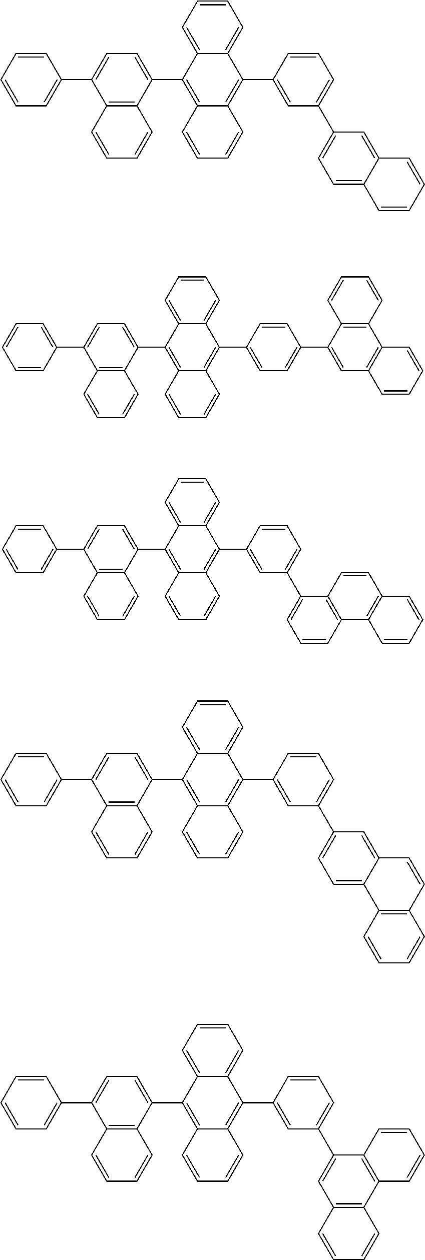

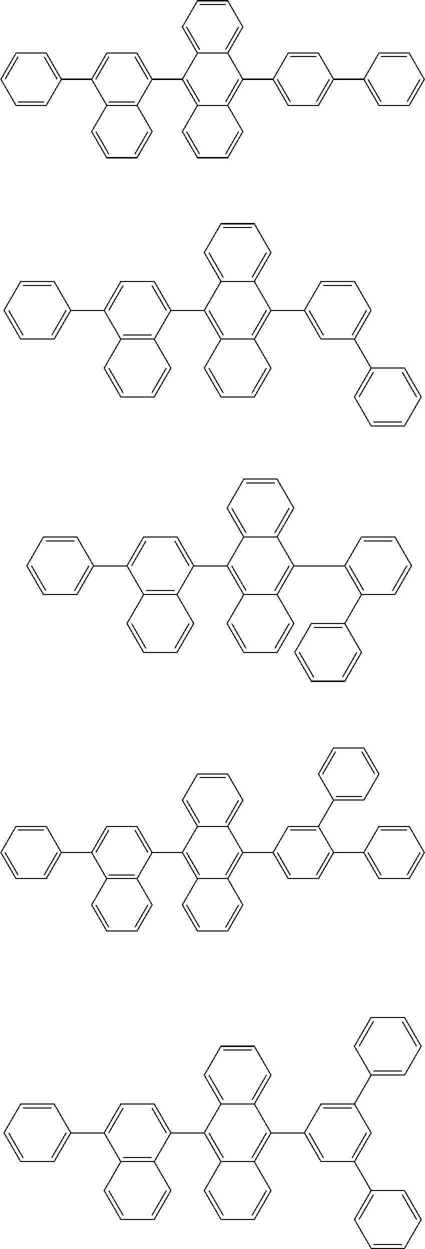

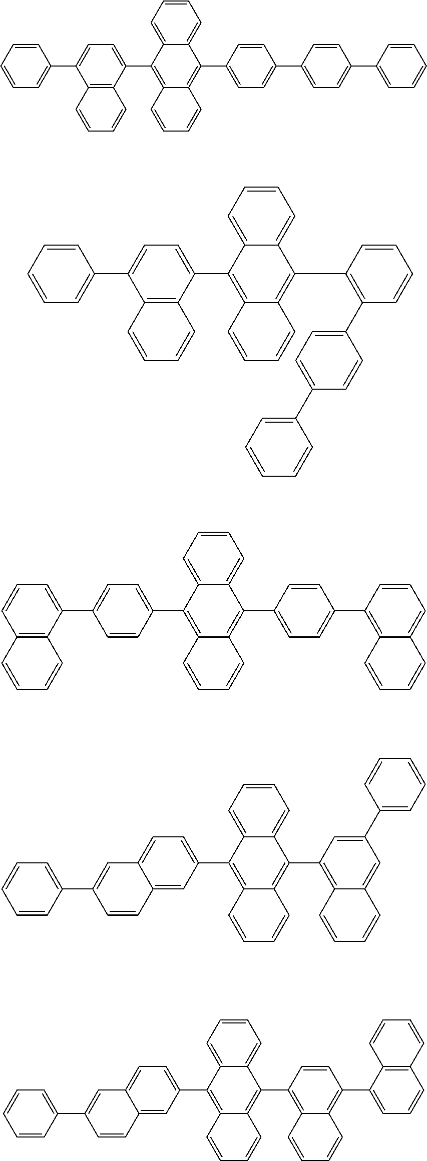

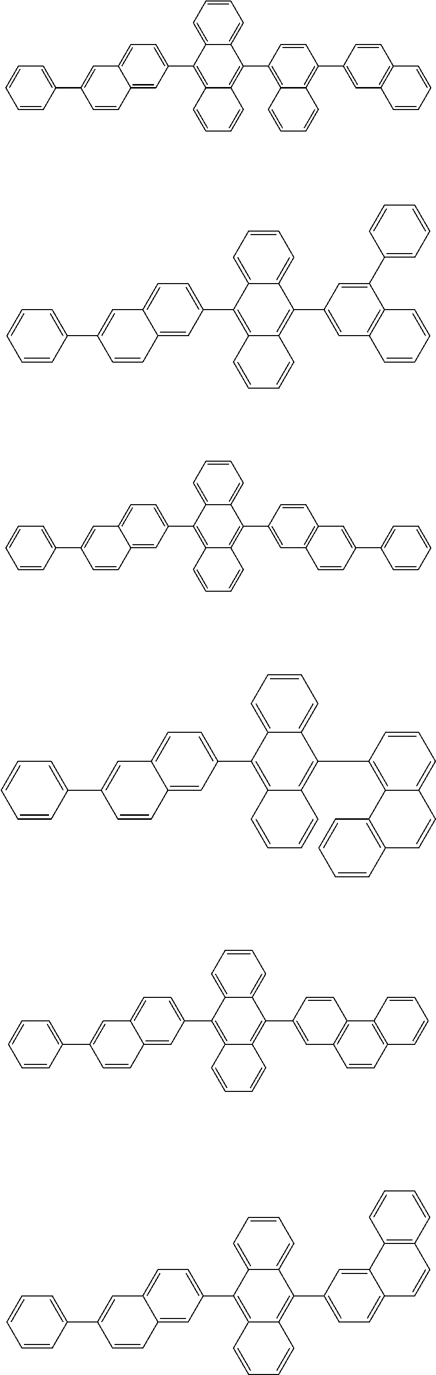

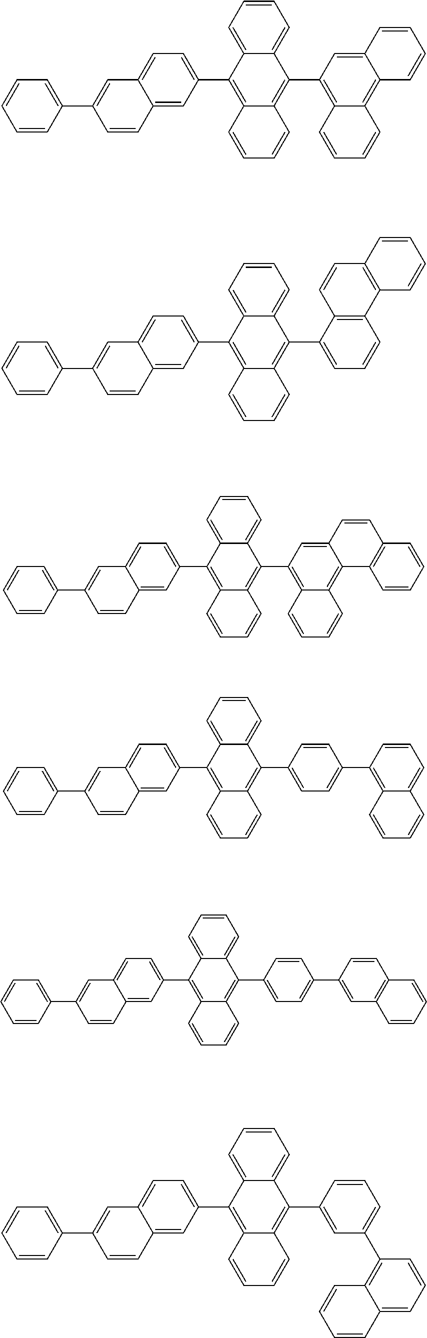

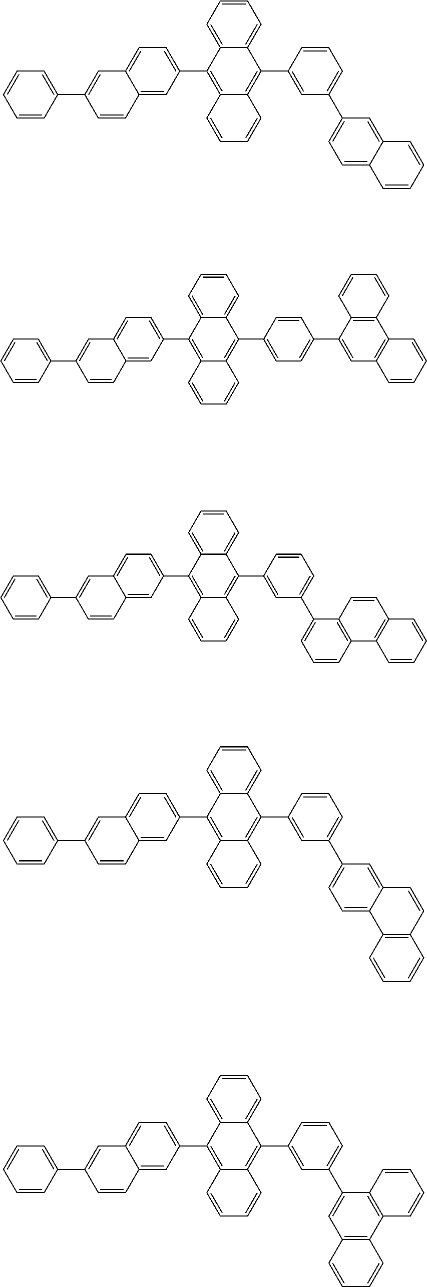

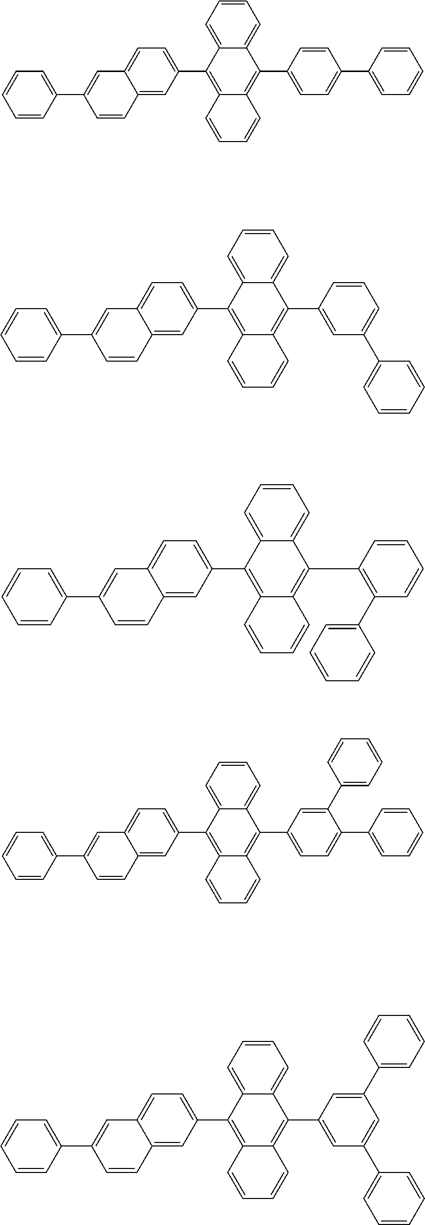

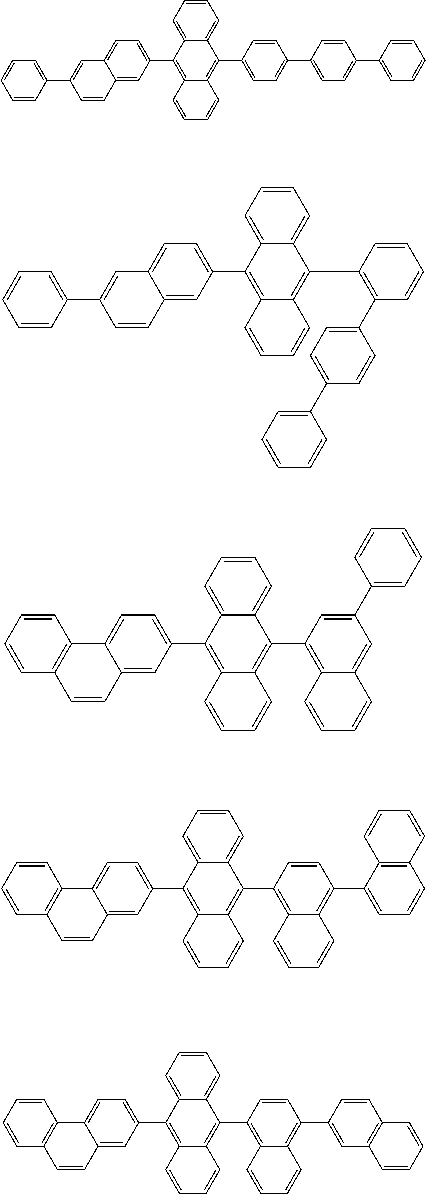

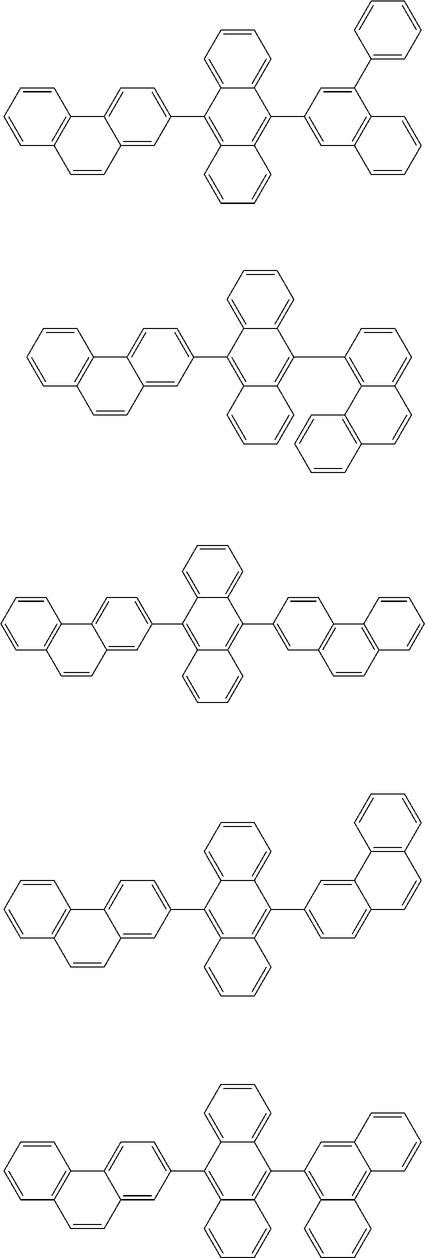

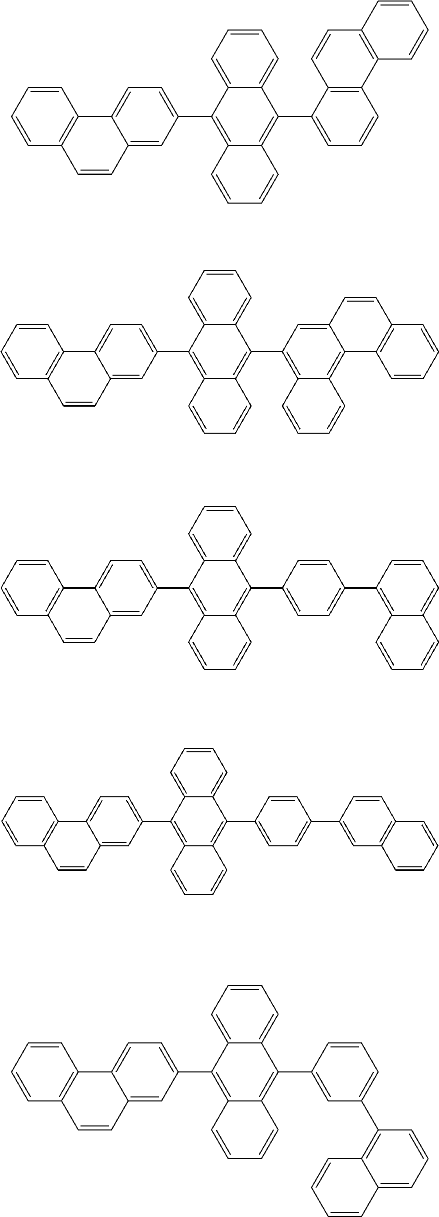

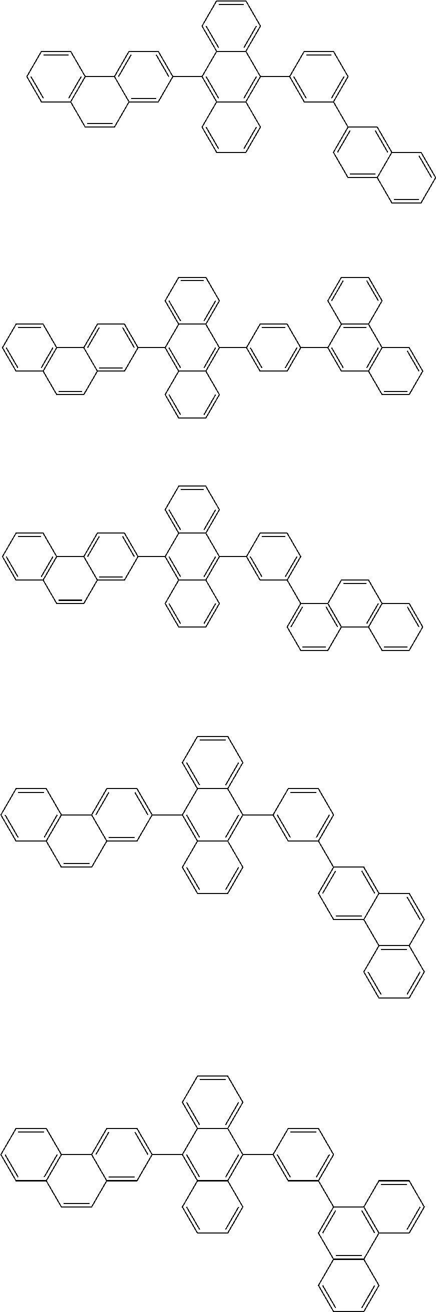

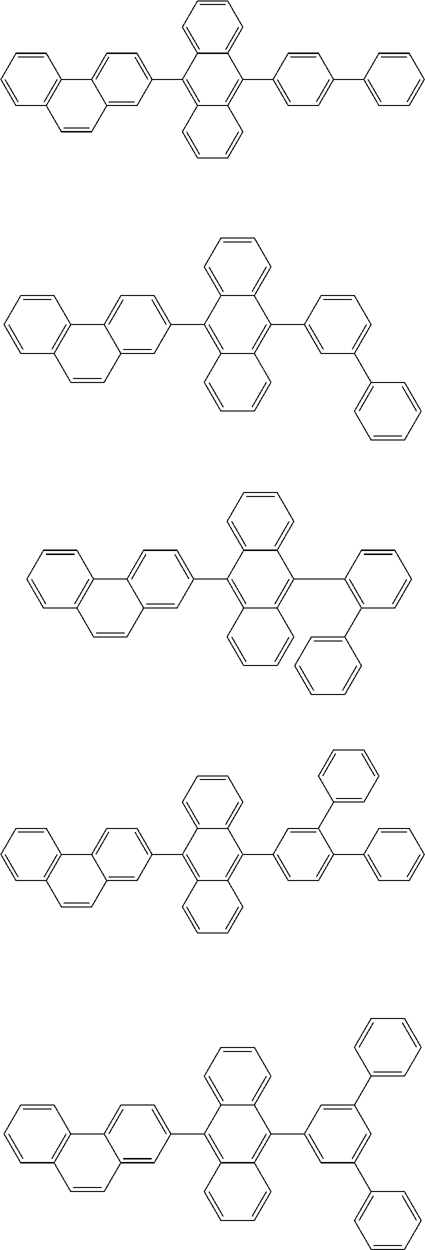

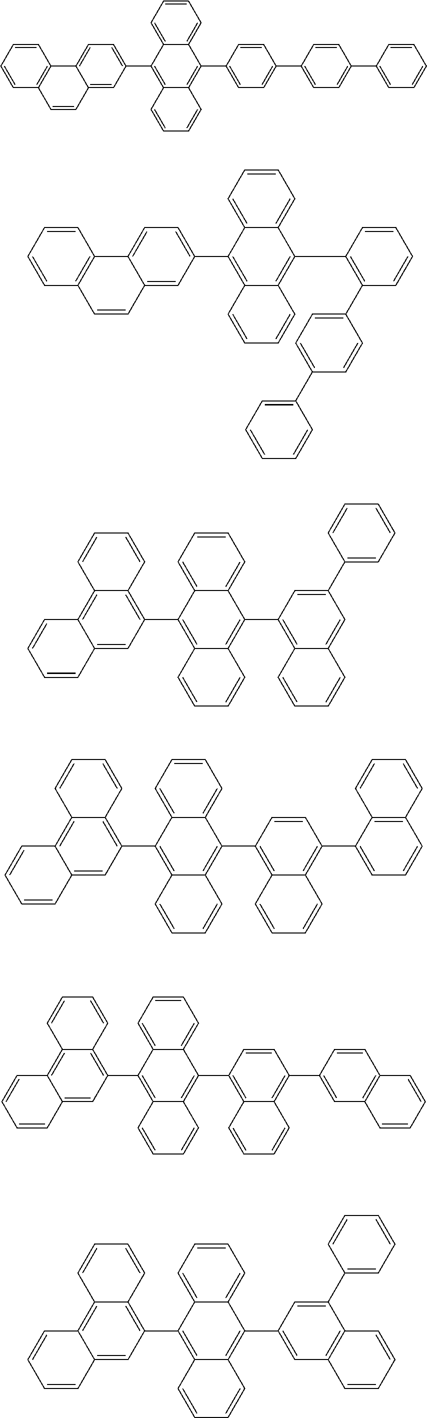

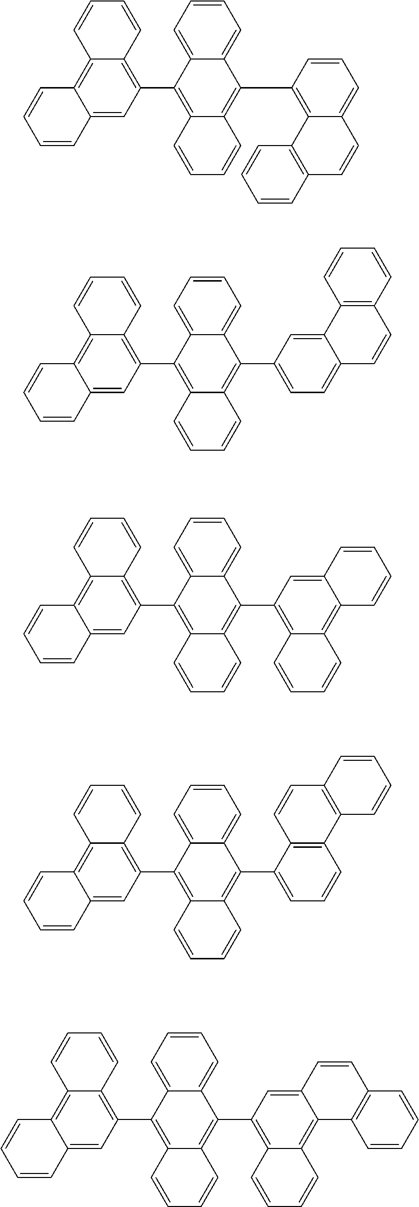

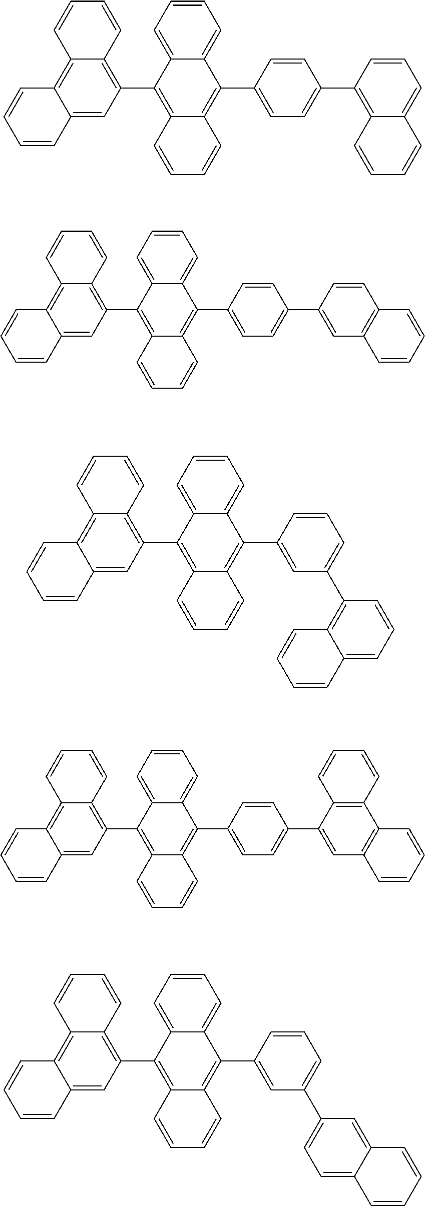

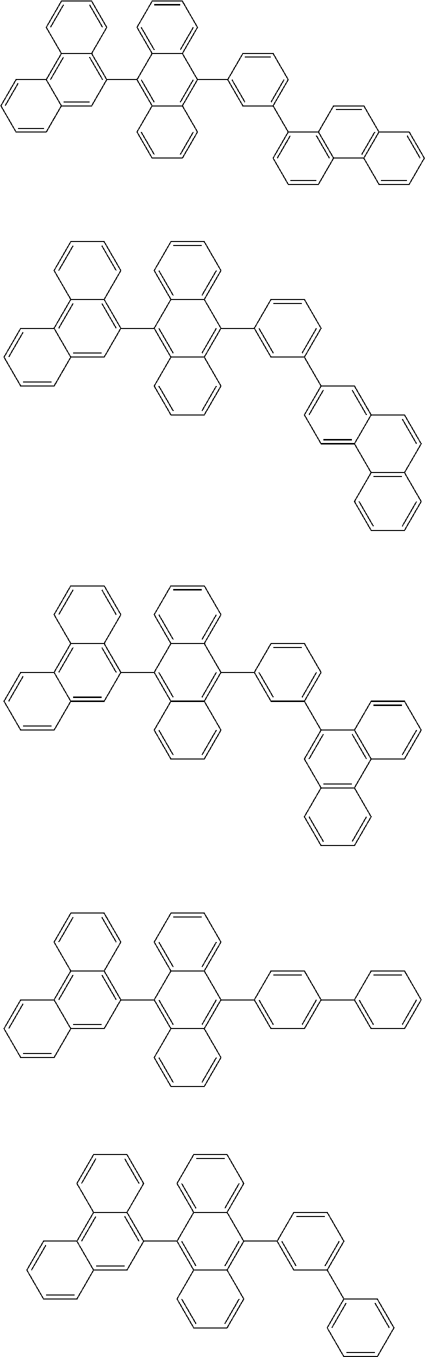

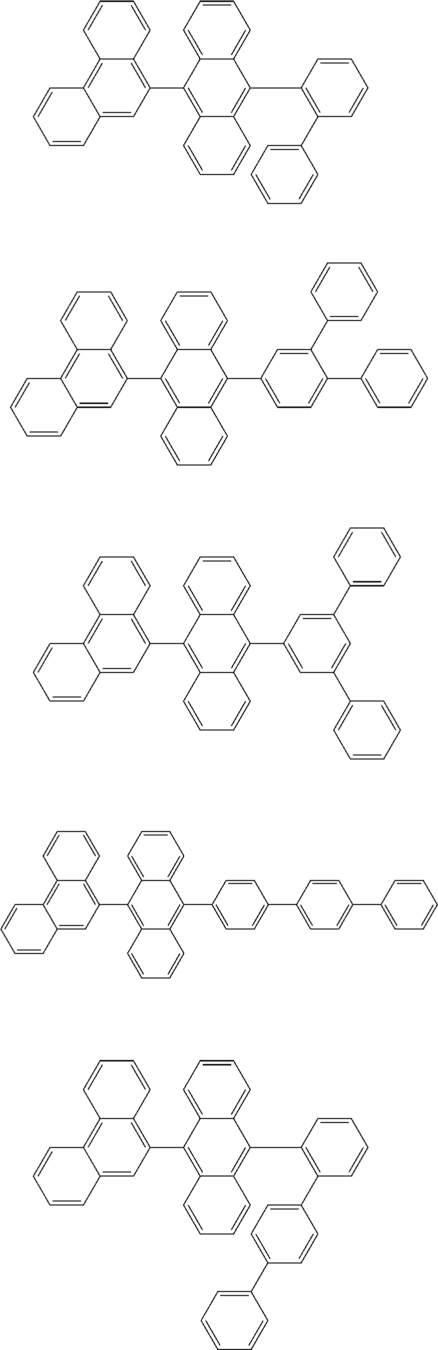

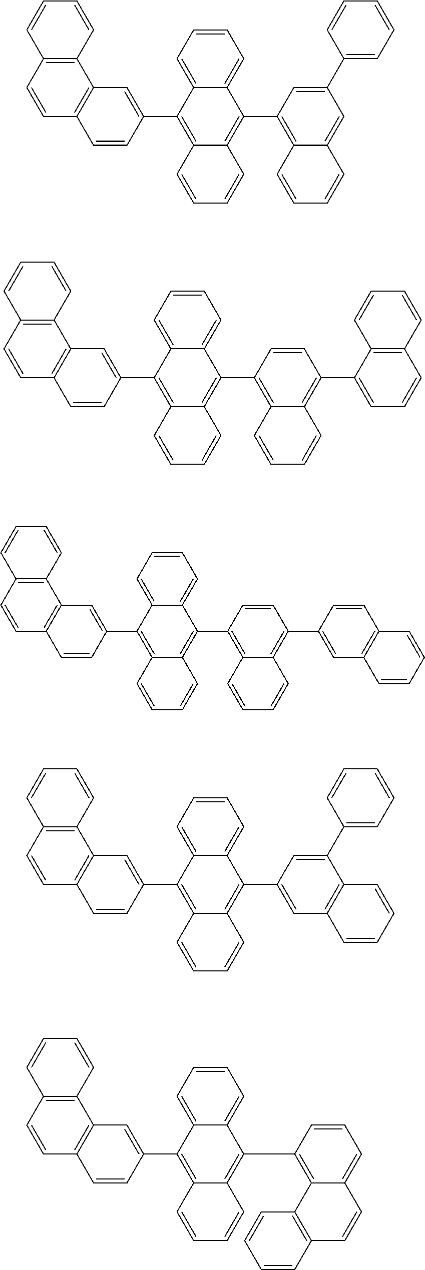

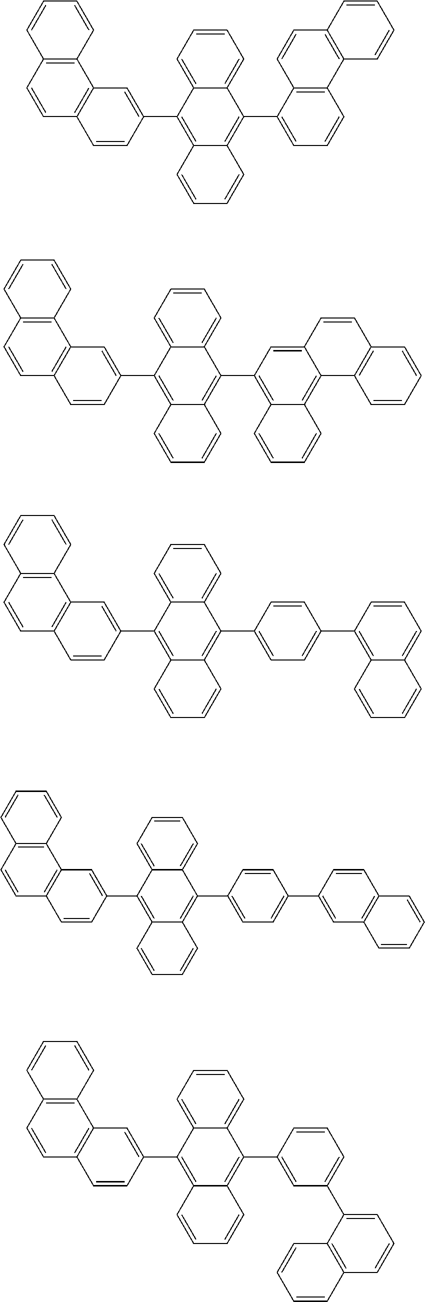

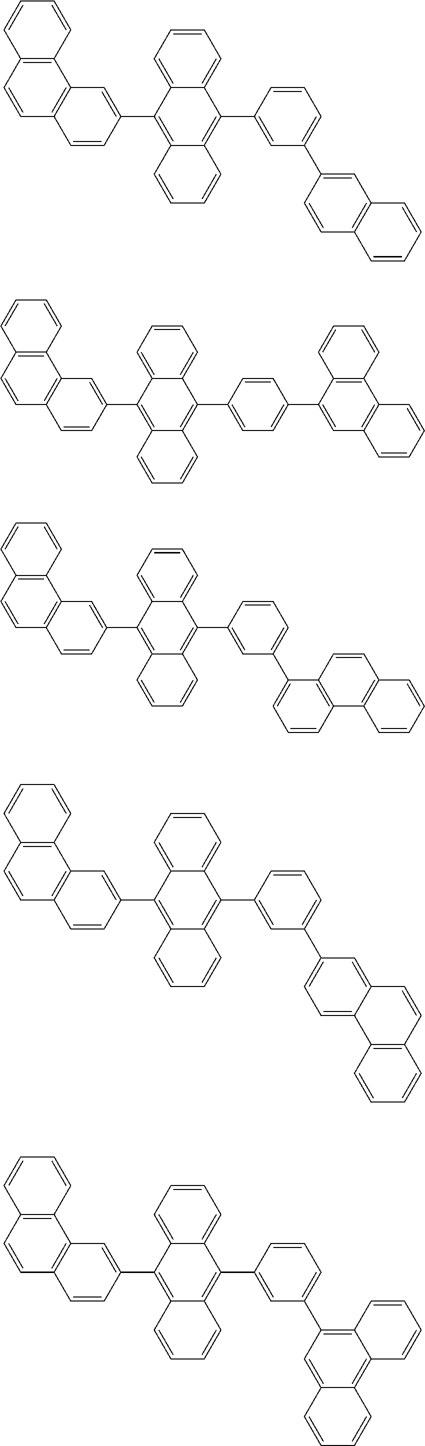

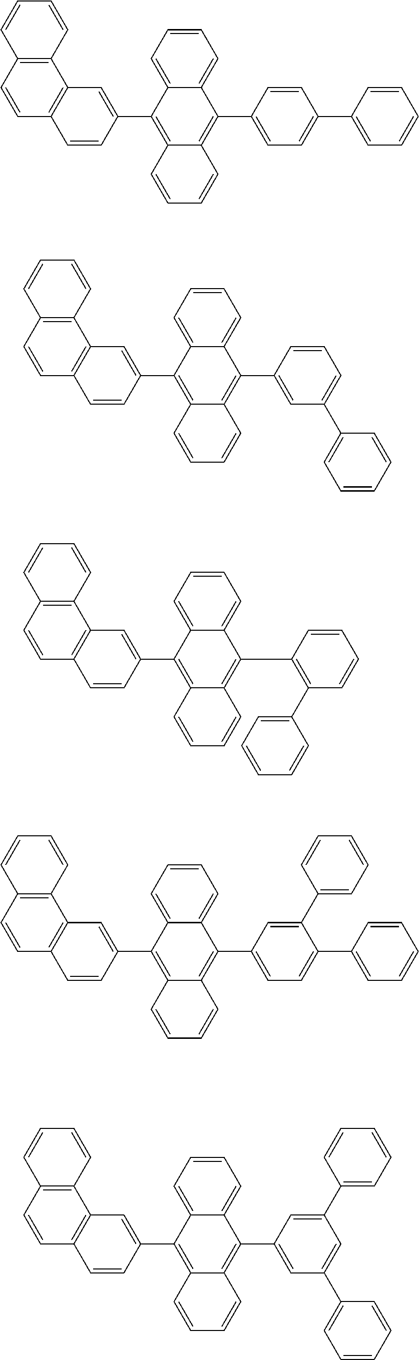

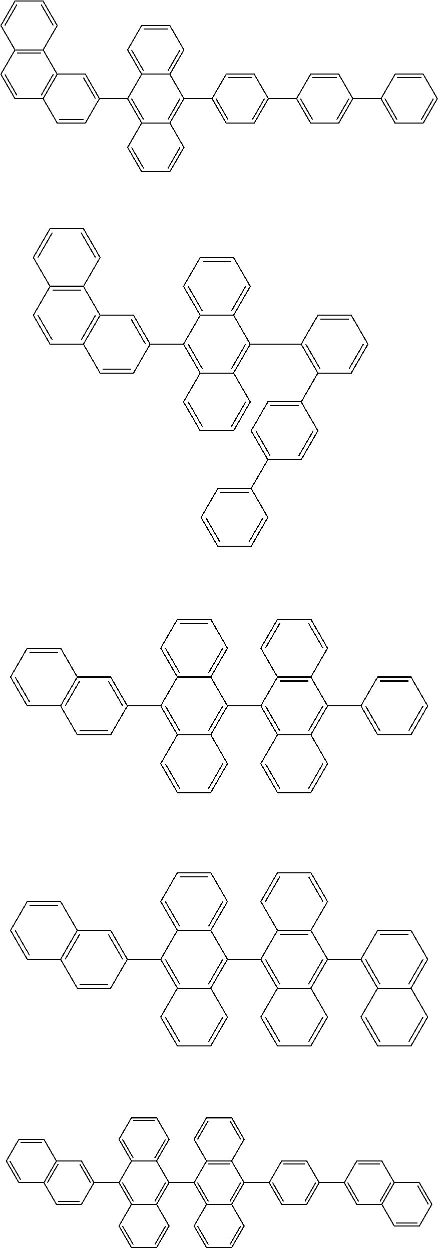

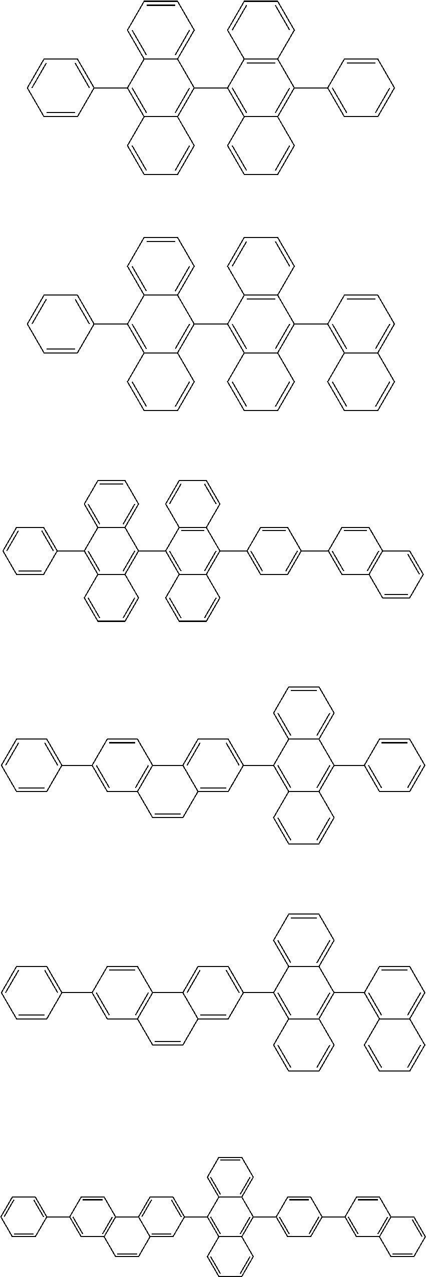

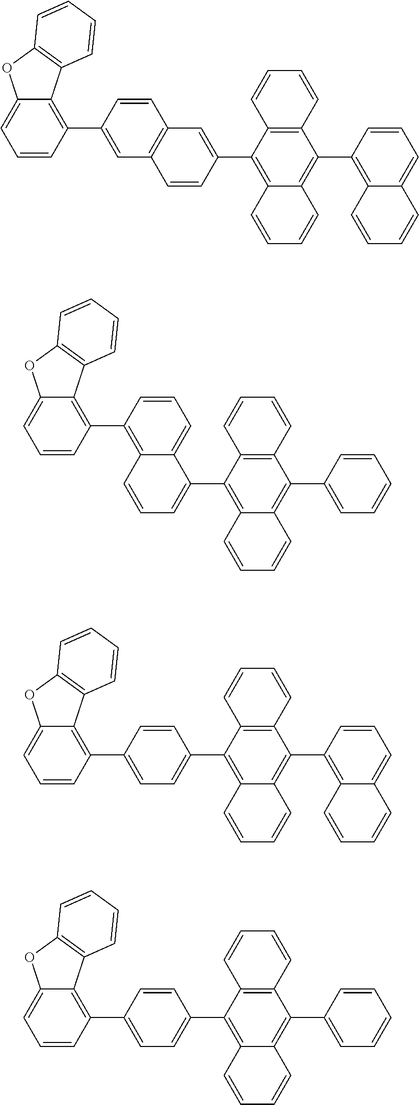 [SMILES: c1ccc2c(-c3c4ccccc4c(-c4ccc5cc(-c6cccc7oc8ccccc8c67)ccc5c4)c4ccccc34)cccc2c1 ; c1ccc(-c2c3ccccc3c(-c3cccc4c(-c5cccc6oc7ccccc7c56)cccc34)c3ccccc23)cc1 ; c1ccc2c(-c3c4ccccc4c(-c4ccc(-c5cccc6oc7ccccc7c56)cc4)c4ccccc34)cccc2c1 ; c1ccc(-c2c3ccccc3c(-c3ccc(-c4cccc5oc6ccccc6c45)cc3)c3ccccc23)cc1]

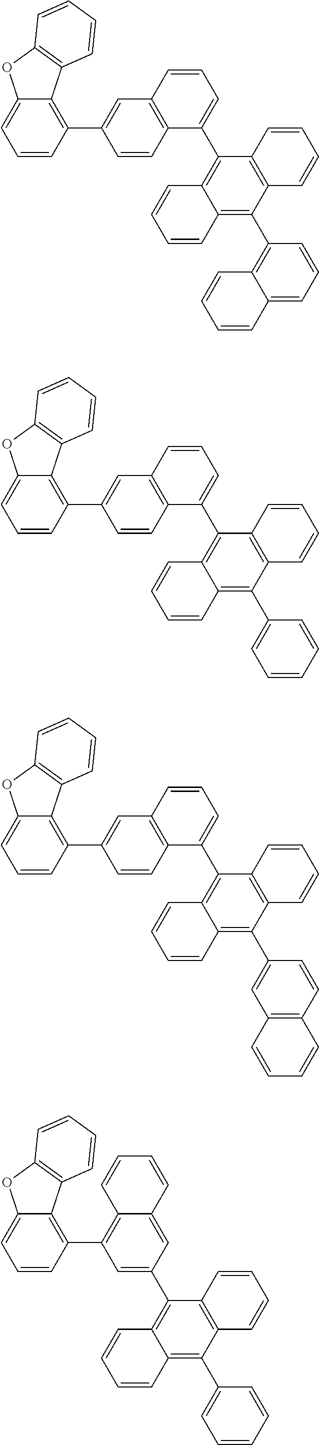 [SMILES: c1ccc2c(-c3c4ccccc4c(-c4cccc5cc(-c6cccc7oc8ccccc8c67)ccc45)c4ccccc34)cccc2c1 ; c1ccc(-c2c3ccccc3c(-c3cccc4cc(-c5cccc6oc7ccccc7c56)ccc34)c3ccccc23)cc1 ; c1ccc2cc(-c3c4ccccc4c(-c4cccc5cc(-c6cccc7oc8ccccc8c67)ccc45)c4ccccc34)ccc2c1 ; c1ccc(-c2c3ccccc3c(-c3cc(-c4cccc5oc6ccccc6c45)c4ccccc4c3)c3ccccc23)cc1]

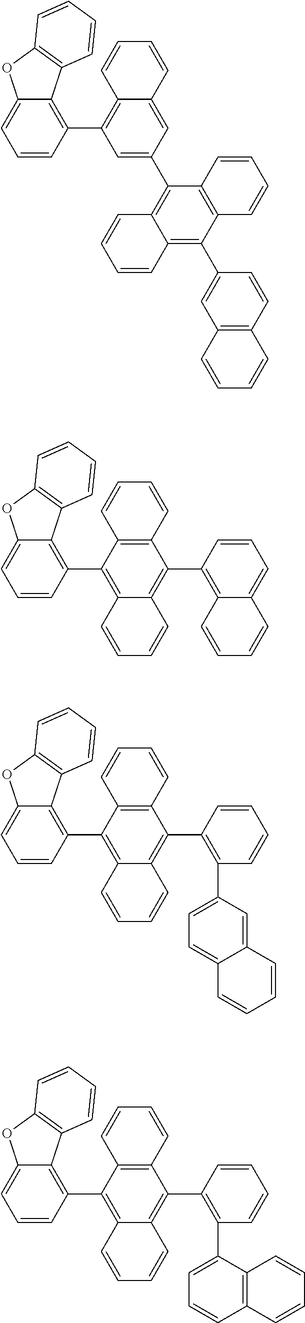 [SMILES: c1ccc2cc(-c3c4ccccc4c(-c4cc(-c5cccc6oc7ccccc7c56)c5ccccc5c4)c4ccccc34)ccc2c1 ; c1ccc2c(-c3c4ccccc4c(-c4cccc5oc6ccccc6c45)c4ccccc34)cccc2c1 ; c1ccc(-c2c3ccccc3c(-c3cccc4oc5ccccc5c34)c3ccccc23)c(-c2ccc3ccccc3c2)c1 ; c1ccc(-c2c3ccccc3c(-c3cccc4oc5ccccc5c34)c3ccccc23)c(-c2cccc3ccccc23)c1]

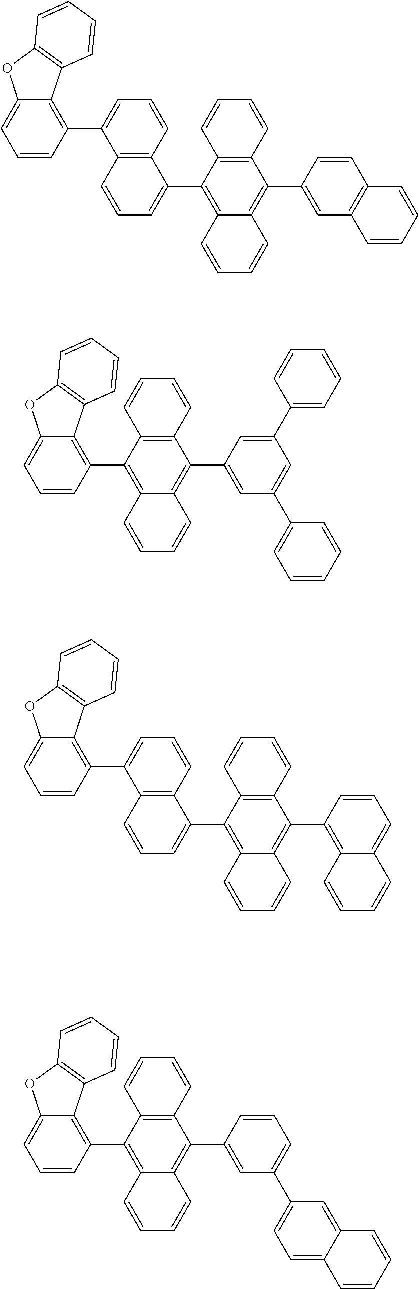 [SMILES: c1ccc2cc(-c3c4ccccc4c(-c4cccc5c(-c6cccc7oc8ccccc8c67)cccc45)c4ccccc34)ccc2c1 ; c1ccc(-c2cc(-c3ccccc3)cc(-c3c4ccccc4c(-c4cccc5oc6ccccc6c45)c4ccccc34)c2)cc1 ; c1ccc2c(-c3c4ccccc4c(-c4cccc5c(-c6cccc7oc8ccccc8c67)cccc45)c4ccccc34)cccc2c1 ; c1cc(-c2ccc3ccccc3c2)cc(-c2c3ccccc3c(-c3cccc4oc5ccccc5c34)c3ccccc23)c1]

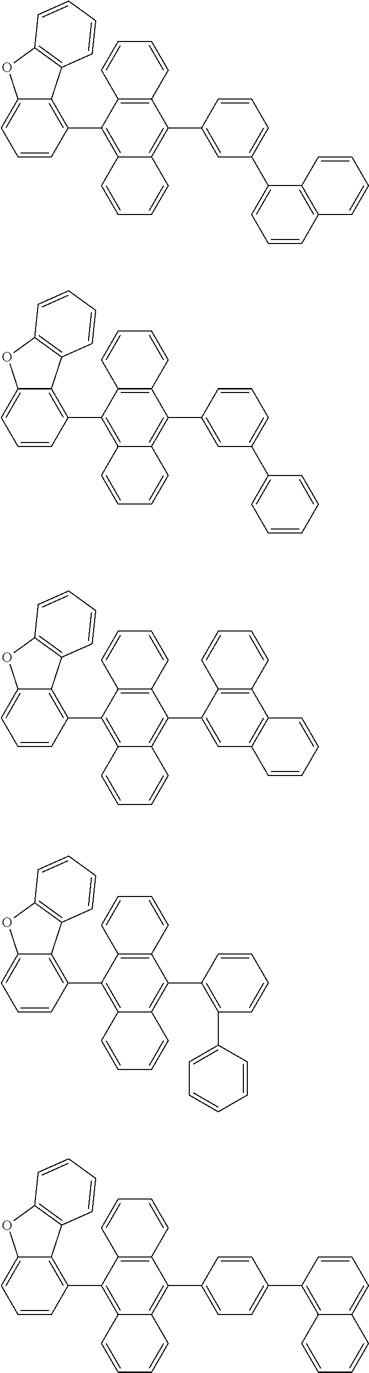 [SMILES: c1cc(-c2cccc3ccccc23)cc(-c2c3ccccc3c(-c3cccc4oc5ccccc5c34)c3ccccc23)c1 ; c1ccc(-c2cccc(-c3c4ccccc4c(-c4cccc5oc6ccccc6c45)c4ccccc34)c2)cc1 ; c1ccc2c(c1)cc(-c1c3ccccc3c(-c3cccc4oc5ccccc5c34)c3ccccc13)c1ccccc12 ; c1ccc(-c2ccccc2-c2c3ccccc3c(-c3cccc4oc5ccccc5c34)c3ccccc23)cc1 ; c1ccc2c(-c3ccc(-c4c5ccccc5c(-c5cccc6oc7ccccc7c56)c5ccccc45)cc3)cccc2c1]

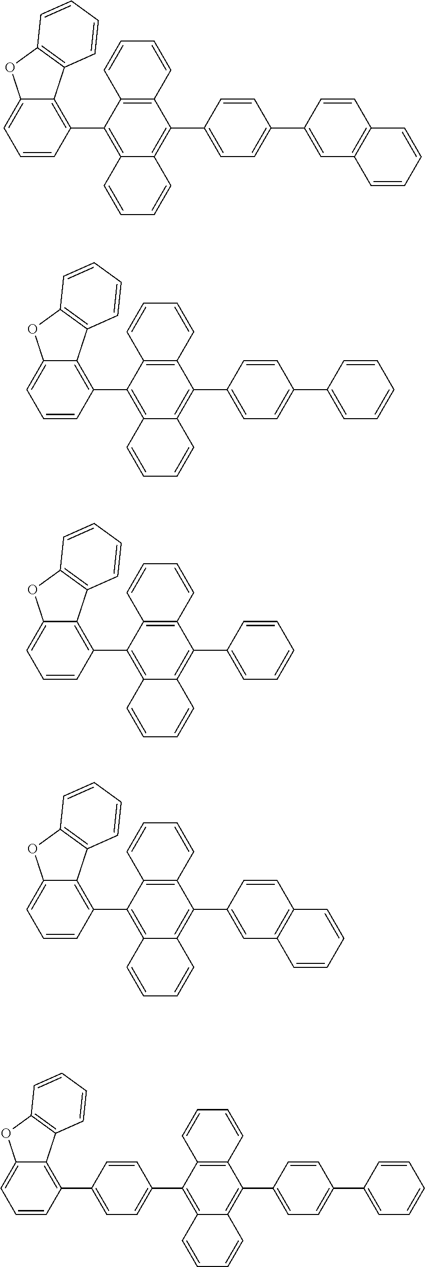 [SMILES: c1ccc2cc(-c3ccc(-c4c5ccccc5c(-c5cccc6oc7ccccc7c56)c5ccccc45)cc3)ccc2c1 ; c1ccc(-c2ccc(-c3c4ccccc4c(-c4cccc5oc6ccccc6c45)c4ccccc34)cc2)cc1 ; c1ccc(-c2c3ccccc3c(-c3cccc4oc5ccccc5c34)c3ccccc23)cc1 ; c1ccc2cc(-c3c4ccccc4c(-c4cccc5oc6ccccc6c45)c4ccccc34)ccc2c1 ; c1ccc(-c2ccc(-c3c4ccccc4c(-c4ccc(-c5cccc6oc7ccccc7c56)cc4)c4ccccc34)cc2)cc1]

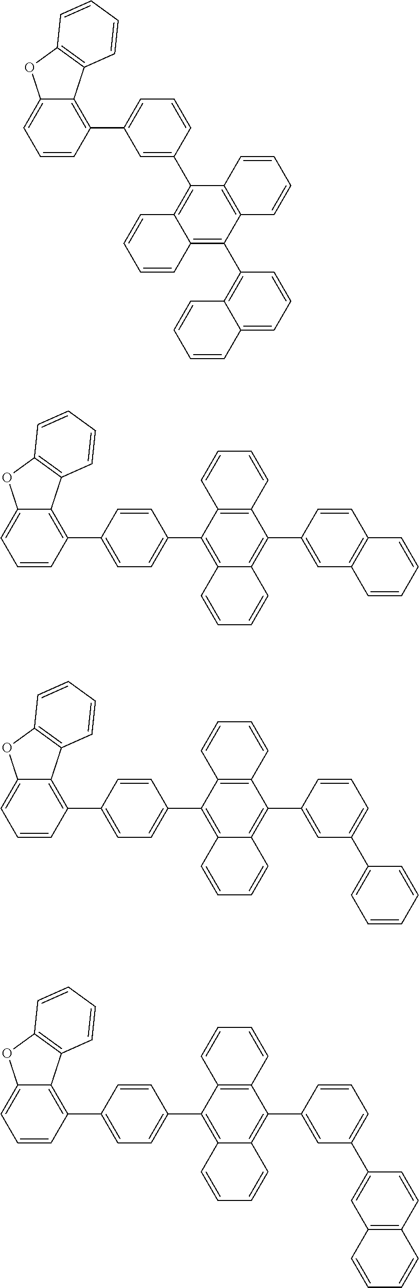 [SMILES: c1cc(-c2c3ccccc3c(-c3cccc4ccccc34)c3ccccc23)cc(-c2cccc3oc4ccccc4c23)c1 ; c1ccc2cc(-c3c4ccccc4c(-c4ccc(-c5cccc6oc7ccccc7c56)cc4)c4ccccc34)ccc2c1 ; c1ccc(-c2cccc(-c3c4ccccc4c(-c4ccc(-c5cccc6oc7ccccc7c56)cc4)c4ccccc34)c2)cc1 ; c1cc(-c2ccc3ccccc3c2)cc(-c2c3ccccc3c(-c3ccc(-c4cccc5oc6ccccc6c45)cc3)c3ccccc23)c1]

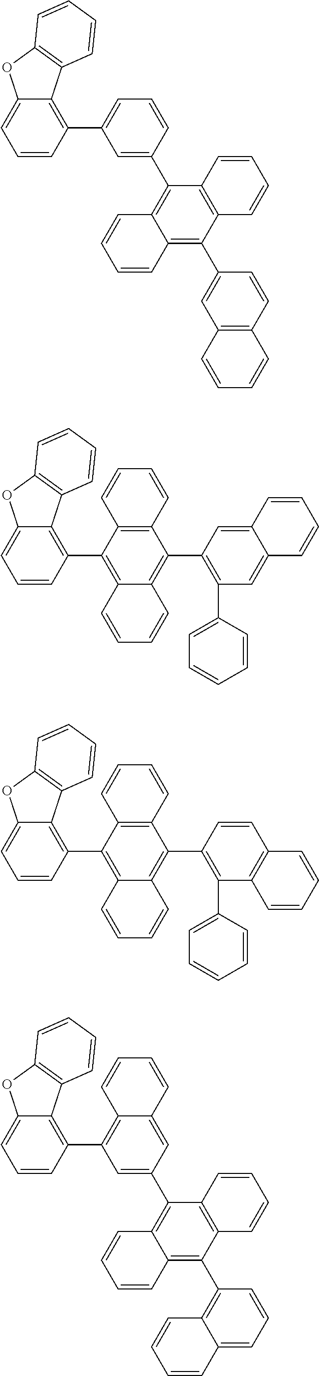 [SMILES: c1cc(-c2c3ccccc3c(-c3ccc4ccccc4c3)c3ccccc23)cc(-c2cccc3oc4ccccc4c23)c1 ; c1ccc(-c2cc3ccccc3cc2-c2c3ccccc3c(-c3cccc4oc5ccccc5c34)c3ccccc23)cc1 ; c1ccc(-c2c(-c3c4ccccc4c(-c4cccc5oc6ccccc6c45)c4ccccc34)ccc3ccccc23)cc1 ; c1ccc2c(-c3c4ccccc4c(-c4cc(-c5cccc6oc7ccccc7c56)c5ccccc5c4)c4ccccc34)cccc2c1]

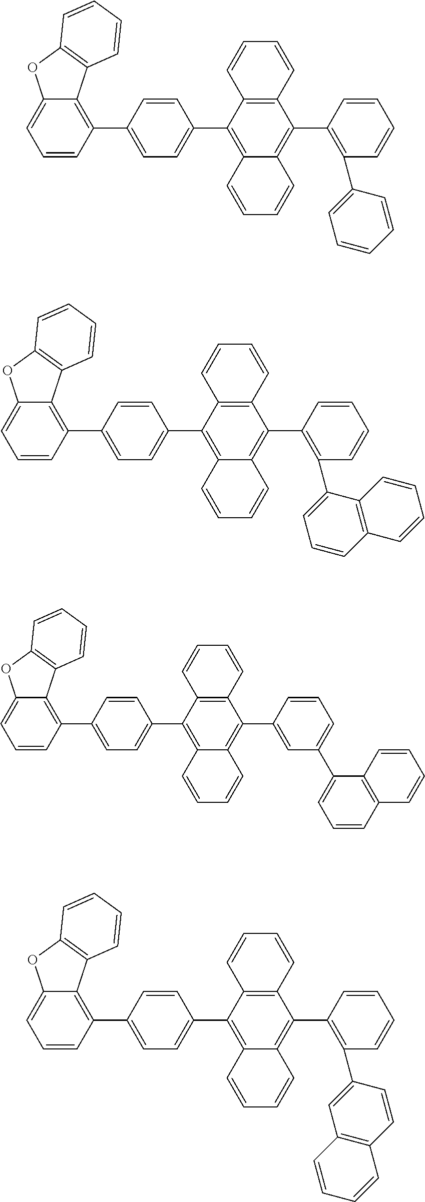 [SMILES: c1ccc(-c2ccccc2-c2c3ccccc3c(-c3ccc(-c4cccc5oc6ccccc6c45)cc3)c3ccccc23)cc1 ; c1ccc(-c2c3ccccc3c(-c3ccc(-c4cccc5oc6ccccc6c45)cc3)c3ccccc23)c(-c2cccc3ccccc23)c1 ; c1cc(-c2cccc3ccccc23)cc(-c2c3ccccc3c(-c3ccc(-c4cccc5oc6ccccc6c45)cc3)c3ccccc23)c1 ; c1ccc(-c2c3ccccc3c(-c3ccc(-c4cccc5oc6ccccc6c45)cc3)c3ccccc23)c(-c2ccc3ccccc3c2)c1]

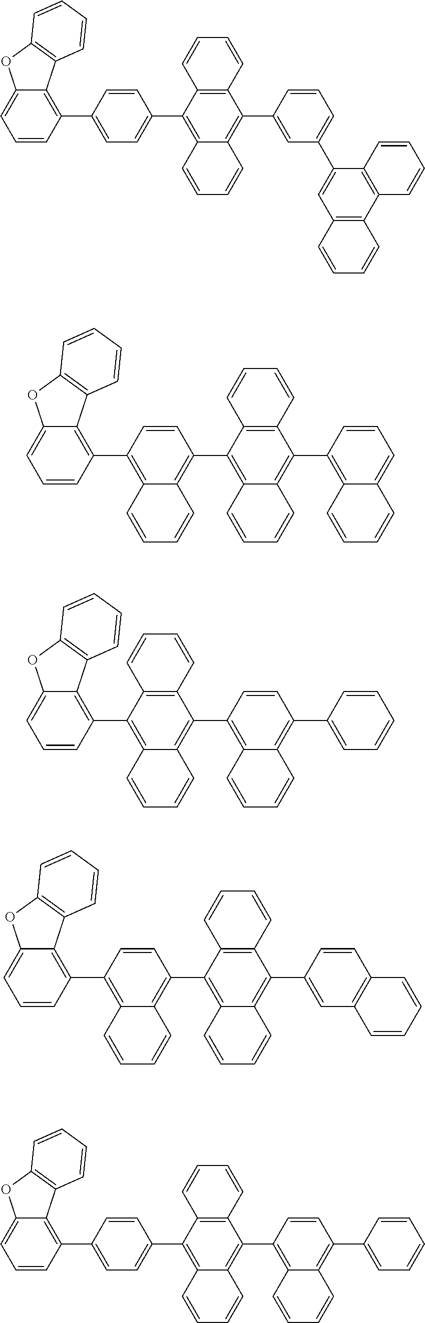 [SMILES: c1cc(-c2c3ccccc3c(-c3ccc(-c4cccc5oc6ccccc6c45)cc3)c3ccccc23)cc(-c2cc3ccccc3c3ccccc23)c1 ; c1ccc2c(-c3c4ccccc4c(-c4ccc(-c5cccc6oc7ccccc7c56)c5ccccc45)c4ccccc34)cccc2c1 ; c1ccc(-c2ccc(-c3c4ccccc4c(-c4cccc5oc6ccccc6c45)c4ccccc34)c3ccccc23)cc1 ; c1ccc2cc(-c3c4ccccc4c(-c4ccc(-c5cccc6oc7ccccc7c56)c5ccccc45)c4ccccc34)ccc2c1 ; c1ccc(-c2ccc(-c3c4ccccc4c(-c4ccc(-c5cccc6oc7ccccc7c56)cc4)c4ccccc34)c3ccccc23)cc1]

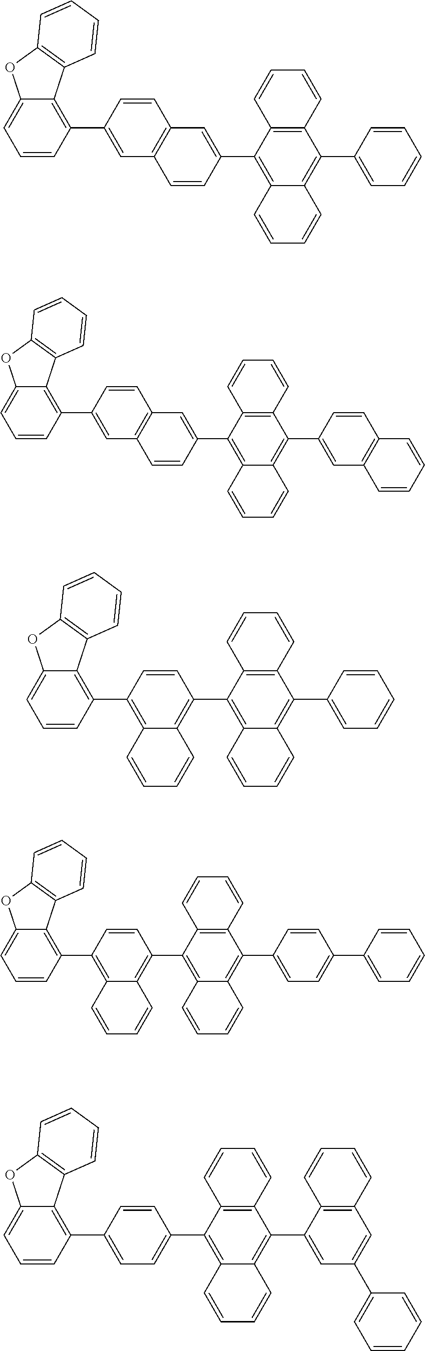 [SMILES: c1ccc(-c2c3ccccc3c(-c3ccc4cc(-c5cccc6oc7ccccc7c56)ccc4c3)c3ccccc23)cc1 ; c1ccc2cc(-c3c4ccccc4c(-c4ccc5cc(-c6cccc7oc8ccccc8c67)ccc5c4)c4ccccc34)ccc2c1 ; c1ccc(-c2c3ccccc3c(-c3ccc(-c4cccc5oc6ccccc6c45)c4ccccc34)c3ccccc23)cc1 ; c1ccc(-c2ccc(-c3c4ccccc4c(-c4ccc(-c5cccc6oc7ccccc7c56)c5ccccc45)c4ccccc34)cc2)cc1 ; c1ccc(-c2cc(-c3c4ccccc4c(-c4ccc(-c5cccc6oc7ccccc7c56)cc4)c4ccccc34)c3ccccc3c2)cc1]

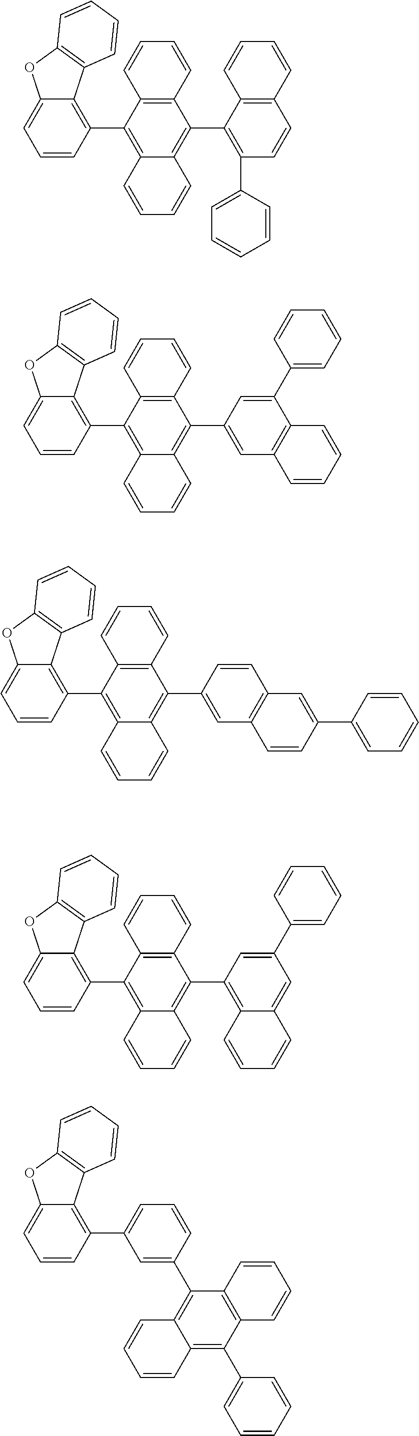 [SMILES: c1ccc(-c2ccc3ccccc3c2-c2c3ccccc3c(-c3cccc4oc5ccccc5c34)c3ccccc23)cc1 ; c1ccc(-c2cc(-c3c4ccccc4c(-c4cccc5oc6ccccc6c45)c4ccccc34)cc3ccccc23)cc1 ; c1ccc(-c2ccc3cc(-c4c5ccccc5c(-c5cccc6oc7ccccc7c56)c5ccccc45)ccc3c2)cc1 ; c1ccc(-c2cc(-c3c4ccccc4c(-c4cccc5oc6ccccc6c45)c4ccccc34)c3ccccc3c2)cc1 ; c1ccc(-c2c3ccccc3c(-c3cccc(-c4cccc5oc6ccccc6c45)c3)c3ccccc23)cc1]

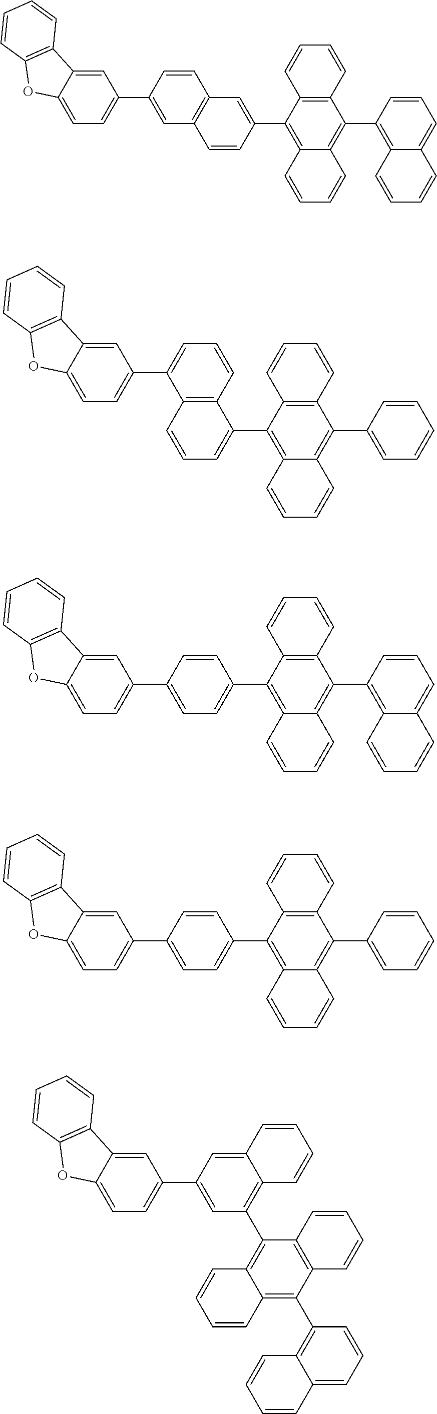 [SMILES: c1ccc2c(-c3c4ccccc4c(-c4ccc5cc(-c6ccc7oc8ccccc8c7c6)ccc5c4)c4ccccc34)cccc2c1 ; c1ccc(-c2c3ccccc3c(-c3cccc4c(-c5ccc6oc7ccccc7c6c5)cccc34)c3ccccc23)cc1 ; c1ccc2c(-c3c4ccccc4c(-c4ccc(-c5ccc6oc7ccccc7c6c5)cc4)c4ccccc34)cccc2c1 ; c1ccc(-c2c3ccccc3c(-c3ccc(-c4ccc5oc6ccccc6c5c4)cc3)c3ccccc23)cc1 ; c1ccc2c(-c3c4ccccc4c(-c4cc(-c5ccc6oc7ccccc7c6c5)cc5ccccc45)c4ccccc34)cccc2c1]

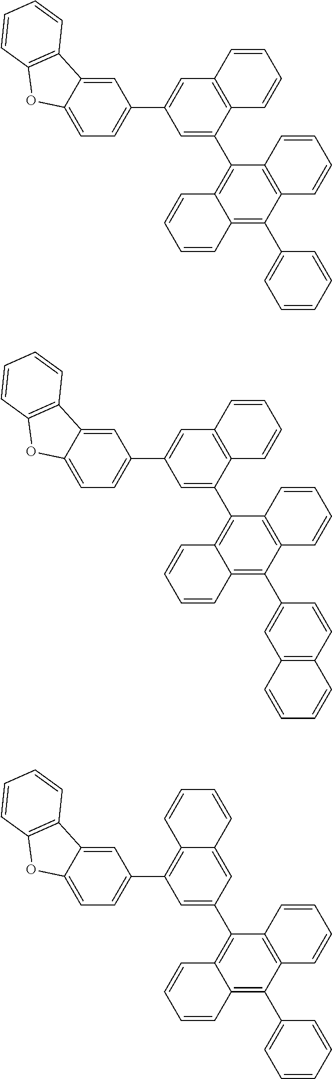 [SMILES: c1ccc(-c2c3ccccc3c(-c3cc(-c4ccc5oc6ccccc6c5c4)cc4ccccc34)c3ccccc23)cc1 ; c1ccc2cc(-c3c4ccccc4c(-c4cc(-c5ccc6oc7ccccc7c6c5)cc5ccccc45)c4ccccc34)ccc2c1 ; c1ccc(-c2c3ccccc3c(-c3cc(-c4ccc5oc6ccccc6c5c4)c4ccccc4c3)c3ccccc23)cc1]

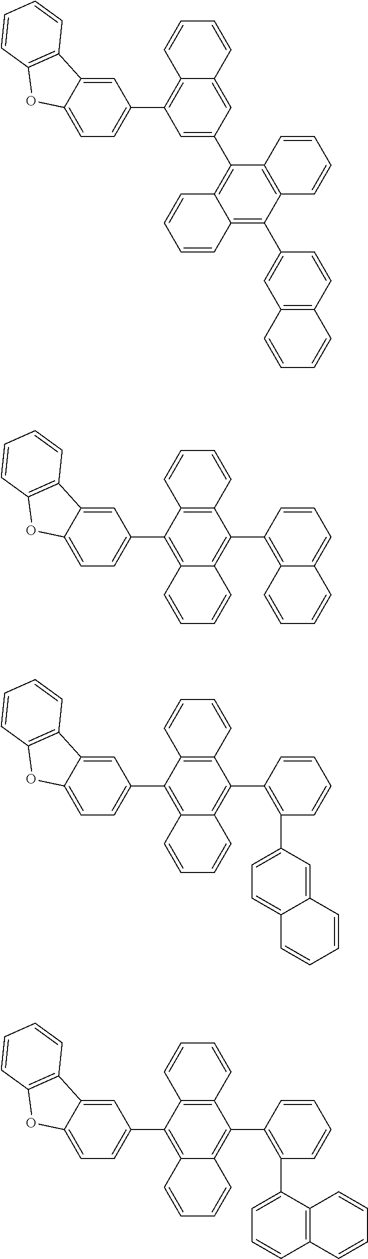 [SMILES: c1ccc2cc(-c3c4ccccc4c(-c4cc(-c5ccc6oc7ccccc7c6c5)c5ccccc5c4)c4ccccc34)ccc2c1 ; c1ccc2c(-c3c4ccccc4c(-c4ccc5oc6ccccc6c5c4)c4ccccc34)cccc2c1 ; c1ccc(-c2c3ccccc3c(-c3ccc4oc5ccccc5c4c3)c3ccccc23)c(-c2ccc3ccccc3c2)c1 ; c1ccc(-c2c3ccccc3c(-c3ccc4oc5ccccc5c4c3)c3ccccc23)c(-c2cccc3ccccc23)c1]

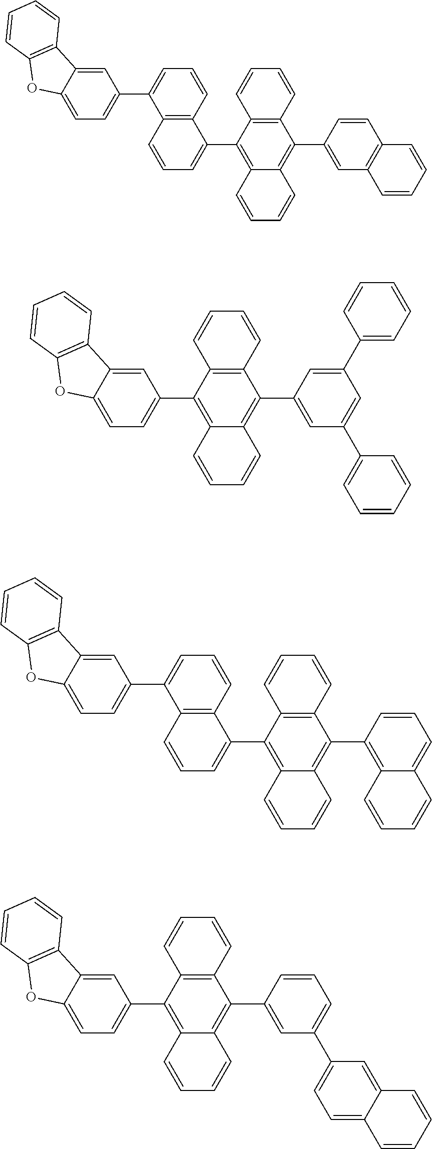 [SMILES: c1ccc2cc(-c3c4ccccc4c(-c4cccc5c(-c6ccc7oc8ccccc8c7c6)cccc45)c4ccccc34)ccc2c1 ; c1ccc(-c2cc(-c3ccccc3)cc(-c3c4ccccc4c(-c4ccc5oc6ccccc6c5c4)c4ccccc34)c2)cc1 ; c1ccc2c(-c3c4ccccc4c(-c4cccc5c(-c6ccc7oc8ccccc8c7c6)cccc45)c4ccccc34)cccc2c1 ; c1cc(-c2ccc3ccccc3c2)cc(-c2c3ccccc3c(-c3ccc4oc5ccccc5c4c3)c3ccccc23)c1]

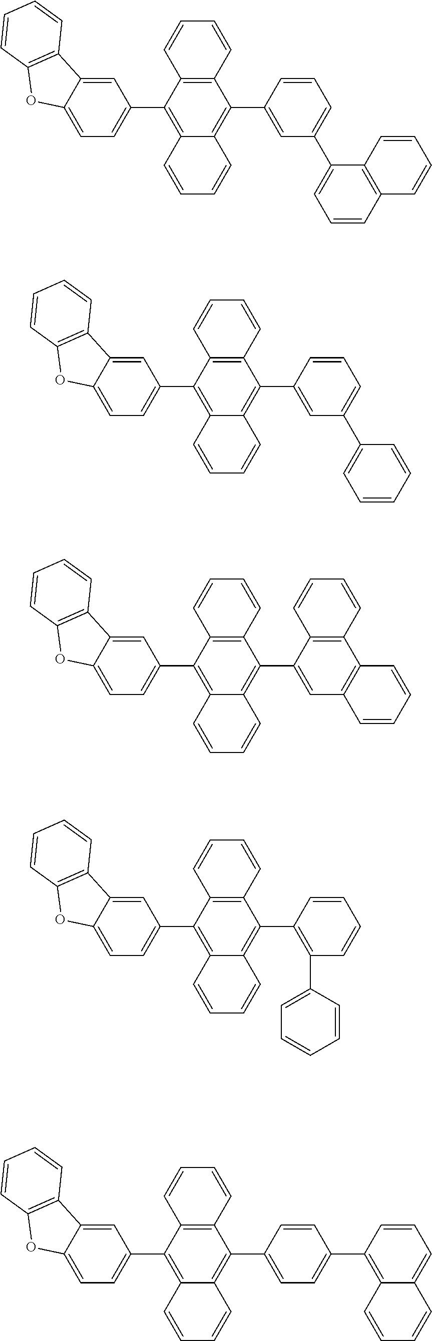 [SMILES: c1cc(-c2cccc3ccccc23)cc(-c2c3ccccc3c(-c3ccc4oc5ccccc5c4c3)c3ccccc23)c1 ; c1ccc(-c2cccc(-c3c4ccccc4c(-c4ccc5oc6ccccc6c5c4)c4ccccc34)c2)cc1 ; c1ccc2c(c1)cc(-c1c3ccccc3c(-c3ccc4oc5ccccc5c4c3)c3ccccc13)c1ccccc12 ; c1ccc(-c2ccccc2-c2c3ccccc3c(-c3ccc4oc5ccccc5c4c3)c3ccccc23)cc1 ; c1ccc2c(-c3ccc(-c4c5ccccc5c(-c5ccc6oc7ccccc7c6c5)c5ccccc45)cc3)cccc2c1]

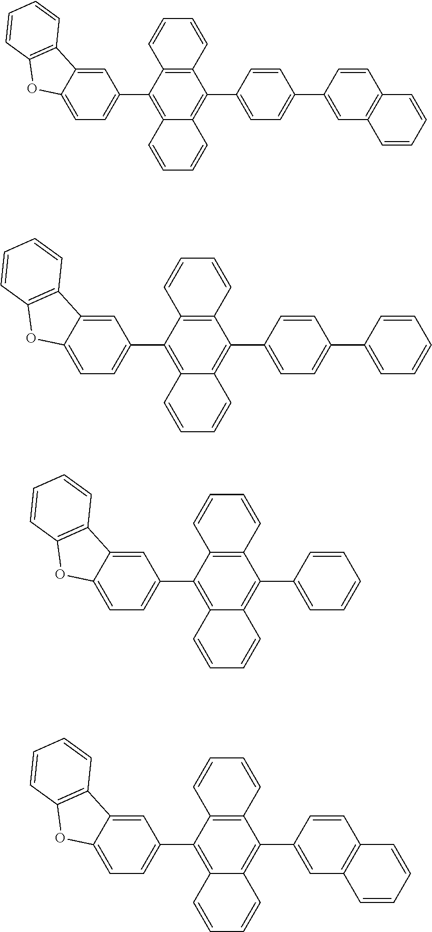 [SMILES: c1ccc2cc(-c3ccc(-c4c5ccccc5c(-c5ccc6oc7ccccc7c6c5)c5ccccc45)cc3)ccc2c1 ; c1ccc(-c2ccc(-c3c4ccccc4c(-c4ccc5oc6ccccc6c5c4)c4ccccc34)cc2)cc1 ; c1ccc(-c2c3ccccc3c(-c3ccc4oc5ccccc5c4c3)c3ccccc23)cc1 ; c1ccc2cc(-c3c4ccccc4c(-c4ccc5oc6ccccc6c5c4)c4ccccc34)ccc2c1]

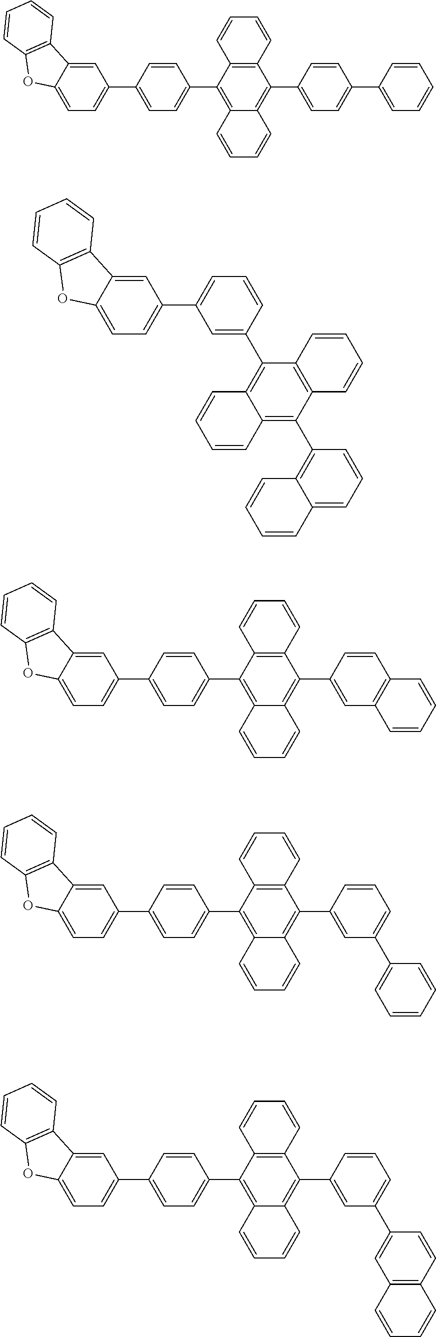 [SMILES: c1ccc(-c2ccc(-c3c4ccccc4c(-c4ccc(-c5ccc6oc7ccccc7c6c5)cc4)c4ccccc34)cc2)cc1 ; c1cc(-c2ccc3oc4ccccc4c3c2)cc(-c2c3ccccc3c(-c3cccc4ccccc34)c3ccccc23)c1 ; c1ccc2cc(-c3c4ccccc4c(-c4ccc(-c5ccc6oc7ccccc7c6c5)cc4)c4ccccc34)ccc2c1 ; c1ccc(-c2cccc(-c3c4ccccc4c(-c4ccc(-c5ccc6oc7ccccc7c6c5)cc4)c4ccccc34)c2)cc1 ; c1cc(-c2ccc3ccccc3c2)cc(-c2c3ccccc3c(-c3ccc(-c4ccc5oc6ccccc6c5c4)cc3)c3ccccc23)c1]

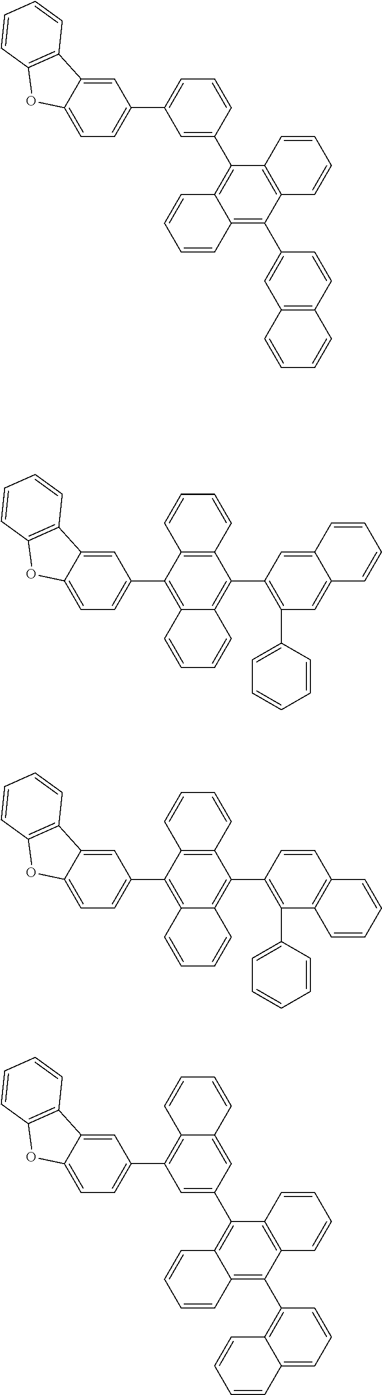 [SMILES: c1cc(-c2ccc3oc4ccccc4c3c2)cc(-c2c3ccccc3c(-c3ccc4ccccc4c3)c3ccccc23)c1 ; c1ccc(-c2cc3ccccc3cc2-c2c3ccccc3c(-c3ccc4oc5ccccc5c4c3)c3ccccc23)cc1 ; c1ccc(-c2c(-c3c4ccccc4c(-c4ccc5oc6ccccc6c5c4)c4ccccc34)ccc3ccccc23)cc1 ; c1ccc2c(-c3ccc4oc5ccccc5c4c3)cc(-c3c4ccccc4c(-c4cccc5ccccc45)c4ccccc34)cc2c1]

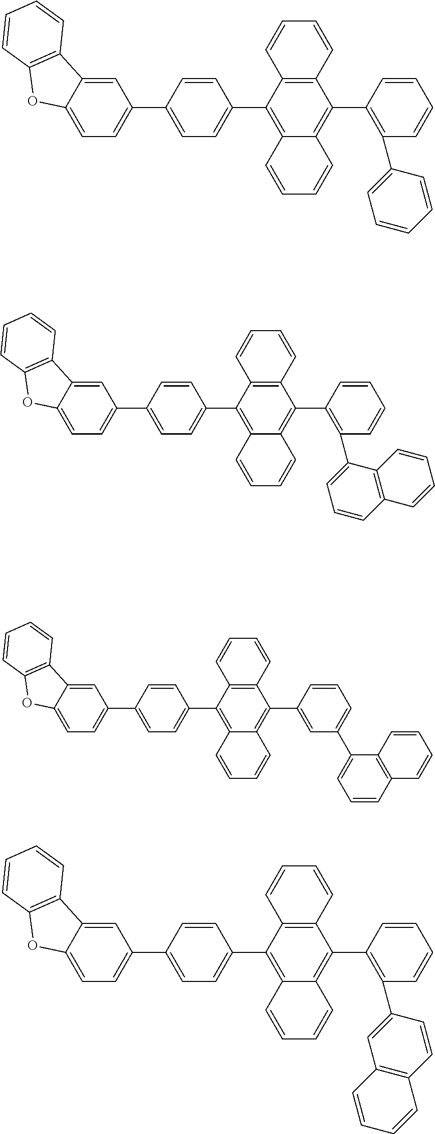 [SMILES: c1ccc(-c2ccccc2-c2c3ccccc3c(-c3ccc(-c4ccc5oc6ccccc6c5c4)cc3)c3ccccc23)cc1 ; c1ccc(-c2c3ccccc3c(-c3ccc(-c4ccc5oc6ccccc6c5c4)cc3)c3ccccc23)c(-c2cccc3ccccc23)c1 ; c1cc(-c2cccc3ccccc23)cc(-c2c3ccccc3c(-c3ccc(-c4ccc5oc6ccccc6c5c4)cc3)c3ccccc23)c1 ; c1ccc(-c2c3ccccc3c(-c3ccc(-c4ccc5oc6ccccc6c5c4)cc3)c3ccccc23)c(-c2ccc3ccccc3c2)c1]

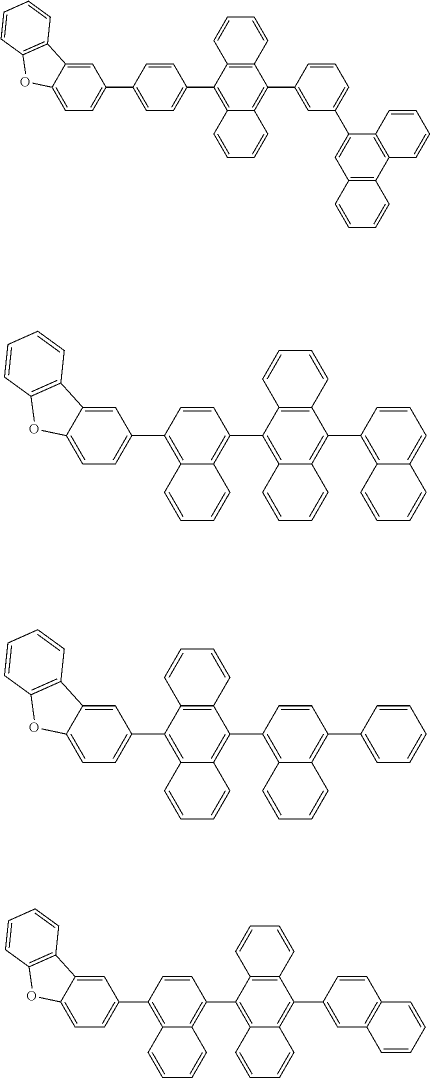 [SMILES: c1cc(-c2c3ccccc3c(-c3ccc(-c4ccc5oc6ccccc6c5c4)cc3)c3ccccc23)cc(-c2cc3ccccc3c3ccccc23)c1 ; c1ccc2c(-c3c4ccccc4c(-c4ccc(-c5ccc6oc7ccccc7c6c5)c5ccccc45)c4ccccc34)cccc2c1 ; c1ccc(-c2ccc(-c3c4ccccc4c(-c4ccc5oc6ccccc6c5c4)c4ccccc34)c3ccccc23)cc1 ; c1ccc2cc(-c3c4ccccc4c(-c4ccc(-c5ccc6oc7ccccc7c6c5)c5ccccc45)c4ccccc34)ccc2c1]

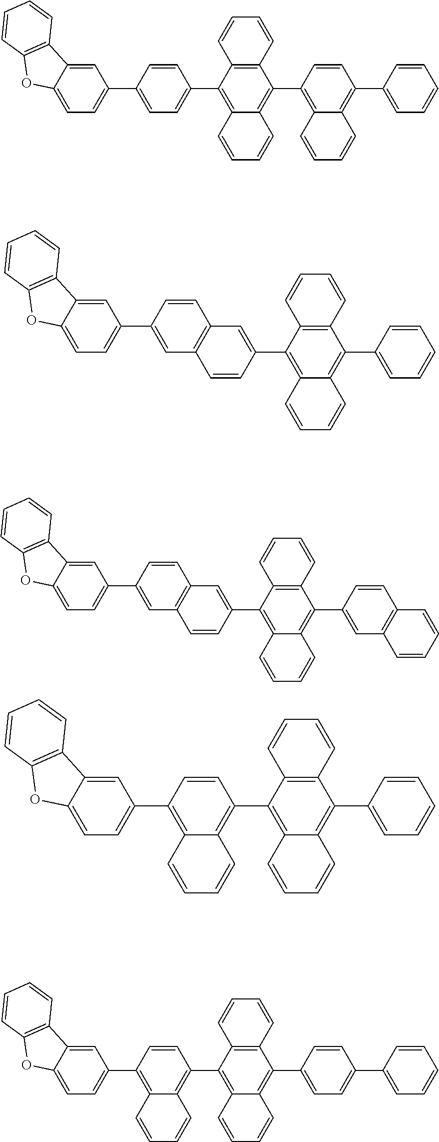 [SMILES: c1ccc(-c2ccc(-c3c4ccccc4c(-c4ccc(-c5ccc6oc7ccccc7c6c5)cc4)c4ccccc34)c3ccccc23)cc1 ; c1ccc(-c2c3ccccc3c(-c3ccc4cc(-c5ccc6oc7ccccc7c6c5)ccc4c3)c3ccccc23)cc1 ; c1ccc2cc(-c3c4ccccc4c(-c4ccc5cc(-c6ccc7oc8ccccc8c7c6)ccc5c4)c4ccccc34)ccc2c1 ; c1ccc(-c2c3ccccc3c(-c3ccc(-c4ccc5oc6ccccc6c5c4)c4ccccc34)c3ccccc23)cc1 ; c1ccc(-c2ccc(-c3c4ccccc4c(-c4ccc(-c5ccc6oc7ccccc7c6c5)c5ccccc45)c4ccccc34)cc2)cc1]

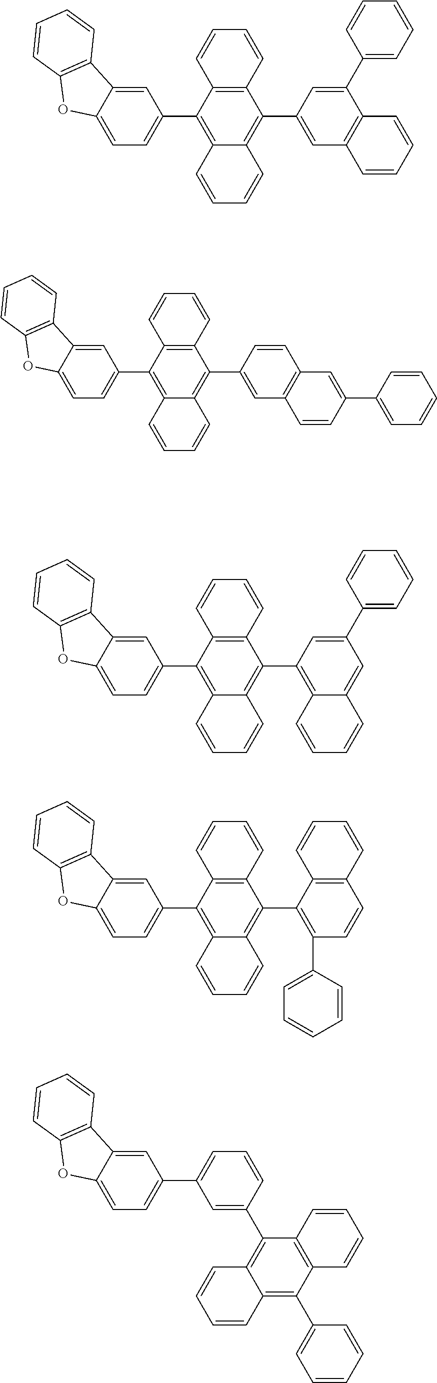 [SMILES: c1ccc(-c2cc(-c3c4ccccc4c(-c4ccc5oc6ccccc6c5c4)c4ccccc34)cc3ccccc23)cc1 ; c1ccc(-c2ccc3cc(-c4c5ccccc5c(-c5ccc6oc7ccccc7c6c5)c5ccccc45)ccc3c2)cc1 ; c1ccc(-c2cc(-c3c4ccccc4c(-c4ccc5oc6ccccc6c5c4)c4ccccc34)c3ccccc3c2)cc1 ; c1ccc(-c2ccc3ccccc3c2-c2c3ccccc3c(-c3ccc4oc5ccccc5c4c3)c3ccccc23)cc1 ; c1ccc(-c2c3ccccc3c(-c3cccc(-c4ccc5oc6ccccc6c5c4)c3)c3ccccc23)cc1]

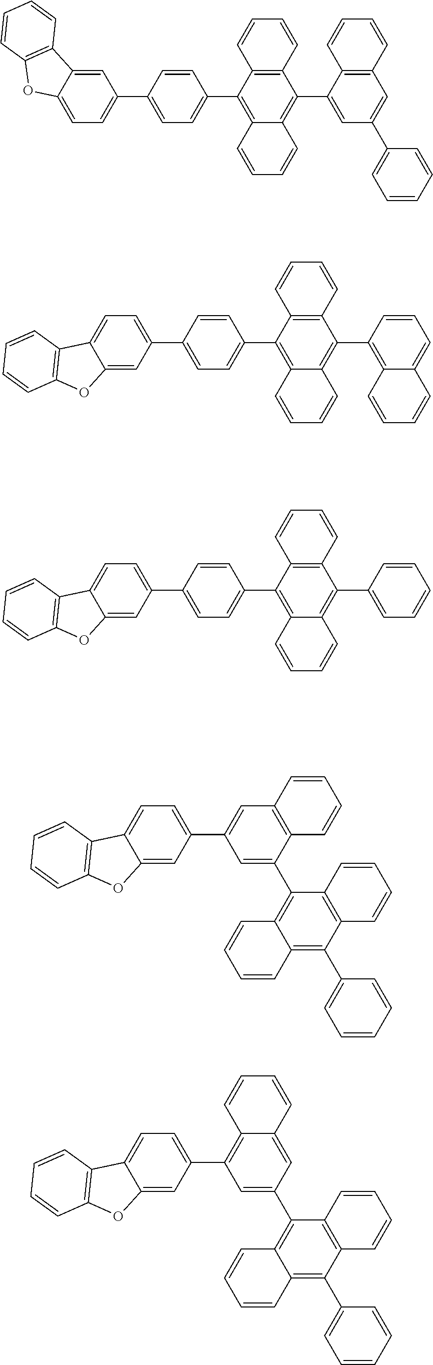 [SMILES: c1ccc(-c2cc(-c3c4ccccc4c(-c4ccc(-c5ccc6oc7ccccc7c6c5)cc4)c4ccccc34)c3ccccc3c2)cc1 ; c1ccc2c(-c3c4ccccc4c(-c4ccc(-c5ccc6c(c5)oc5ccccc56)cc4)c4ccccc34)cccc2c1 ; c1ccc(-c2c3ccccc3c(-c3ccc(-c4ccc5c(c4)oc4ccccc45)cc3)c3ccccc23)cc1 ; c1ccc(-c2c3ccccc3c(-c3cc(-c4ccc5c(c4)oc4ccccc45)cc4ccccc34)c3ccccc23)cc1 ; c1ccc(-c2c3ccccc3c(-c3cc(-c4ccc5c(c4)oc4ccccc45)c4ccccc4c3)c3ccccc23)cc1]

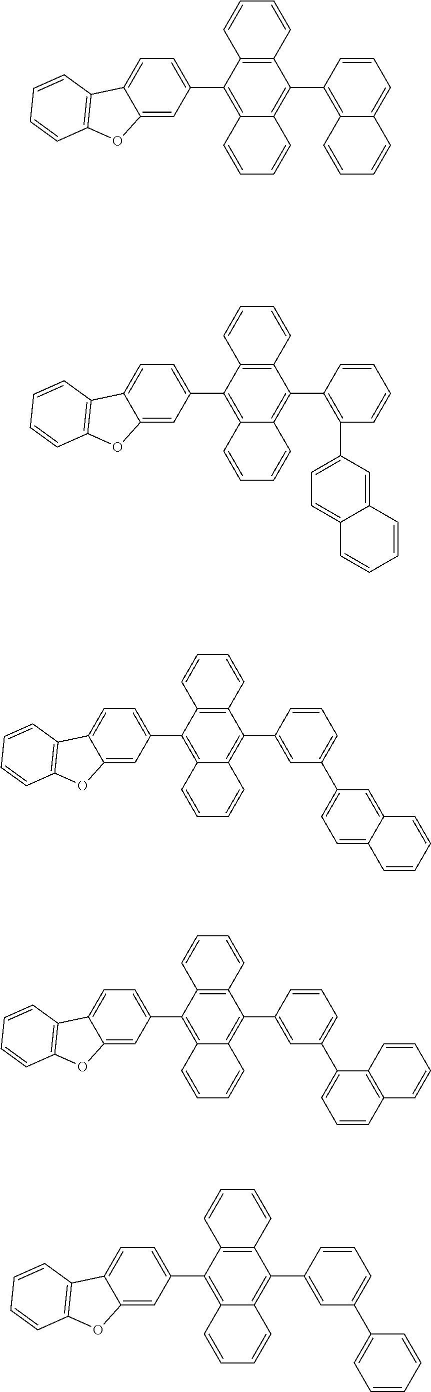 [SMILES: c1ccc2c(-c3c4ccccc4c(-c4ccc5c(c4)oc4ccccc45)c4ccccc34)cccc2c1 ; c1ccc(-c2c3ccccc3c(-c3ccc4c(c3)oc3ccccc34)c3ccccc23)c(-c2ccc3ccccc3c2)c1 ; c1cc(-c2ccc3ccccc3c2)cc(-c2c3ccccc3c(-c3ccc4c(c3)oc3ccccc34)c3ccccc23)c1 ; c1cc(-c2cccc3ccccc23)cc(-c2c3ccccc3c(-c3ccc4c(c3)oc3ccccc34)c3ccccc23)c1 ; c1ccc(-c2cccc(-c3c4ccccc4c(-c4ccc5c(c4)oc4ccccc45)c4ccccc34)c2)cc1]

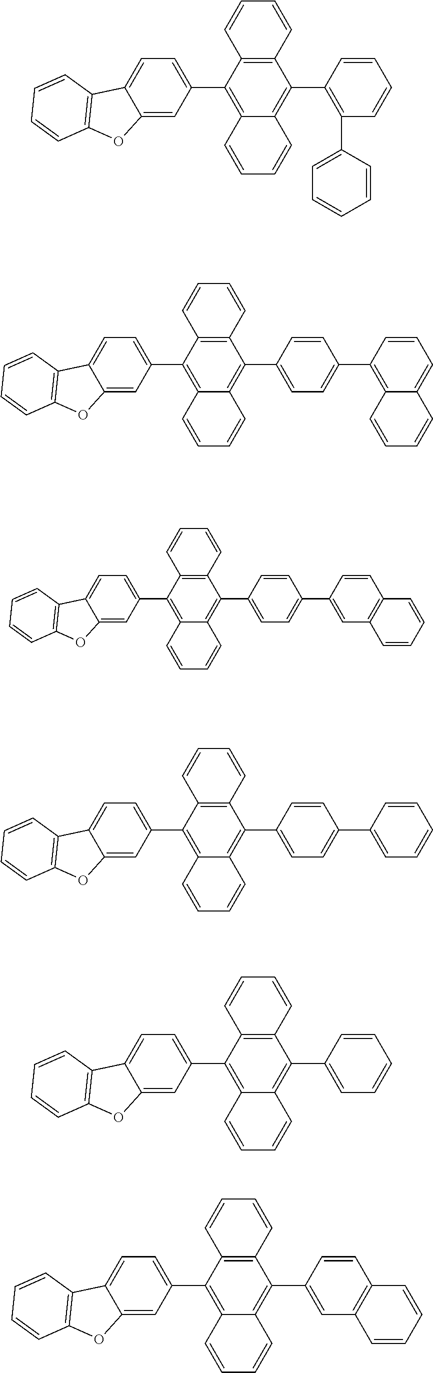 [SMILES: c1ccc(-c2ccccc2-c2c3ccccc3c(-c3ccc4c(c3)oc3ccccc34)c3ccccc23)cc1 ; c1ccc2c(-c3ccc(-c4c5ccccc5c(-c5ccc6c(c5)oc5ccccc56)c5ccccc45)cc3)cccc2c1 ; c1ccc2cc(-c3ccc(-c4c5ccccc5c(-c5ccc6c(c5)oc5ccccc56)c5ccccc45)cc3)ccc2c1 ; c1ccc(-c2ccc(-c3c4ccccc4c(-c4ccc5c(c4)oc4ccccc45)c4ccccc34)cc2)cc1 ; c1ccc(-c2c3ccccc3c(-c3ccc4c(c3)oc3ccccc34)c3ccccc23)cc1 ; c1ccc2cc(-c3c4ccccc4c(-c4ccc5c(c4)oc4ccccc45)c4ccccc34)ccc2c1]

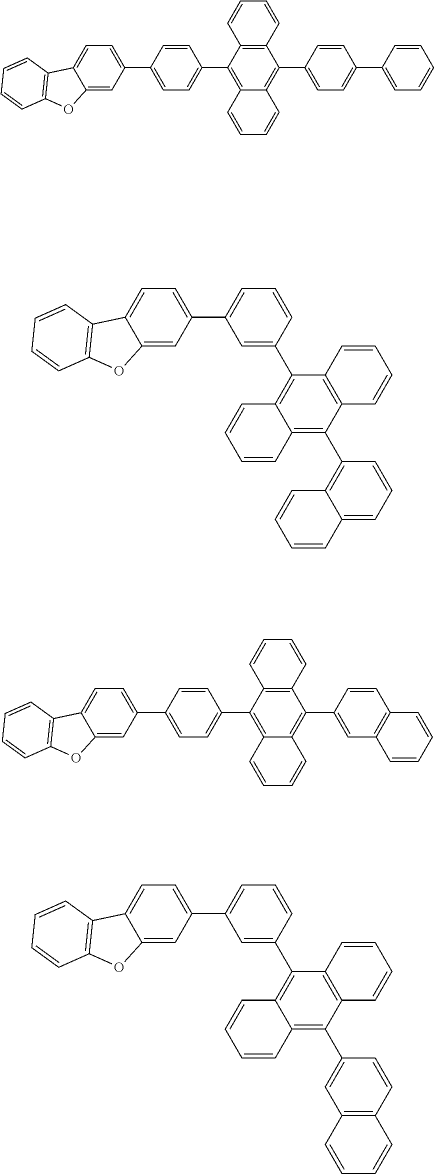 [SMILES: c1ccc(-c2ccc(-c3c4ccccc4c(-c4ccc(-c5ccc6c(c5)oc5ccccc56)cc4)c4ccccc34)cc2)cc1 ; c1cc(-c2ccc3c(c2)oc2ccccc23)cc(-c2c3ccccc3c(-c3cccc4ccccc34)c3ccccc23)c1 ; c1ccc2cc(-c3c4ccccc4c(-c4ccc(-c5ccc6c(c5)oc5ccccc56)cc4)c4ccccc34)ccc2c1 ; c1cc(-c2ccc3c(c2)oc2ccccc23)cc(-c2c3ccccc3c(-c3ccc4ccccc4c3)c3ccccc23)c1]

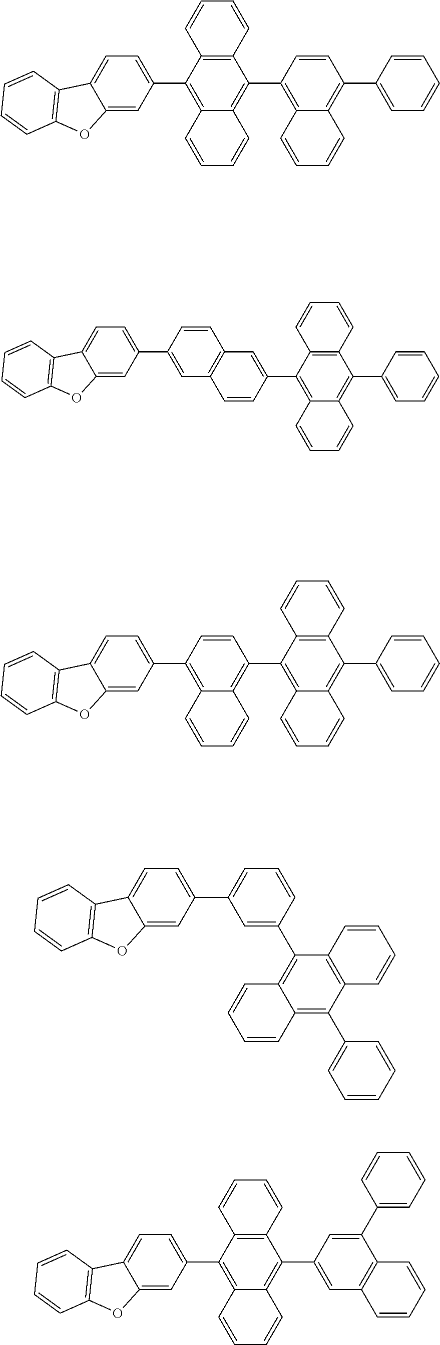 [SMILES: c1ccc(-c2ccc(-c3c4ccccc4c(-c4ccc5c(c4)oc4ccccc45)c4ccccc34)c3ccccc23)cc1 ; c1ccc(-c2c3ccccc3c(-c3ccc4cc(-c5ccc6c(c5)oc5ccccc56)ccc4c3)c3ccccc23)cc1 ; c1ccc(-c2c3ccccc3c(-c3ccc(-c4ccc5c(c4)oc4ccccc45)c4ccccc34)c3ccccc23)cc1 ; c1ccc(-c2c3ccccc3c(-c3cccc(-c4ccc5c(c4)oc4ccccc45)c3)c3ccccc23)cc1 ; c1ccc(-c2cc(-c3c4ccccc4c(-c4ccc5c(c4)oc4ccccc45)c4ccccc34)cc3ccccc23)cc1]

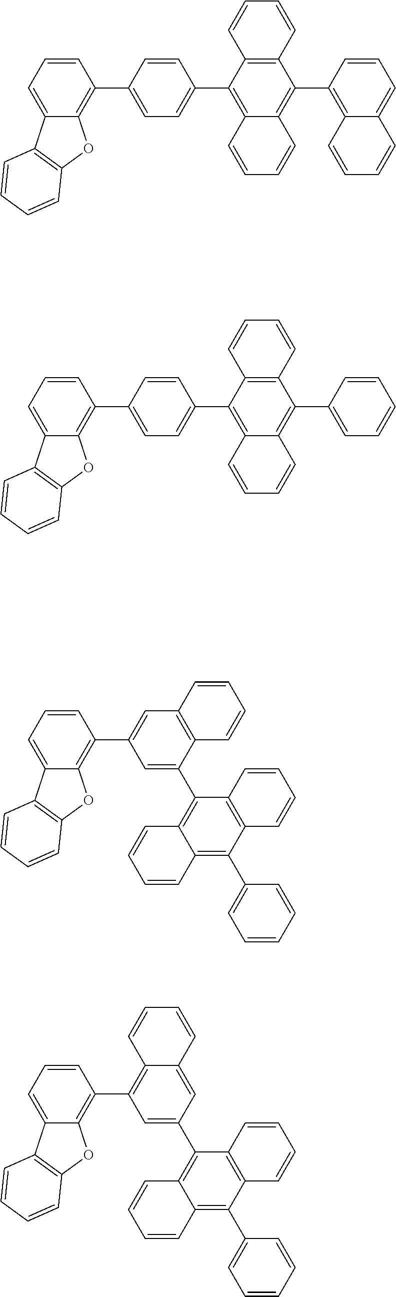 [SMILES: c1ccc2c(-c3c4ccccc4c(-c4ccc(-c5cccc6c5oc5ccccc56)cc4)c4ccccc34)cccc2c1 ; c1ccc(-c2c3ccccc3c(-c3ccc(-c4cccc5c4oc4ccccc45)cc3)c3ccccc23)cc1 ; c1ccc(-c2c3ccccc3c(-c3cc(-c4cccc5c4oc4ccccc45)cc4ccccc34)c3ccccc23)cc1 ; c1ccc(-c2c3ccccc3c(-c3cc(-c4cccc5c4oc4ccccc45)c4ccccc4c3)c3ccccc23)cc1]

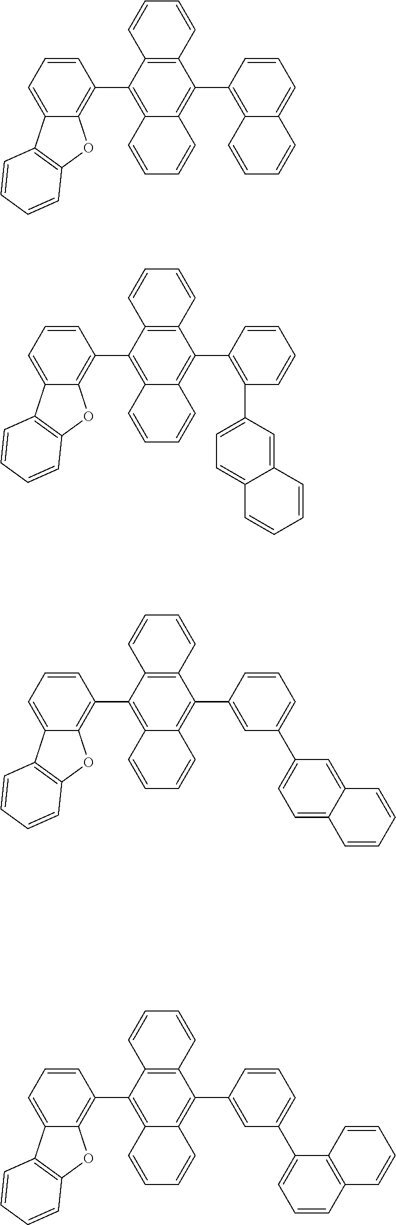 [SMILES: c1ccc2c(-c3c4ccccc4c(-c4cccc5c4oc4ccccc45)c4ccccc34)cccc2c1 ; c1ccc(-c2c3ccccc3c(-c3cccc4c3oc3ccccc34)c3ccccc23)c(-c2ccc3ccccc3c2)c1 ; c1cc(-c2ccc3ccccc3c2)cc(-c2c3ccccc3c(-c3cccc4c3oc3ccccc34)c3ccccc23)c1 ; c1cc(-c2cccc3ccccc23)cc(-c2c3ccccc3c(-c3cccc4c3oc3ccccc34)c3ccccc23)c1]

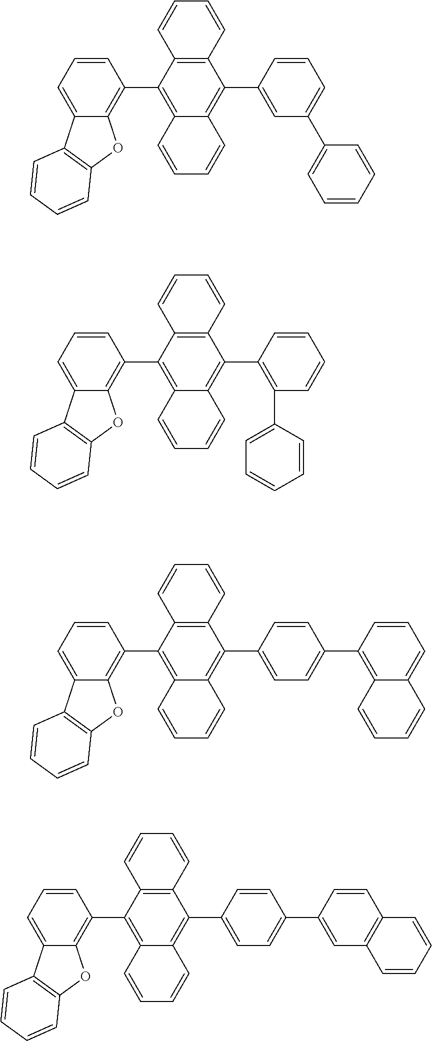 [SMILES: c1ccc(-c2cccc(-c3c4ccccc4c(-c4cccc5c4oc4ccccc45)c4ccccc34)c2)cc1 ; c1ccc(-c2ccccc2-c2c3ccccc3c(-c3cccc4c3oc3ccccc34)c3ccccc23)cc1 ; c1ccc2c(-c3ccc(-c4c5ccccc5c(-c5cccc6c5oc5ccccc56)c5ccccc45)cc3)cccc2c1 ; c1ccc2cc(-c3ccc(-c4c5ccccc5c(-c5cccc6c5oc5ccccc56)c5ccccc45)cc3)ccc2c1]

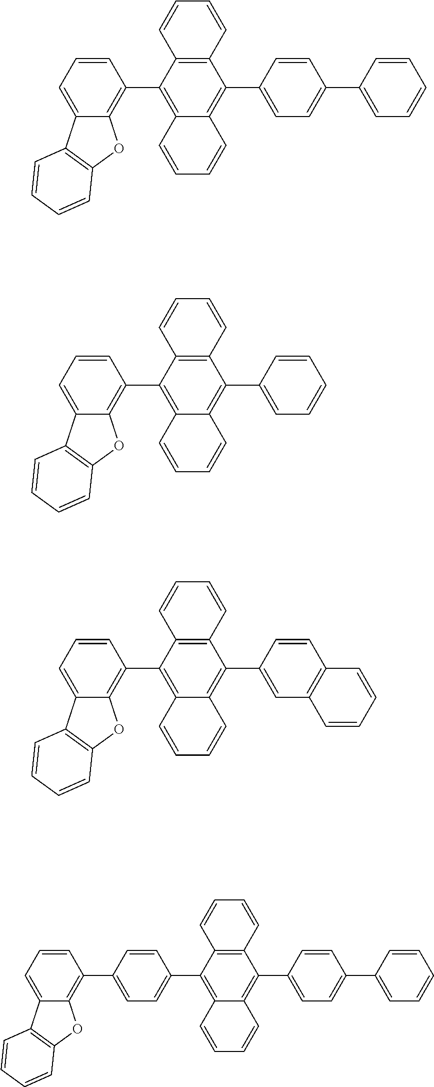 [SMILES: c1ccc(-c2ccc(-c3c4ccccc4c(-c4cccc5c4oc4ccccc45)c4ccccc34)cc2)cc1 ; c1ccc(-c2c3ccccc3c(-c3cccc4c3oc3ccccc34)c3ccccc23)cc1 ; c1ccc2cc(-c3c4ccccc4c(-c4cccc5c4oc4ccccc45)c4ccccc34)ccc2c1 ; c1ccc(-c2ccc(-c3c4ccccc4c(-c4ccc(-c5cccc6c5oc5ccccc56)cc4)c4ccccc34)cc2)cc1]

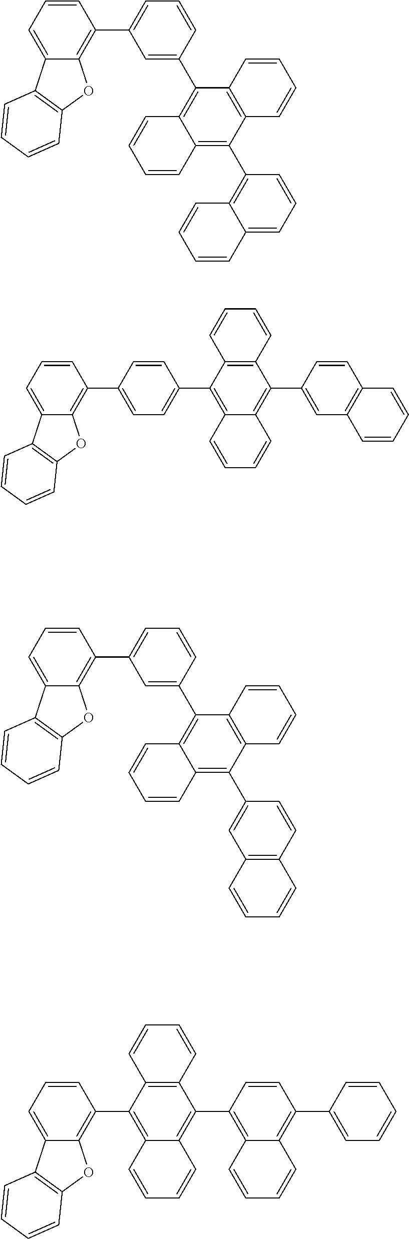 [SMILES: c1cc(-c2c3ccccc3c(-c3cccc4ccccc34)c3ccccc23)cc(-c2cccc3c2oc2ccccc23)c1 ; c1ccc2cc(-c3c4ccccc4c(-c4ccc(-c5cccc6c5oc5ccccc56)cc4)c4ccccc34)ccc2c1 ; c1cc(-c2c3ccccc3c(-c3ccc4ccccc4c3)c3ccccc23)cc(-c2cccc3c2oc2ccccc23)c1 ; c1ccc(-c2ccc(-c3c4ccccc4c(-c4cccc5c4oc4ccccc45)c4ccccc34)c3ccccc23)cc1]

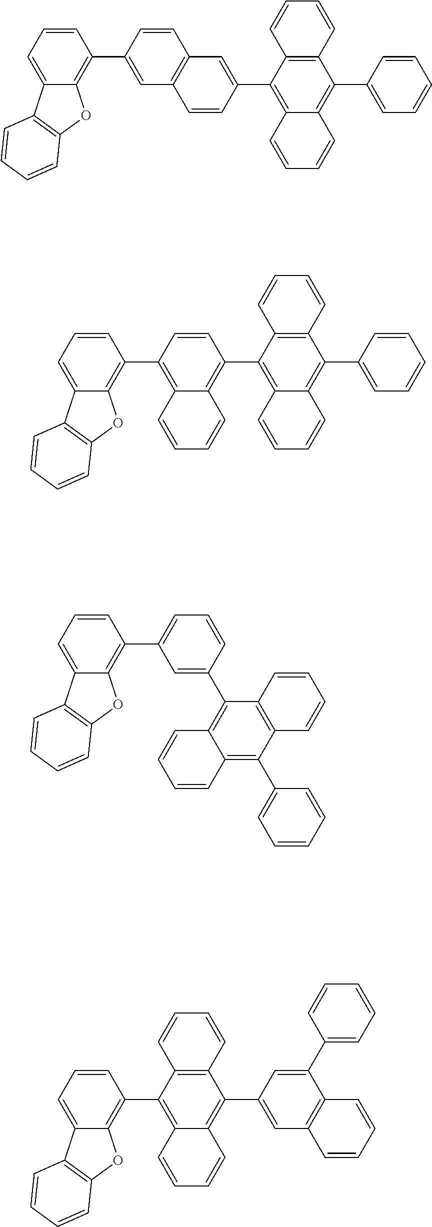 [SMILES: c1ccc(-c2c3ccccc3c(-c3ccc4cc(-c5cccc6c5oc5ccccc56)ccc4c3)c3ccccc23)cc1 ; c1ccc(-c2c3ccccc3c(-c3ccc(-c4cccc5c4oc4ccccc45)c4ccccc34)c3ccccc23)cc1 ; c1ccc(-c2c3ccccc3c(-c3cccc(-c4cccc5c4oc4ccccc45)c3)c3ccccc23)cc1 ; c1ccc(-c2cc(-c3c4ccccc4c(-c4cccc5c4oc4ccccc45)c4ccccc34)cc3ccccc23)cc1]

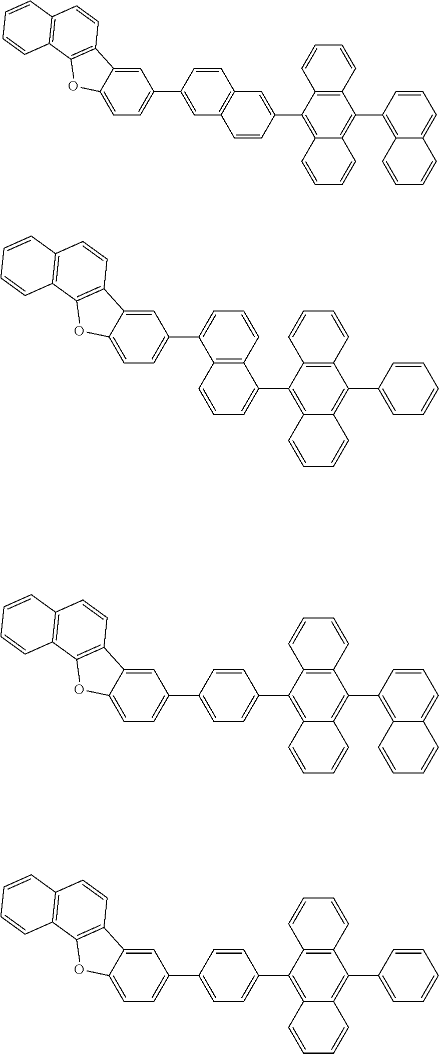 [SMILES: c1ccc2c(-c3c4ccccc4c(-c4ccc5cc(-c6ccc7oc8c9ccccc9ccc8c7c6)ccc5c4)c4ccccc34)cccc2c1 ; c1ccc(-c2c3ccccc3c(-c3cccc4c(-c5ccc6oc7c8ccccc8ccc7c6c5)cccc34)c3ccccc23)cc1 ; c1ccc2c(-c3c4ccccc4c(-c4ccc(-c5ccc6oc7c8ccccc8ccc7c6c5)cc4)c4ccccc34)cccc2c1 ; c1ccc(-c2c3ccccc3c(-c3ccc(-c4ccc5oc6c7ccccc7ccc6c5c4)cc3)c3ccccc23)cc1]

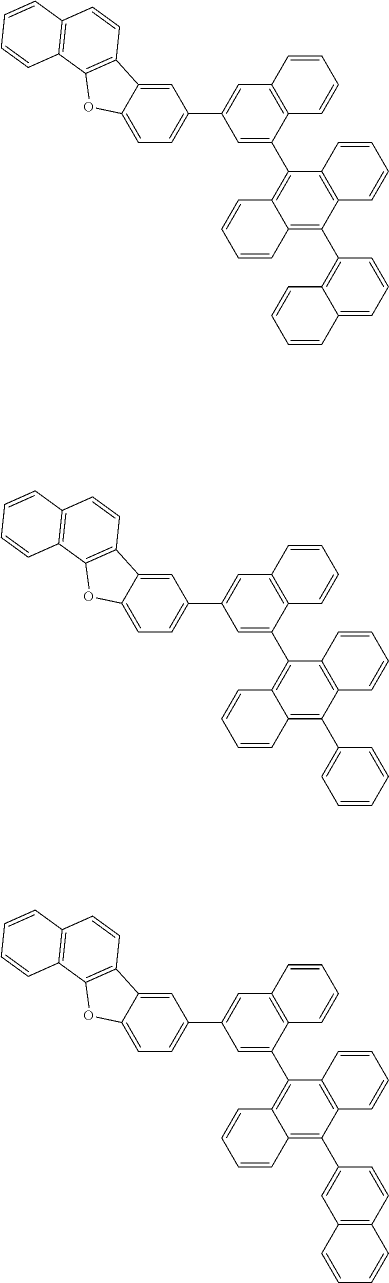 [SMILES: c1ccc2c(-c3c4ccccc4c(-c4cc(-c5ccc6oc7c8ccccc8ccc7c6c5)cc5ccccc45)c4ccccc34)cccc2c1 ; c1ccc(-c2c3ccccc3c(-c3cc(-c4ccc5oc6c7ccccc7ccc6c5c4)cc4ccccc34)c3ccccc23)cc1 ; c1ccc2cc(-c3c4ccccc4c(-c4cc(-c5ccc6oc7c8ccccc8ccc7c6c5)cc5ccccc45)c4ccccc34)ccc2c1]

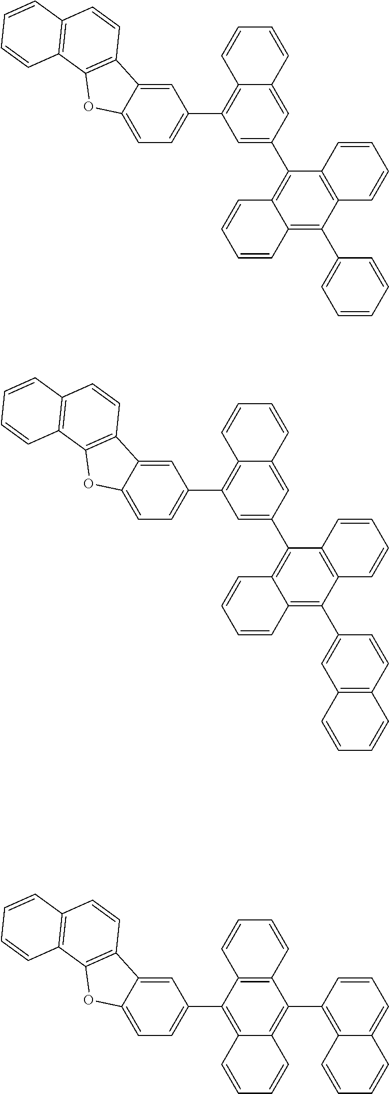 [SMILES: c1ccc(-c2c3ccccc3c(-c3cc(-c4ccc5oc6c7ccccc7ccc6c5c4)c4ccccc4c3)c3ccccc23)cc1 ; c1ccc2cc(-c3c4ccccc4c(-c4cc(-c5ccc6oc7c8ccccc8ccc7c6c5)c5ccccc5c4)c4ccccc34)ccc2c1 ; c1ccc2c(-c3c4ccccc4c(-c4ccc5oc6c7ccccc7ccc6c5c4)c4ccccc34)cccc2c1]

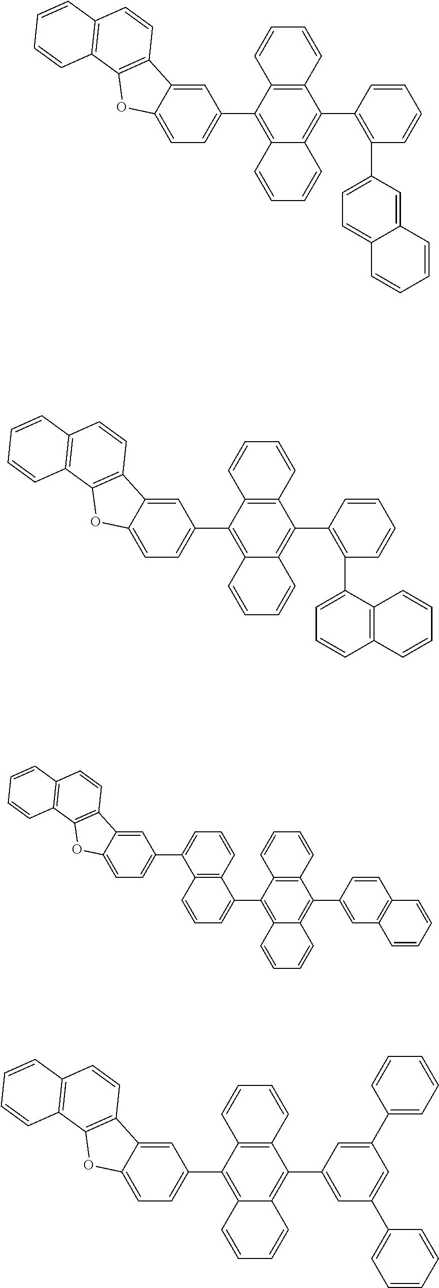 [SMILES: c1ccc(-c2c3ccccc3c(-c3ccc4oc5c6ccccc6ccc5c4c3)c3ccccc23)c(-c2ccc3ccccc3c2)c1 ; c1ccc(-c2c3ccccc3c(-c3ccc4oc5c6ccccc6ccc5c4c3)c3ccccc23)c(-c2cccc3ccccc23)c1 ; c1ccc2cc(-c3c4ccccc4c(-c4cccc5c(-c6ccc7oc8c9ccccc9ccc8c7c6)cccc45)c4ccccc34)ccc2c1 ; c1ccc(-c2cc(-c3ccccc3)cc(-c3c4ccccc4c(-c4ccc5oc6c7ccccc7ccc6c5c4)c4ccccc34)c2)cc1]

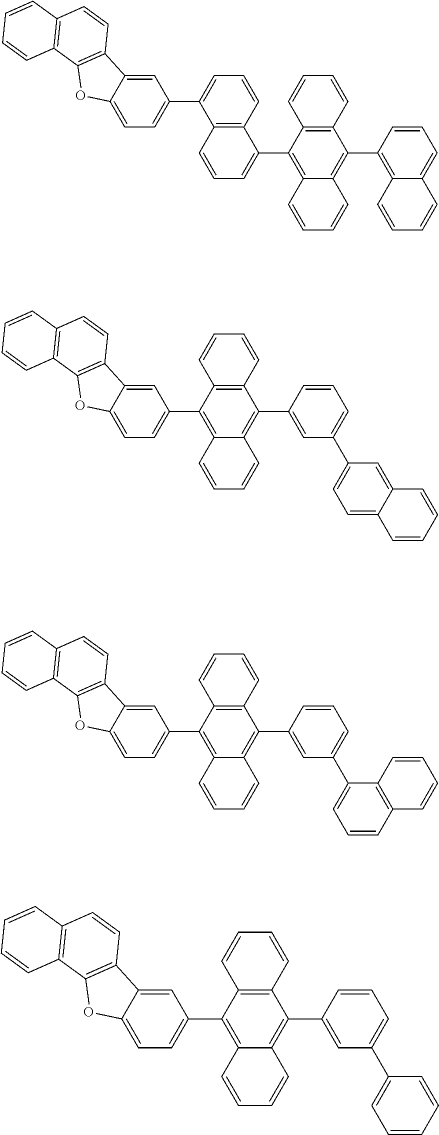 [SMILES: c1ccc2c(-c3c4ccccc4c(-c4cccc5c(-c6ccc7oc8c9ccccc9ccc8c7c6)cccc45)c4ccccc34)cccc2c1 ; c1cc(-c2ccc3ccccc3c2)cc(-c2c3ccccc3c(-c3ccc4oc5c6ccccc6ccc5c4c3)c3ccccc23)c1 ; c1cc(-c2cccc3ccccc23)cc(-c2c3ccccc3c(-c3ccc4oc5c6ccccc6ccc5c4c3)c3ccccc23)c1 ; c1ccc(-c2cccc(-c3c4ccccc4c(-c4ccc5oc6c7ccccc7ccc6c5c4)c4ccccc34)c2)cc1]

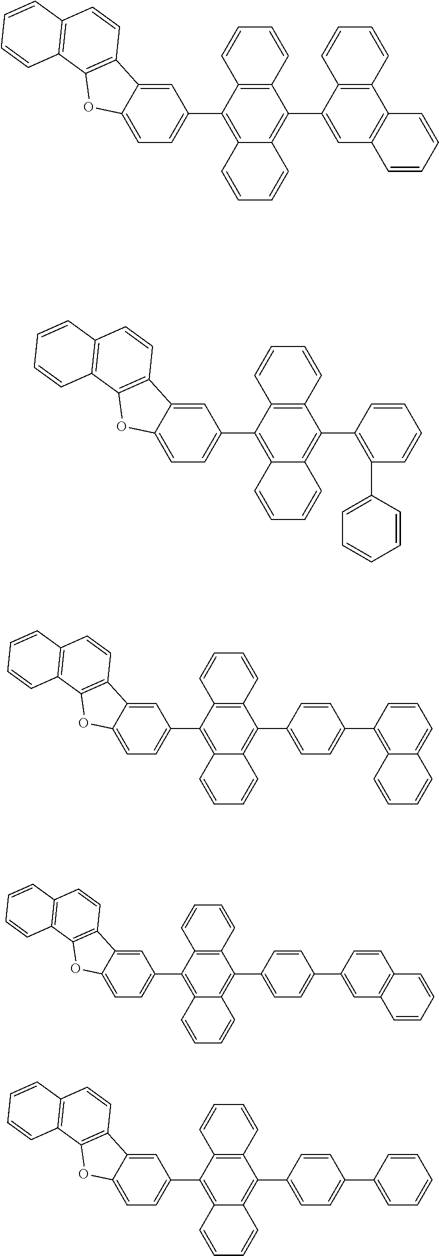 [SMILES: c1ccc2c(c1)cc(-c1c3ccccc3c(-c3ccc4oc5c6ccccc6ccc5c4c3)c3ccccc13)c1ccccc12 ; c1ccc(-c2ccccc2-c2c3ccccc3c(-c3ccc4oc5c6ccccc6ccc5c4c3)c3ccccc23)cc1 ; c1ccc2c(-c3ccc(-c4c5ccccc5c(-c5ccc6oc7c8ccccc8ccc7c6c5)c5ccccc45)cc3)cccc2c1 ; c1ccc2cc(-c3ccc(-c4c5ccccc5c(-c5ccc6oc7c8ccccc8ccc7c6c5)c5ccccc45)cc3)ccc2c1 ; c1ccc(-c2ccc(-c3c4ccccc4c(-c4ccc5oc6c7ccccc7ccc6c5c4)c4ccccc34)cc2)cc1]

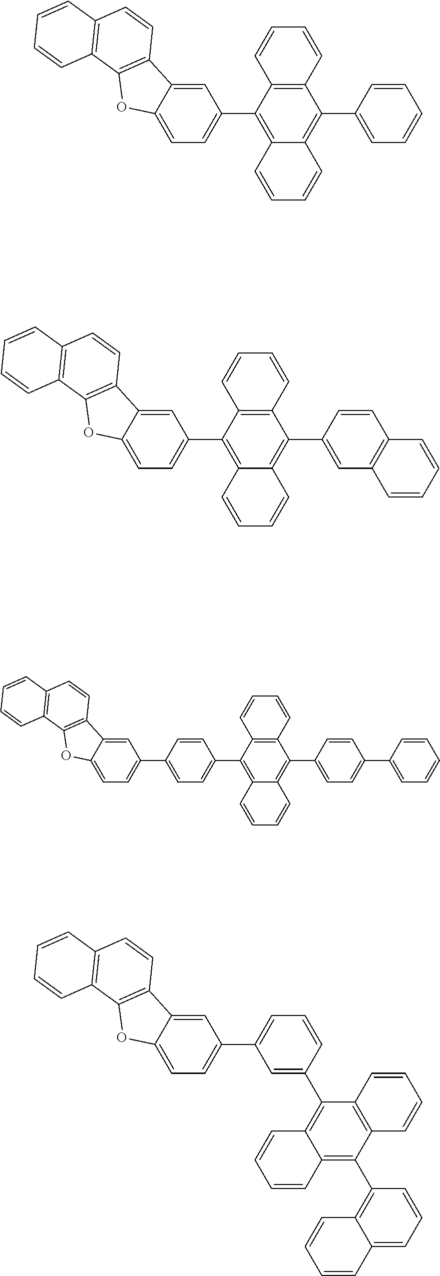 [SMILES: c1ccc(-c2c3ccccc3c(-c3ccc4oc5c6ccccc6ccc5c4c3)c3ccccc23)cc1 ; c1ccc2cc(-c3c4ccccc4c(-c4ccc5oc6c7ccccc7ccc6c5c4)c4ccccc34)ccc2c1 ; c1ccc(-c2ccc(-c3c4ccccc4c(-c4ccc(-c5ccc6oc7c8ccccc8ccc7c6c5)cc4)c4ccccc34)cc2)cc1 ; c1cc(-c2ccc3oc4c5ccccc5ccc4c3c2)cc(-c2c3ccccc3c(-c3cccc4ccccc34)c3ccccc23)c1]

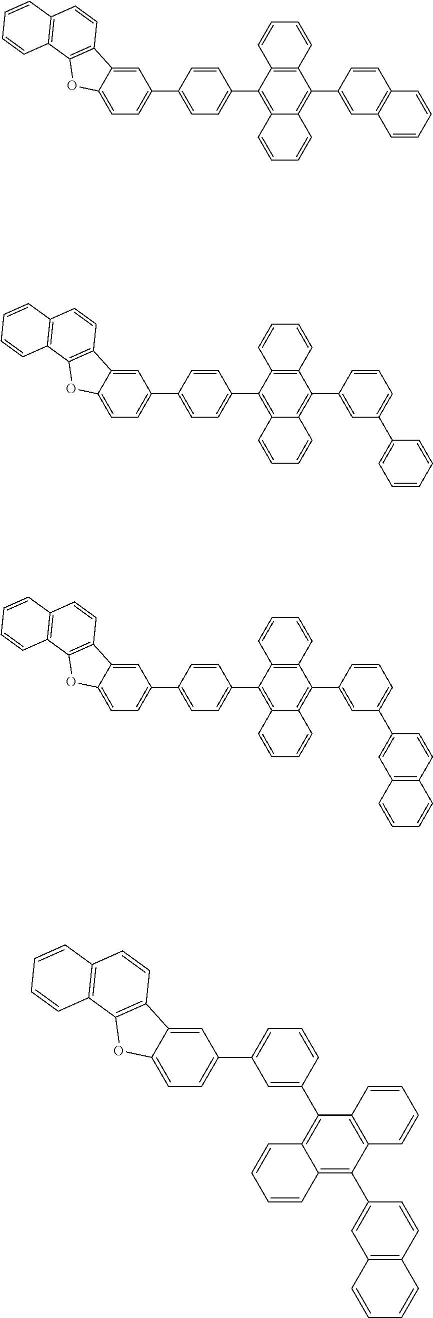 [SMILES: c1ccc2cc(-c3c4ccccc4c(-c4ccc(-c5ccc6oc7c8ccccc8ccc7c6c5)cc4)c4ccccc34)ccc2c1 ; c1ccc(-c2cccc(-c3c4ccccc4c(-c4ccc(-c5ccc6oc7c8ccccc8ccc7c6c5)cc4)c4ccccc34)c2)cc1 ; c1cc(-c2ccc3ccccc3c2)cc(-c2c3ccccc3c(-c3ccc(-c4ccc5oc6c7ccccc7ccc6c5c4)cc3)c3ccccc23)c1 ; c1cc(-c2ccc3oc4c5ccccc5ccc4c3c2)cc(-c2c3ccccc3c(-c3ccc4ccccc4c3)c3ccccc23)c1]

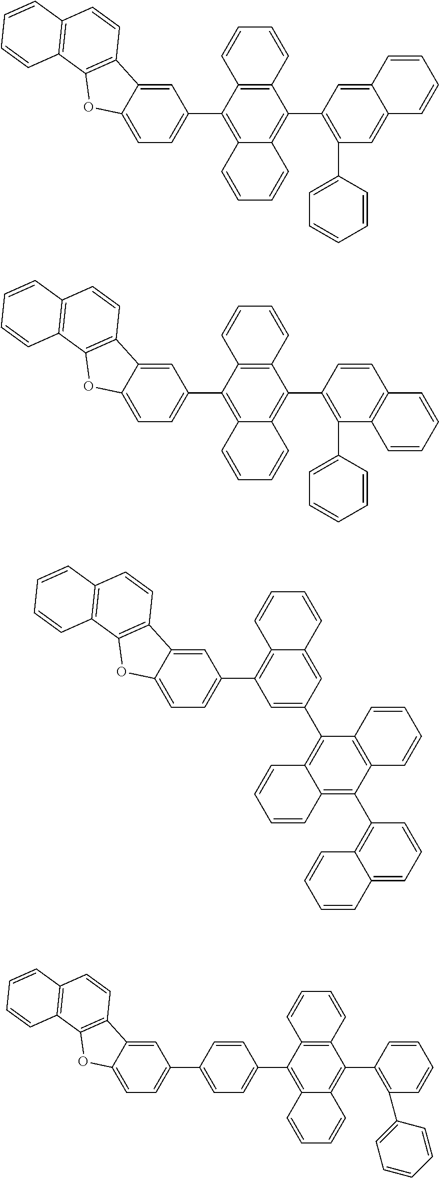 [SMILES: c1ccc(-c2cc3ccccc3cc2-c2c3ccccc3c(-c3ccc4oc5c6ccccc6ccc5c4c3)c3ccccc23)cc1 ; c1ccc(-c2c(-c3c4ccccc4c(-c4ccc5oc6c7ccccc7ccc6c5c4)c4ccccc34)ccc3ccccc23)cc1 ; c1ccc2c(-c3ccc4oc5c6ccccc6ccc5c4c3)cc(-c3c4ccccc4c(-c4cccc5ccccc45)c4ccccc34)cc2c1 ; c1ccc(-c2ccccc2-c2c3ccccc3c(-c3ccc(-c4ccc5oc6c7ccccc7ccc6c5c4)cc3)c3ccccc23)cc1]

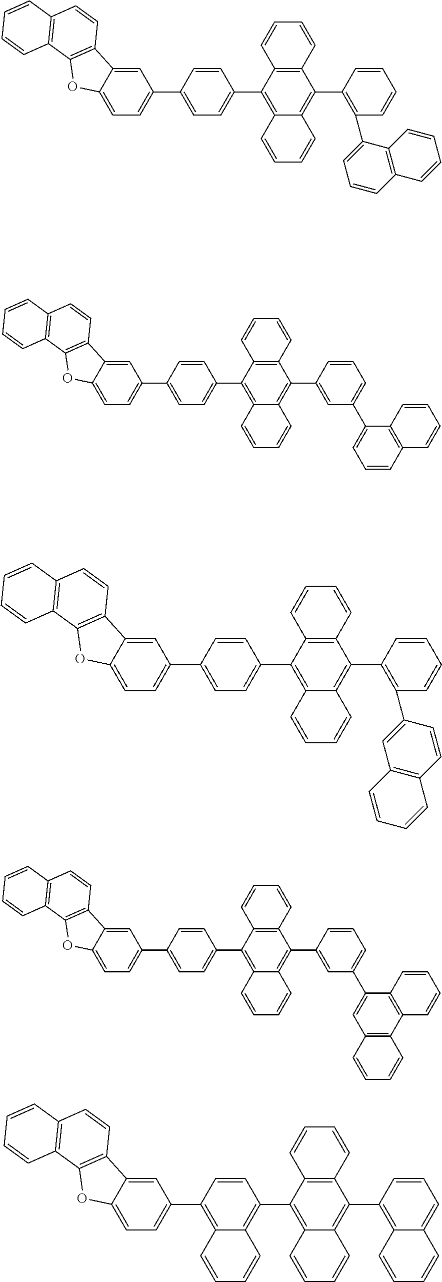 [SMILES: c1ccc(-c2c3ccccc3c(-c3ccc(-c4ccc5oc6c7ccccc7ccc6c5c4)cc3)c3ccccc23)c(-c2cccc3ccccc23)c1 ; c1cc(-c2cccc3ccccc23)cc(-c2c3ccccc3c(-c3ccc(-c4ccc5oc6c7ccccc7ccc6c5c4)cc3)c3ccccc23)c1 ; c1ccc(-c2c3ccccc3c(-c3ccc(-c4ccc5oc6c7ccccc7ccc6c5c4)cc3)c3ccccc23)c(-c2ccc3ccccc3c2)c1 ; c1cc(-c2c3ccccc3c(-c3ccc(-c4ccc5oc6c7ccccc7ccc6c5c4)cc3)c3ccccc23)cc(-c2cc3ccccc3c3ccccc23)c1 ; c1ccc2c(-c3c4ccccc4c(-c4ccc(-c5ccc6oc7c8ccccc8ccc7c6c5)c5ccccc45)c4ccccc34)cccc2c1]

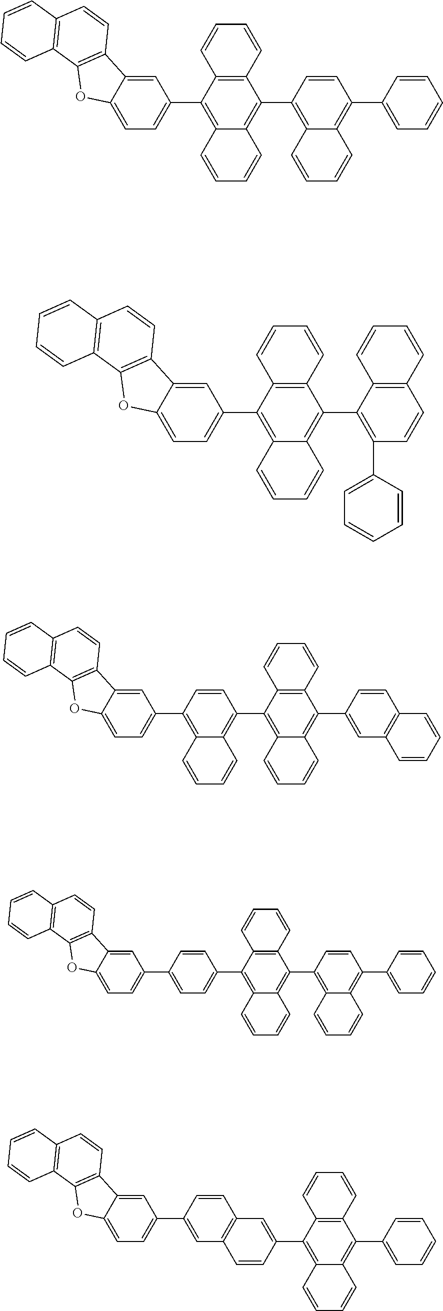 [SMILES: c1ccc(-c2ccc(-c3c4ccccc4c(-c4ccc5oc6c7ccccc7ccc6c5c4)c4ccccc34)c3ccccc23)cc1 ; c1ccc(-c2ccc3ccccc3c2-c2c3ccccc3c(-c3ccc4oc5c6ccccc6ccc5c4c3)c3ccccc23)cc1 ; c1ccc2cc(-c3c4ccccc4c(-c4ccc(-c5ccc6oc7c8ccccc8ccc7c6c5)c5ccccc45)c4ccccc34)ccc2c1 ; c1ccc(-c2ccc(-c3c4ccccc4c(-c4ccc(-c5ccc6oc7c8ccccc8ccc7c6c5)cc4)c4ccccc34)c3ccccc23)cc1 ; c1ccc(-c2c3ccccc3c(-c3ccc4cc(-c5ccc6oc7c8ccccc8ccc7c6c5)ccc4c3)c3ccccc23)cc1]

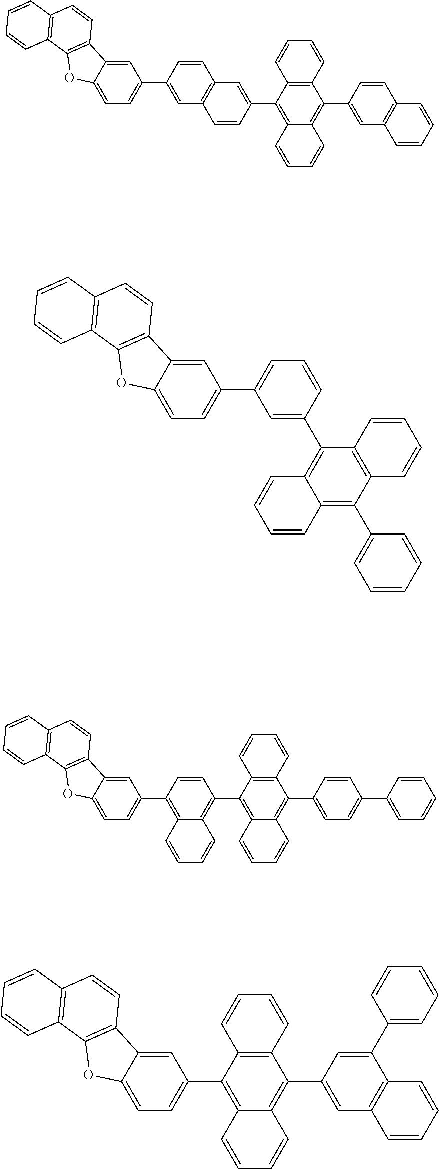 [SMILES: c1ccc2cc(-c3c4ccccc4c(-c4ccc5cc(-c6ccc7oc8c9ccccc9ccc8c7c6)ccc5c4)c4ccccc34)ccc2c1 ; c1ccc(-c2c3ccccc3c(-c3cccc(-c4ccc5oc6c7ccccc7ccc6c5c4)c3)c3ccccc23)cc1 ; c1ccc(-c2ccc(-c3c4ccccc4c(-c4ccc(-c5ccc6oc7c8ccccc8ccc7c6c5)c5ccccc45)c4ccccc34)cc2)cc1 ; c1ccc(-c2cc(-c3c4ccccc4c(-c4ccc5oc6c7ccccc7ccc6c5c4)c4ccccc34)cc3ccccc23)cc1]

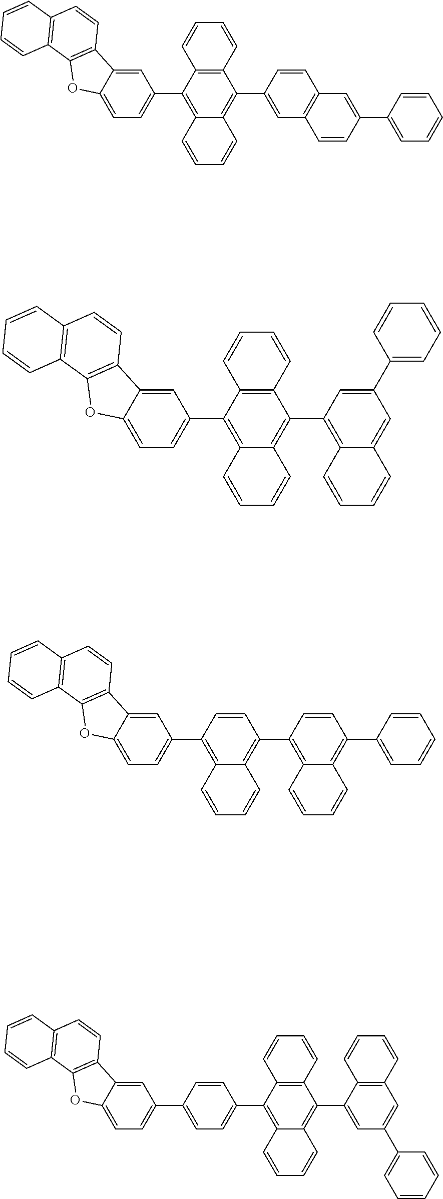 [SMILES: c1ccc(-c2ccc3cc(-c4c5ccccc5c(-c5ccc6oc7c8ccccc8ccc7c6c5)c5ccccc45)ccc3c2)cc1 ; c1ccc(-c2cc(-c3c4ccccc4c(-c4ccc5oc6c7ccccc7ccc6c5c4)c4ccccc34)c3ccccc3c2)cc1 ; c1ccc(-c2ccc(-c3ccc(-c4ccc5oc6c7ccccc7ccc6c5c4)c4ccccc34)c3ccccc23)cc1 ; c1ccc(-c2cc(-c3c4ccccc4c(-c4ccc(-c5ccc6oc7c8ccccc8ccc7c6c5)cc4)c4ccccc34)c3ccccc3c2)cc1]

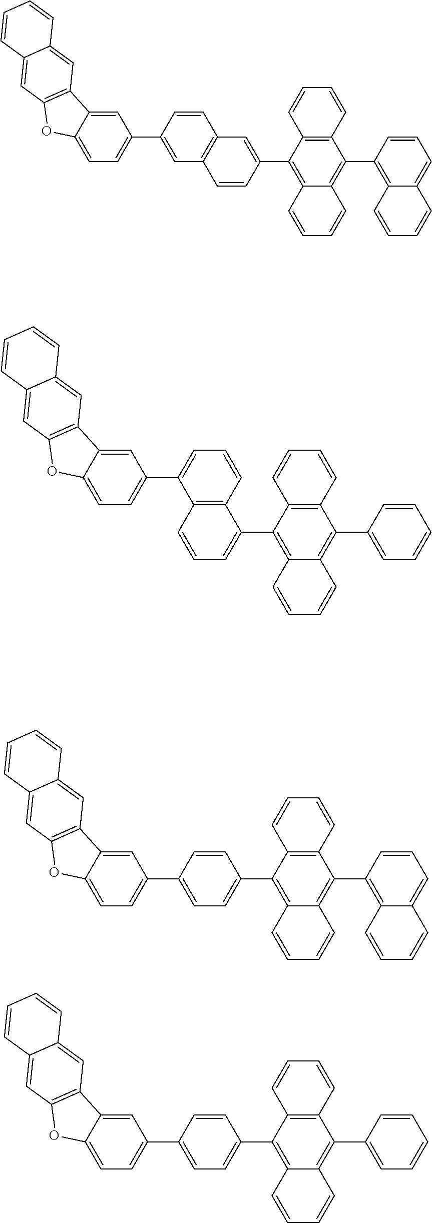 [SMILES: c1ccc2cc3c(cc2c1)oc1ccc(-c2ccc4cc(-c5c6ccccc6c(-c6cccc7ccccc67)c6ccccc56)ccc4c2)cc13 ; c1ccc(-c2c3ccccc3c(-c3cccc4c(-c5ccc6oc7cc8ccccc8cc7c6c5)cccc34)c3ccccc23)cc1 ; c1ccc2cc3c(cc2c1)oc1ccc(-c2ccc(-c4c5ccccc5c(-c5cccc6ccccc56)c5ccccc45)cc2)cc13 ; c1ccc(-c2c3ccccc3c(-c3ccc(-c4ccc5oc6cc7ccccc7cc6c5c4)cc3)c3ccccc23)cc1]

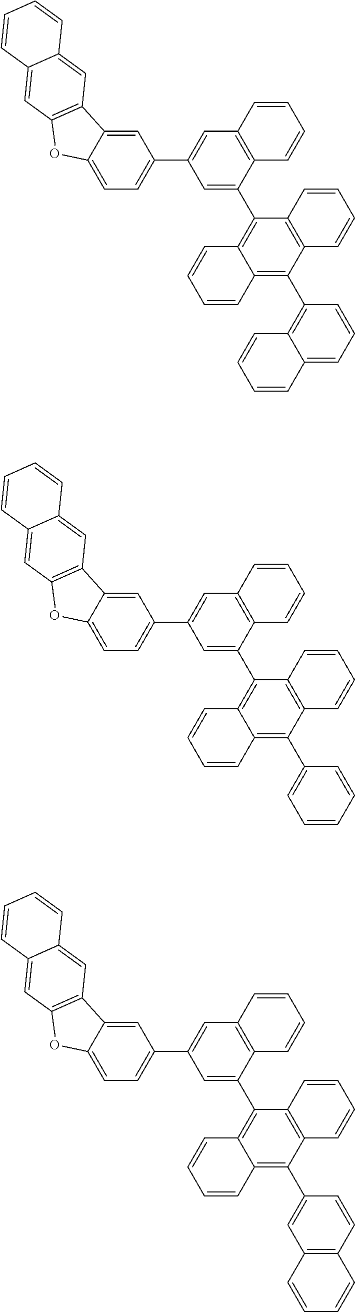 [SMILES: c1ccc2cc3c(cc2c1)oc1ccc(-c2cc(-c4c5ccccc5c(-c5cccc6ccccc56)c5ccccc45)c4ccccc4c2)cc13 ; c1ccc(-c2c3ccccc3c(-c3cc(-c4ccc5oc6cc7ccccc7cc6c5c4)cc4ccccc34)c3ccccc23)cc1 ; c1ccc2cc(-c3c4ccccc4c(-c4cc(-c5ccc6oc7cc8ccccc8cc7c6c5)cc5ccccc45)c4ccccc34)ccc2c1]

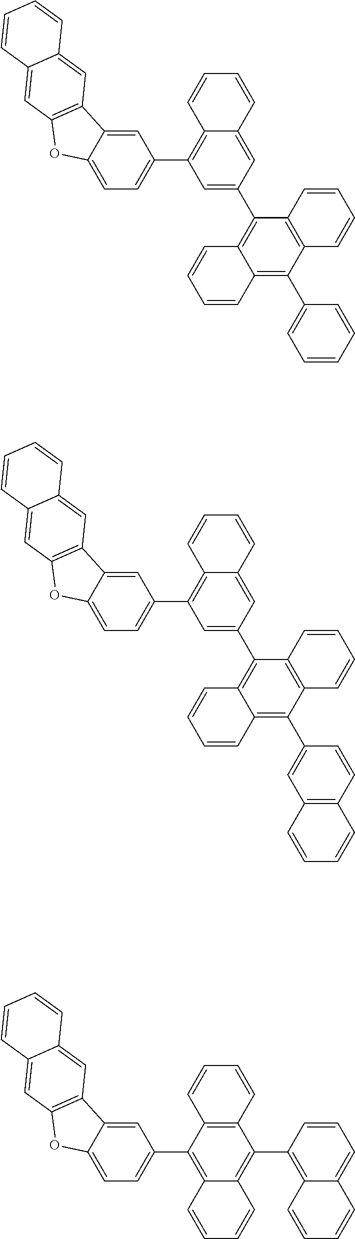 [SMILES: c1ccc(-c2c3ccccc3c(-c3cc(-c4ccc5oc6cc7ccccc7cc6c5c4)c4ccccc4c3)c3ccccc23)cc1 ; c1ccc2cc(-c3c4ccccc4c(-c4cc(-c5ccc6oc7cc8ccccc8cc7c6c5)c5ccccc5c4)c4ccccc34)ccc2c1 ; c1ccc2cc3c(cc2c1)oc1ccc(-c2c4ccccc4c(-c4cccc5ccccc45)c4ccccc24)cc13]

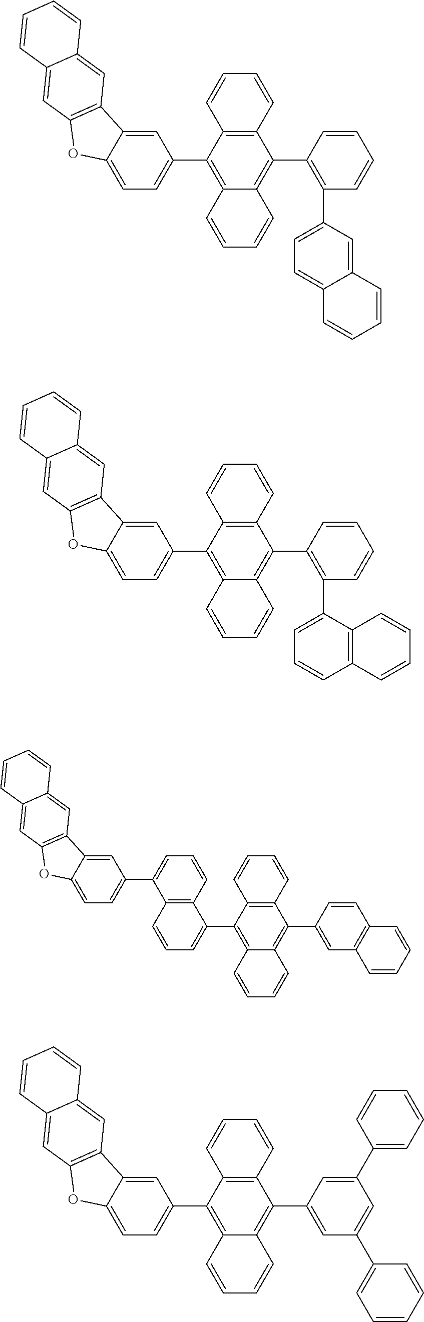 [SMILES: c1ccc(-c2c3ccccc3c(-c3ccc4oc5cc6ccccc6cc5c4c3)c3ccccc23)c(-c2ccc3ccccc3c2)c1 ; c1ccc(-c2c3ccccc3c(-c3ccc4oc5cc6ccccc6cc5c4c3)c3ccccc23)c(-c2cccc3ccccc23)c1 ; c1ccc2cc(-c3c4ccccc4c(-c4cccc5c(-c6ccc7oc8cc9ccccc9cc8c7c6)cccc45)c4ccccc34)ccc2c1 ; c1ccc(-c2cc(-c3ccccc3)cc(-c3c4ccccc4c(-c4ccc5oc6cc7ccccc7cc6c5c4)c4ccccc34)c2)cc1]

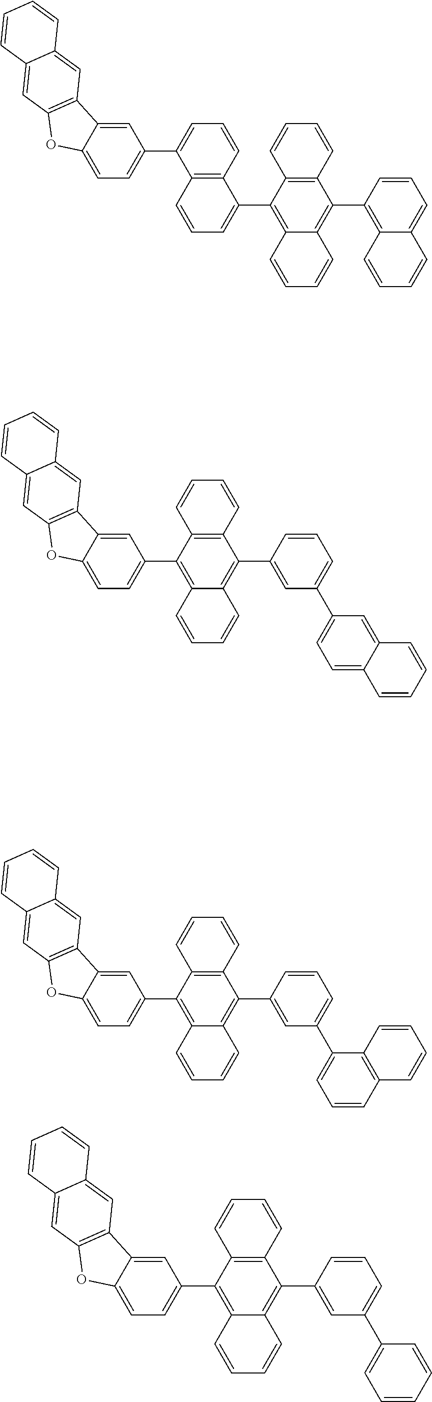 [SMILES: c1ccc2cc3c(cc2c1)oc1ccc(-c2cccc4c(-c5c6ccccc6c(-c6cccc7ccccc67)c6ccccc56)cccc24)cc13 ; c1cc(-c2ccc3ccccc3c2)cc(-c2c3ccccc3c(-c3ccc4oc5cc6ccccc6cc5c4c3)c3ccccc23)c1 ; c1cc(-c2cccc3ccccc23)cc(-c2c3ccccc3c(-c3ccc4oc5cc6ccccc6cc5c4c3)c3ccccc23)c1 ; c1ccc(-c2cccc(-c3c4ccccc4c(-c4ccc5oc6cc7ccccc7cc6c5c4)c4ccccc34)c2)cc1]

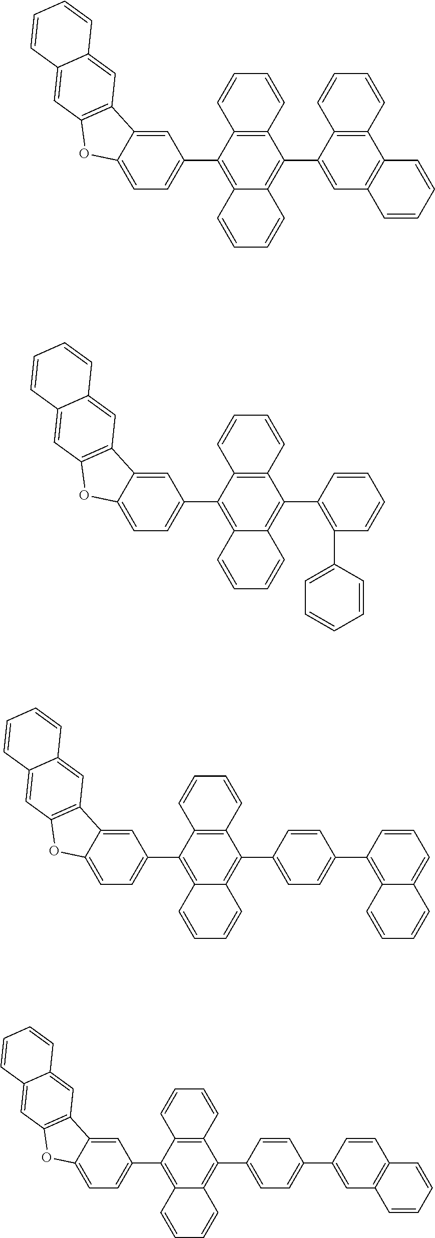 [SMILES: c1ccc2cc3c(cc2c1)oc1ccc(-c2c4ccccc4c(-c4cc5ccccc5c5ccccc45)c4ccccc24)cc13 ; c1ccc(-c2ccccc2-c2c3ccccc3c(-c3ccc4oc5cc6ccccc6cc5c4c3)c3ccccc23)cc1 ; c1ccc2cc3c(cc2c1)oc1ccc(-c2c4ccccc4c(-c4ccc(-c5cccc6ccccc56)cc4)c4ccccc24)cc13 ; c1ccc2cc(-c3ccc(-c4c5ccccc5c(-c5ccc6oc7cc8ccccc8cc7c6c5)c5ccccc45)cc3)ccc2c1]

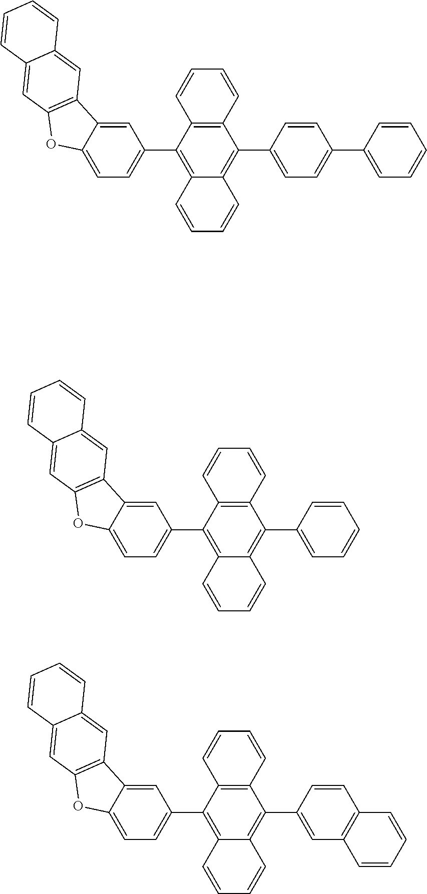 [SMILES: c1ccc(-c2ccc(-c3c4ccccc4c(-c4ccc5oc6cc7ccccc7cc6c5c4)c4ccccc34)cc2)cc1 ; c1ccc(-c2c3ccccc3c(-c3ccc4oc5cc6ccccc6cc5c4c3)c3ccccc23)cc1 ; c1ccc2cc(-c3c4ccccc4c(-c4ccc5oc6cc7ccccc7cc6c5c4)c4ccccc34)ccc2c1]

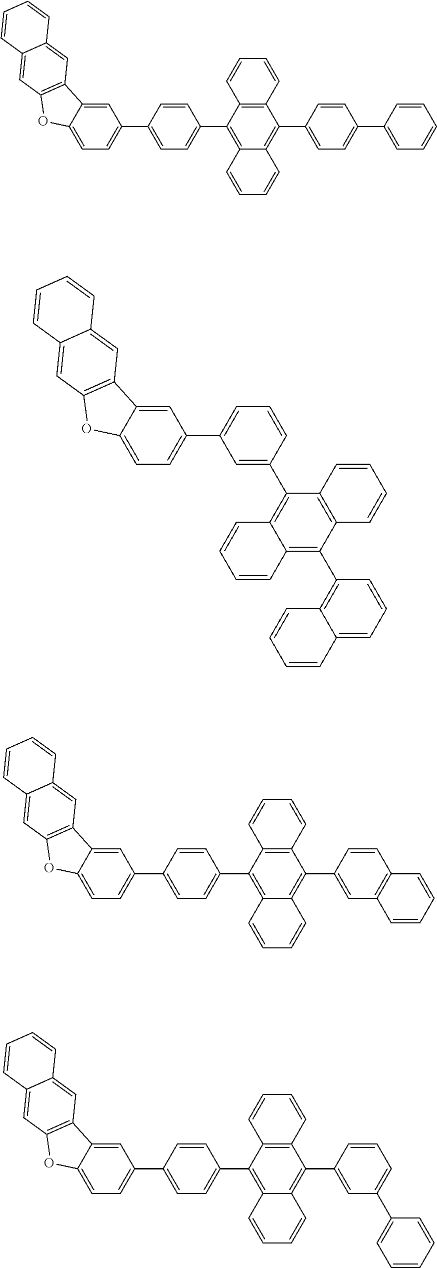 [SMILES: c1ccc(-c2ccc(-c3c4ccccc4c(-c4ccc(-c5ccc6oc7cc8ccccc8cc7c6c5)cc4)c4ccccc34)cc2)cc1 ; c1cc(-c2ccc3oc4cc5ccccc5cc4c3c2)cc(-c2c3ccccc3c(-c3cccc4ccccc34)c3ccccc23)c1 ; c1ccc2cc(-c3c4ccccc4c(-c4ccc(-c5ccc6oc7cc8ccccc8cc7c6c5)cc4)c4ccccc34)ccc2c1 ; c1ccc(-c2cccc(-c3c4ccccc4c(-c4ccc(-c5ccc6oc7cc8ccccc8cc7c6c5)cc4)c4ccccc34)c2)cc1]

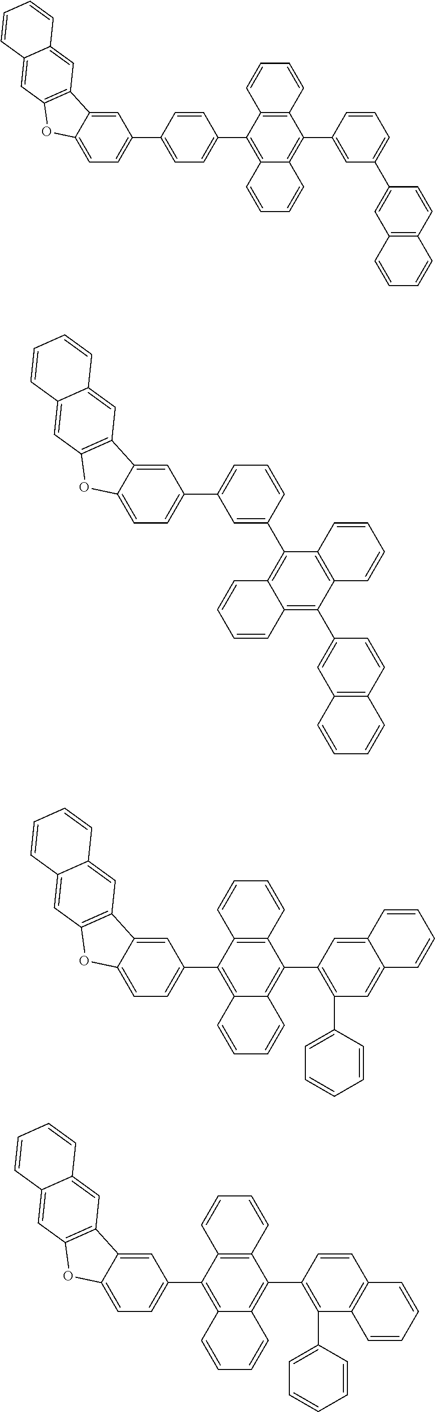 [SMILES: c1cc(-c2ccc3ccccc3c2)cc(-c2c3ccccc3c(-c3ccc(-c4ccc5oc6cc7ccccc7cc6c5c4)cc3)c3ccccc23)c1 ; c1cc(-c2ccc3oc4cc5ccccc5cc4c3c2)cc(-c2c3ccccc3c(-c3ccc4ccccc4c3)c3ccccc23)c1 ; c1ccc(-c2cc3ccccc3cc2-c2c3ccccc3c(-c3ccc4oc5cc6ccccc6cc5c4c3)c3ccccc23)cc1 ; c1ccc(-c2c(-c3c4ccccc4c(-c4ccc5oc6cc7ccccc7cc6c5c4)c4ccccc34)ccc3ccccc23)cc1]

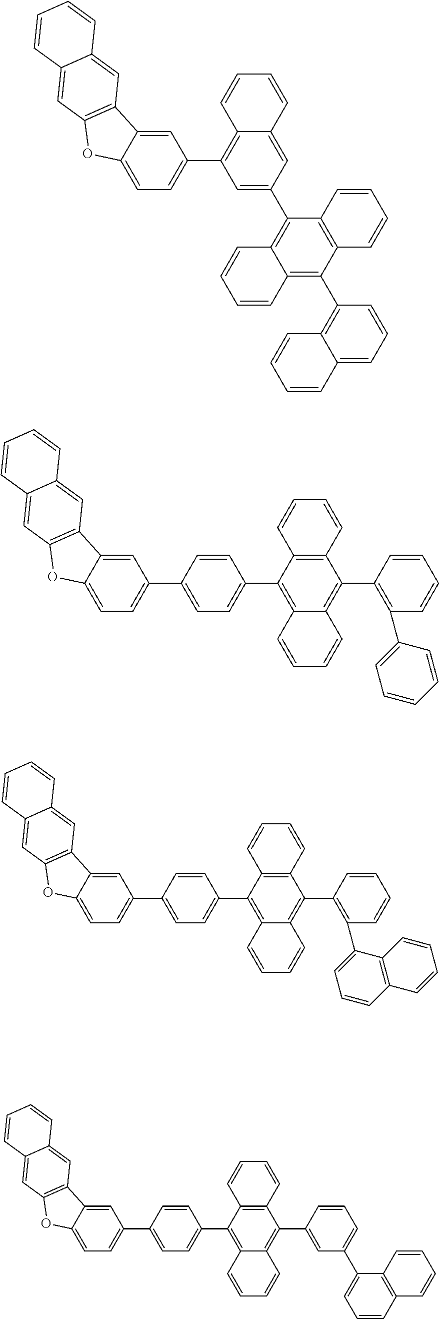 [SMILES: c1ccc2cc3c(cc2c1)oc1ccc(-c2cc(-c4c5ccccc5c(-c5cccc6ccccc56)c5ccccc45)cc4ccccc24)cc13 ; c1ccc(-c2ccccc2-c2c3ccccc3c(-c3ccc(-c4ccc5oc6cc7ccccc7cc6c5c4)cc3)c3ccccc23)cc1 ; c1ccc(-c2c3ccccc3c(-c3ccc(-c4ccc5oc6cc7ccccc7cc6c5c4)cc3)c3ccccc23)c(-c2cccc3ccccc23)c1 ; c1cc(-c2cccc3ccccc23)cc(-c2c3ccccc3c(-c3ccc(-c4ccc5oc6cc7ccccc7cc6c5c4)cc3)c3ccccc23)c1]

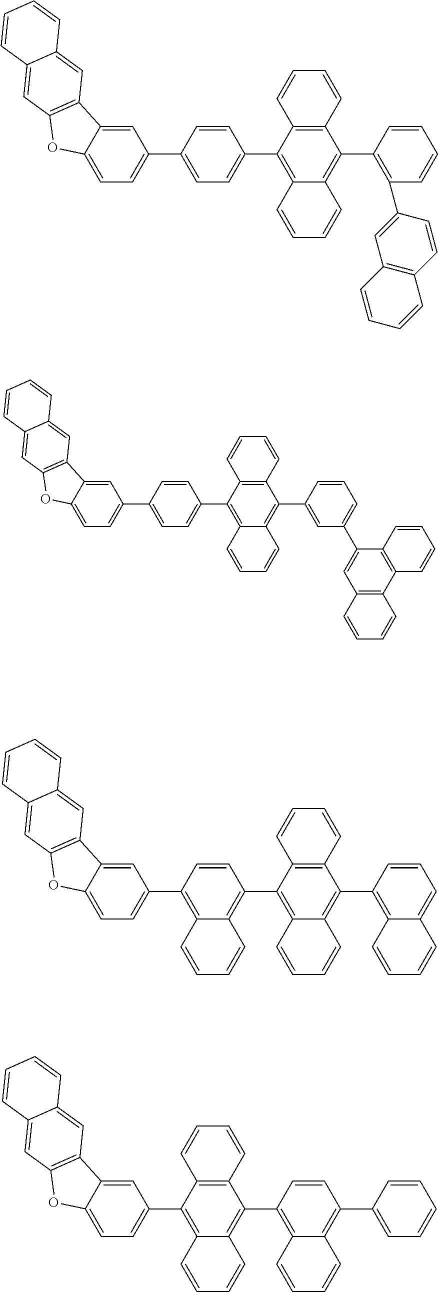 [SMILES: c1ccc(-c2c3ccccc3c(-c3ccc(-c4ccc5oc6cc7ccccc7cc6c5c4)cc3)c3ccccc23)c(-c2ccc3ccccc3c2)c1 ; c1cc(-c2c3ccccc3c(-c3ccc(-c4ccc5oc6cc7ccccc7cc6c5c4)cc3)c3ccccc23)cc(-c2cc3ccccc3c3ccccc23)c1 ; c1ccc2cc3c(cc2c1)oc1ccc(-c2ccc(-c4c5ccccc5c(-c5cccc6ccccc56)c5ccccc45)c4ccccc24)cc13 ; c1ccc(-c2ccc(-c3c4ccccc4c(-c4ccc5oc6cc7ccccc7cc6c5c4)c4ccccc34)c3ccccc23)cc1]

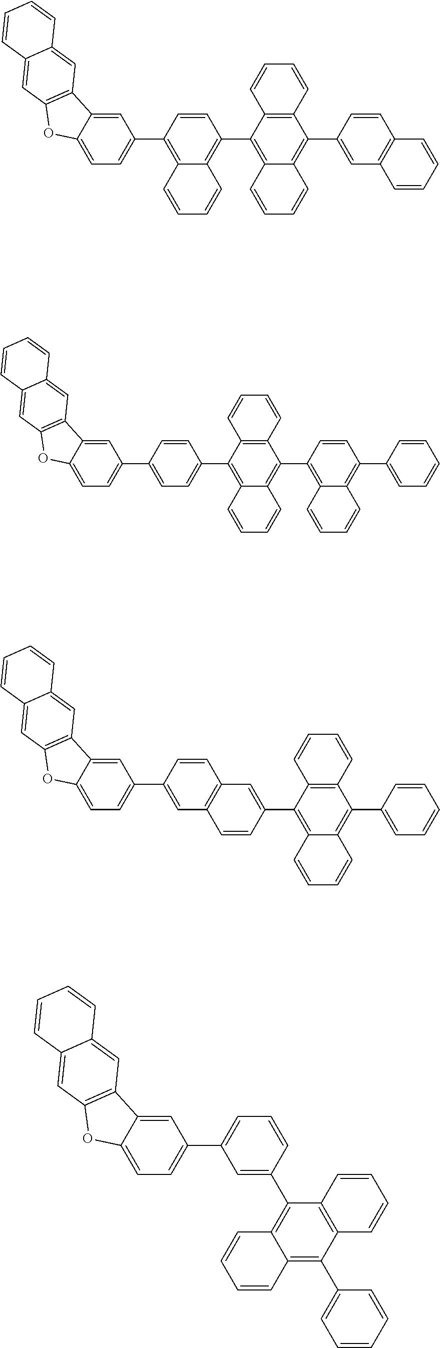 [SMILES: c1ccc2cc(-c3c4ccccc4c(-c4ccc(-c5ccc6oc7cc8ccccc8cc7c6c5)c5ccccc45)c4ccccc34)ccc2c1 ; c1ccc(-c2ccc(-c3c4ccccc4c(-c4ccc(-c5ccc6oc7cc8ccccc8cc7c6c5)cc4)c4ccccc34)c3ccccc23)cc1 ; c1ccc(-c2c3ccccc3c(-c3ccc4cc(-c5ccc6oc7cc8ccccc8cc7c6c5)ccc4c3)c3ccccc23)cc1 ; c1ccc(-c2c3ccccc3c(-c3cccc(-c4ccc5oc6cc7ccccc7cc6c5c4)c3)c3ccccc23)cc1]

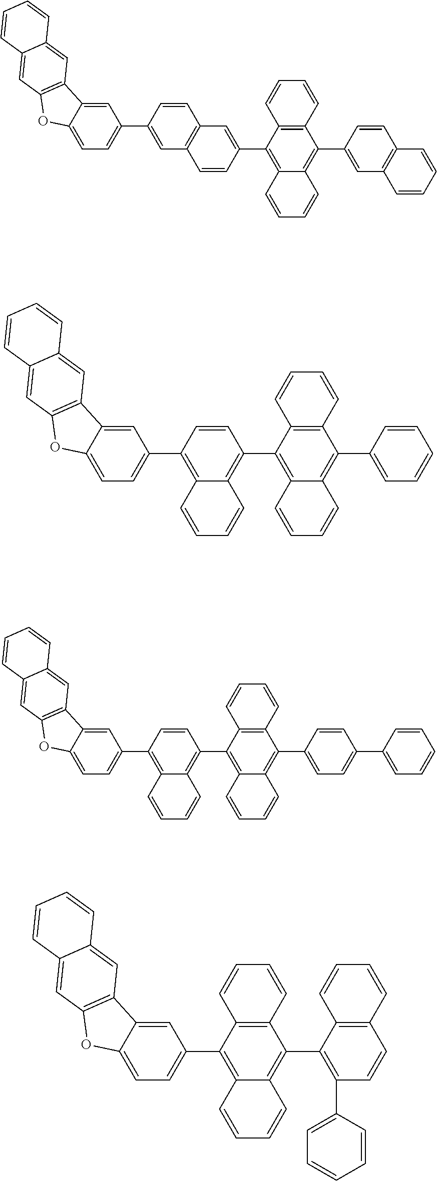 [SMILES: c1ccc2cc(-c3c4ccccc4c(-c4ccc5cc(-c6ccc7oc8cc9ccccc9cc8c7c6)ccc5c4)c4ccccc34)ccc2c1 ; c1ccc(-c2c3ccccc3c(-c3ccc(-c4ccc5oc6cc7ccccc7cc6c5c4)c4ccccc34)c3ccccc23)cc1 ; c1ccc(-c2ccc(-c3c4ccccc4c(-c4ccc(-c5ccc6oc7cc8ccccc8cc7c6c5)c5ccccc45)c4ccccc34)cc2)cc1 ; c1ccc(-c2ccc3ccccc3c2-c2c3ccccc3c(-c3ccc4oc5cc6ccccc6cc5c4c3)c3ccccc23)cc1]

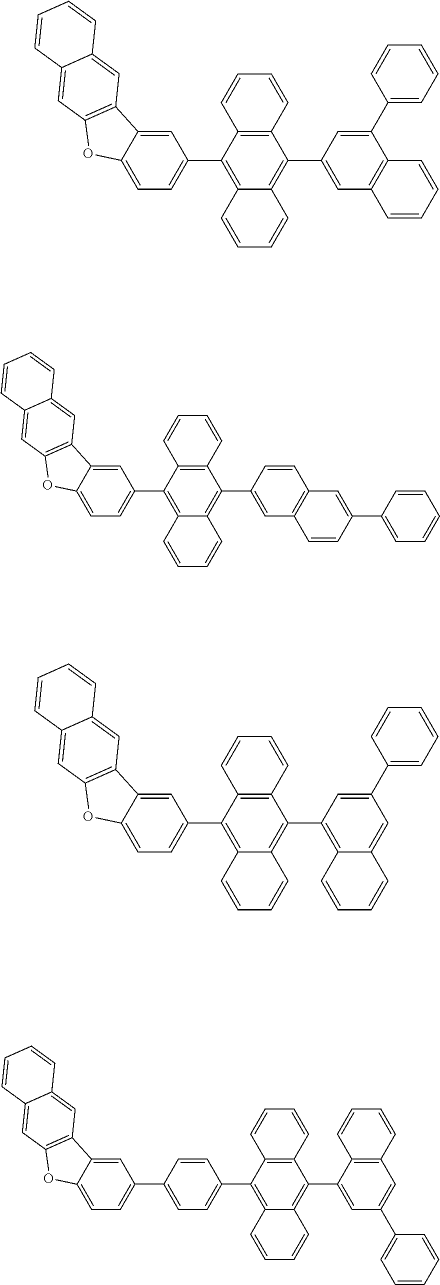 [SMILES: c1ccc(-c2cc(-c3c4ccccc4c(-c4ccc5oc6cc7ccccc7cc6c5c4)c4ccccc34)cc3ccccc23)cc1 ; c1ccc(-c2ccc3cc(-c4c5ccccc5c(-c5ccc6oc7cc8ccccc8cc7c6c5)c5ccccc45)ccc3c2)cc1 ; c1ccc(-c2cc(-c3c4ccccc4c(-c4ccc5oc6cc7ccccc7cc6c5c4)c4ccccc34)c3ccccc3c2)cc1 ; c1ccc(-c2cc(-c3c4ccccc4c(-c4ccc(-c5ccc6oc7cc8ccccc8cc7c6c5)cc4)c4ccccc34)c3ccccc3c2)cc1]

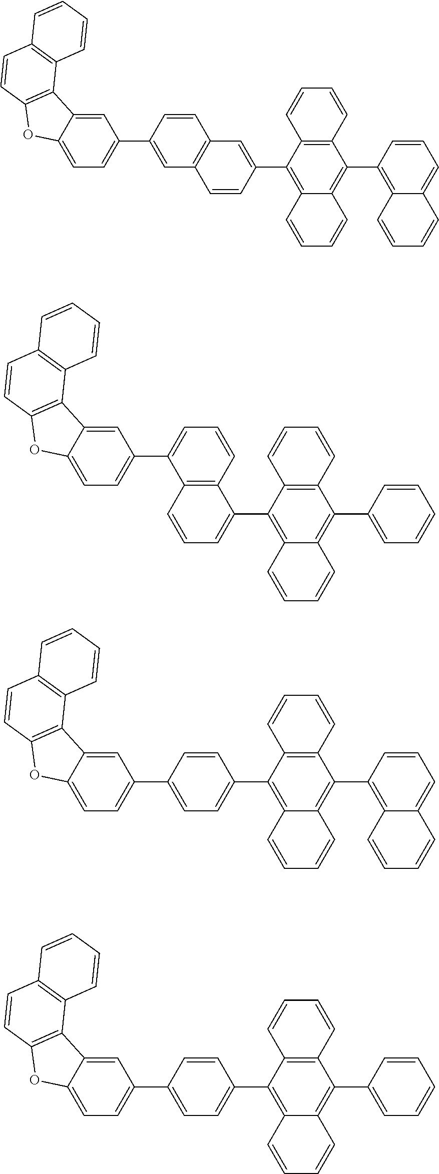 [SMILES: c1ccc2c(-c3c4ccccc4c(-c4ccc5cc(-c6ccc7oc8ccc9ccccc9c8c7c6)ccc5c4)c4ccccc34)cccc2c1 ; c1ccc(-c2c3ccccc3c(-c3cccc4c(-c5ccc6oc7ccc8ccccc8c7c6c5)cccc34)c3ccccc23)cc1 ; c1ccc2c(-c3c4ccccc4c(-c4ccc(-c5ccc6oc7ccc8ccccc8c7c6c5)cc4)c4ccccc34)cccc2c1 ; c1ccc(-c2c3ccccc3c(-c3ccc(-c4ccc5oc6ccc7ccccc7c6c5c4)cc3)c3ccccc23)cc1]

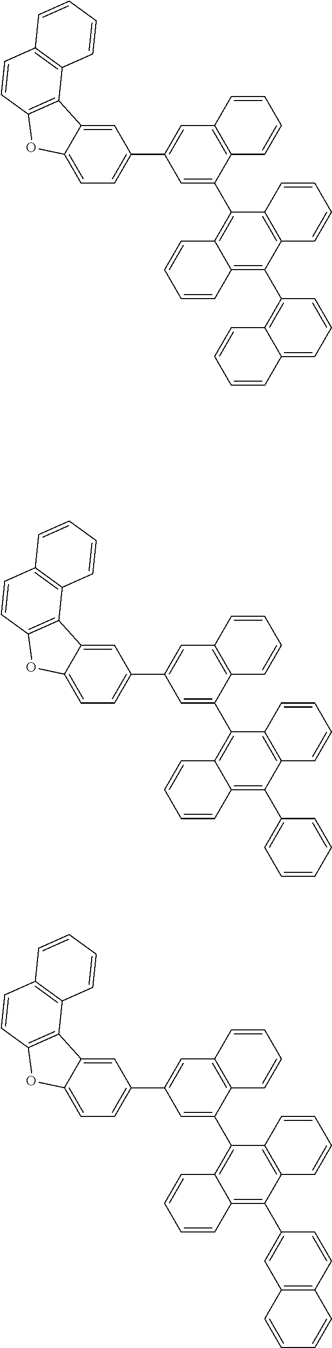 [SMILES: c1ccc2c(-c3c4ccccc4c(-c4cc(-c5ccc6oc7ccc8ccccc8c7c6c5)cc5ccccc45)c4ccccc34)cccc2c1 ; c1ccc(-c2c3ccccc3c(-c3cc(-c4ccc5oc6ccc7ccccc7c6c5c4)cc4ccccc34)c3ccccc23)cc1 ; c1ccc2cc(-c3c4ccccc4c(-c4cc(-c5ccc6oc7ccc8ccccc8c7c6c5)cc5ccccc45)c4ccccc34)ccc2c1]

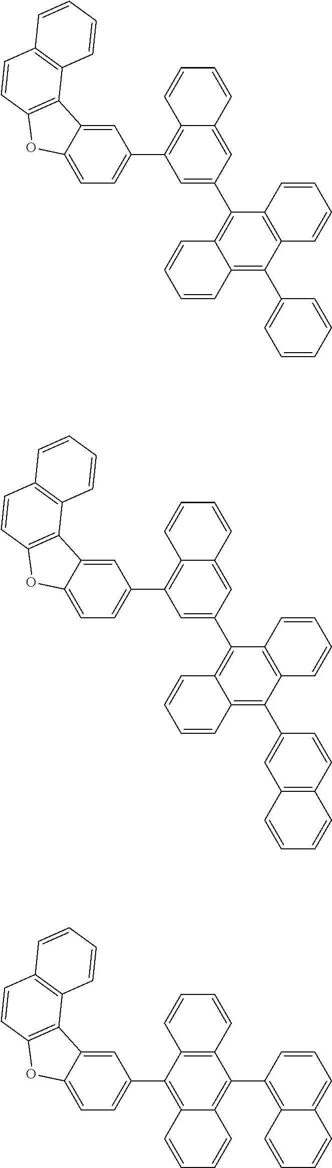 [SMILES: c1ccc(-c2c3ccccc3c(-c3cc(-c4ccc5oc6ccc7ccccc7c6c5c4)c4ccccc4c3)c3ccccc23)cc1 ; c1ccc2cc(-c3c4ccccc4c(-c4cc(-c5ccc6oc7ccc8ccccc8c7c6c5)c5ccccc5c4)c4ccccc34)ccc2c1 ; c1ccc2c(-c3c4ccccc4c(-c4ccc5oc6ccc7ccccc7c6c5c4)c4ccccc34)cccc2c1]

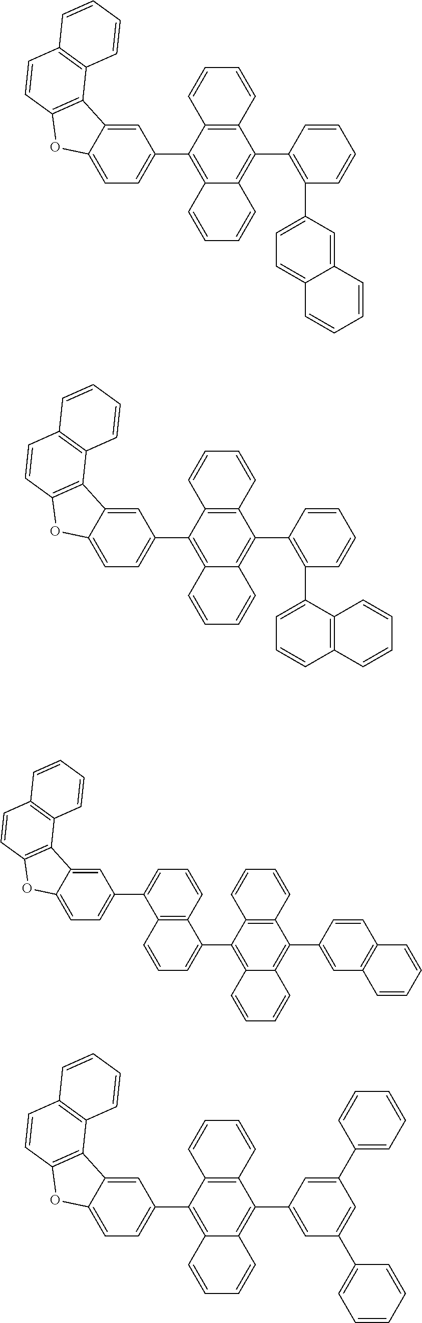 [SMILES: c1ccc(-c2c3ccccc3c(-c3ccc4oc5ccc6ccccc6c5c4c3)c3ccccc23)c(-c2ccc3ccccc3c2)c1 ; c1ccc(-c2c3ccccc3c(-c3ccc4oc5ccc6ccccc6c5c4c3)c3ccccc23)c(-c2cccc3ccccc23)c1 ; c1ccc2cc(-c3c4ccccc4c(-c4cccc5c(-c6ccc7oc8ccc9ccccc9c8c7c6)cccc45)c4ccccc34)ccc2c1 ; c1ccc(-c2cc(-c3ccccc3)cc(-c3c4ccccc4c(-c4ccc5oc6ccc7ccccc7c6c5c4)c4ccccc34)c2)cc1]

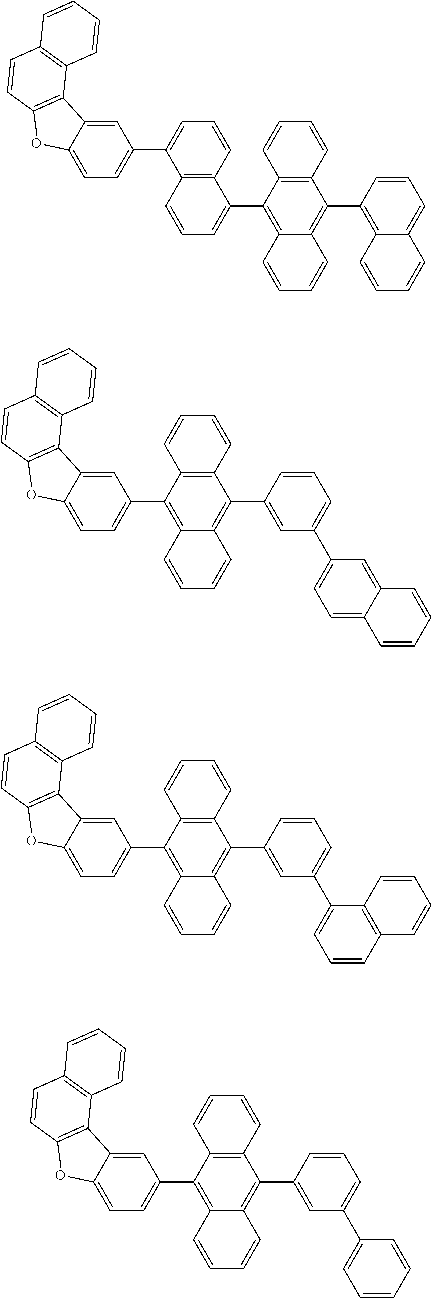 [SMILES: c1ccc2c(-c3c4ccccc4c(-c4cccc5c(-c6ccc7oc8ccc9ccccc9c8c7c6)cccc45)c4ccccc34)cccc2c1 ; c1cc(-c2ccc3ccccc3c2)cc(-c2c3ccccc3c(-c3ccc4oc5ccc6ccccc6c5c4c3)c3ccccc23)c1 ; c1cc(-c2cccc3ccccc23)cc(-c2c3ccccc3c(-c3ccc4oc5ccc6ccccc6c5c4c3)c3ccccc23)c1 ; c1ccc(-c2cccc(-c3c4ccccc4c(-c4ccc5oc6ccc7ccccc7c6c5c4)c4ccccc34)c2)cc1]

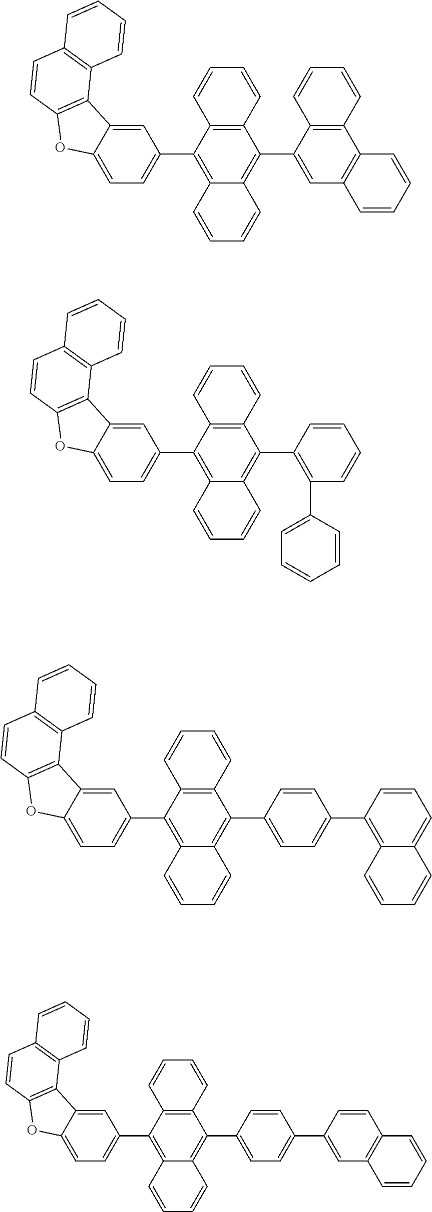 [SMILES: c1ccc2c(c1)cc(-c1c3ccccc3c(-c3ccc4oc5ccc6ccccc6c5c4c3)c3ccccc13)c1ccccc12 ; c1ccc(-c2ccccc2-c2c3ccccc3c(-c3ccc4oc5ccc6ccccc6c5c4c3)c3ccccc23)cc1 ; c1ccc2c(-c3ccc(-c4c5ccccc5c(-c5ccc6oc7ccc8ccccc8c7c6c5)c5ccccc45)cc3)cccc2c1 ; c1ccc2cc(-c3ccc(-c4c5ccccc5c(-c5ccc6oc7ccc8ccccc8c7c6c5)c5ccccc45)cc3)ccc2c1]

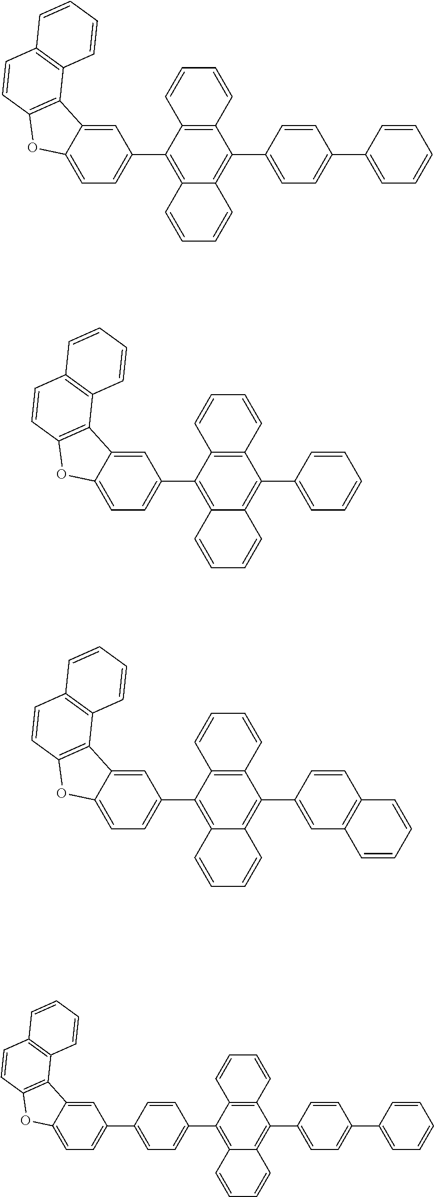 [SMILES: c1ccc(-c2ccc(-c3c4ccccc4c(-c4ccc5oc6ccc7ccccc7c6c5c4)c4ccccc34)cc2)cc1 ; c1ccc(-c2c3ccccc3c(-c3ccc4oc5ccc6ccccc6c5c4c3)c3ccccc23)cc1 ; c1ccc2cc(-c3c4ccccc4c(-c4ccc5oc6ccc7ccccc7c6c5c4)c4ccccc34)ccc2c1 ; c1ccc(-c2ccc(-c3c4ccccc4c(-c4ccc(-c5ccc6oc7ccc8ccccc8c7c6c5)cc4)c4ccccc34)cc2)cc1]

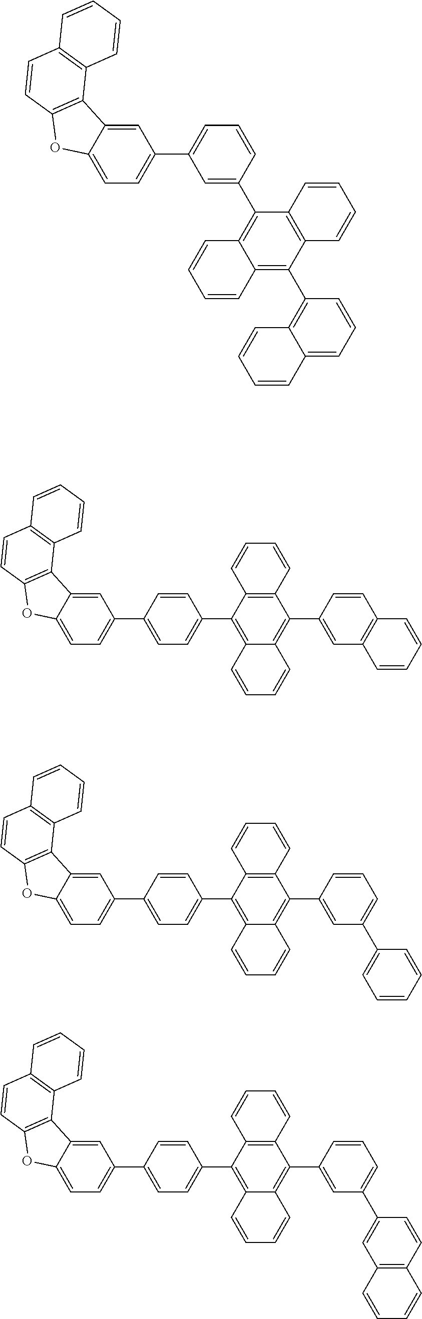 [SMILES: c1cc(-c2ccc3oc4ccc5ccccc5c4c3c2)cc(-c2c3ccccc3c(-c3cccc4ccccc34)c3ccccc23)c1 ; c1ccc2cc(-c3c4ccccc4c(-c4ccc(-c5ccc6oc7ccc8ccccc8c7c6c5)cc4)c4ccccc34)ccc2c1 ; c1ccc(-c2cccc(-c3c4ccccc4c(-c4ccc(-c5ccc6oc7ccc8ccccc8c7c6c5)cc4)c4ccccc34)c2)cc1 ; c1cc(-c2ccc3ccccc3c2)cc(-c2c3ccccc3c(-c3ccc(-c4ccc5oc6ccc7ccccc7c6c5c4)cc3)c3ccccc23)c1]

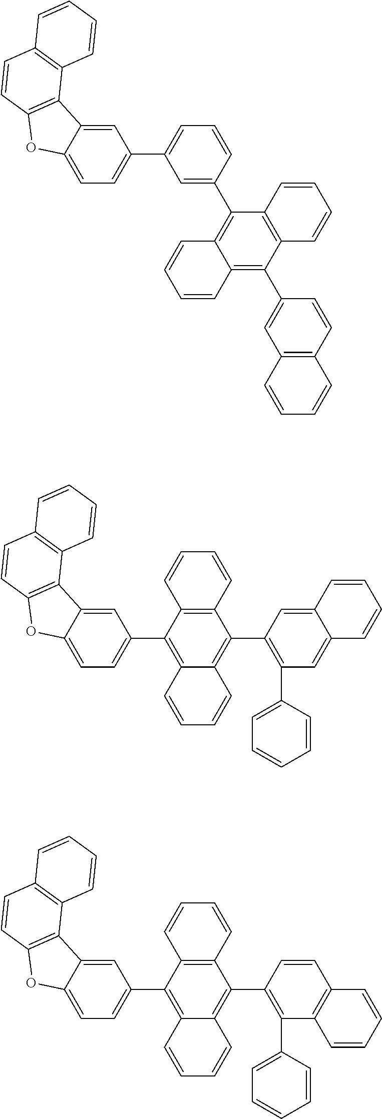 [SMILES: c1cc(-c2ccc3oc4ccc5ccccc5c4c3c2)cc(-c2c3ccccc3c(-c3ccc4ccccc4c3)c3ccccc23)c1 ; c1ccc(-c2cc3ccccc3cc2-c2c3ccccc3c(-c3ccc4oc5ccc6ccccc6c5c4c3)c3ccccc23)cc1 ; c1ccc(-c2c(-c3c4ccccc4c(-c4ccc5oc6ccc7ccccc7c6c5c4)c4ccccc34)ccc3ccccc23)cc1]

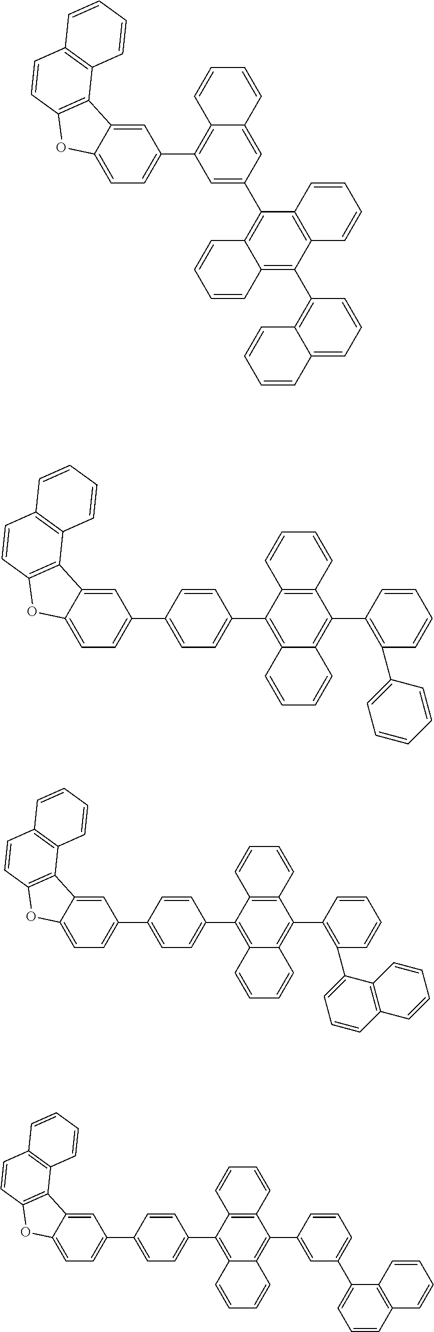 [SMILES: c1ccc2c(-c3ccc4oc5ccc6ccccc6c5c4c3)cc(-c3c4ccccc4c(-c4cccc5ccccc45)c4ccccc34)cc2c1 ; c1ccc(-c2ccccc2-c2c3ccccc3c(-c3ccc(-c4ccc5oc6ccc7ccccc7c6c5c4)cc3)c3ccccc23)cc1 ; c1ccc(-c2c3ccccc3c(-c3ccc(-c4ccc5oc6ccc7ccccc7c6c5c4)cc3)c3ccccc23)c(-c2cccc3ccccc23)c1 ; c1cc(-c2cccc3ccccc23)cc(-c2c3ccccc3c(-c3ccc(-c4ccc5oc6ccc7ccccc7c6c5c4)cc3)c3ccccc23)c1]

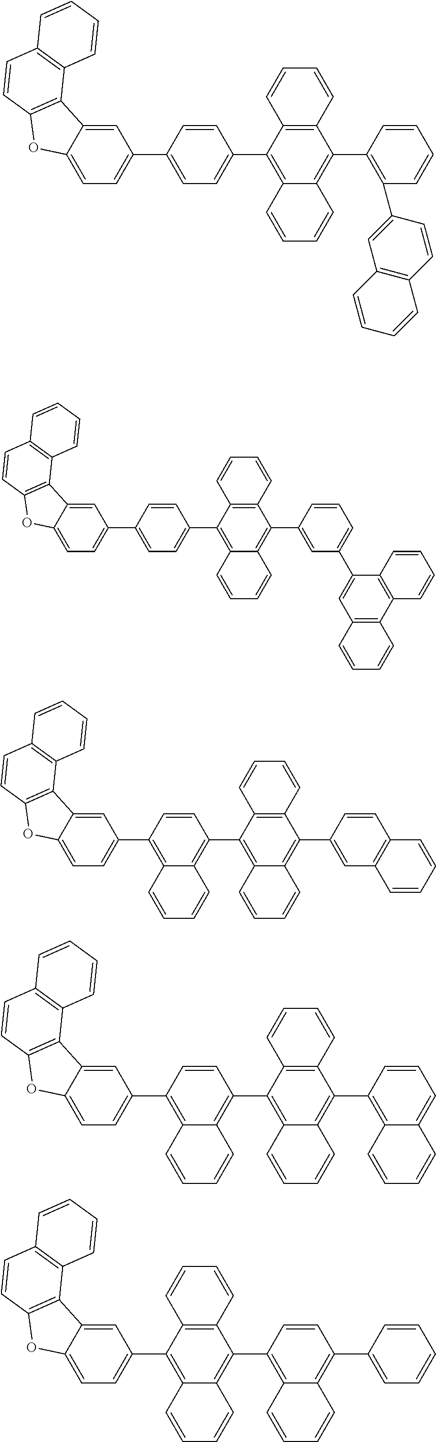 [SMILES: c1ccc(-c2c3ccccc3c(-c3ccc(-c4ccc5oc6ccc7ccccc7c6c5c4)cc3)c3ccccc23)c(-c2ccc3ccccc3c2)c1 ; c1cc(-c2c3ccccc3c(-c3ccc(-c4ccc5oc6ccc7ccccc7c6c5c4)cc3)c3ccccc23)cc(-c2cc3ccccc3c3ccccc23)c1 ; c1ccc2cc(-c3c4ccccc4c(-c4ccc(-c5ccc6oc7ccc8ccccc8c7c6c5)c5ccccc45)c4ccccc34)ccc2c1 ; c1ccc2c(-c3c4ccccc4c(-c4ccc(-c5ccc6oc7ccc8ccccc8c7c6c5)c5ccccc45)c4ccccc34)cccc2c1 ; c1ccc(-c2ccc(-c3c4ccccc4c(-c4ccc5oc6ccc7ccccc7c6c5c4)c4ccccc34)c3ccccc23)cc1]

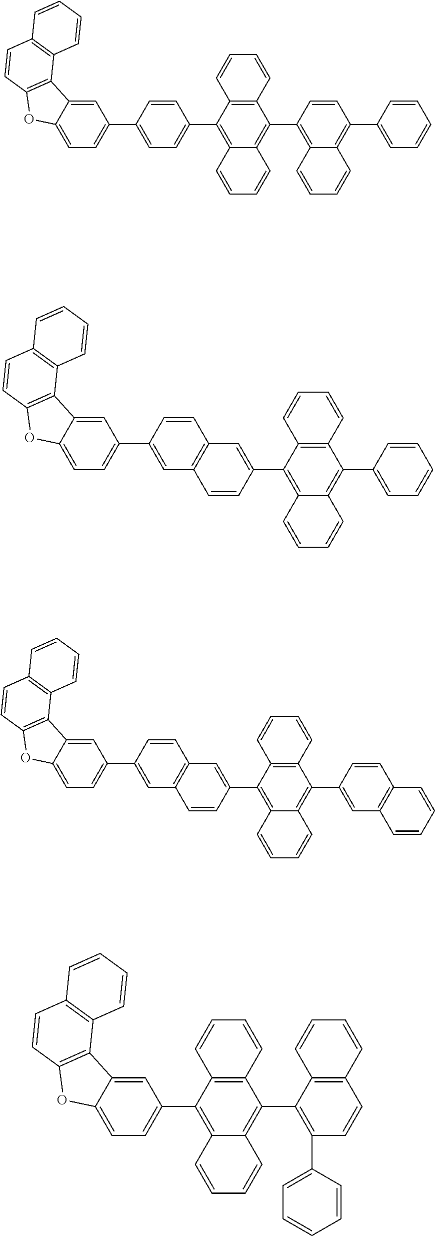 [SMILES: c1ccc(-c2ccc(-c3c4ccccc4c(-c4ccc(-c5ccc6oc7ccc8ccccc8c7c6c5)cc4)c4ccccc34)c3ccccc23)cc1 ; c1ccc(-c2c3ccccc3c(-c3ccc4cc(-c5ccc6oc7ccc8ccccc8c7c6c5)ccc4c3)c3ccccc23)cc1 ; c1ccc2cc(-c3c4ccccc4c(-c4ccc5cc(-c6ccc7oc8ccc9ccccc9c8c7c6)ccc5c4)c4ccccc34)ccc2c1 ; c1ccc(-c2ccc3ccccc3c2-c2c3ccccc3c(-c3ccc4oc5ccc6ccccc6c5c4c3)c3ccccc23)cc1]

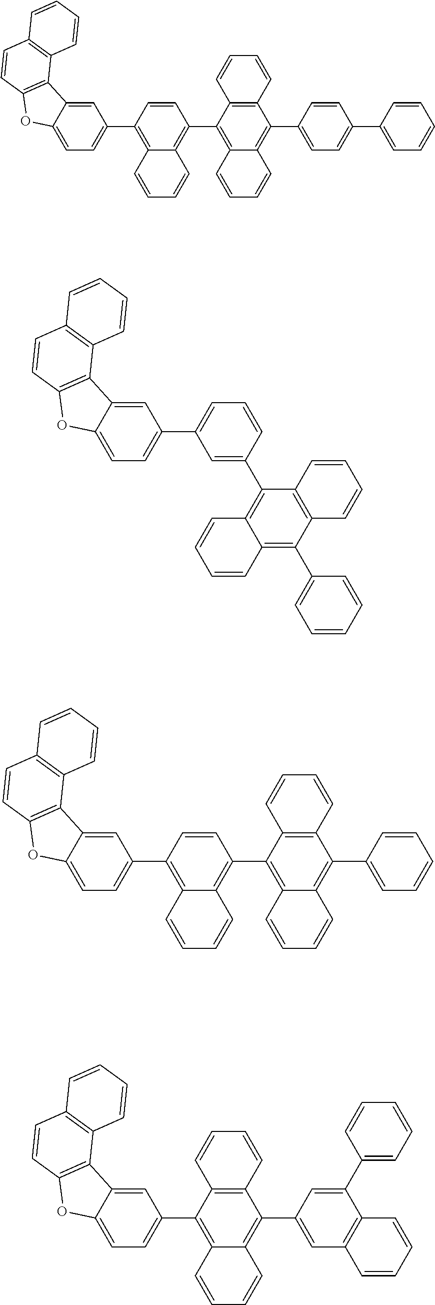 [SMILES: c1ccc(-c2ccc(-c3c4ccccc4c(-c4ccc(-c5ccc6oc7ccc8ccccc8c7c6c5)c5ccccc45)c4ccccc34)cc2)cc1 ; c1ccc(-c2c3ccccc3c(-c3cccc(-c4ccc5oc6ccc7ccccc7c6c5c4)c3)c3ccccc23)cc1 ; c1ccc(-c2c3ccccc3c(-c3ccc(-c4ccc5oc6ccc7ccccc7c6c5c4)c4ccccc34)c3ccccc23)cc1 ; c1ccc(-c2cc(-c3c4ccccc4c(-c4ccc5oc6ccc7ccccc7c6c5c4)c4ccccc34)cc3ccccc23)cc1]

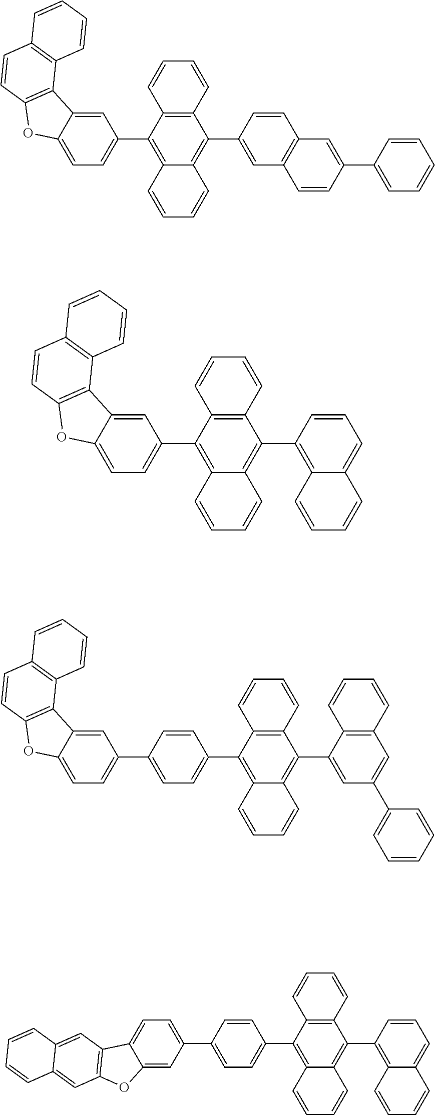 [SMILES: c1ccc(-c2ccc3cc(-c4c5ccccc5c(-c5ccc6oc7ccc8ccccc8c7c6c5)c5ccccc45)ccc3c2)cc1 ; c1ccc2c(-c3c4ccccc4c(-c4ccc5oc6ccc7ccccc7c6c5c4)c4ccccc34)cccc2c1 ; c1ccc(-c2cc(-c3c4ccccc4c(-c4ccc(-c5ccc6oc7ccc8ccccc8c7c6c5)cc4)c4ccccc34)c3ccccc3c2)cc1 ; c1ccc2cc3c(cc2c1)oc1cc(-c2ccc(-c4c5ccccc5c(-c5cccc6ccccc56)c5ccccc45)cc2)ccc13]

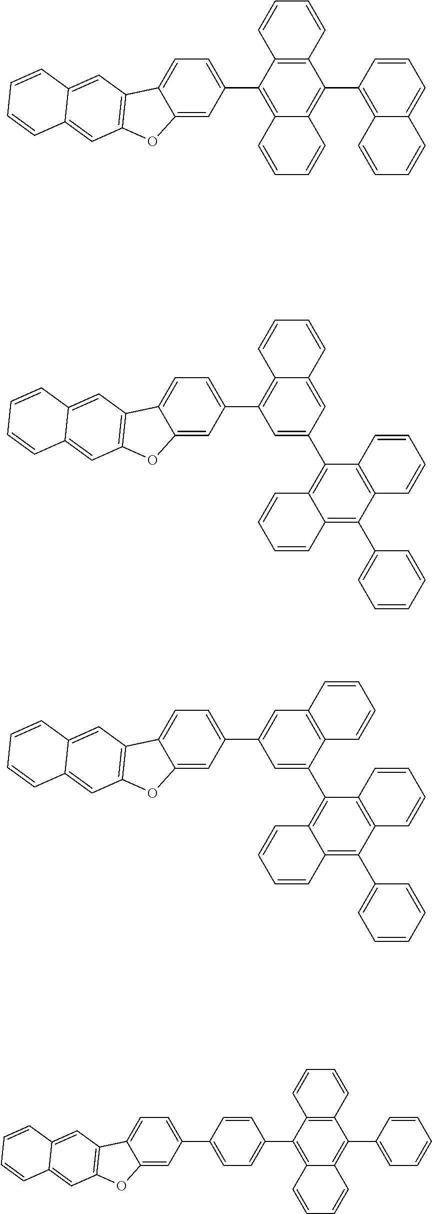 [SMILES: c1ccc2cc3c(cc2c1)oc1cc(-c2c4ccccc4c(-c4cccc5ccccc45)c4ccccc24)ccc13 ; c1ccc(-c2c3ccccc3c(-c3cc(-c4ccc5c(c4)oc4cc6ccccc6cc45)c4ccccc4c3)c3ccccc23)cc1 ; c1ccc(-c2c3ccccc3c(-c3cc(-c4ccc5c(c4)oc4cc6ccccc6cc45)cc4ccccc34)c3ccccc23)cc1 ; c1ccc(-c2c3ccccc3c(-c3ccc(-c4ccc5c(c4)oc4cc6ccccc6cc45)cc3)c3ccccc23)cc1]

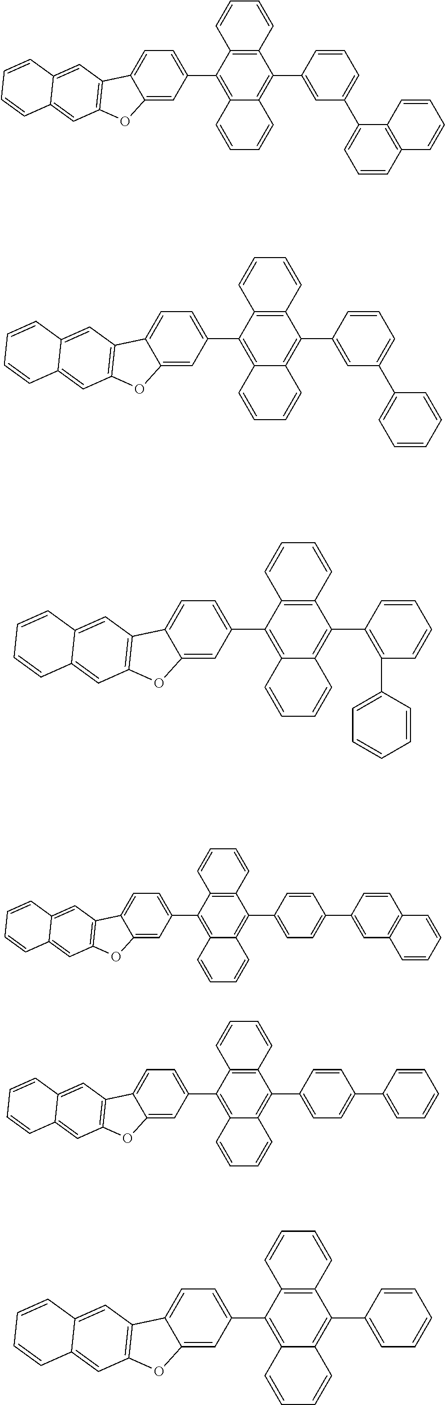 [SMILES: c1cc(-c2cccc3ccccc23)cc(-c2c3ccccc3c(-c3ccc4c(c3)oc3cc5ccccc5cc34)c3ccccc23)c1 ; c1ccc(-c2cccc(-c3c4ccccc4c(-c4ccc5c(c4)oc4cc6ccccc6cc45)c4ccccc34)c2)cc1 ; c1ccc(-c2ccccc2-c2c3ccccc3c(-c3ccc4c(c3)oc3cc5ccccc5cc34)c3ccccc23)cc1 ; c1ccc2cc(-c3ccc(-c4c5ccccc5c(-c5ccc6c(c5)oc5cc7ccccc7cc56)c5ccccc45)cc3)ccc2c1 ; c1ccc(-c2ccc(-c3c4ccccc4c(-c4ccc5c(c4)oc4cc6ccccc6cc45)c4ccccc34)cc2)cc1 ; c1ccc(-c2c3ccccc3c(-c3ccc4c(c3)oc3cc5ccccc5cc34)c3ccccc23)cc1]

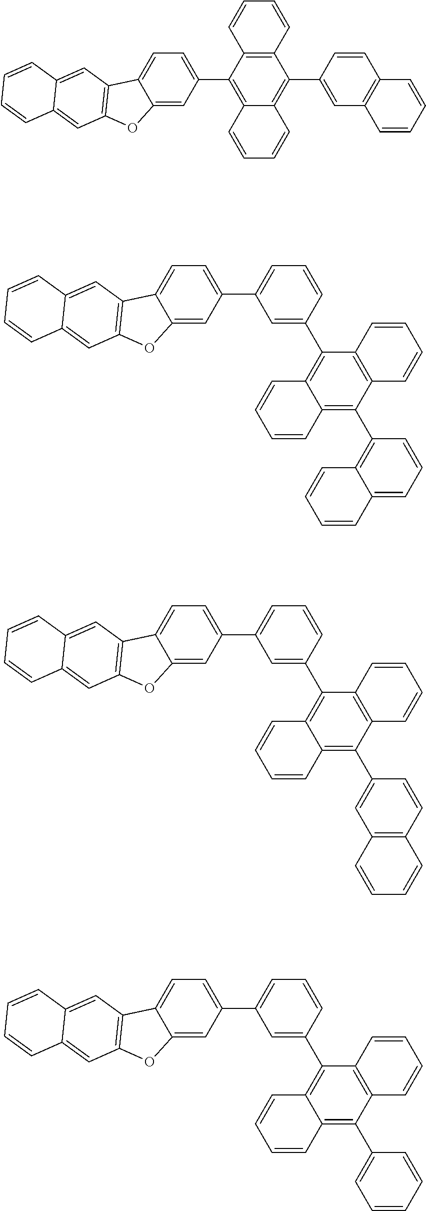 [SMILES: c1ccc2cc(-c3c4ccccc4c(-c4ccc5c(c4)oc4cc6ccccc6cc45)c4ccccc34)ccc2c1 ; c1cc(-c2ccc3c(c2)oc2cc4ccccc4cc23)cc(-c2c3ccccc3c(-c3cccc4ccccc34)c3ccccc23)c1 ; c1cc(-c2ccc3c(c2)oc2cc4ccccc4cc23)cc(-c2c3ccccc3c(-c3ccc4ccccc4c3)c3ccccc23)c1 ; c1ccc(-c2c3ccccc3c(-c3cccc(-c4ccc5c(c4)oc4cc6ccccc6cc45)c3)c3ccccc23)cc1]

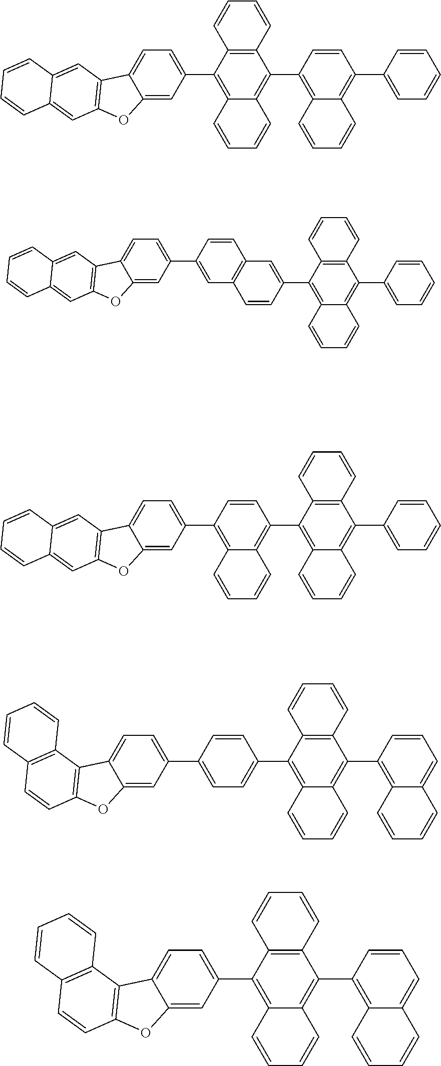 [SMILES: c1ccc(-c2ccc(-c3c4ccccc4c(-c4ccc5c(c4)oc4cc6ccccc6cc45)c4ccccc34)c3ccccc23)cc1 ; c1ccc(-c2c3ccccc3c(-c3ccc4cc(-c5ccc6c(c5)oc5cc7ccccc7cc56)ccc4c3)c3ccccc23)cc1 ; c1ccc(-c2c3ccccc3c(-c3ccc(-c4ccc5c(c4)oc4cc6ccccc6cc45)c4ccccc34)c3ccccc23)cc1 ; c1ccc2c(-c3c4ccccc4c(-c4ccc(-c5ccc6c(c5)oc5ccc7ccccc7c56)cc4)c4ccccc34)cccc2c1 ; c1ccc2c(-c3c4ccccc4c(-c4ccc5c(c4)oc4ccc6ccccc6c45)c4ccccc34)cccc2c1]

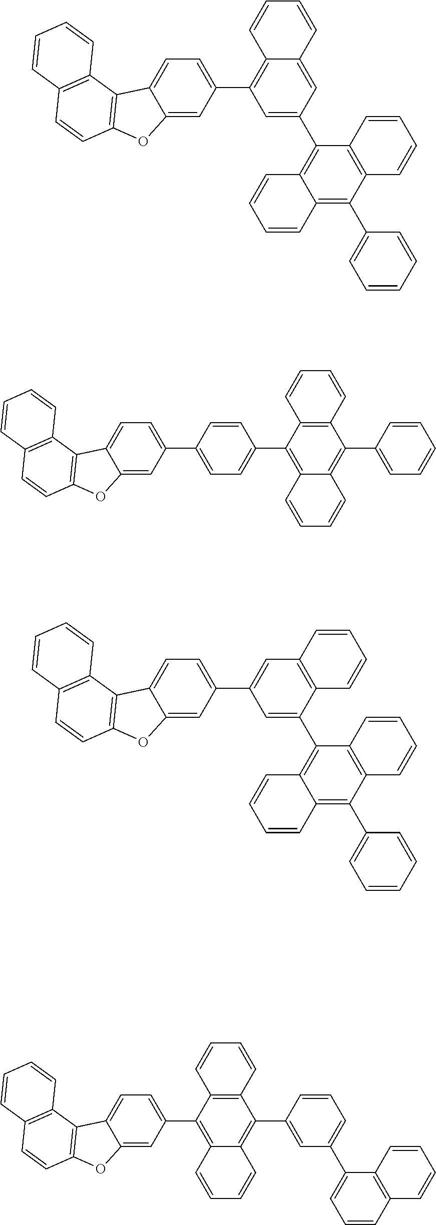 [SMILES: c1ccc(-c2c3ccccc3c(-c3cc(-c4ccc5c(c4)oc4ccc6ccccc6c45)c4ccccc4c3)c3ccccc23)cc1 ; c1ccc(-c2c3ccccc3c(-c3ccc(-c4ccc5c(c4)oc4ccc6ccccc6c45)cc3)c3ccccc23)cc1 ; c1ccc(-c2c3ccccc3c(-c3cc(-c4ccc5c(c4)oc4ccc6ccccc6c45)cc4ccccc34)c3ccccc23)cc1 ; c1cc(-c2cccc3ccccc23)cc(-c2c3ccccc3c(-c3ccc4c(c3)oc3ccc5ccccc5c34)c3ccccc23)c1]

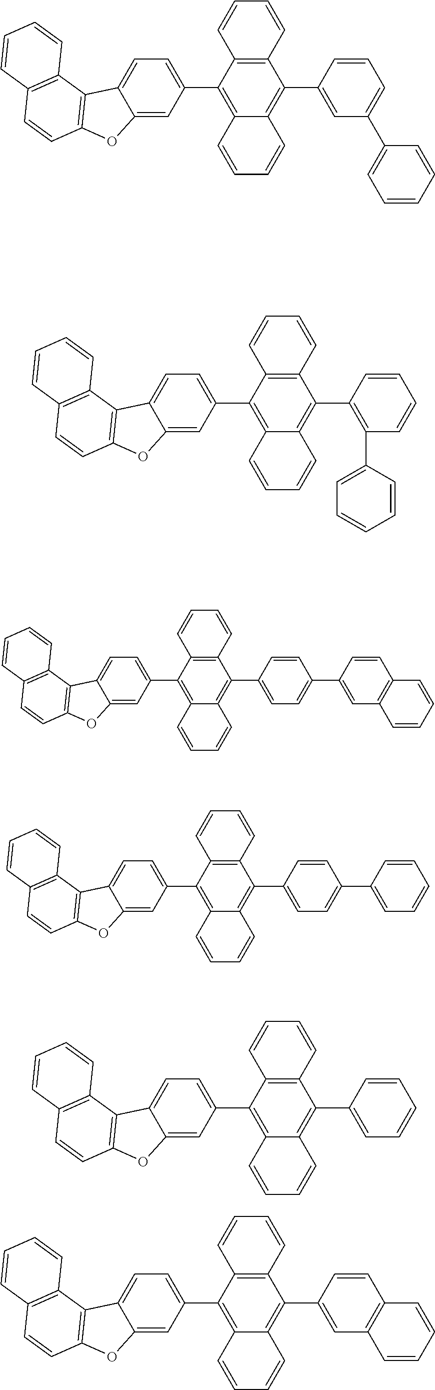

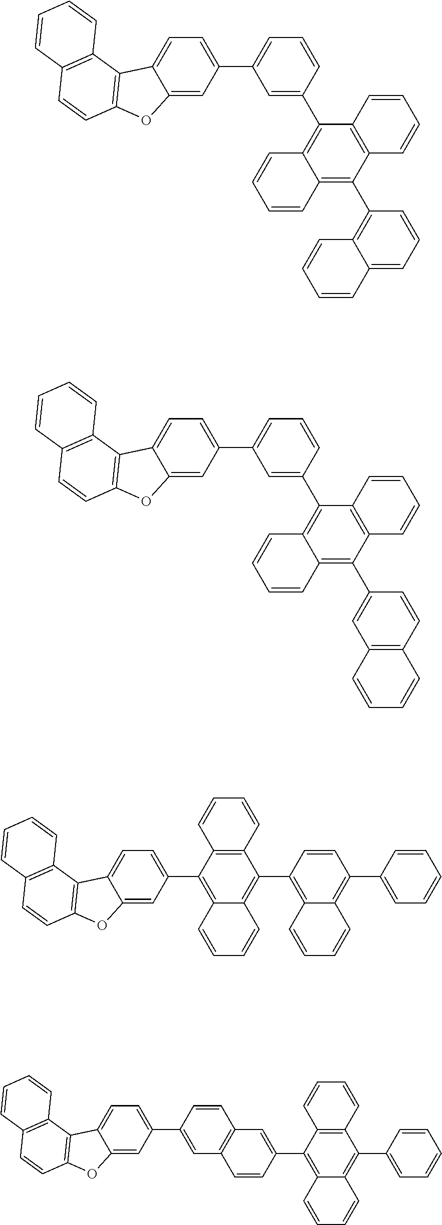 [SMILES: c1cc(-c2ccc3c(c2)oc2ccc4ccccc4c23)cc(-c2c3ccccc3c(-c3cccc4ccccc34)c3ccccc23)c1 ; c1cc(-c2ccc3c(c2)oc2ccc4ccccc4c23)cc(-c2c3ccccc3c(-c3ccc4ccccc4c3)c3ccccc23)c1 ; c1ccc(-c2ccc(-c3c4ccccc4c(-c4ccc5c(c4)oc4ccc6ccccc6c45)c4ccccc34)c3ccccc23)cc1 ; c1ccc(-c2c3ccccc3c(-c3ccc4cc(-c5ccc6c(c5)oc5ccc7ccccc7c56)ccc4c3)c3ccccc23)cc1]

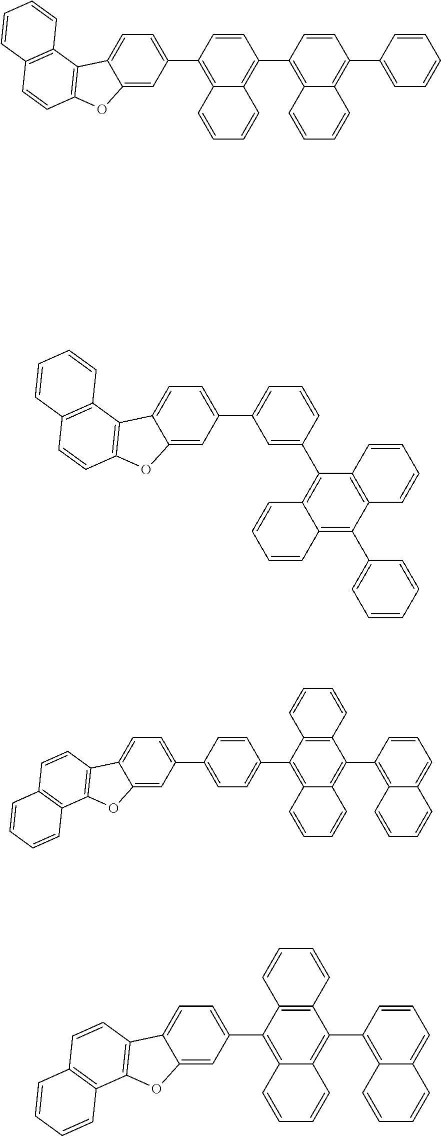 [SMILES: c1ccc(-c2ccc(-c3ccc(-c4ccc5c(c4)oc4ccc6ccccc6c45)c4ccccc34)c3ccccc23)cc1 ; c1ccc(-c2c3ccccc3c(-c3cccc(-c4ccc5c(c4)oc4ccc6ccccc6c45)c3)c3ccccc23)cc1 ; c1ccc2c(-c3c4ccccc4c(-c4ccc(-c5ccc6c(c5)oc5c7ccccc7ccc65)cc4)c4ccccc34)cccc2c1 ; c1ccc2c(-c3c4ccccc4c(-c4ccc5c(c4)oc4c6ccccc6ccc54)c4ccccc34)cccc2c1]

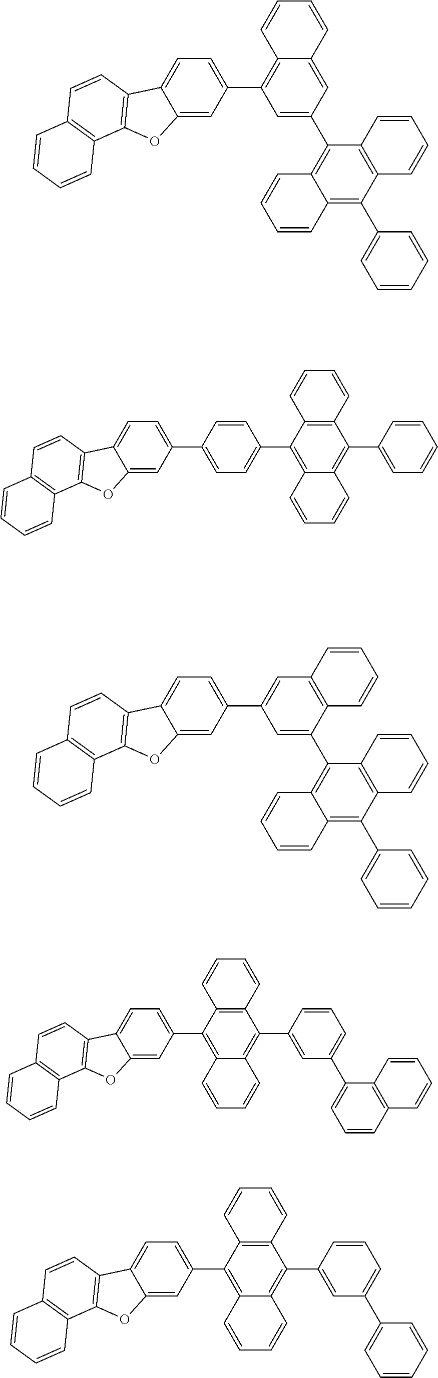 [SMILES: c1ccc(-c2c3ccccc3c(-c3cc(-c4ccc5c(c4)oc4c6ccccc6ccc54)c4ccccc4c3)c3ccccc23)cc1 ; c1ccc(-c2c3ccccc3c(-c3ccc(-c4ccc5c(c4)oc4c6ccccc6ccc54)cc3)c3ccccc23)cc1 ; c1ccc(-c2c3ccccc3c(-c3cc(-c4ccc5c(c4)oc4c6ccccc6ccc54)cc4ccccc34)c3ccccc23)cc1 ; c1cc(-c2cccc3ccccc23)cc(-c2c3ccccc3c(-c3ccc4c(c3)oc3c5ccccc5ccc43)c3ccccc23)c1 ; c1ccc(-c2cccc(-c3c4ccccc4c(-c4ccc5c(c4)oc4c6ccccc6ccc54)c4ccccc34)c2)cc1]

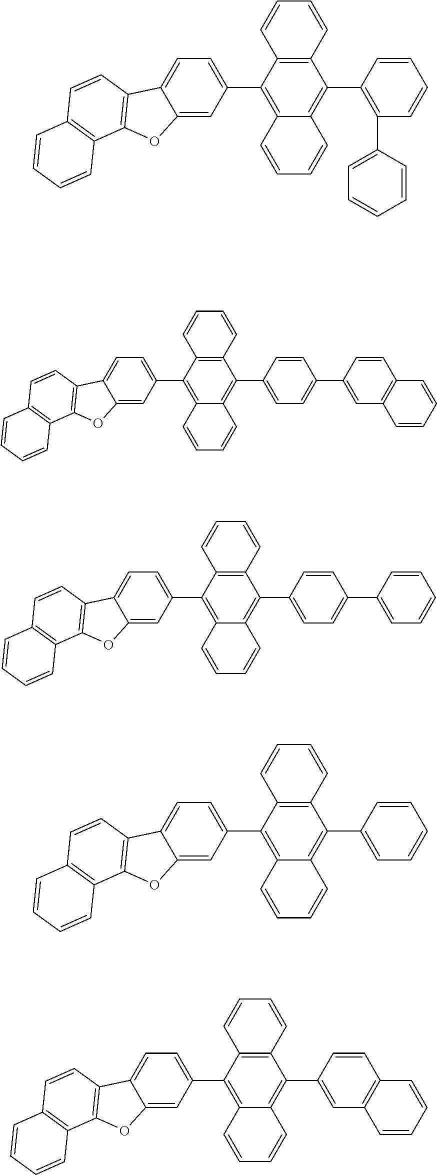 [SMILES: c1ccc(-c2ccccc2-c2c3ccccc3c(-c3ccc4c(c3)oc3c5ccccc5ccc43)c3ccccc23)cc1 ; c1ccc2cc(-c3ccc(-c4c5ccccc5c(-c5ccc6c(c5)oc5c7ccccc7ccc65)c5ccccc45)cc3)ccc2c1 ; c1ccc(-c2ccc(-c3c4ccccc4c(-c4ccc5c(c4)oc4c6ccccc6ccc54)c4ccccc34)cc2)cc1 ; c1ccc(-c2c3ccccc3c(-c3ccc4c(c3)oc3c5ccccc5ccc43)c3ccccc23)cc1 ; c1ccc2cc(-c3c4ccccc4c(-c4ccc5c(c4)oc4c6ccccc6ccc54)c4ccccc34)ccc2c1]

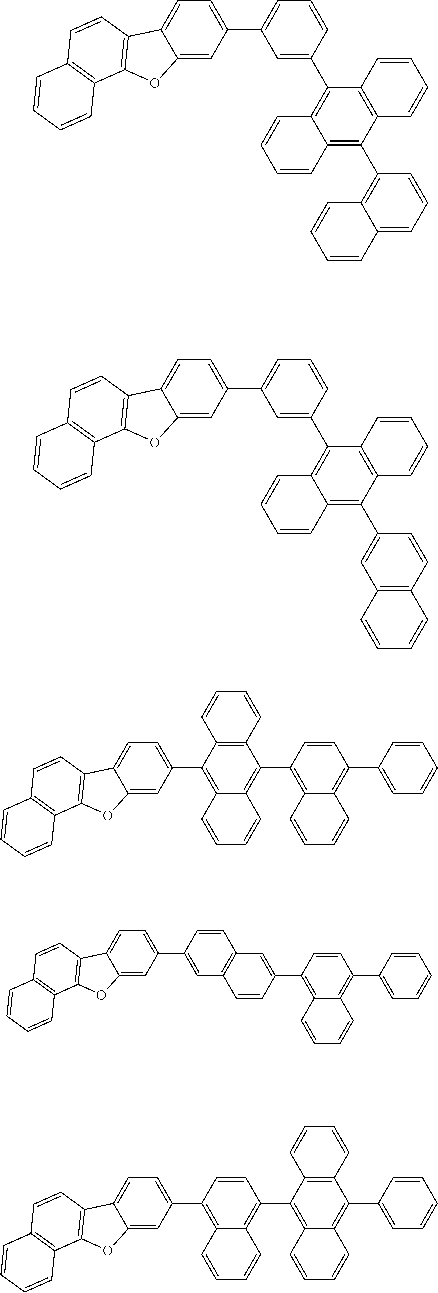 [SMILES: c1cc(-c2ccc3c(c2)oc2c4ccccc4ccc32)cc(-c2c3ccccc3c(-c3cccc4ccccc34)c3ccccc23)c1 ; c1cc(-c2ccc3c(c2)oc2c4ccccc4ccc32)cc(-c2c3ccccc3c(-c3ccc4ccccc4c3)c3ccccc23)c1 ; c1ccc(-c2ccc(-c3c4ccccc4c(-c4ccc5c(c4)oc4c6ccccc6ccc54)c4ccccc34)c3ccccc23)cc1 ; c1ccc(-c2ccc(-c3ccc4cc(-c5ccc6c(c5)oc5c7ccccc7ccc65)ccc4c3)c3ccccc23)cc1 ; c1ccc(-c2c3ccccc3c(-c3ccc(-c4ccc5c(c4)oc4c6ccccc6ccc54)c4ccccc34)c3ccccc23)cc1]

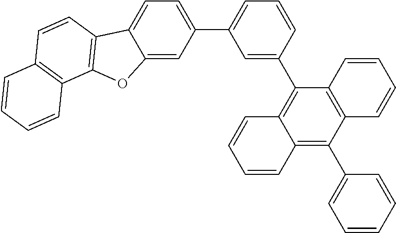 [SMILES: c1ccc(-c2c3ccccc3c(-c3cccc(-c4ccc5c(c4)oc4c6ccccc6ccc54)c3)c3ccccc23)cc1]

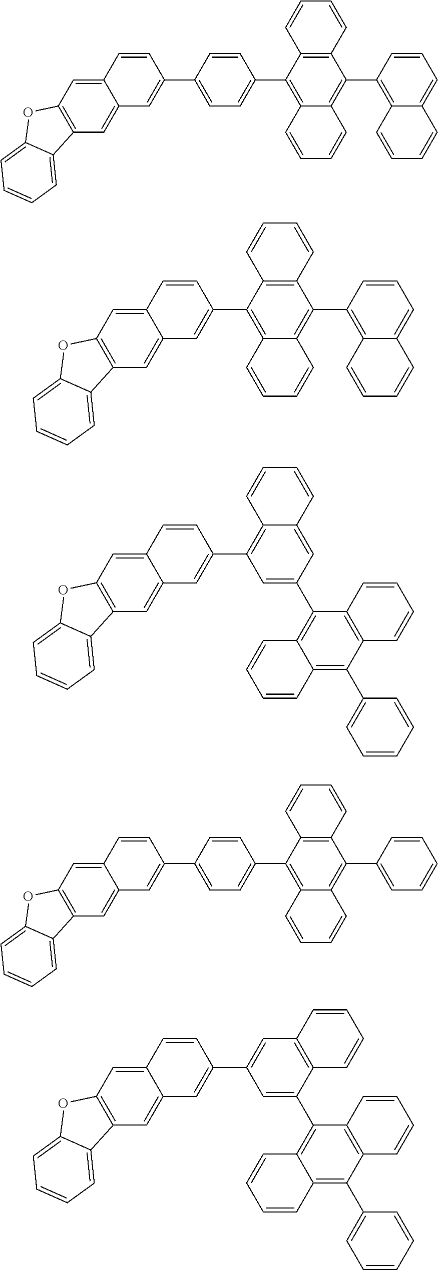

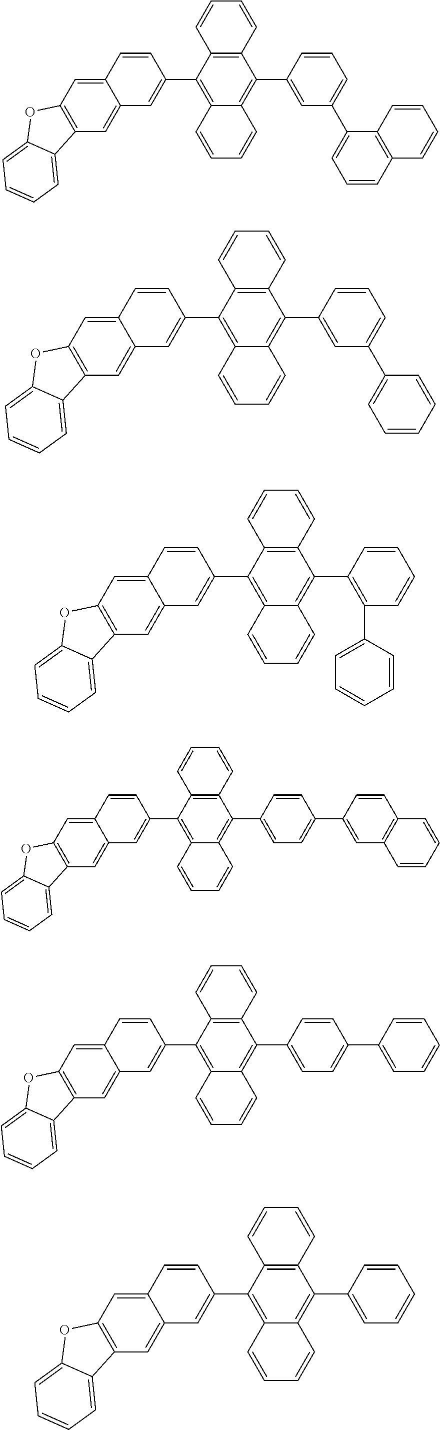 [SMILES: c1cc(-c2cccc3ccccc23)cc(-c2c3ccccc3c(-c3ccc4cc5oc6ccccc6c5cc4c3)c3ccccc23)c1 ; c1ccc(-c2cccc(-c3c4ccccc4c(-c4ccc5cc6oc7ccccc7c6cc5c4)c4ccccc34)c2)cc1 ; c1ccc(-c2ccccc2-c2c3ccccc3c(-c3ccc4cc5oc6ccccc6c5cc4c3)c3ccccc23)cc1 ; c1ccc2cc(-c3ccc(-c4c5ccccc5c(-c5ccc6cc7oc8ccccc8c7cc6c5)c5ccccc45)cc3)ccc2c1 ; c1ccc(-c2ccc(-c3c4ccccc4c(-c4ccc5cc6oc7ccccc7c6cc5c4)c4ccccc34)cc2)cc1 ; c1ccc(-c2c3ccccc3c(-c3ccc4cc5oc6ccccc6c5cc4c3)c3ccccc23)cc1]

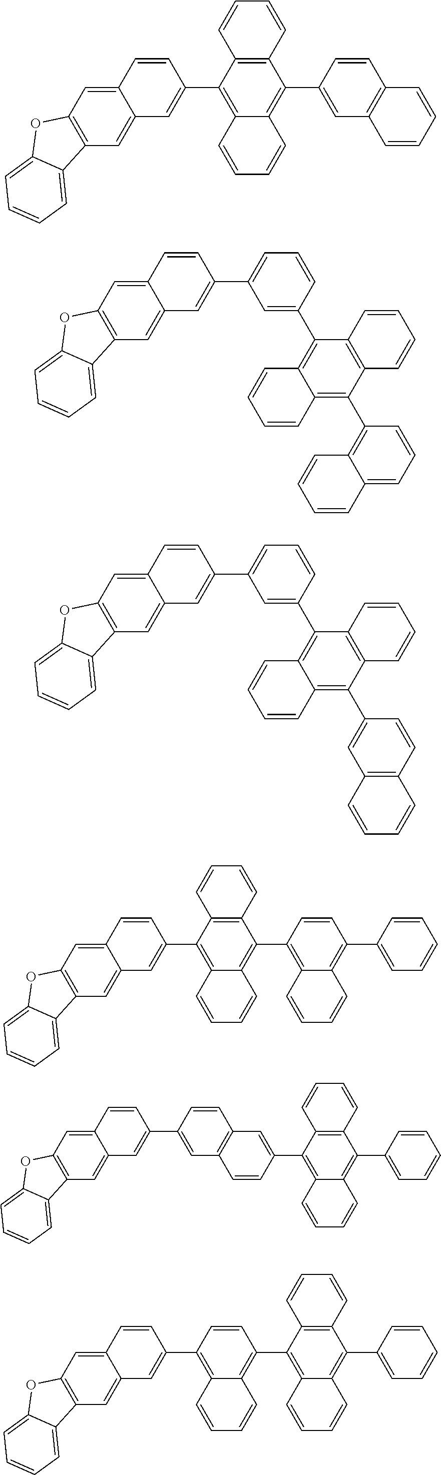

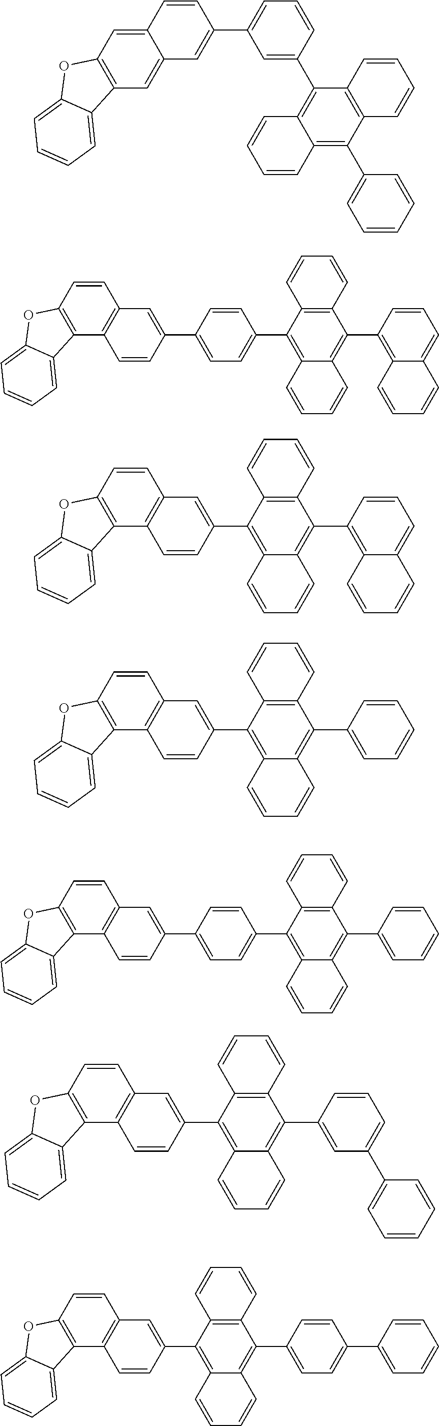

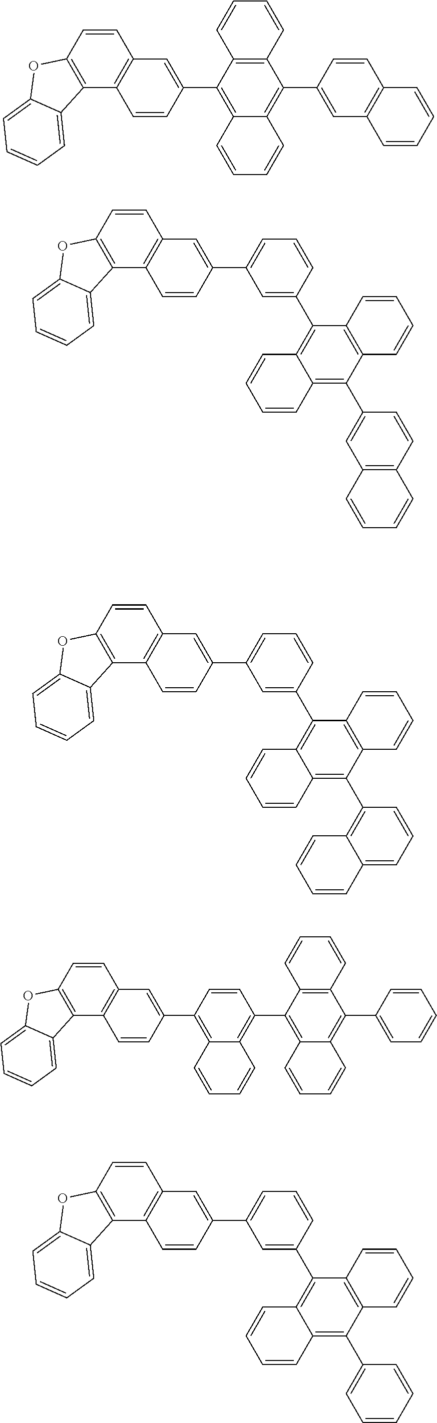

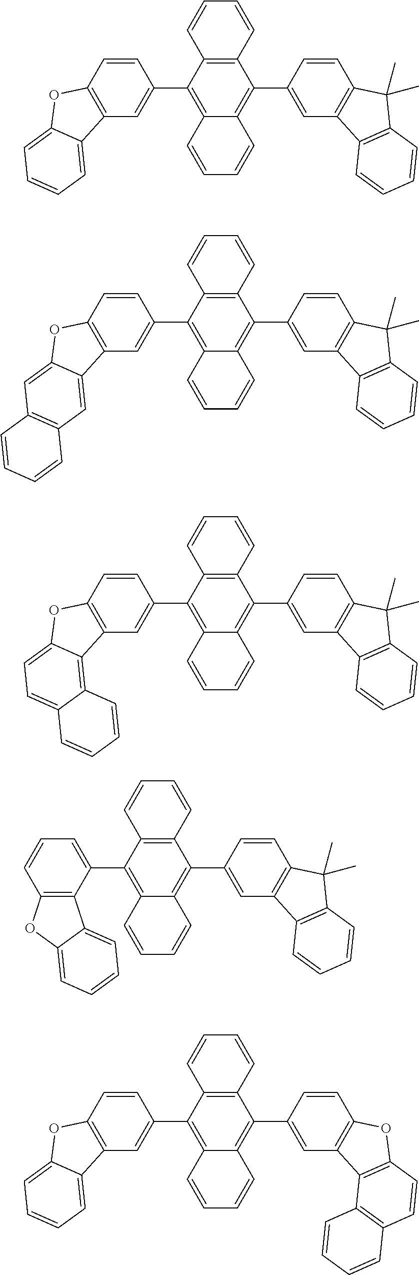 [SMILES: CC1(C)c2ccccc2-c2cc(-c3c4ccccc4c(-c4ccc5oc6ccccc6c5c4)c4ccccc34)ccc21 ; CC1(C)c2ccccc2-c2cc(-c3c4ccccc4c(-c4ccc5oc6cc7ccccc7cc6c5c4)c4ccccc34)ccc21 ; CC1(C)c2ccccc2-c2cc(-c3c4ccccc4c(-c4ccc5oc6ccc7ccccc7c6c5c4)c4ccccc34)ccc21 ; CC1(C)c2ccccc2-c2cc(-c3c4ccccc4c(-c4cccc5oc6ccccc6c45)c4ccccc34)ccc21 ; c1ccc2c(c1)ccc1oc3ccc(-c4c5ccccc5c(-c5ccc6oc7ccccc7c6c5)c5ccccc45)cc3c12]

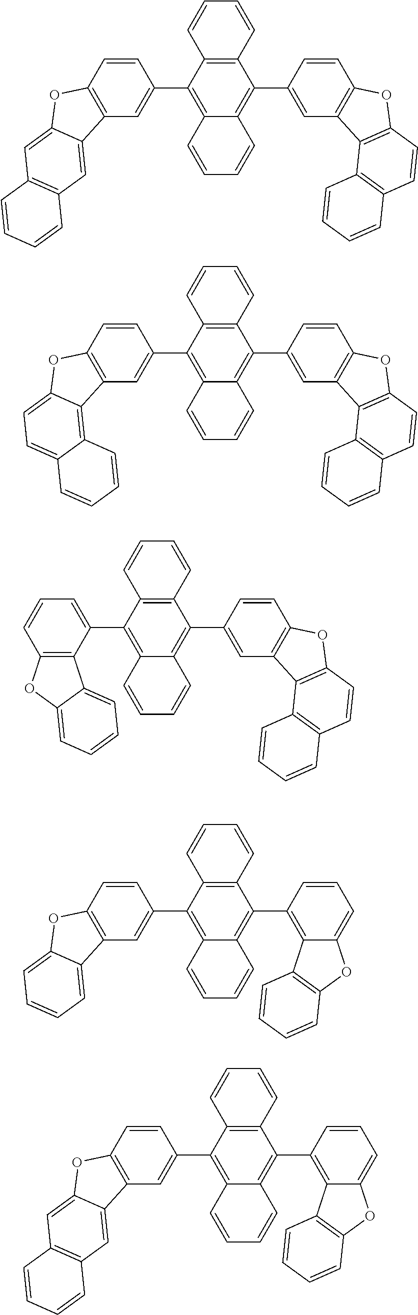

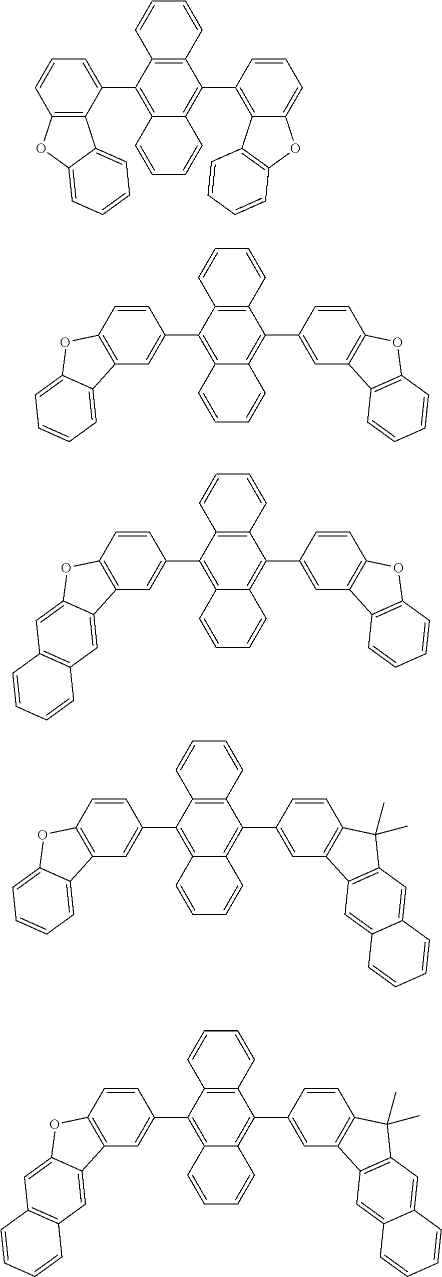

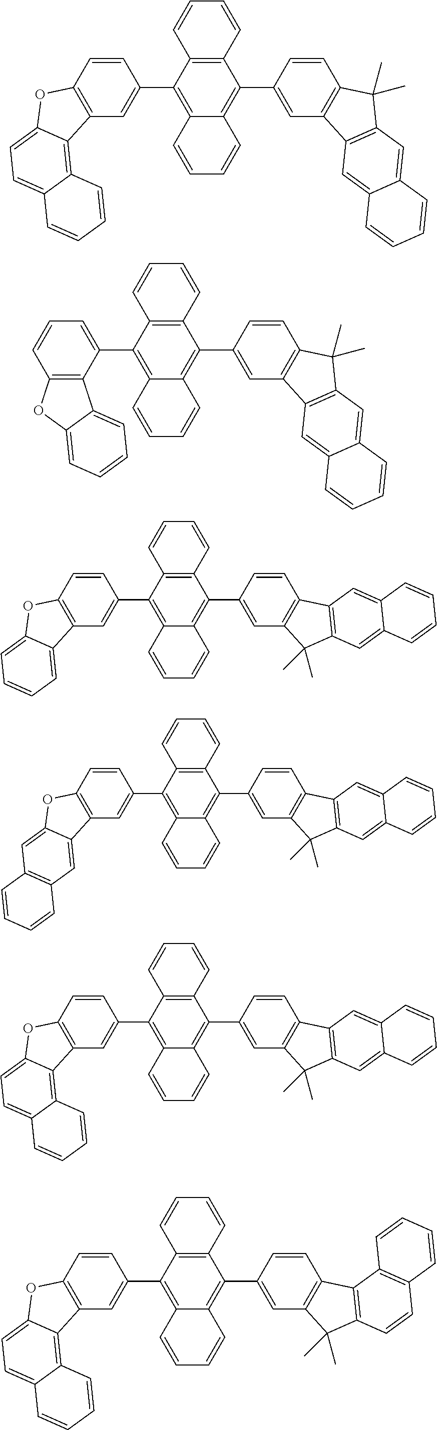

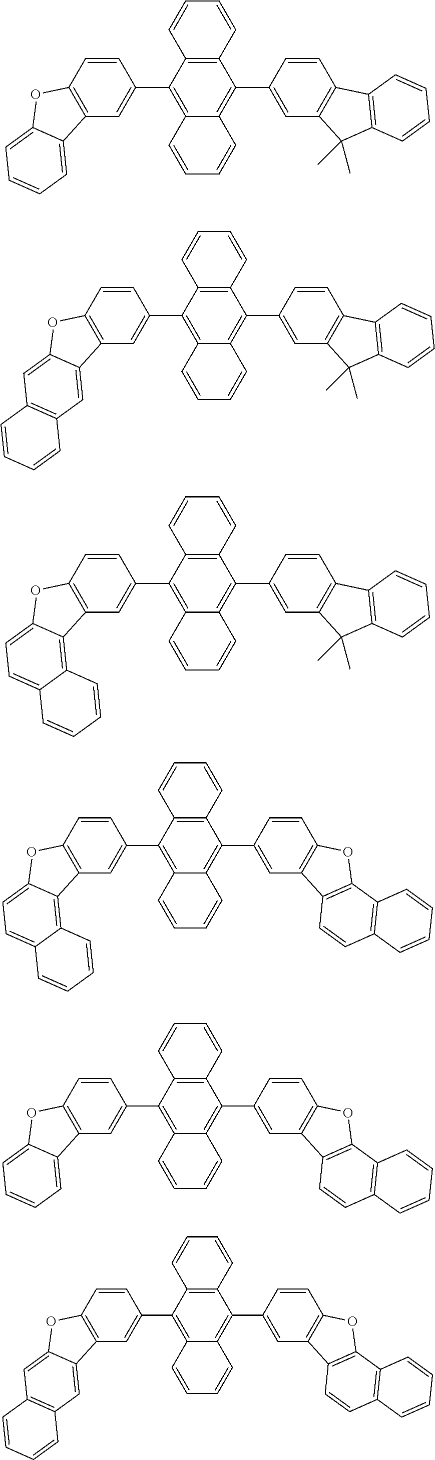 [SMILES: CC1(C)c2ccccc2-c2ccc(-c3c4ccccc4c(-c4ccc5oc6ccccc6c5c4)c4ccccc34)cc21 ; CC1(C)c2ccccc2-c2ccc(-c3c4ccccc4c(-c4ccc5oc6cc7ccccc7cc6c5c4)c4ccccc34)cc21 ; CC1(C)c2ccccc2-c2ccc(-c3c4ccccc4c(-c4ccc5oc6ccc7ccccc7c6c5c4)c4ccccc34)cc21 ; c1ccc2c(c1)ccc1c3cc(-c4c5ccccc5c(-c5ccc6oc7ccc8ccccc8c7c6c5)c5ccccc45)ccc3oc21 ; c1ccc2c(c1)ccc1c3cc(-c4c5ccccc5c(-c5ccc6oc7ccccc7c6c5)c5ccccc45)ccc3oc21 ; c1ccc2cc3c(cc2c1)oc1ccc(-c2c4ccccc4c(-c4ccc5oc6c7ccccc7ccc6c5c4)c4ccccc24)cc13]

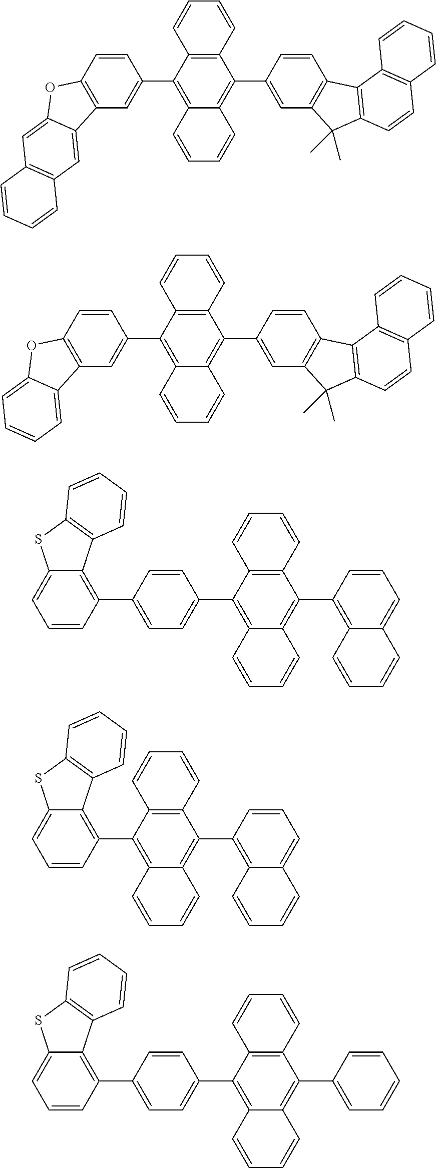 [SMILES: CC1(C)c2cc(-c3c4ccccc4c(-c4ccc5oc6cc7ccccc7cc6c5c4)c4ccccc34)ccc2-c2c1ccc1ccccc21 ; CC1(C)c2cc(-c3c4ccccc4c(-c4ccc5oc6ccccc6c5c4)c4ccccc34)ccc2-c2c1ccc1ccccc21 ; c1ccc2c(-c3c4ccccc4c(-c4ccc(-c5cccc6sc7ccccc7c56)cc4)c4ccccc34)cccc2c1 ; c1ccc2c(-c3c4ccccc4c(-c4cccc5sc6ccccc6c45)c4ccccc34)cccc2c1 ; c1ccc(-c2c3ccccc3c(-c3ccc(-c4cccc5sc6ccccc6c45)cc3)c3ccccc23)cc1]

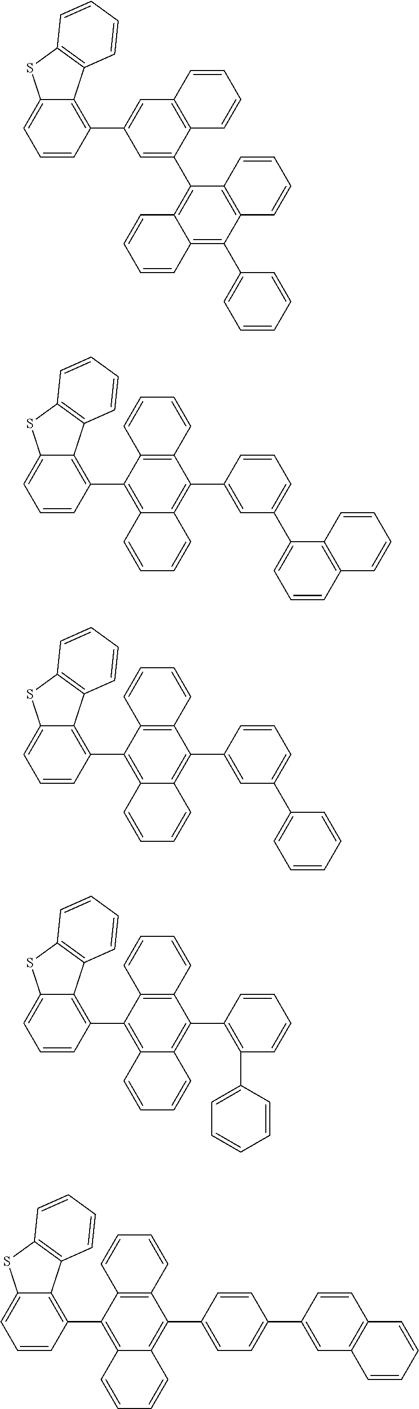 [SMILES: c1ccc(-c2c3ccccc3c(-c3cc(-c4cccc5sc6ccccc6c45)cc4ccccc34)c3ccccc23)cc1 ; c1cc(-c2cccc3ccccc23)cc(-c2c3ccccc3c(-c3cccc4sc5ccccc5c34)c3ccccc23)c1 ; c1ccc(-c2cccc(-c3c4ccccc4c(-c4cccc5sc6ccccc6c45)c4ccccc34)c2)cc1 ; c1ccc(-c2ccccc2-c2c3ccccc3c(-c3cccc4sc5ccccc5c34)c3ccccc23)cc1 ; c1ccc2cc(-c3ccc(-c4c5ccccc5c(-c5cccc6sc7ccccc7c56)c5ccccc45)cc3)ccc2c1]

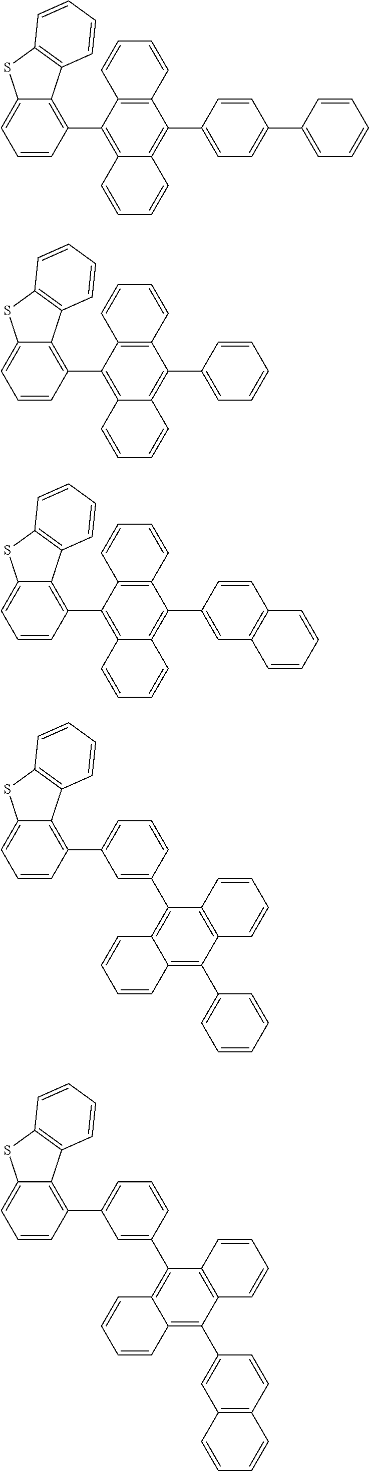 [SMILES: c1ccc(-c2ccc(-c3c4ccccc4c(-c4cccc5sc6ccccc6c45)c4ccccc34)cc2)cc1 ; c1ccc(-c2c3ccccc3c(-c3cccc4sc5ccccc5c34)c3ccccc23)cc1 ; c1ccc2cc(-c3c4ccccc4c(-c4cccc5sc6ccccc6c45)c4ccccc34)ccc2c1 ; c1ccc(-c2c3ccccc3c(-c3cccc(-c4cccc5sc6ccccc6c45)c3)c3ccccc23)cc1 ; c1cc(-c2c3ccccc3c(-c3ccc4ccccc4c3)c3ccccc23)cc(-c2cccc3sc4ccccc4c23)c1]

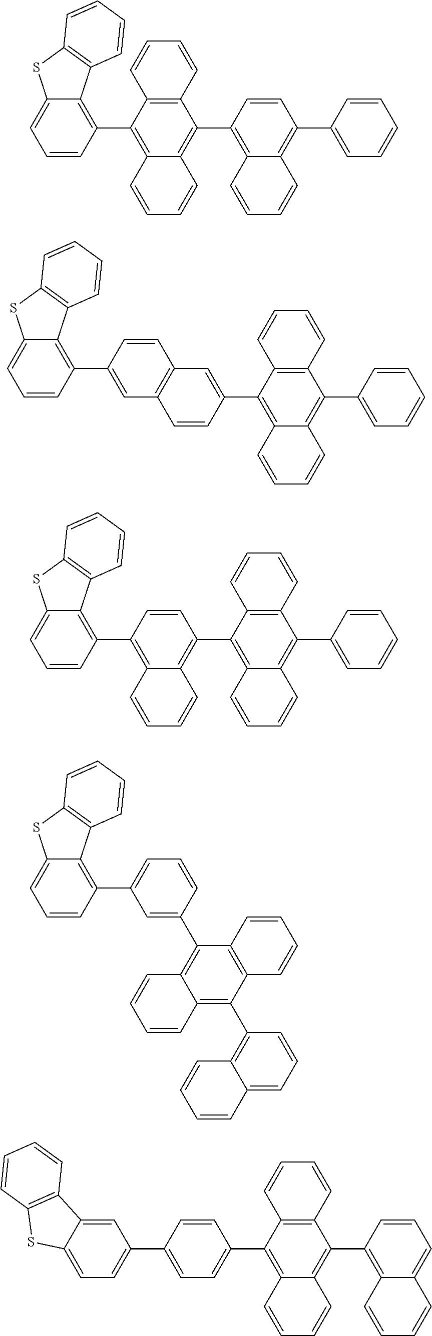 [SMILES: c1ccc(-c2ccc(-c3c4ccccc4c(-c4cccc5sc6ccccc6c45)c4ccccc34)c3ccccc23)cc1 ; c1ccc(-c2c3ccccc3c(-c3ccc4cc(-c5cccc6sc7ccccc7c56)ccc4c3)c3ccccc23)cc1 ; c1ccc(-c2c3ccccc3c(-c3ccc(-c4cccc5sc6ccccc6c45)c4ccccc34)c3ccccc23)cc1 ; c1cc(-c2c3ccccc3c(-c3cccc4ccccc34)c3ccccc23)cc(-c2cccc3sc4ccccc4c23)c1 ; c1ccc2c(-c3c4ccccc4c(-c4ccc(-c5ccc6sc7ccccc7c6c5)cc4)c4ccccc34)cccc2c1]

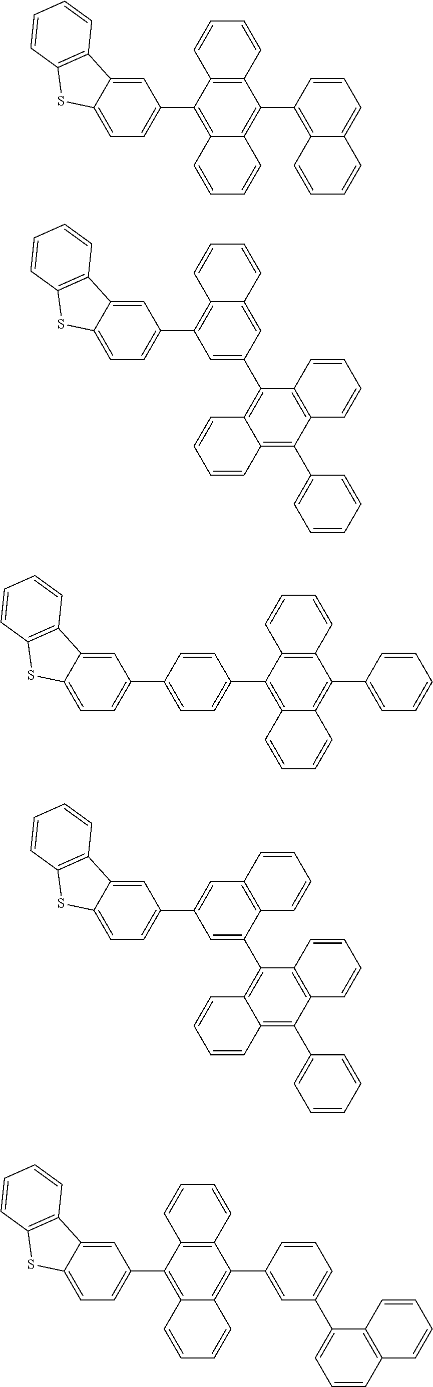 [SMILES: c1ccc2c(-c3c4ccccc4c(-c4ccc5sc6ccccc6c5c4)c4ccccc34)cccc2c1 ; c1ccc(-c2c3ccccc3c(-c3cc(-c4ccc5sc6ccccc6c5c4)c4ccccc4c3)c3ccccc23)cc1 ; c1ccc(-c2c3ccccc3c(-c3ccc(-c4ccc5sc6ccccc6c5c4)cc3)c3ccccc23)cc1 ; c1ccc(-c2c3ccccc3c(-c3cc(-c4ccc5sc6ccccc6c5c4)cc4ccccc34)c3ccccc23)cc1 ; c1cc(-c2cccc3ccccc23)cc(-c2c3ccccc3c(-c3ccc4sc5ccccc5c4c3)c3ccccc23)c1]

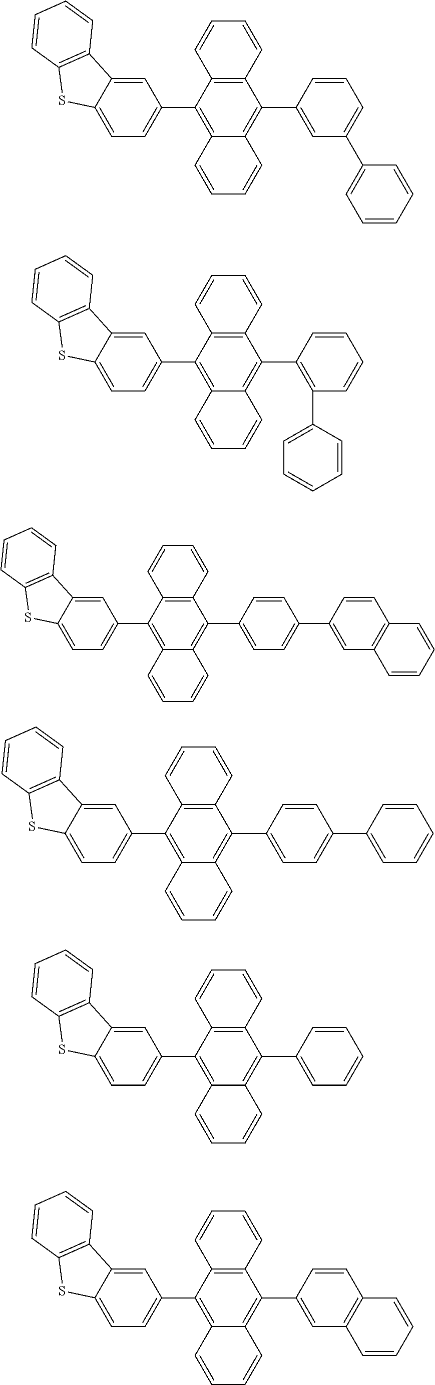

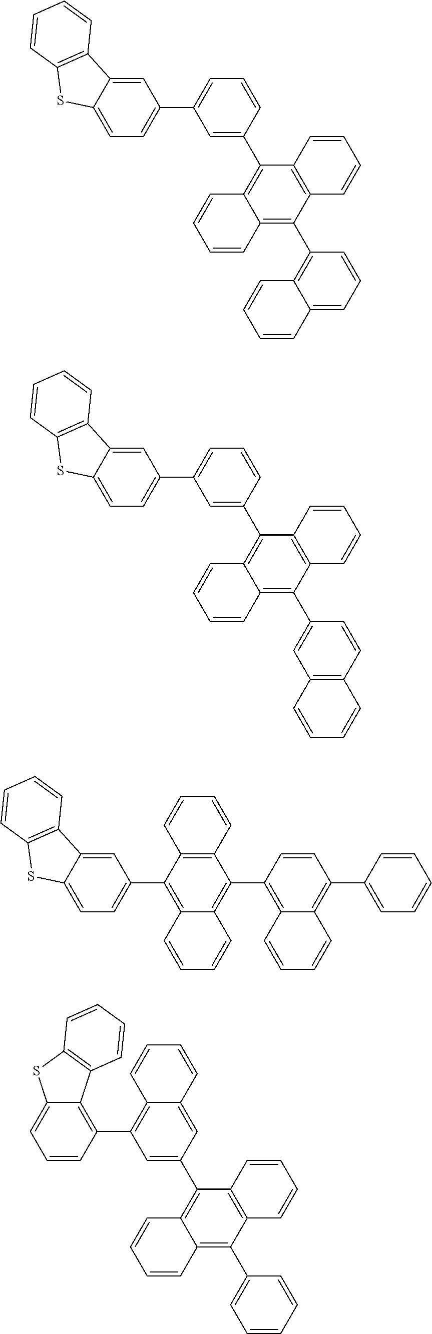 [SMILES: c1cc(-c2ccc3sc4ccccc4c3c2)cc(-c2c3ccccc3c(-c3cccc4ccccc34)c3ccccc23)c1 ; c1cc(-c2ccc3sc4ccccc4c3c2)cc(-c2c3ccccc3c(-c3ccc4ccccc4c3)c3ccccc23)c1 ; c1ccc(-c2ccc(-c3c4ccccc4c(-c4ccc5sc6ccccc6c5c4)c4ccccc34)c3ccccc23)cc1 ; c1ccc(-c2c3ccccc3c(-c3cc(-c4cccc5sc6ccccc6c45)c4ccccc4c3)c3ccccc23)cc1]

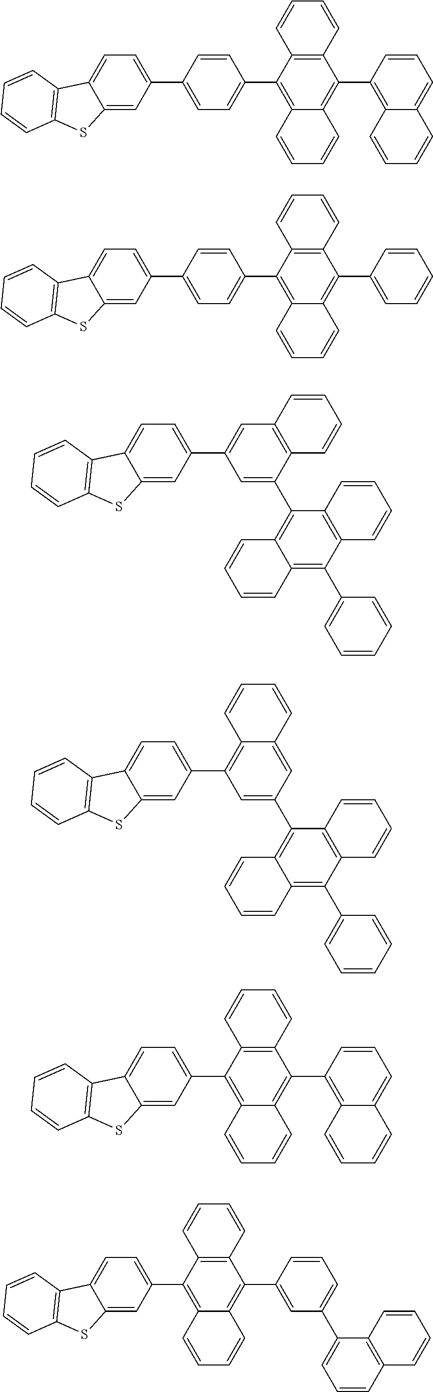

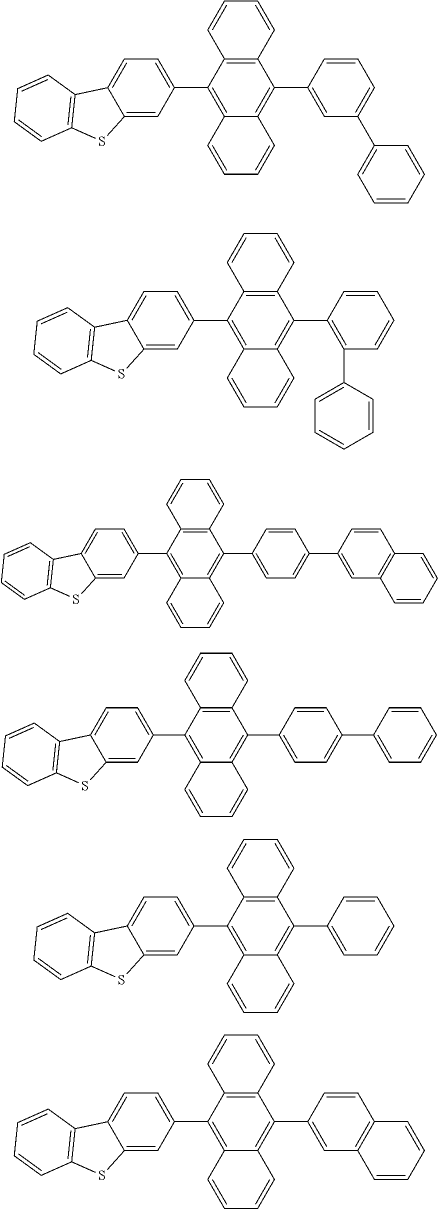

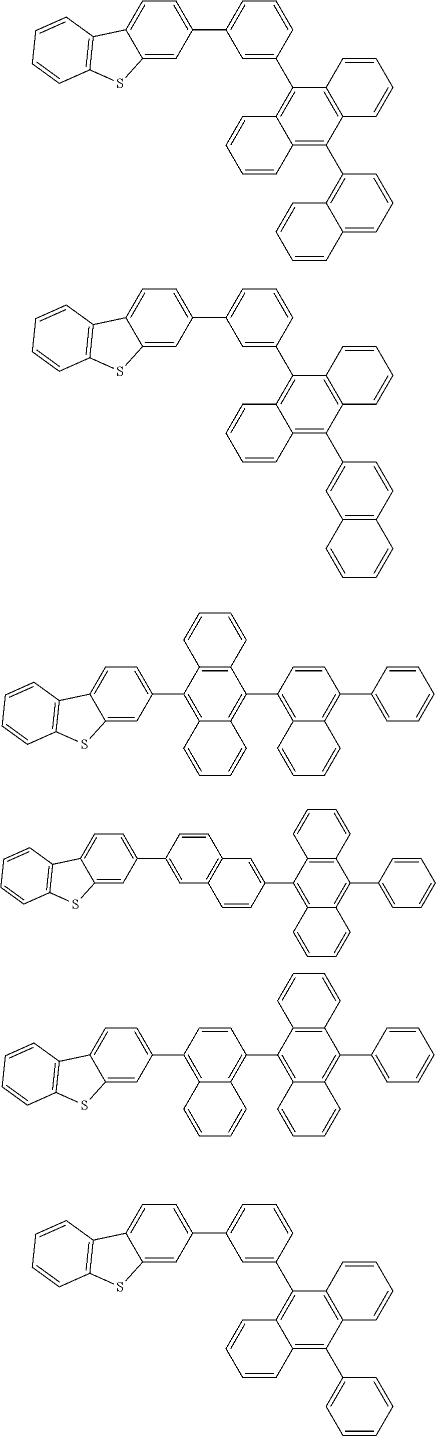

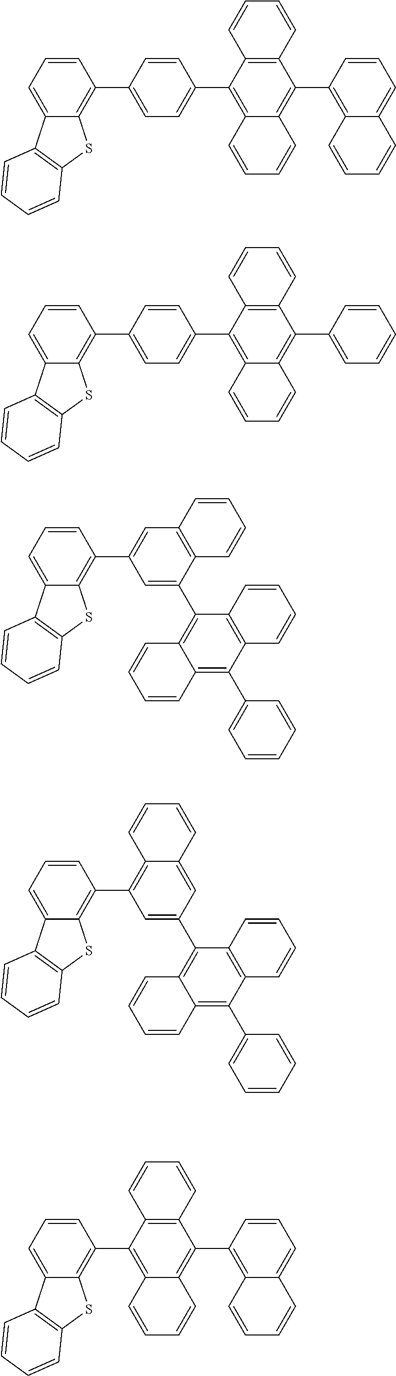 [SMILES: c1ccc2c(-c3c4ccccc4c(-c4ccc(-c5cccc6c5sc5ccccc56)cc4)c4ccccc34)cccc2c1 ; c1ccc(-c2c3ccccc3c(-c3ccc(-c4cccc5c4sc4ccccc45)cc3)c3ccccc23)cc1 ; c1ccc(-c2c3ccccc3c(-c3cc(-c4cccc5c4sc4ccccc45)cc4ccccc34)c3ccccc23)cc1 ; c1ccc(-c2c3ccccc3c(-c3cc(-c4cccc5c4sc4ccccc45)c4ccccc4c3)c3ccccc23)cc1 ; c1ccc2c(-c3c4ccccc4c(-c4cccc5c4sc4ccccc45)c4ccccc34)cccc2c1]

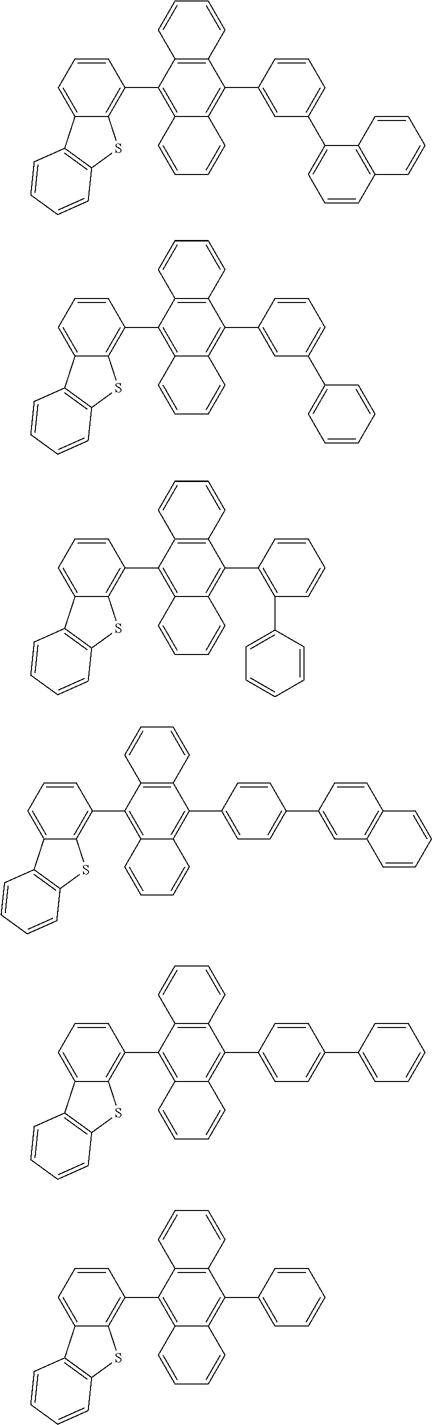 [SMILES: c1cc(-c2cccc3ccccc23)cc(-c2c3ccccc3c(-c3cccc4c3sc3ccccc34)c3ccccc23)c1 ; c1ccc(-c2cccc(-c3c4ccccc4c(-c4cccc5c4sc4ccccc45)c4ccccc34)c2)cc1 ; c1ccc(-c2ccccc2-c2c3ccccc3c(-c3cccc4c3sc3ccccc34)c3ccccc23)cc1 ; c1ccc2cc(-c3ccc(-c4c5ccccc5c(-c5cccc6c5sc5ccccc56)c5ccccc45)cc3)ccc2c1 ; c1ccc(-c2ccc(-c3c4ccccc4c(-c4cccc5c4sc4ccccc45)c4ccccc34)cc2)cc1 ; c1ccc(-c2c3ccccc3c(-c3cccc4c3sc3ccccc34)c3ccccc23)cc1]

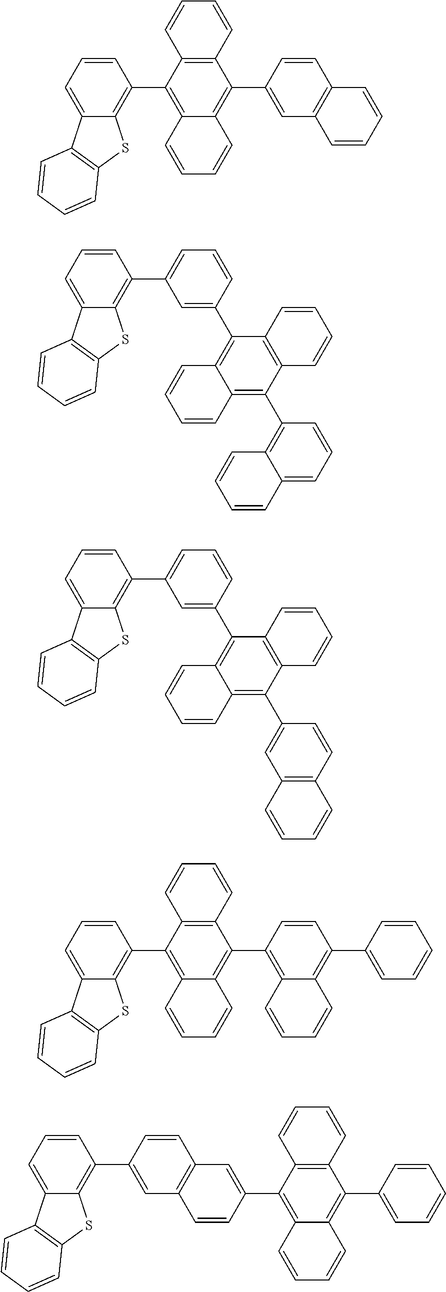

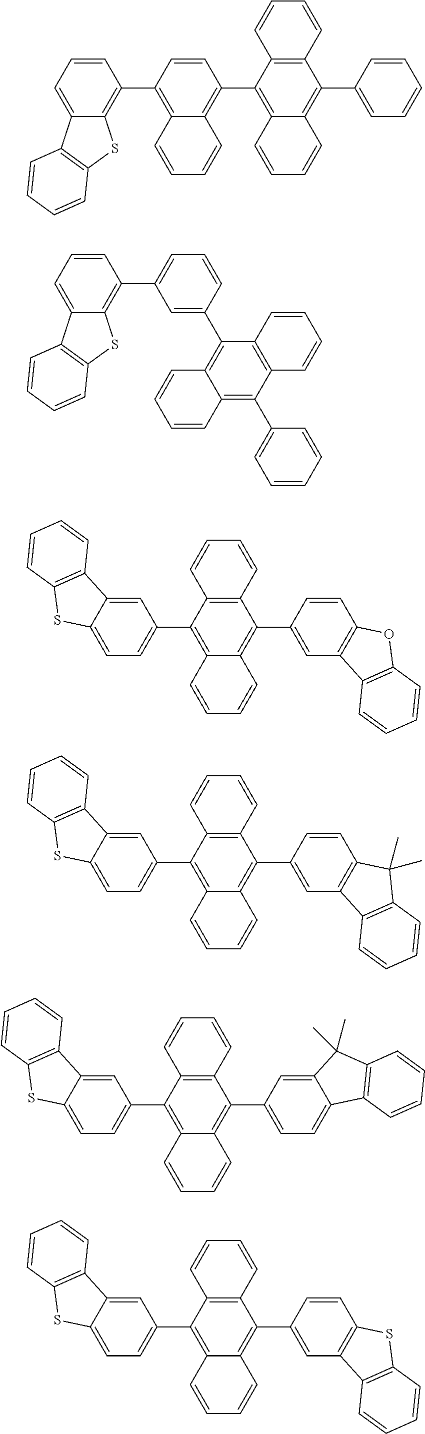

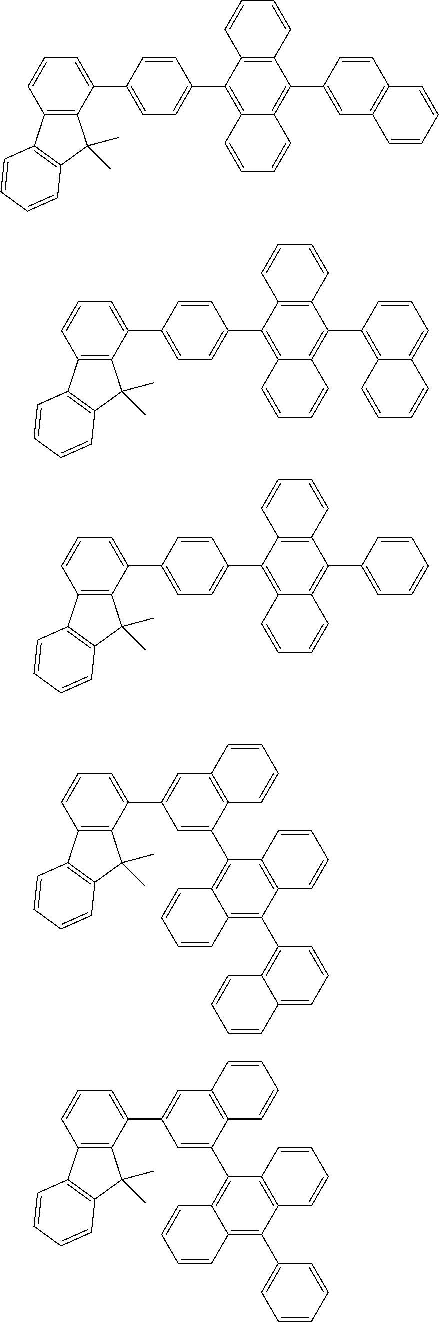

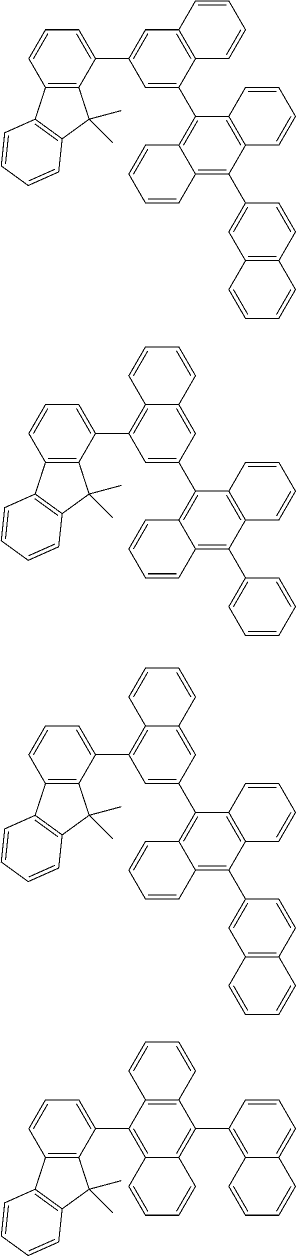

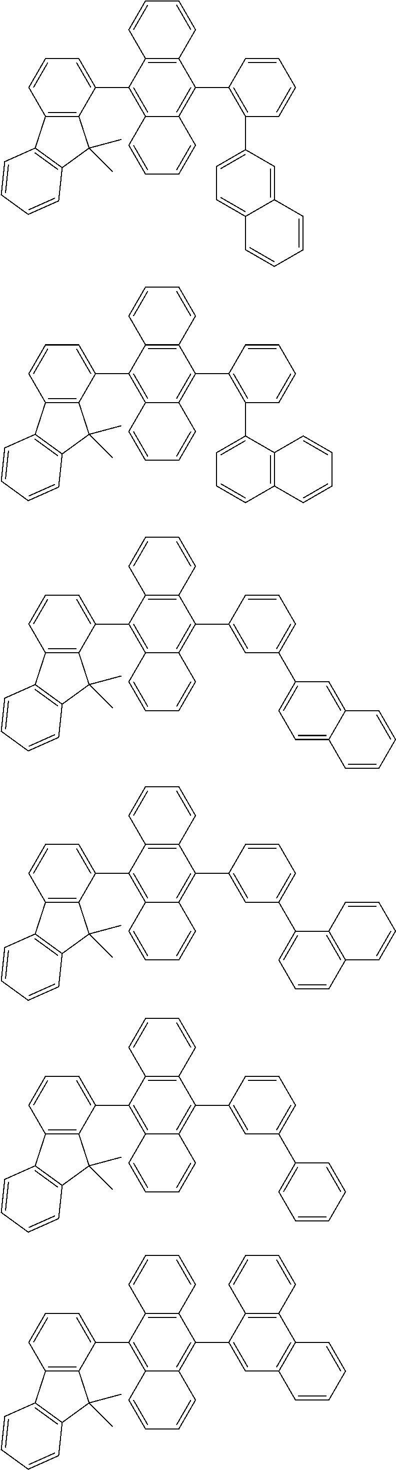

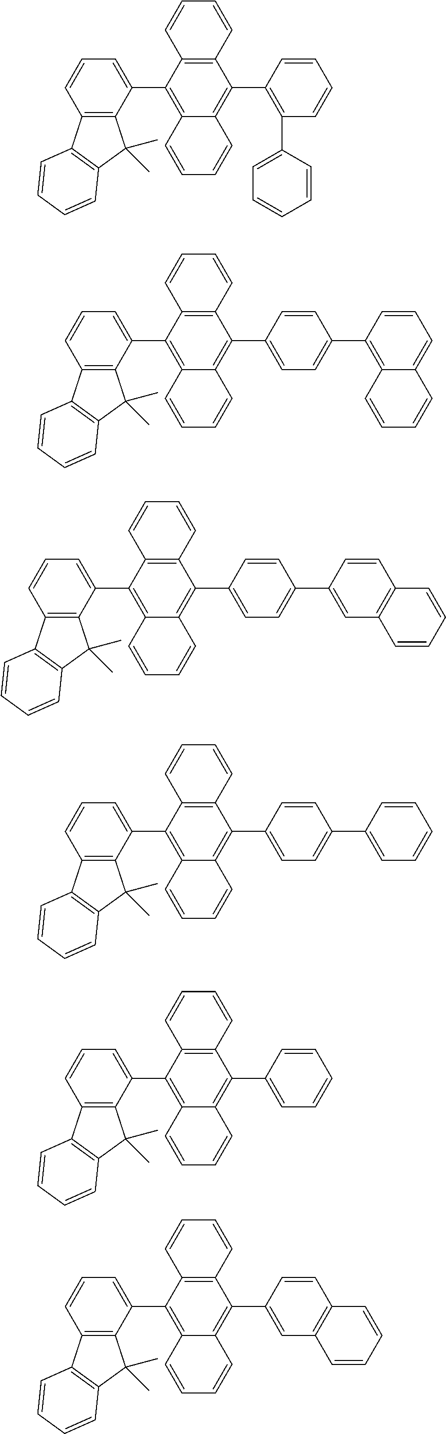

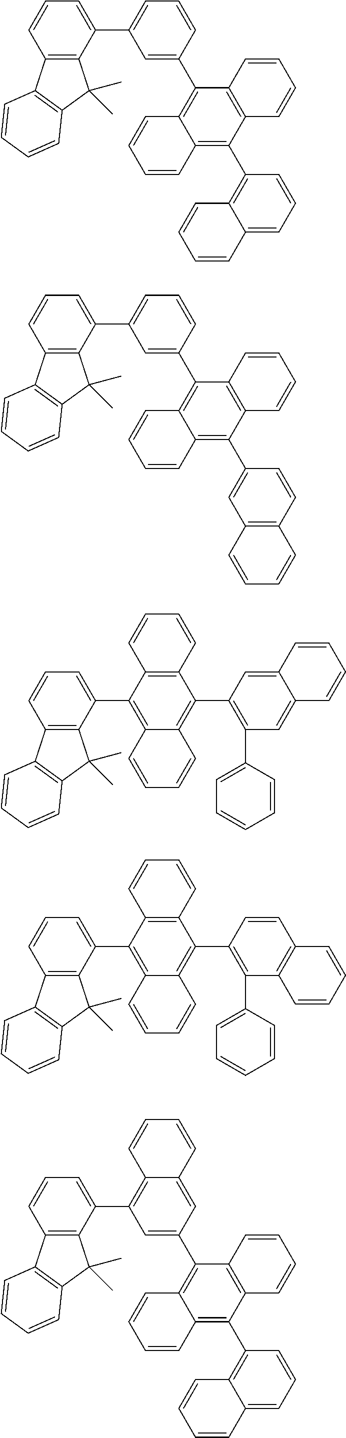

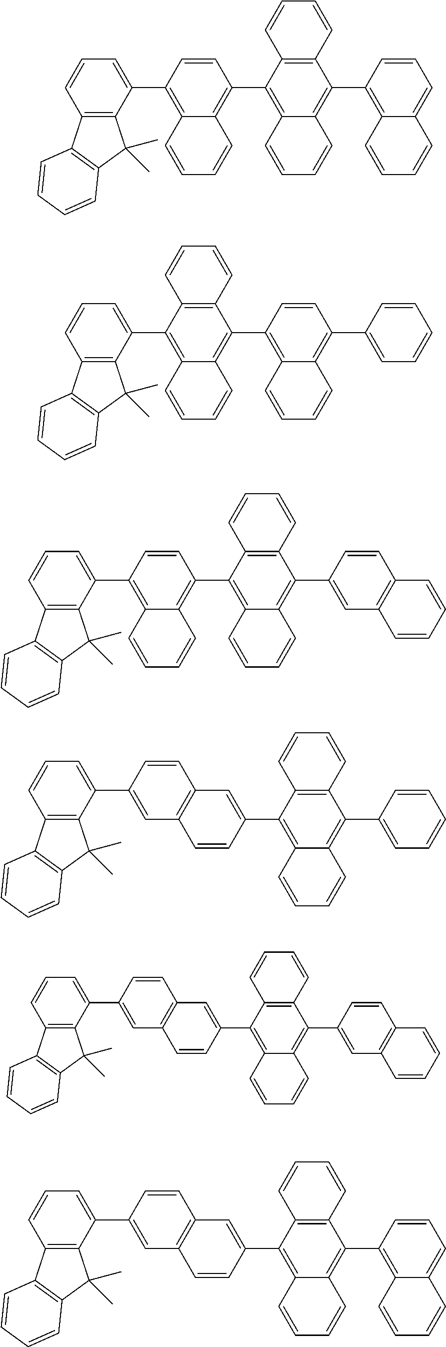

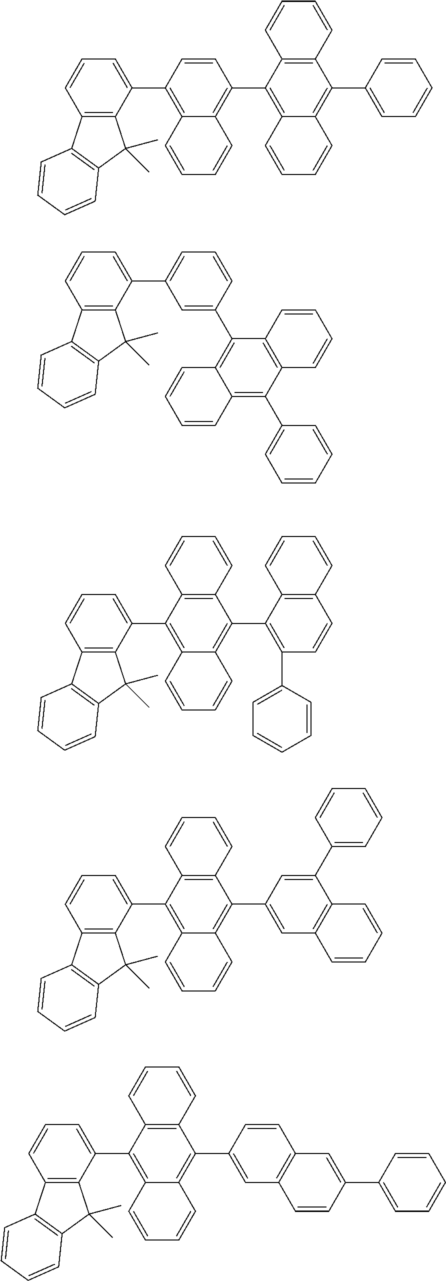

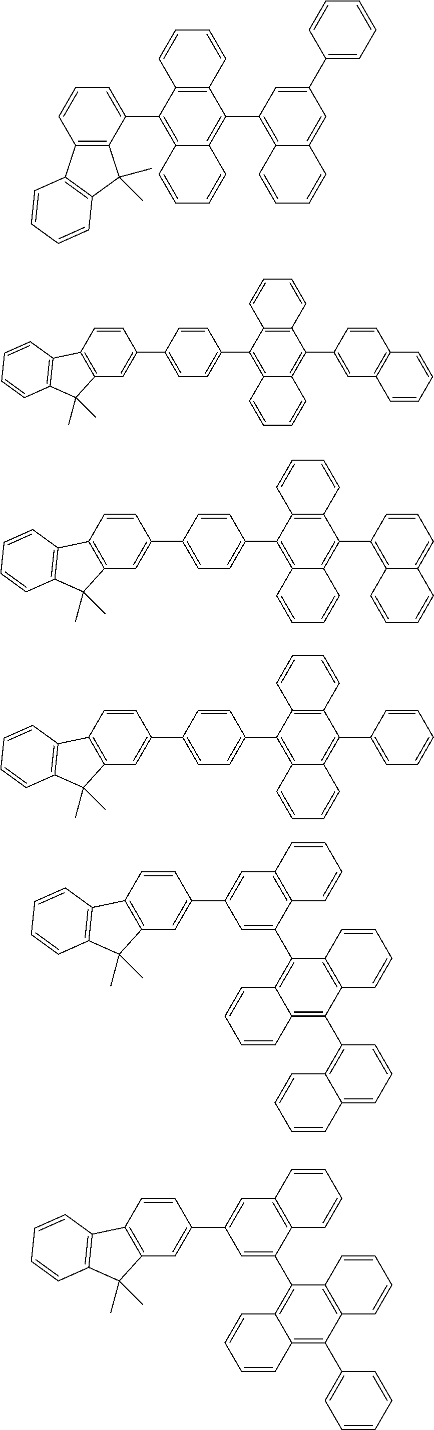

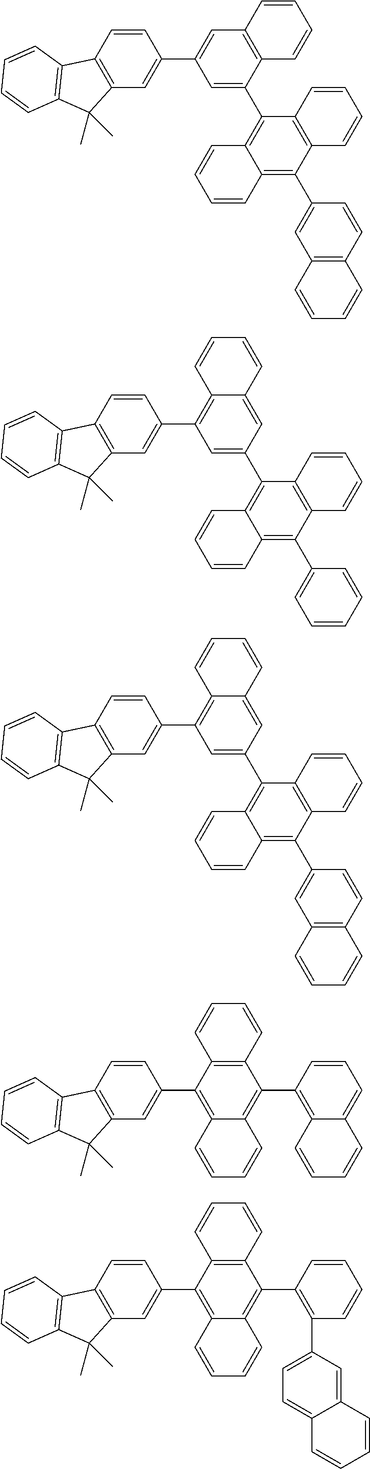

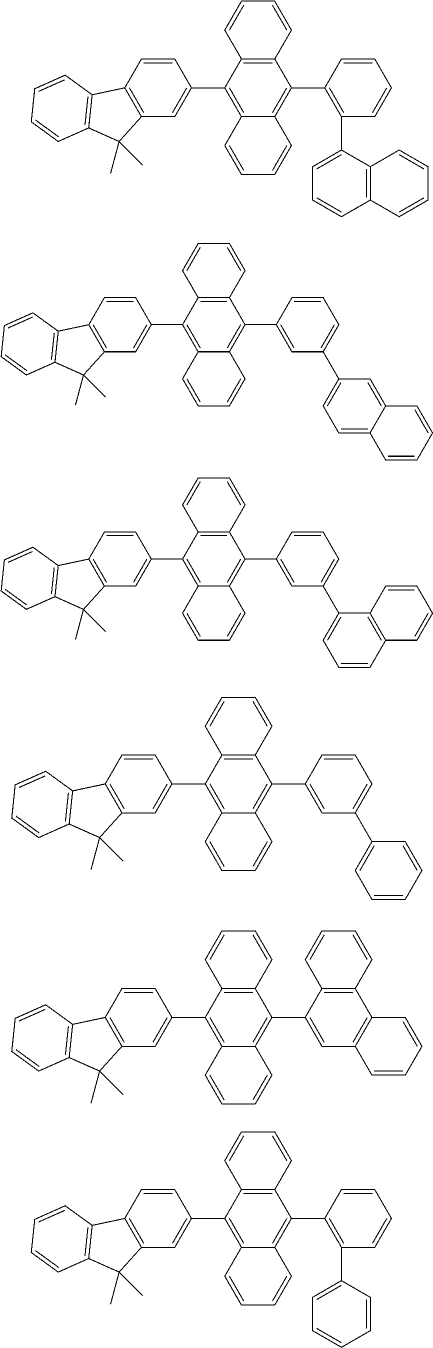

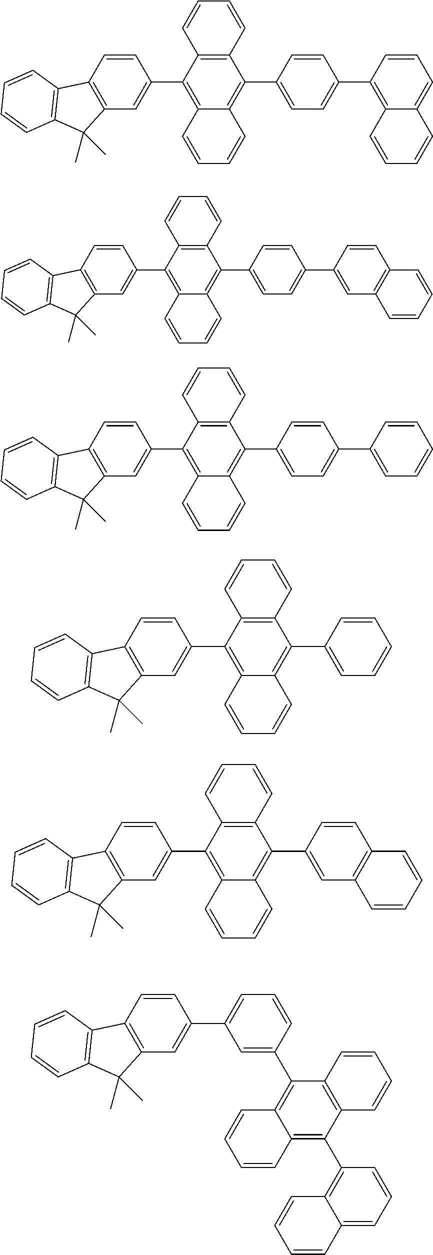

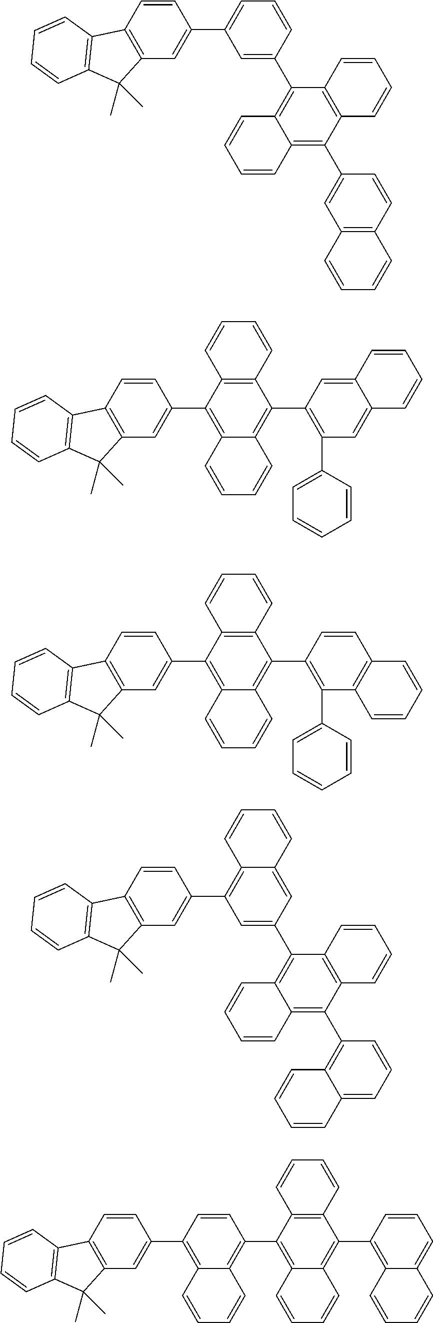

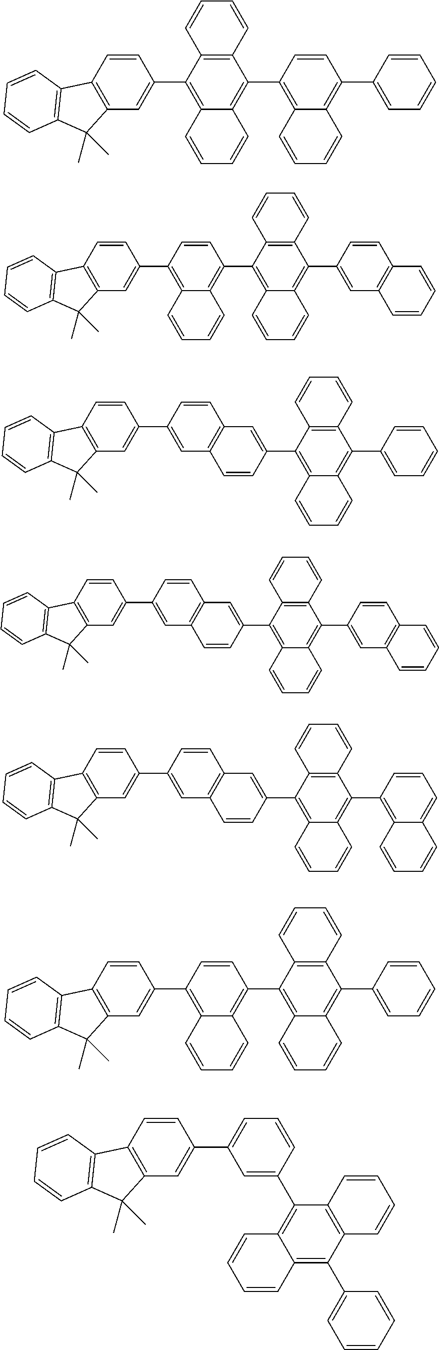

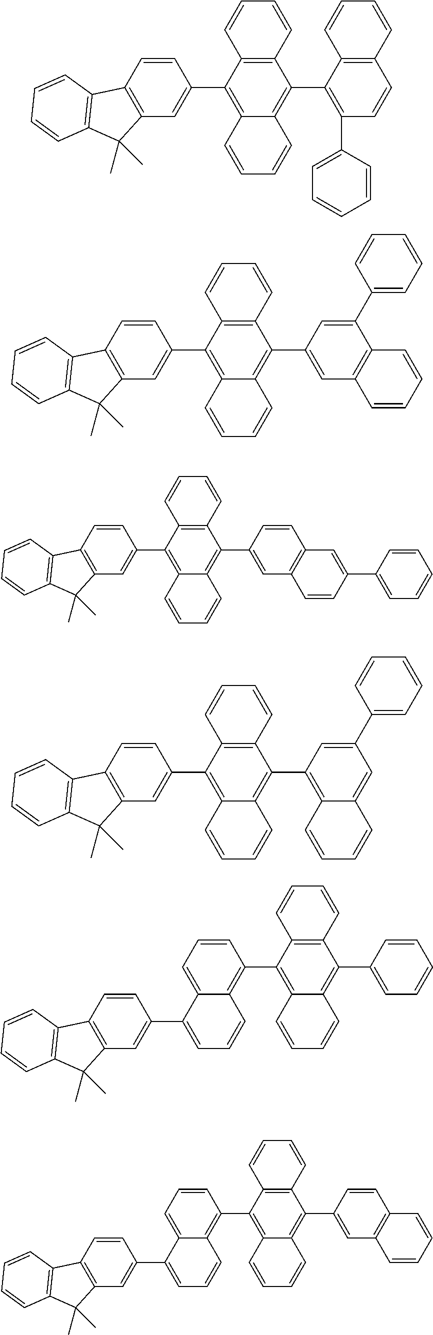

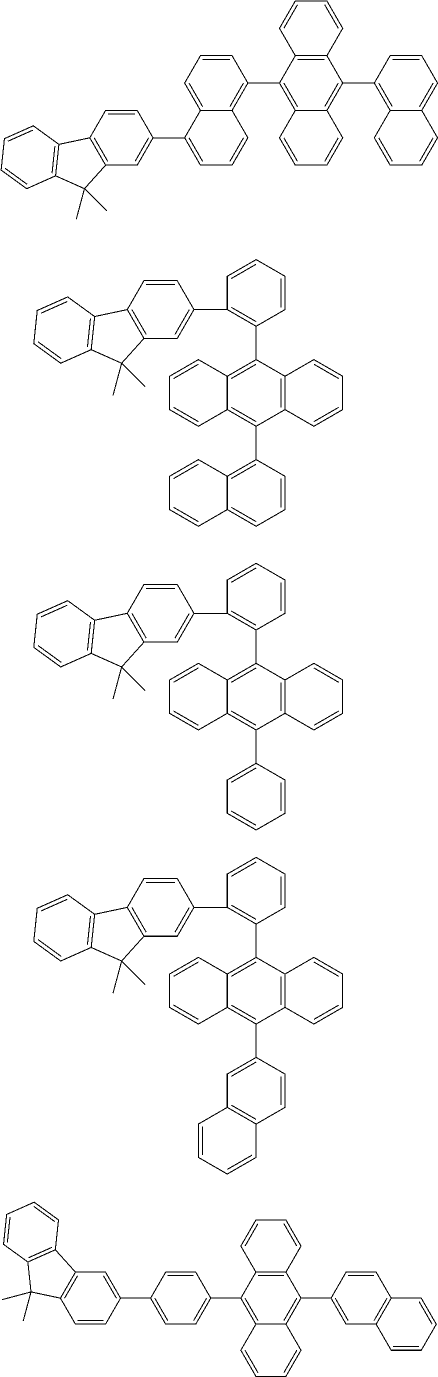

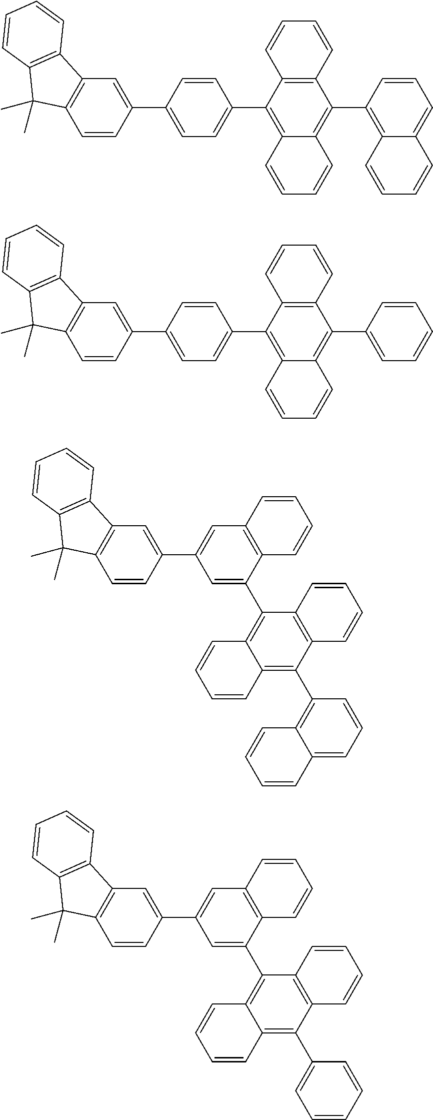

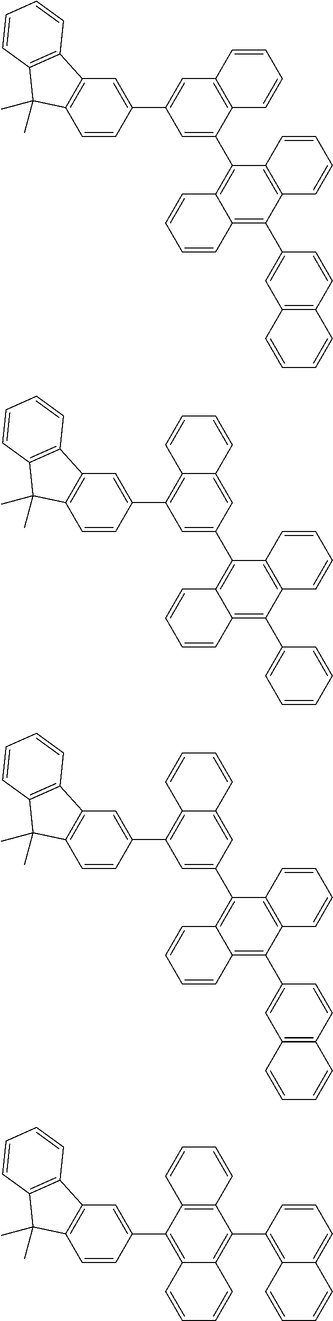

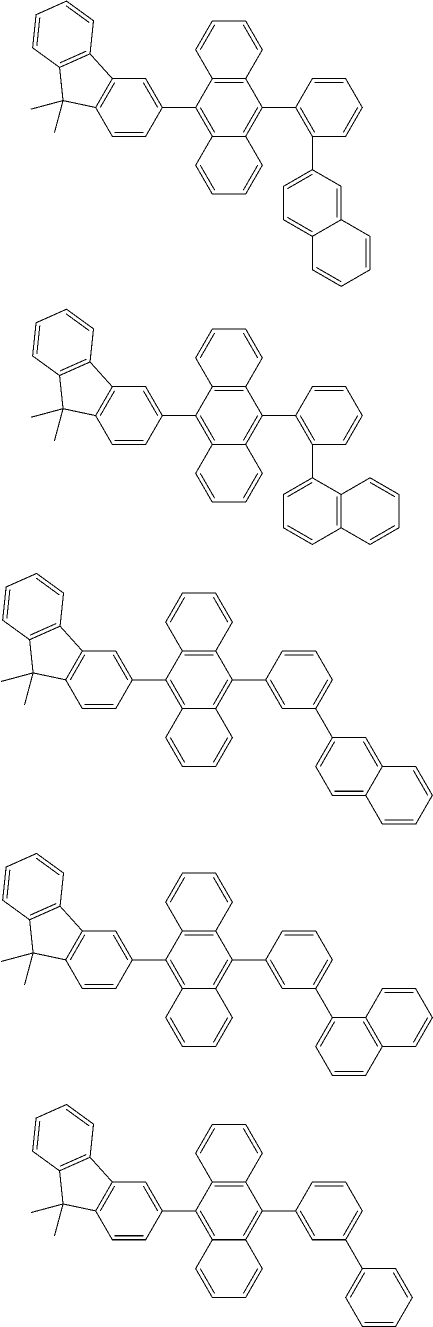

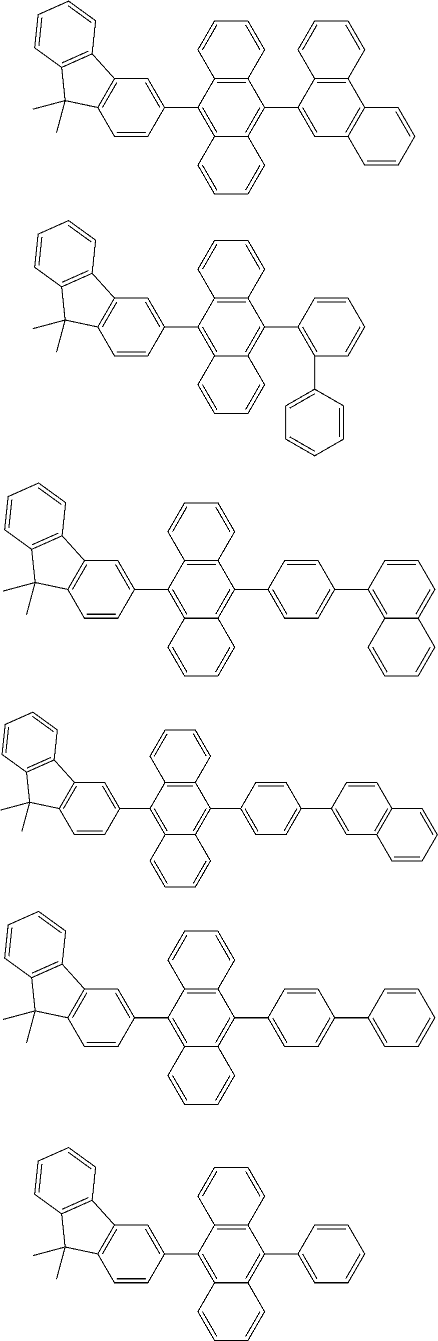

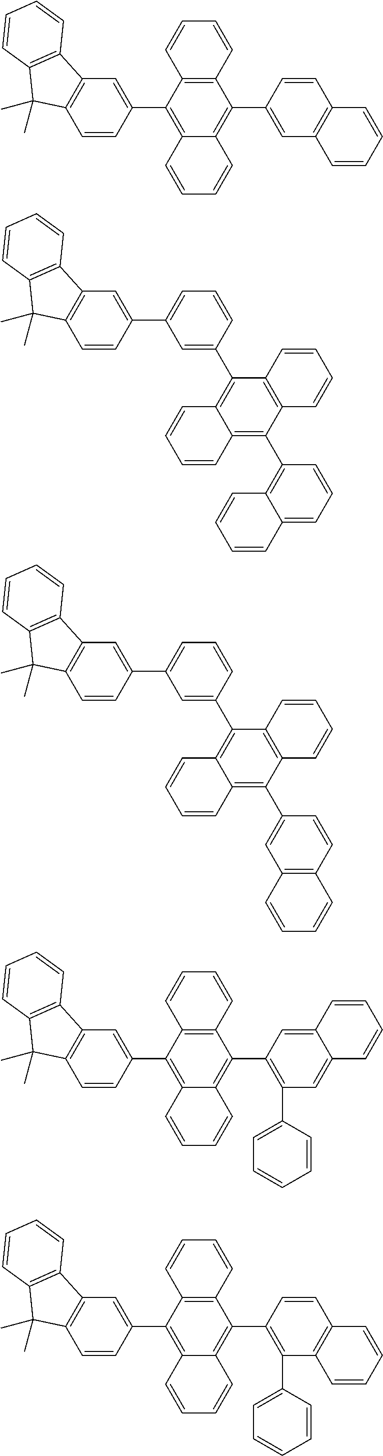

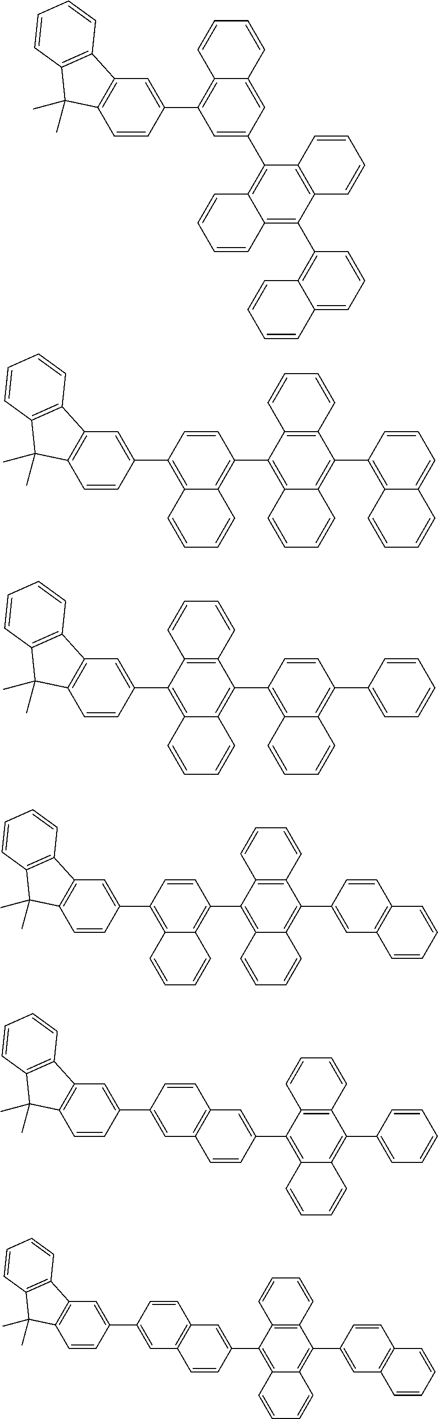

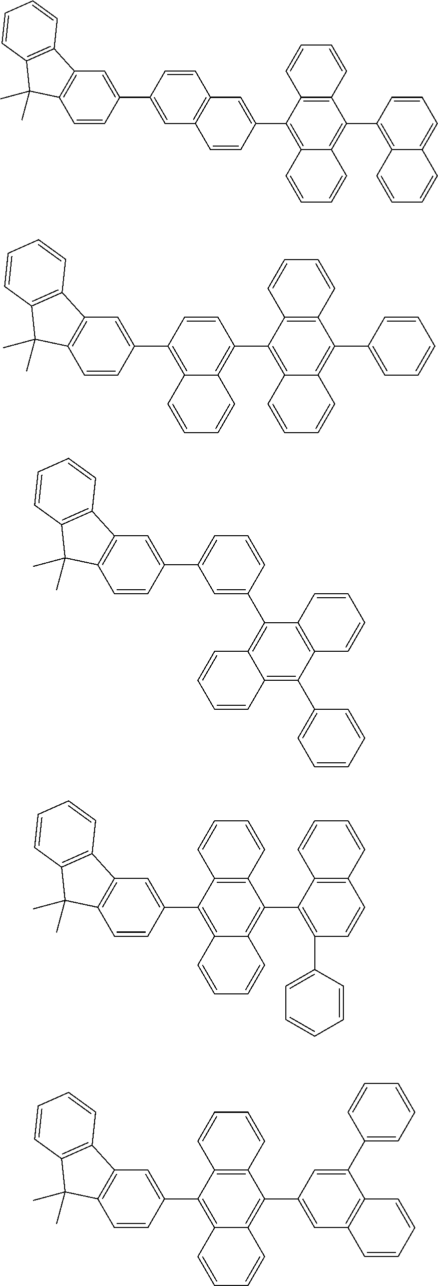

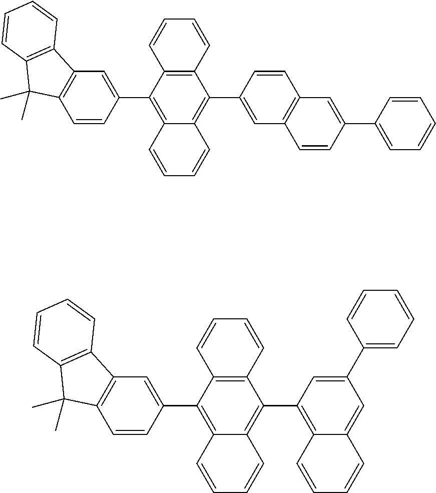

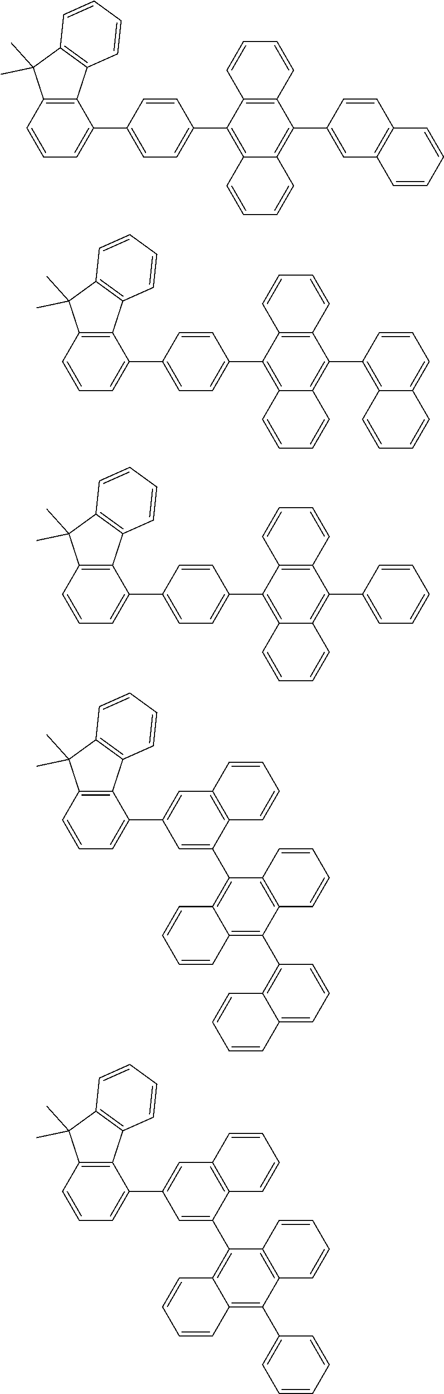

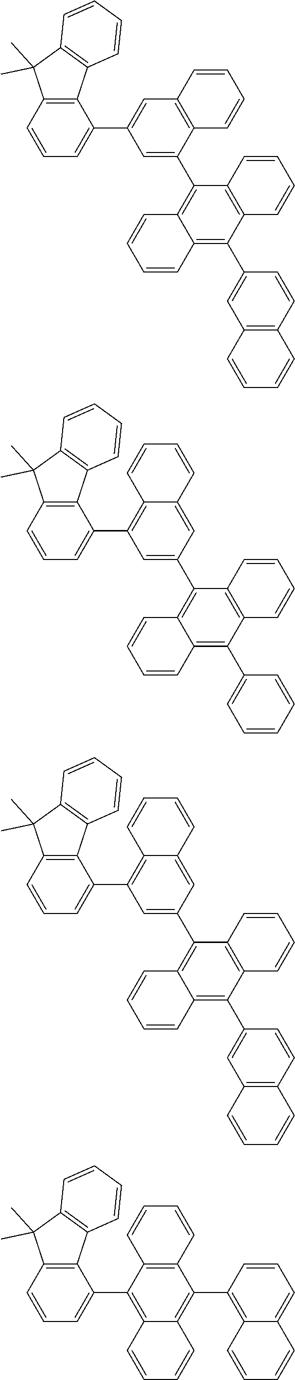

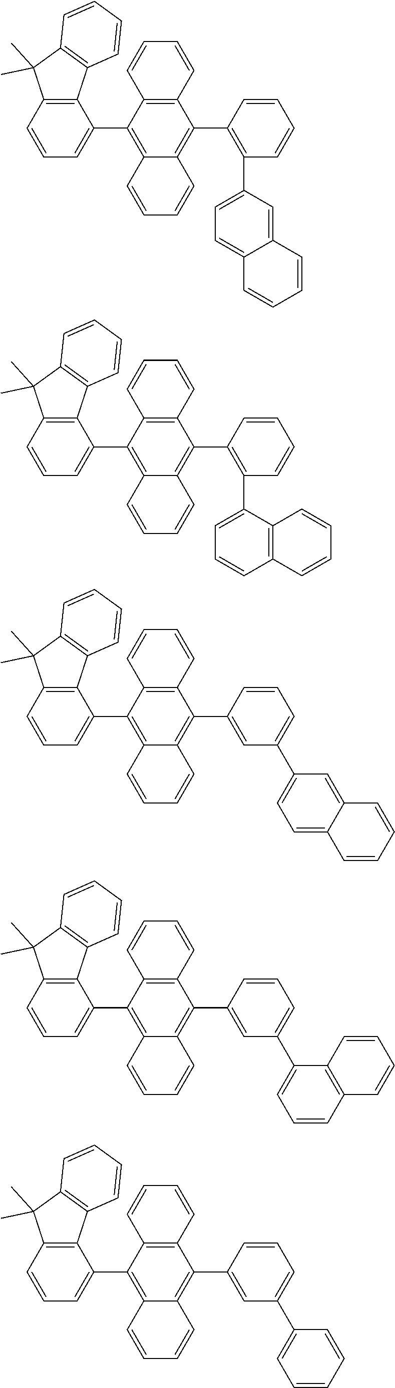

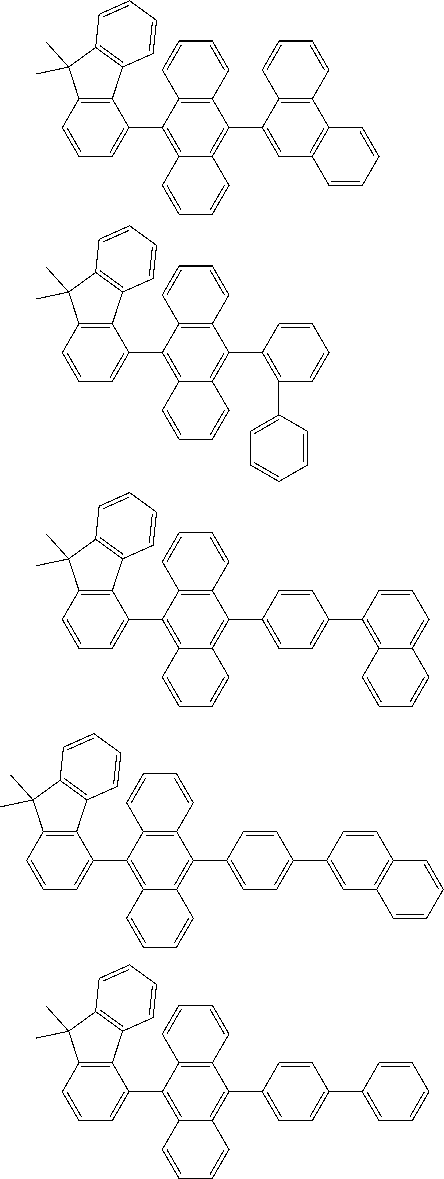

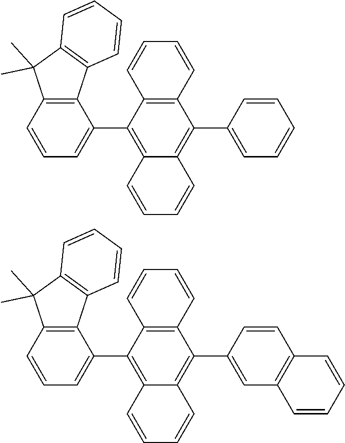

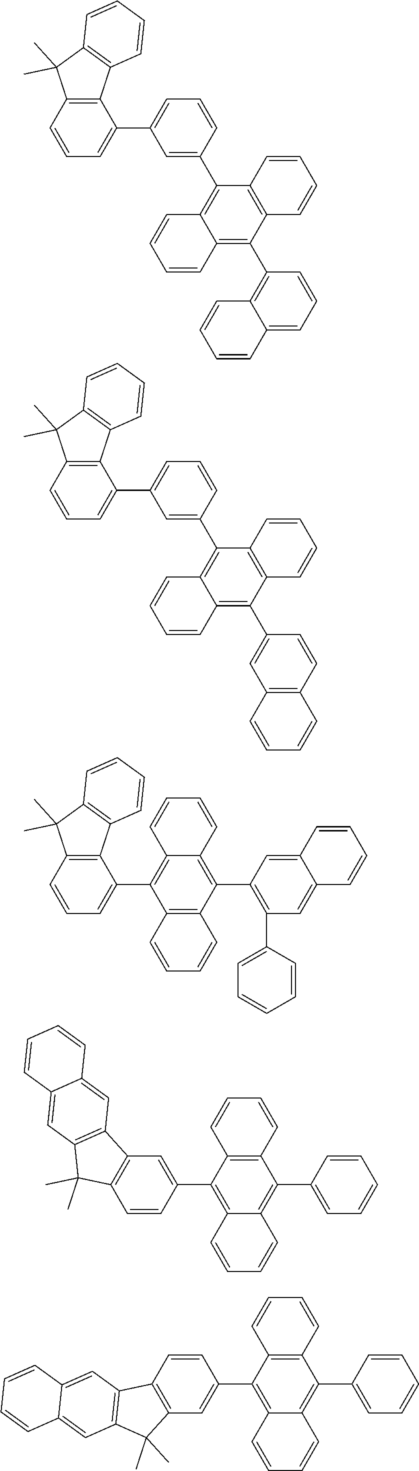

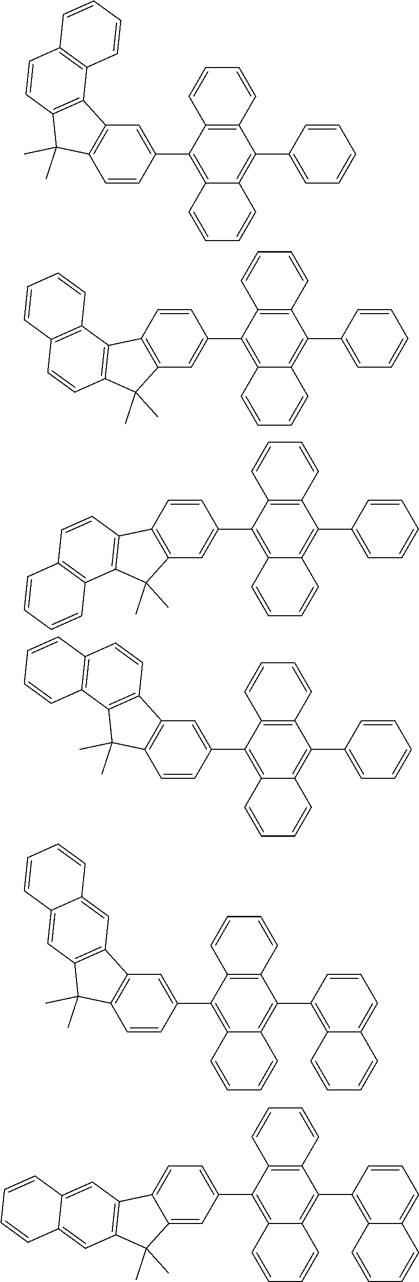

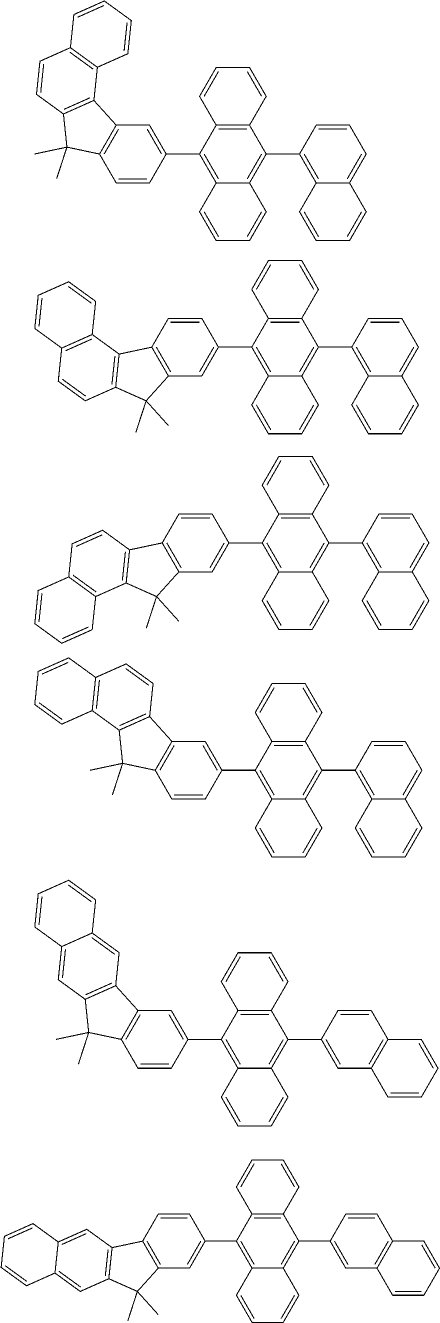

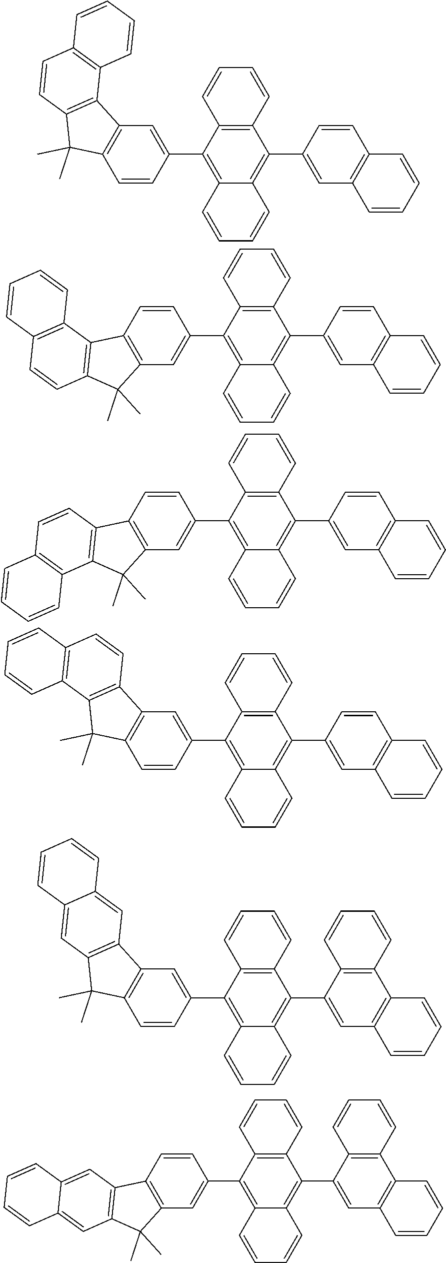

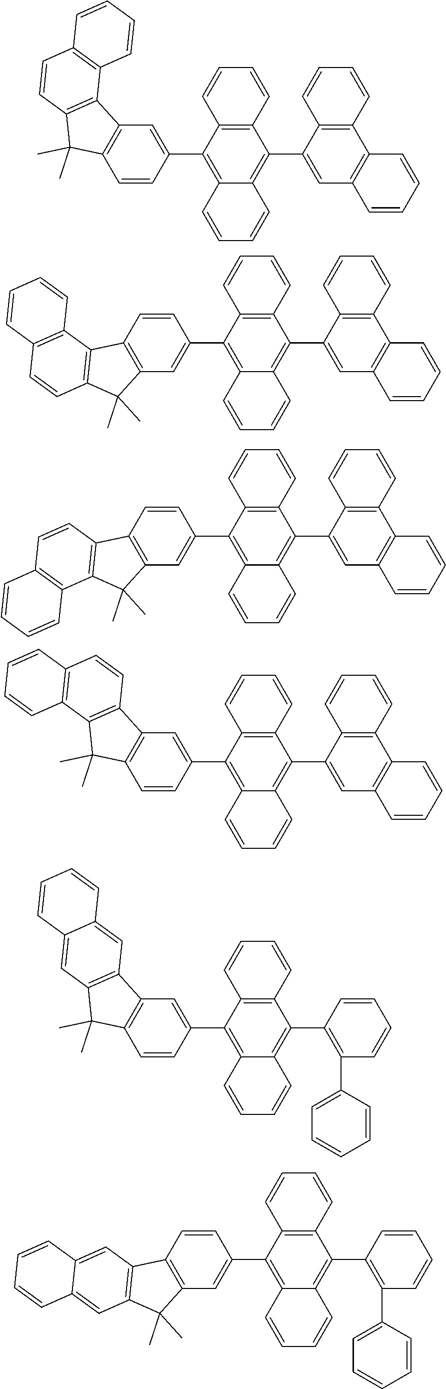

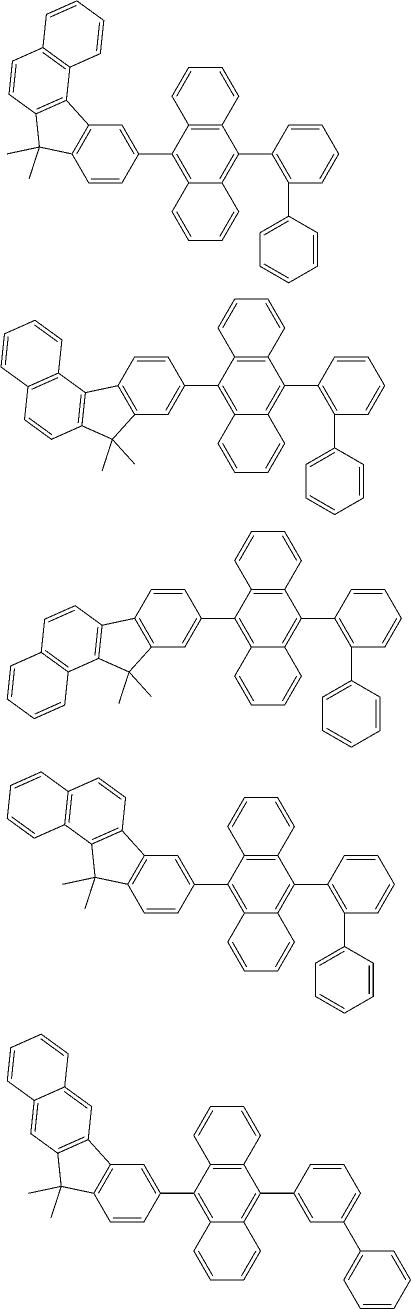

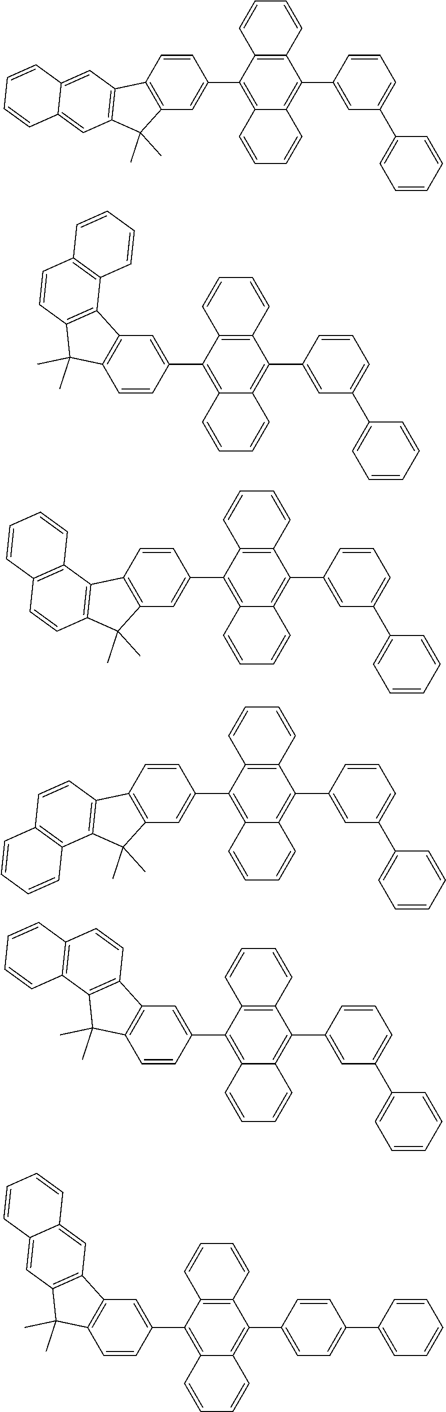

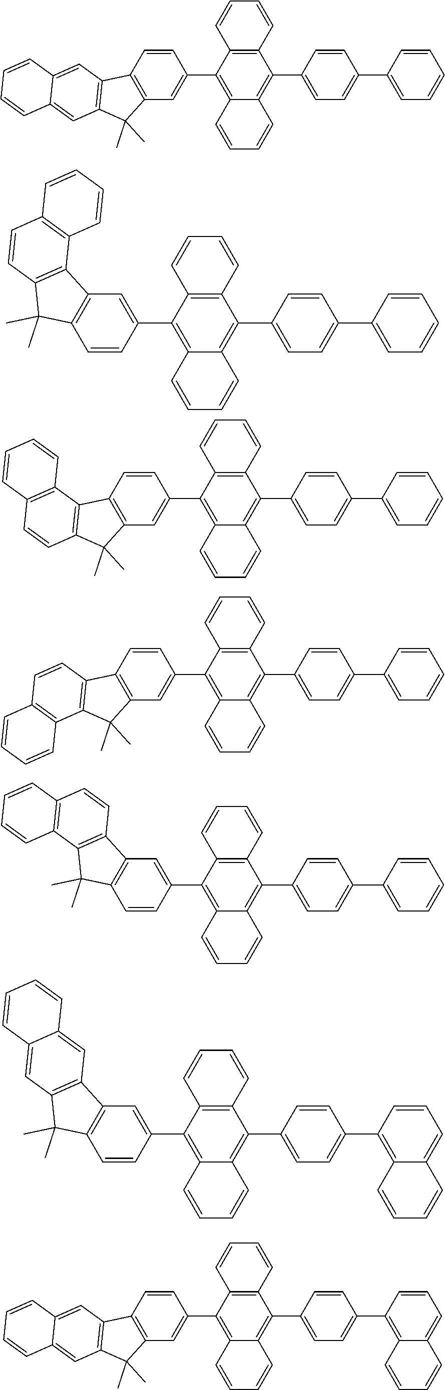

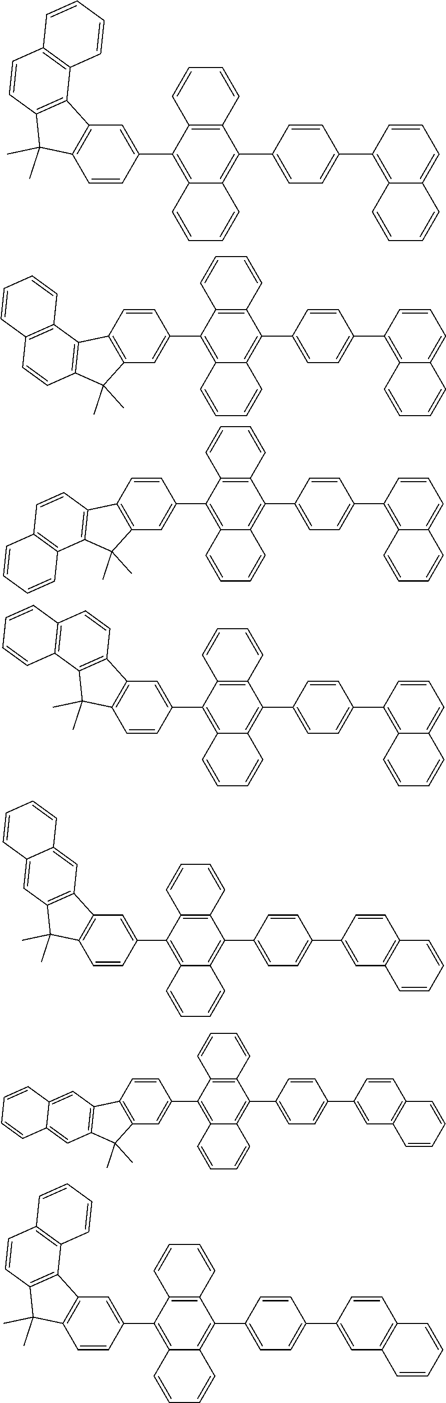

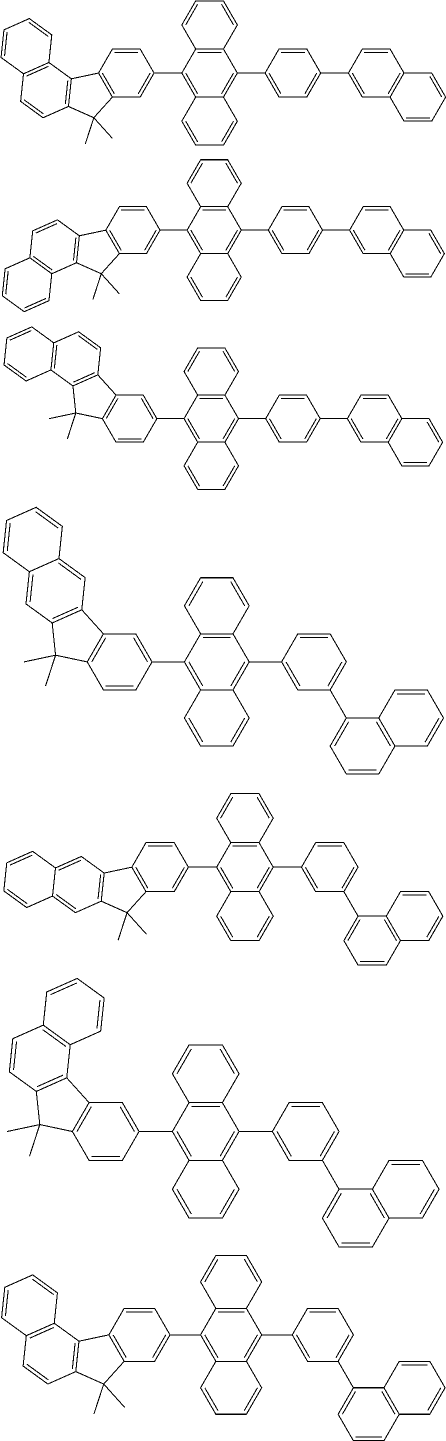

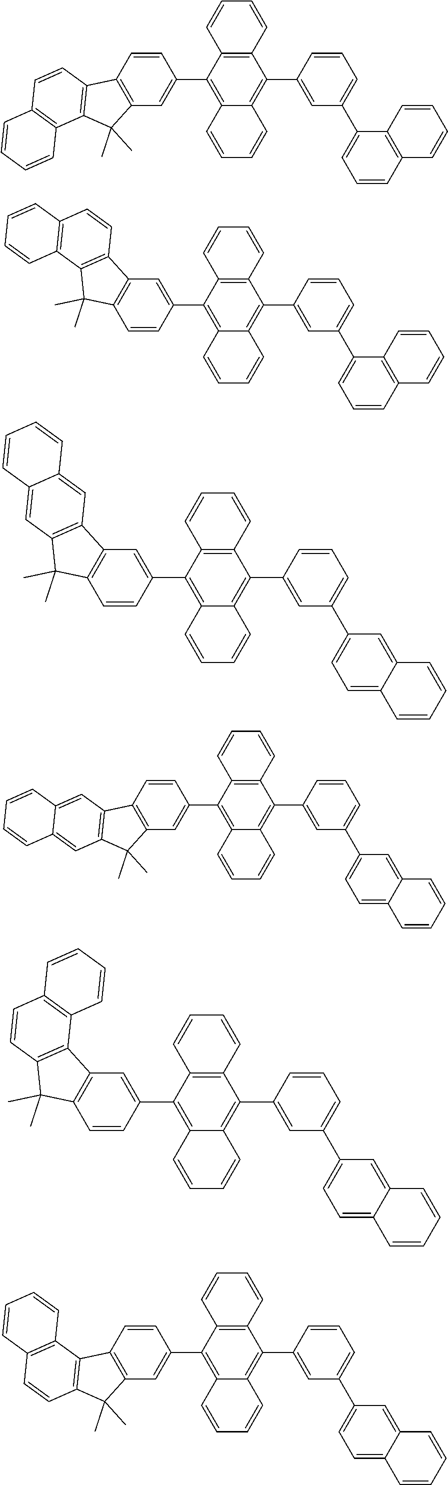

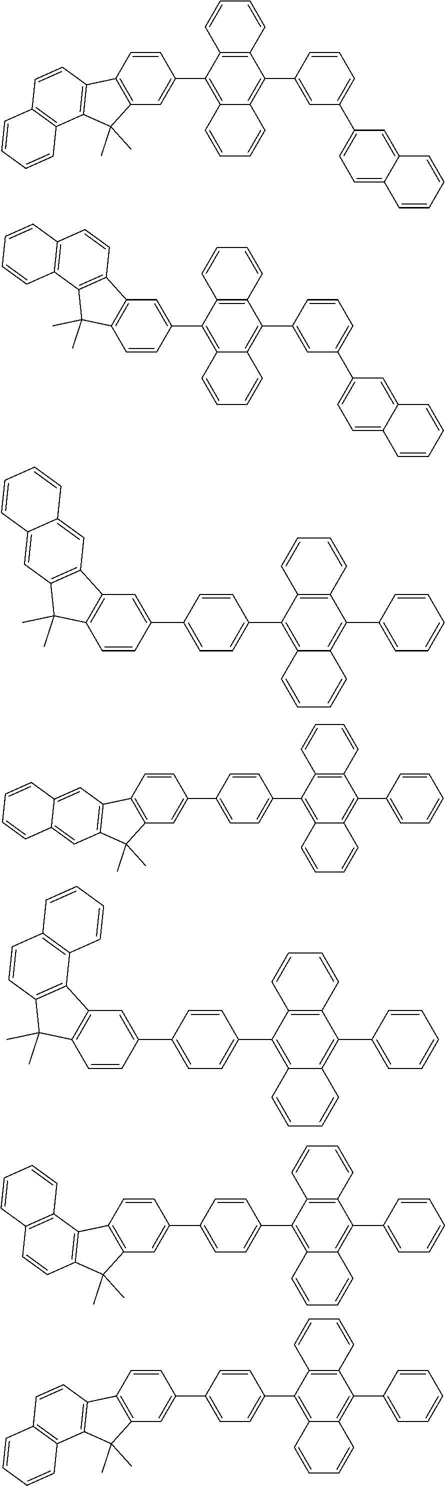

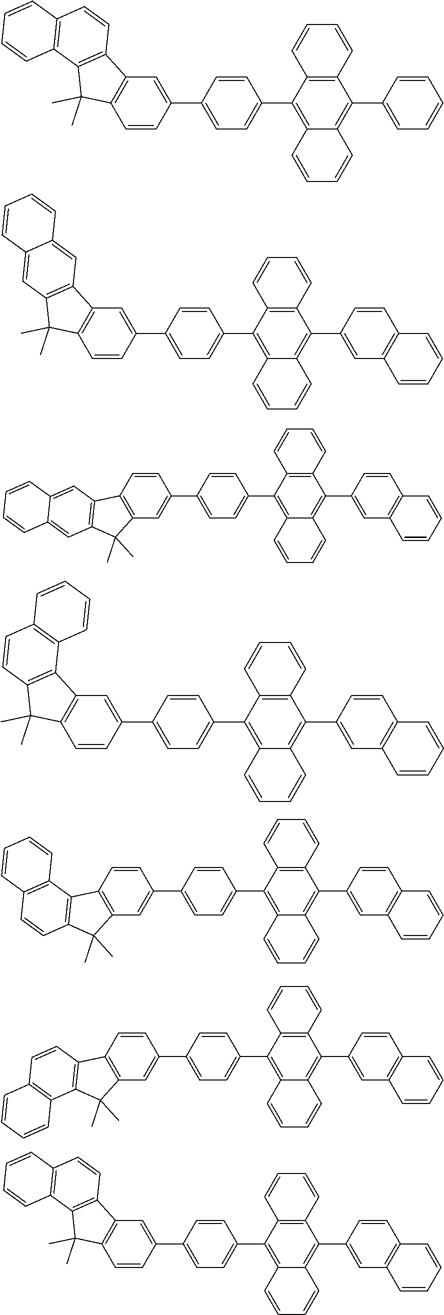

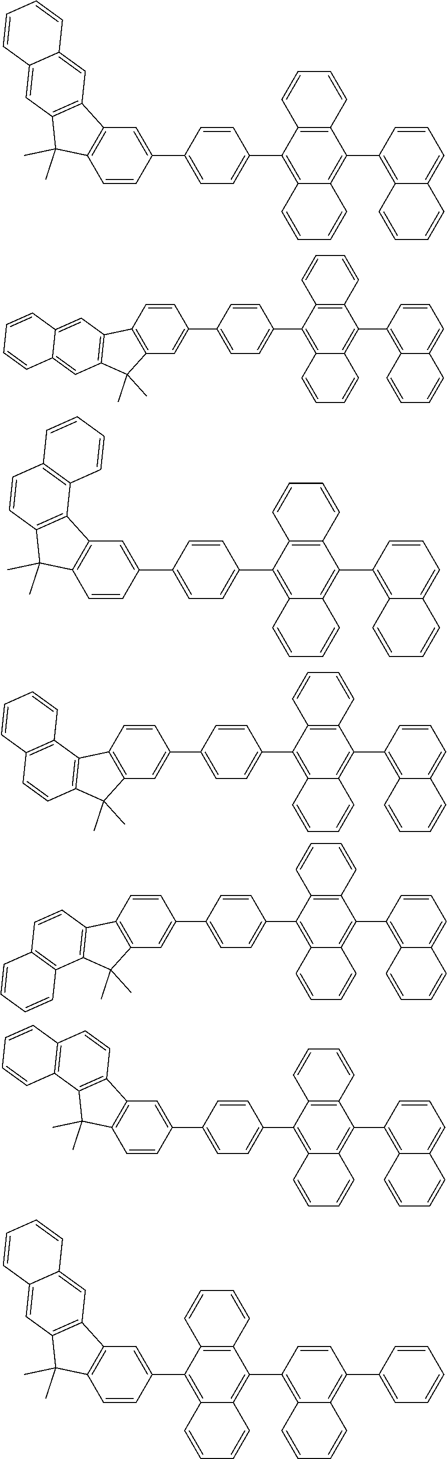

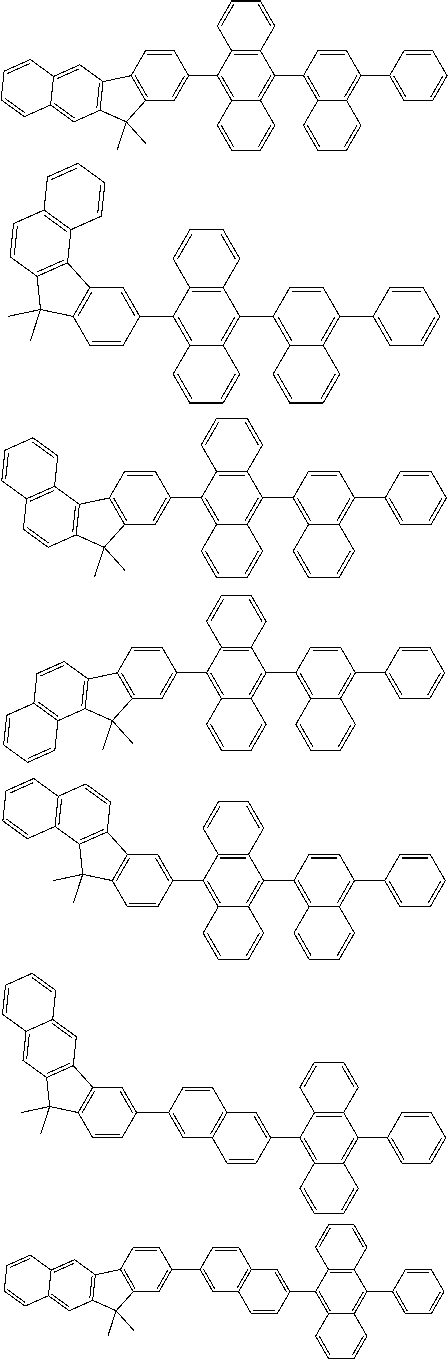

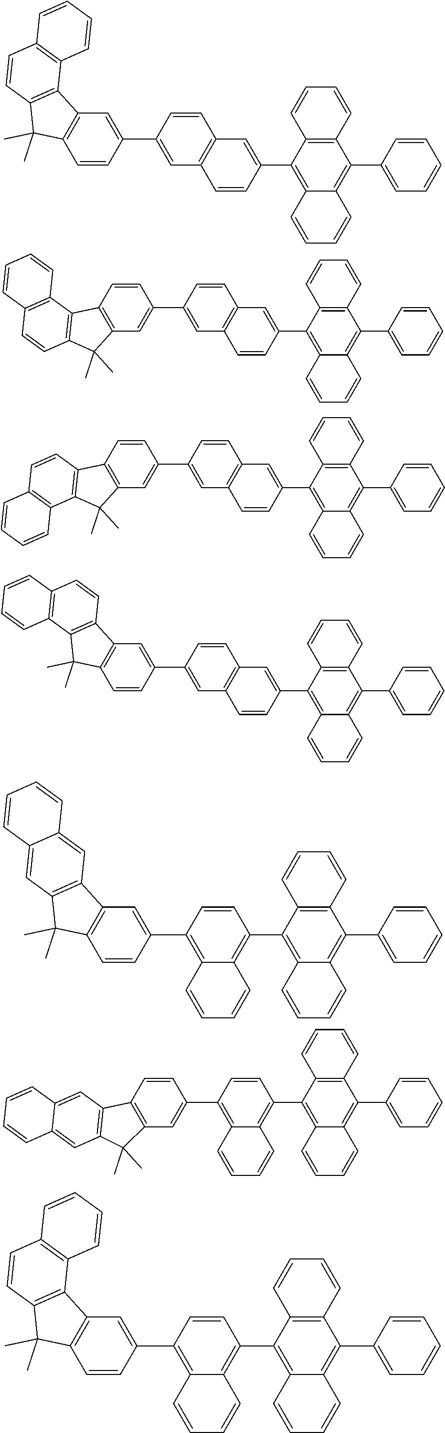

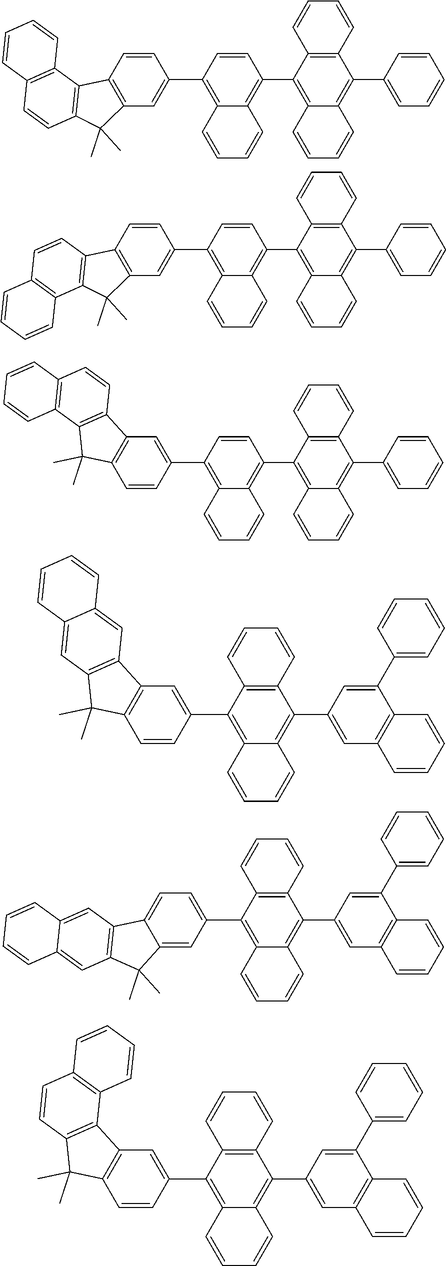

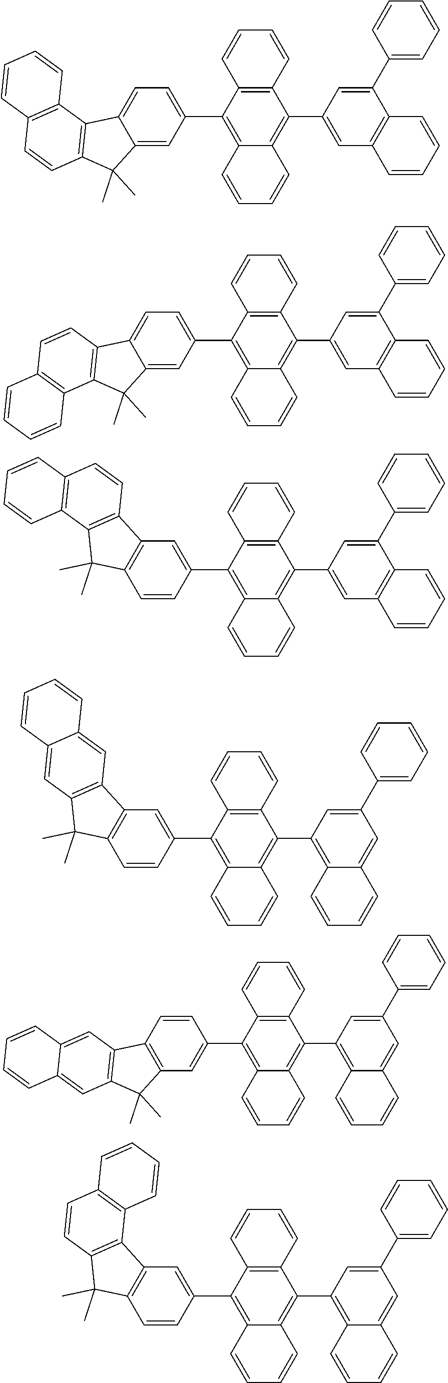

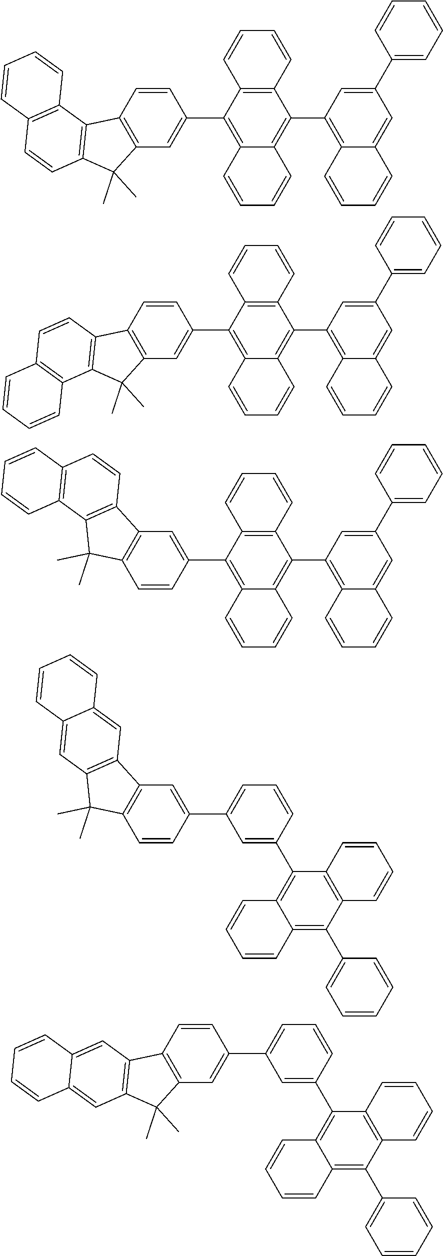

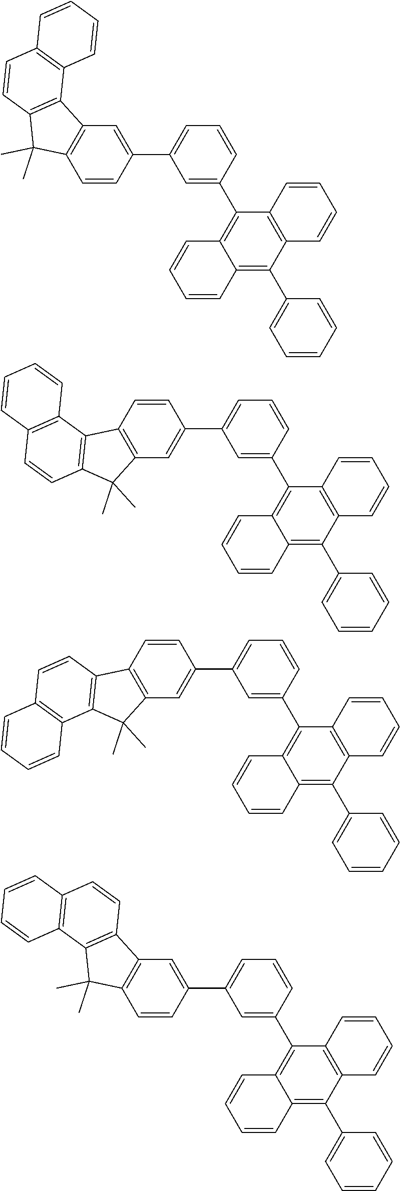

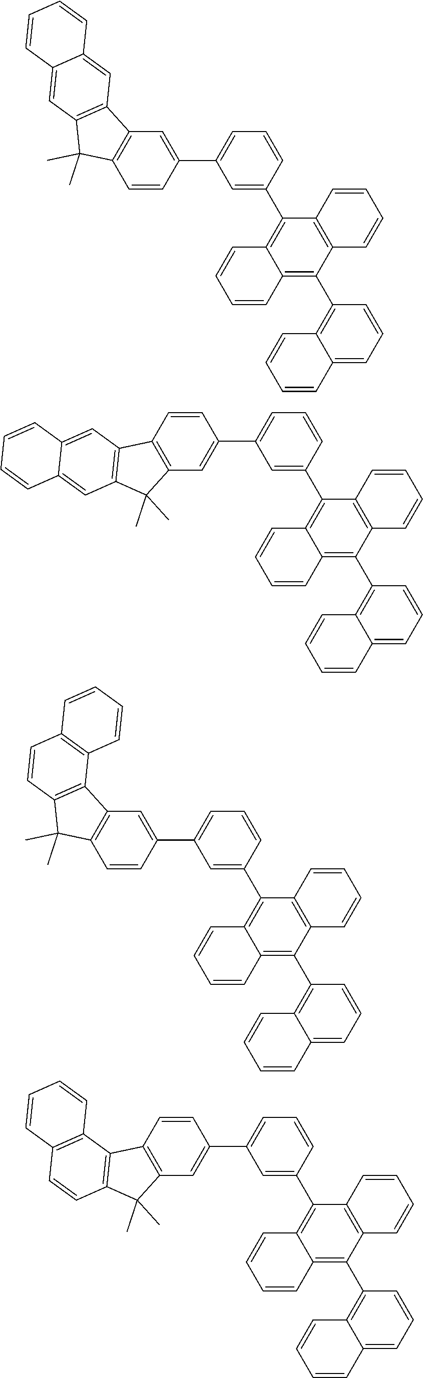

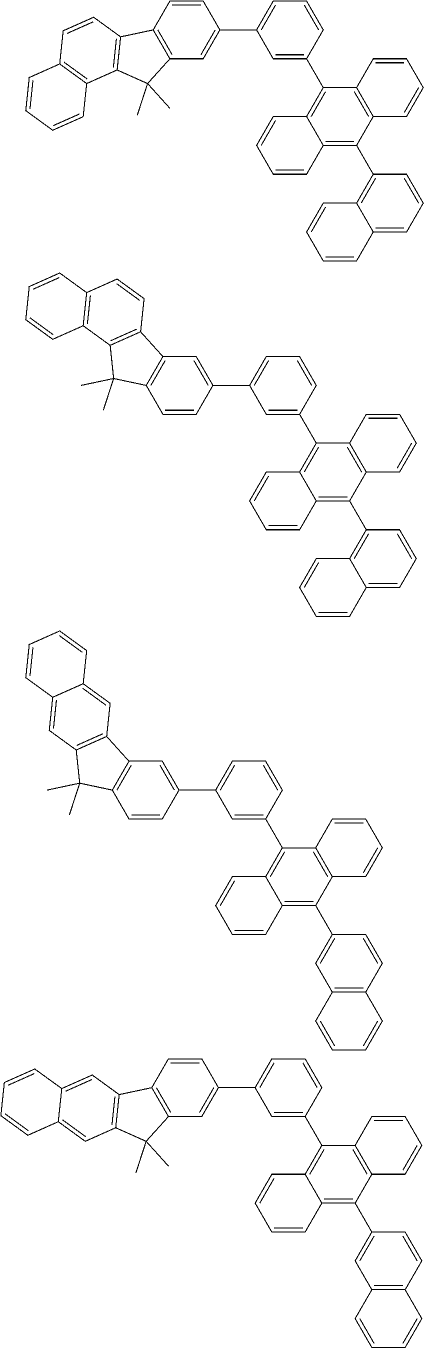

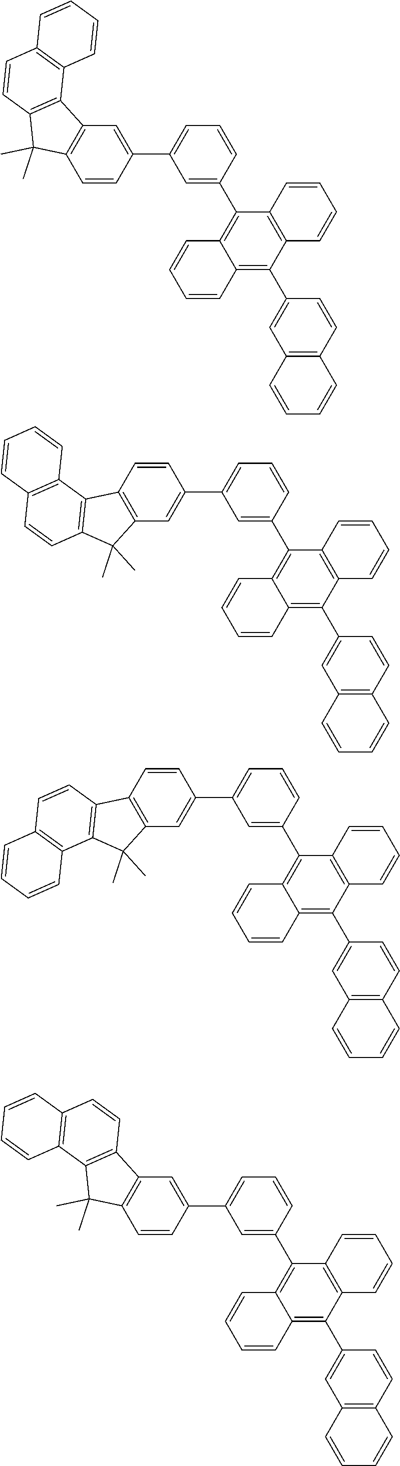

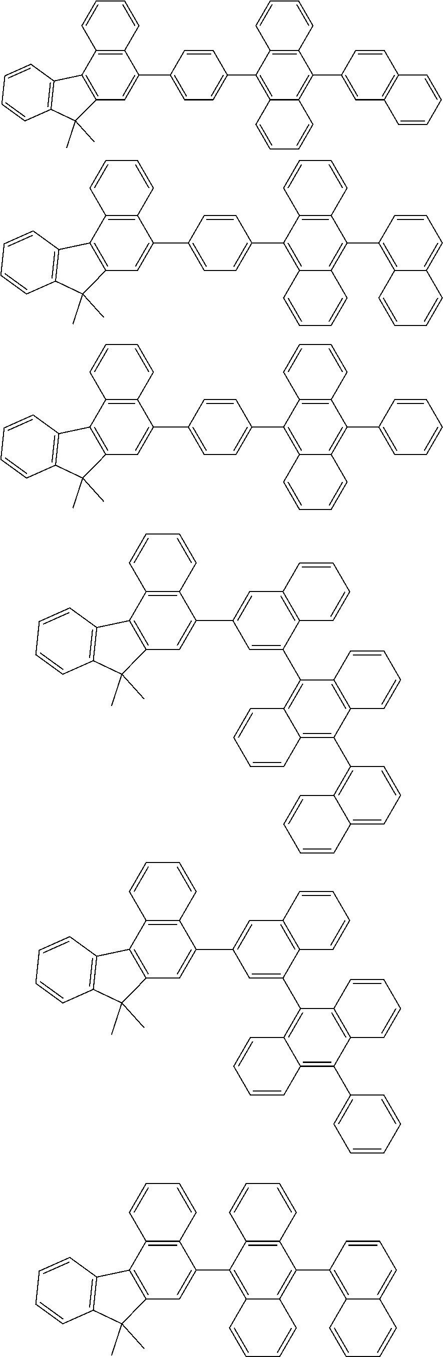

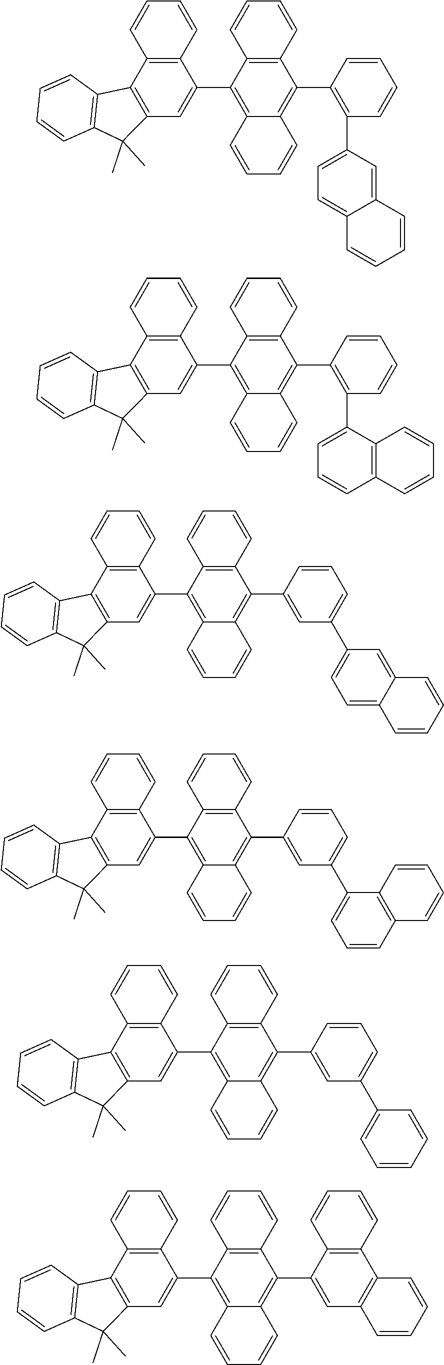

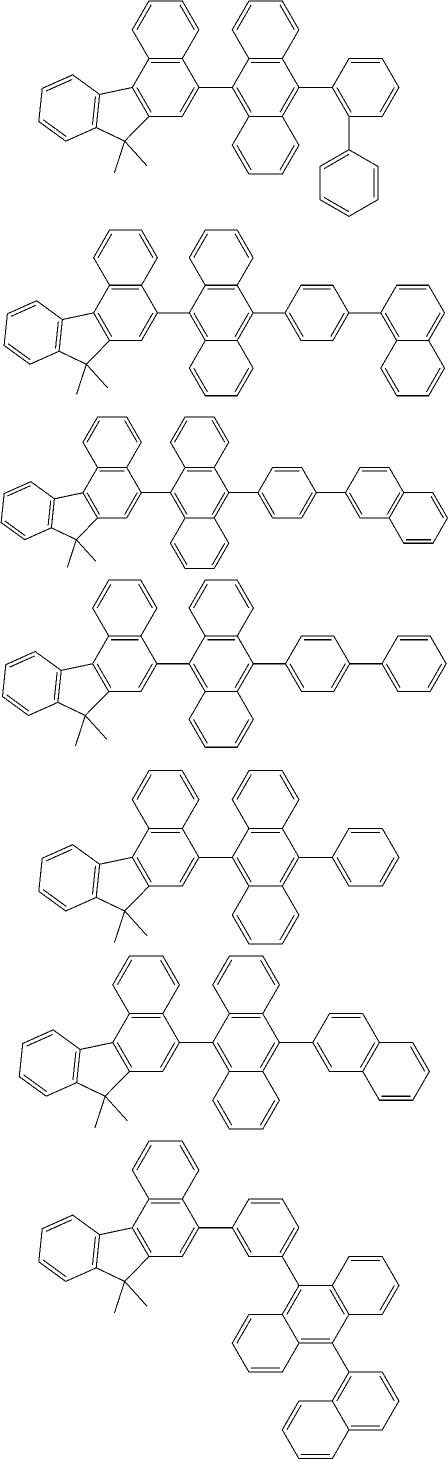

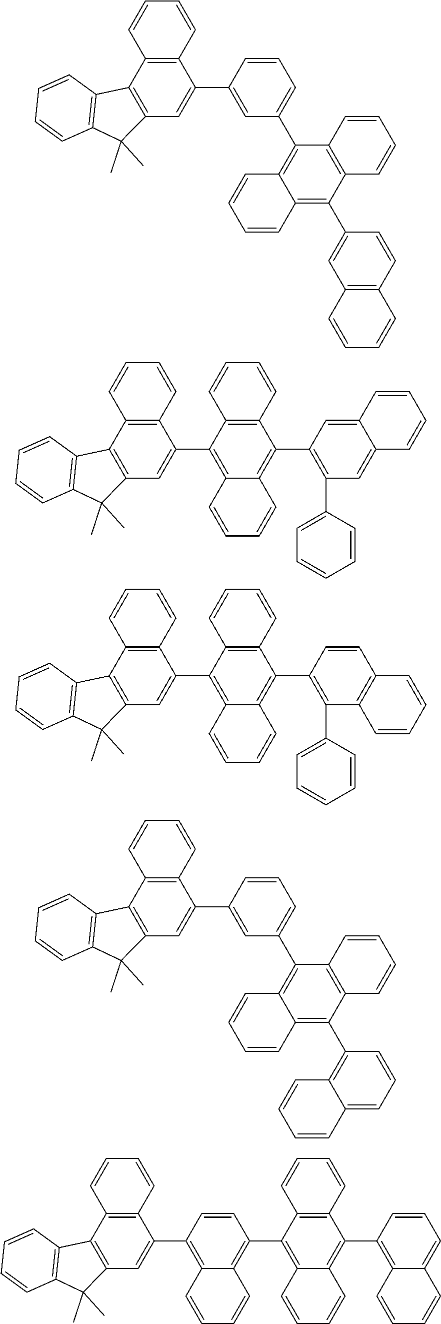

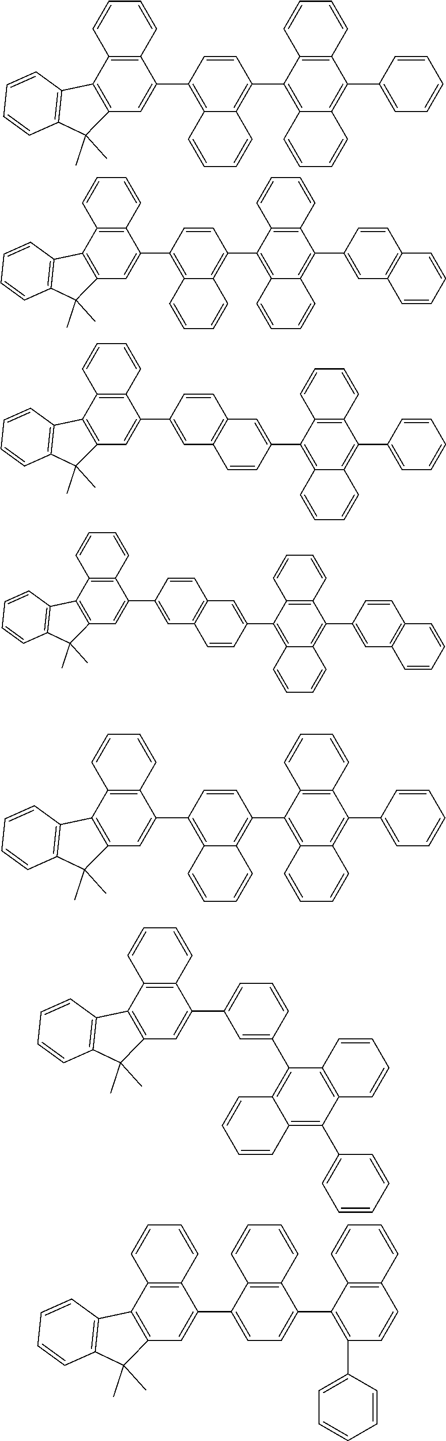

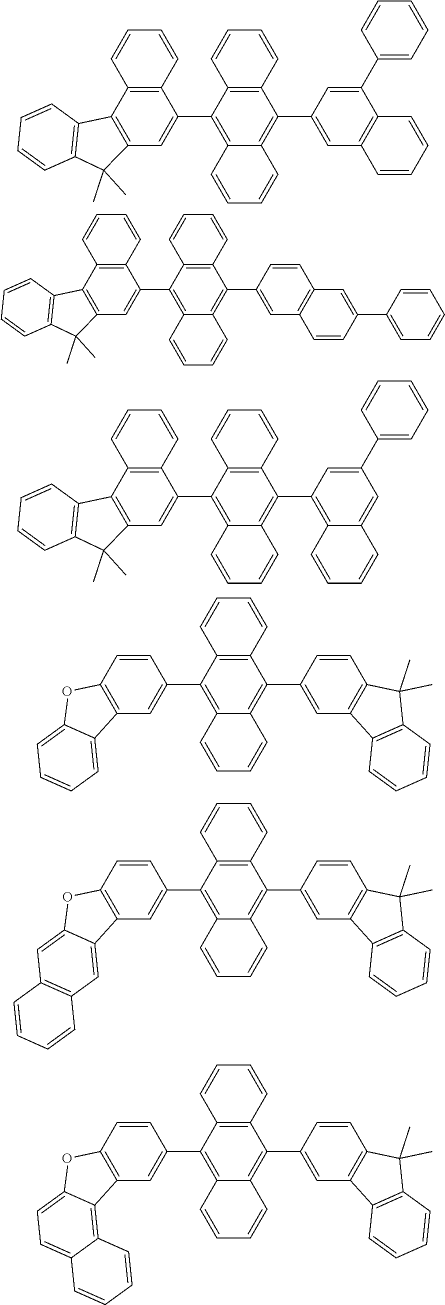

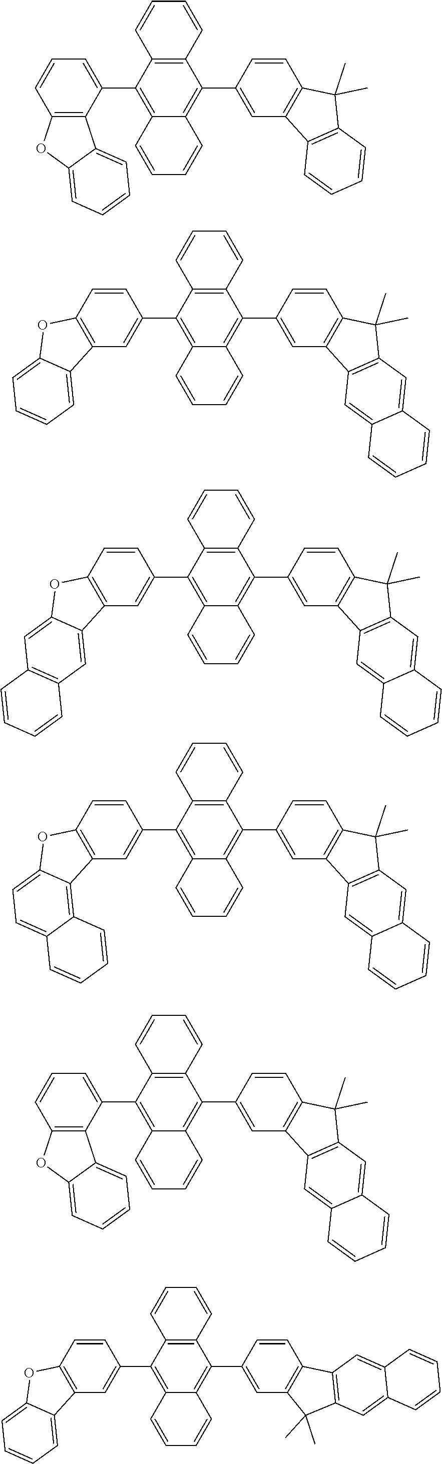 [SMILES: CC1(C)c2ccccc2-c2cc(-c3c4ccccc4c(-c4cccc5oc6ccccc6c45)c4ccccc34)ccc21 ; CC1(C)c2ccc(-c3c4ccccc4c(-c4ccc5oc6ccccc6c5c4)c4ccccc34)cc2-c2cc3ccccc3cc21 ; CC1(C)c2ccc(-c3c4ccccc4c(-c4ccc5oc6cc7ccccc7cc6c5c4)c4ccccc34)cc2-c2cc3ccccc3cc21 ; CC1(C)c2ccc(-c3c4ccccc4c(-c4ccc5oc6ccc7ccccc7c6c5c4)c4ccccc34)cc2-c2cc3ccccc3cc21 ; CC1(C)c2ccc(-c3c4ccccc4c(-c4cccc5oc6ccccc6c45)c4ccccc34)cc2-c2cc3ccccc3cc21 ; CC1(C)c2cc(-c3c4ccccc4c(-c4ccc5oc6ccccc6c5c4)c4ccccc34)ccc2-c2cc3ccccc3cc21]

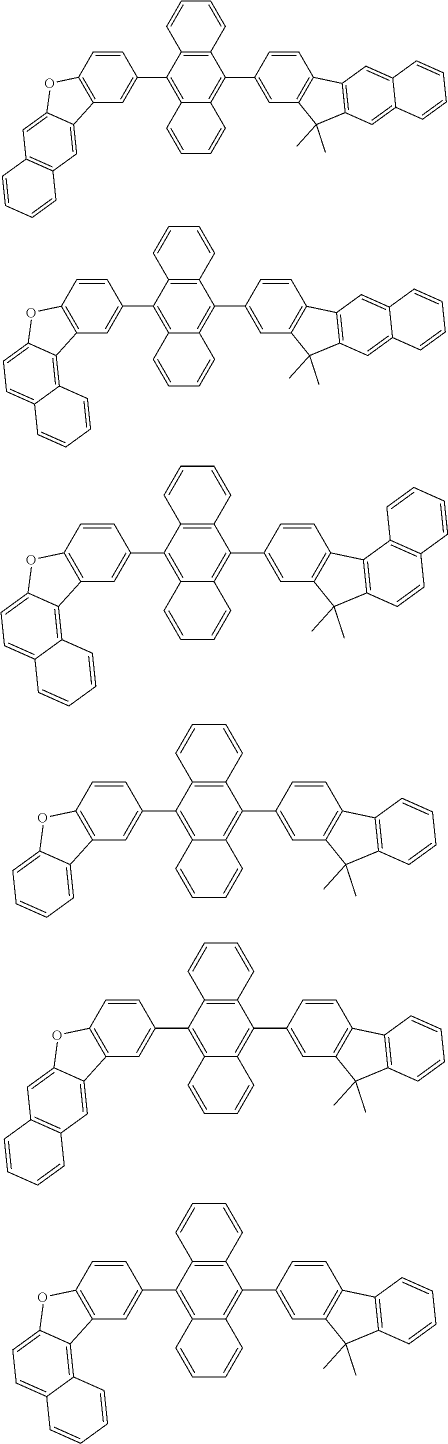 [SMILES: CC1(C)c2cc(-c3c4ccccc4c(-c4ccc5oc6cc7ccccc7cc6c5c4)c4ccccc34)ccc2-c2cc3ccccc3cc21 ; CC1(C)c2cc(-c3c4ccccc4c(-c4ccc5oc6ccc7ccccc7c6c5c4)c4ccccc34)ccc2-c2cc3ccccc3cc21 ; CC1(C)c2cc(-c3c4ccccc4c(-c4ccc5oc6ccc7ccccc7c6c5c4)c4ccccc34)ccc2-c2c1ccc1ccccc21 ; CC1(C)c2ccccc2-c2ccc(-c3c4ccccc4c(-c4ccc5oc6ccccc6c5c4)c4ccccc34)cc21 ; CC1(C)c2ccccc2-c2ccc(-c3c4ccccc4c(-c4ccc5oc6cc7ccccc7cc6c5c4)c4ccccc34)cc21 ; CC1(C)c2ccccc2-c2ccc(-c3c4ccccc4c(-c4ccc5oc6ccc7ccccc7c6c5c4)c4ccccc34)cc21]

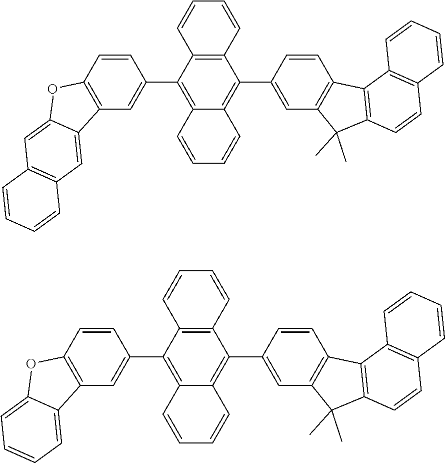 [SMILES: CC1(C)c2cc(-c3c4ccccc4c(-c4ccc5oc6cc7ccccc7cc6c5c4)c4ccccc34)ccc2-c2c1ccc1ccccc21 ; CC1(C)c2cc(-c3c4ccccc4c(-c4ccc5oc6ccccc6c5c4)c4ccccc34)ccc2-c2c1ccc1ccccc21]

Arrangement(s) of an organic EL device 1 will be detailed below. The codes will be sometimes omitted in the description below.

### Substrate

The substrate is used as a support for the organic EL device. For instance, glass, quartz, plastics and the like are usable for the substrate. A flexible substrate is also usable. The flexible substrate is a bendable substrate, which is exemplified by a plastic substrate. Examples of the material for the plastic substrate include polycarbonate, polyarylate, polyethersulfone, polypropylene, polyester, polyvinyl fluoride, polyvinyl chloride, polyimide, and polyethylene naphthalate. Moreover, an inorganic vapor deposition film is also usable.

### Anode

Metal having a large work function (specifically, 4.0 eV or more), an alloy, an electrically conductive compound and a mixture thereof are preferably used as the anode formed on the substrate. Specific examples of the material include ITO (Indium Tin Oxide), indium oxide-tin oxide containing silicon or silicon oxide, indium oxide-zinc oxide, indium oxide containing tungsten oxide and zinc oxide, and graphene. In addition, gold (Au), platinum (Pt), nickel (Ni), tungsten (W), chrome (Cr), molybdenum (Mo), iron (Fe), cobalt (Co), copper (Cu), palladium (Pd), titanium (Ti), and nitrides of a metal material (e.g., titanium nitride) are usable.

The material is typically formed into a film by a sputtering method. For instance, the indium oxide-zinc oxide can be formed into a film by the sputtering method using a target in which zinc oxide in a range from 1 mass % to 10 mass % is added to indium oxide. Moreover, for instance, the indium oxide containing tungsten oxide and zinc oxide can be formed by the sputtering method using a target in which tungsten oxide in a range from 0.5 mass % to 5 mass % and zinc oxide in a range from 0.1 mass % to 1 mass % are added to indium oxide. In addition, the anode may be formed by a vacuum deposition method, a coating method, an inkjet method, a spin coating method or the like.

Among the organic layers formed on the anode, since the hole injecting layer adjacent to the anode is formed of a composite material into which holes are easily injectable irrespective of the work function of the anode, a material usable as an electrode material (e.g., metal, an alloy, an electroconductive compound, a mixture thereof, and the elements belonging to the group 1 or 2 of the periodic table) is also usable for the anode.

A material having a small work function such as elements belonging to Groups 1 and 2 in the periodic table of the elements, specifically, an alkali metal such as lithium (Li) and cesium (Cs), an alkaline earth metal such as magnesium (Mg), calcium (Ca) and strontium (Sr), alloys (e.g., MgAg and AlLi) including the alkali metal or the alkaline earth metal, a rare earth metal such as europium (Eu) and ytterbium (Yb), alloys including the rare earth metal are also usable for the anode. It should be noted that the vacuum deposition method and the sputtering method are usable for forming the anode using the alkali metal, alkaline earth metal and the alloy thereof. Further, when a silver paste is used for the anode, the coating method and the inkjet method are usable.

### Cathode

It is preferable to use metal, an alloy, an electroconductive compound, and a mixture thereof, which have a small work function (specifically, 3.8 eV or less) for the cathode. Examples of the material for the cathode include elements belonging to Groups 1 and 2 in the periodic table of the elements, specifically, the alkali metal such as lithium (Li) and cesium (Cs), the alkaline earth metal such as magnesium (Mg), calcium (Ca) and strontium (Sr), alloys (e.g., MgAg and AlLi) including the alkali metal or the alkaline earth metal, the rare earth metal such as europium (Eu) and ytterbium (Yb), and alloys including the rare earth metal.

It should be noted that the vacuum deposition method and the sputtering method are usable for forming the cathode using the alkali metal, alkaline earth metal and the alloy thereof. Further, when a silver paste is used for the cathode, the coating method and the inkjet method are usable.

By providing the electron injecting layer, various conductive materials such as Al, Ag, ITO, graphene, and indium oxide-tin oxide containing silicon or silicon oxide may be used for forming the cathode regardless of the work function. The conductive materials can be formed into a film using the sputtering method, inkjet method, spin coating method and the like.

### Hole Injecting Layer

The hole injecting layer is a layer containing a substance exhibiting a high hole injectability. Examples of the substance exhibiting a high hole injectability include molybdenum oxide, titanium oxide, vanadium oxide, rhenium oxide, ruthenium oxide, chrome oxide, zirconium oxide, hafnium oxide, tantalum oxide, silver oxide, tungsten oxide, and manganese oxide.

In addition, the examples of the highly hole-injectable substance further include: an aromatic amine compound, which is a low-molecule organic compound, such that 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-{4-[N′-(3-methylphenyl)-N′-phenylamino]phenyl}-N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazole-3-yl)-N-phenylamino]-9-phenylcarbazole (abbreviation: PCzPCA1), 3,6-bis[N-(9-phenylcarbazole-3-yl)-N-phenylamino]-9-phenylcarbazole (abbreviation: PCzPCA2), and 3-[N-(1-naphthyl)-N-(9-phenylcarbazole-3-yl)amino]-9-phenylcarbazole (abbreviation: PCzPCN1); and dipyrazino[2,34:20,30-h]quinoxaline-2,3,6,7,10,11-hexacarbonitrile (HAT-CN).

In addition, a high polymer compound (e.g., oligomer, dendrimer and polymer) is usable as the substance exhibiting a high hole injectability. Examples of the high-molecule compound include poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide] (abbreviation: PTP DMA), and poly[N, N′-bis(4-butylphenyl)-N, N′-bis(phenyl)benzidine] (abbreviation: Poly-TPD). Moreover, an acid-added high polymer compound such as poly(3,4-ethylenedioxythiophene)/poly(styrene sulfonic acid) (PEDOT/PSS) and polyaniline/poly(styrene sulfonic acid)(PAni/PSS) are also usable.

### Hole Transporting Layer

The hole transporting layer is a layer containing a highly hole-transporting substance. An aromatic amine compound, carbazole derivative, anthracene derivative and the like are usable for the hole transporting layer. Specific examples of a material for the hole transporting layer include 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4-phenyl-4′-(9-phenylfluorene-9-yl)triphenylamine (abbreviation: BAFLP), 4,4′-bis[N-(9,9-dimethylfluorene-2-yl)-N-phenylamino]biphenyl (abbreviation: DFLDPBi), 4,4′,4″-tris(N, N-diphenylamino)triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluorene-2-yl)-N-phenylamino]biphenyl (abbreviation: BSPB). The above-described substances mostly have a hole mobility of 10⁻⁶ cm²/(V·s) or more.

For the hole transporting layer, a carbazole derivative such as CBP, 9-[4-(N-carbazolyl)]phenyl-10-phenylanthracene (CzPA), and 9-phenyl-3-[4-(10-phenyl-9-anthryl)phenyl]-9H-carbazole (PCzPA) and an anthracene derivative such as t-BuDNA, DNA, and DPAnth may be used. A high polymer compound such as poly(N-vinylcarbazole) (abbreviation: PVK) and poly(4-vinyltriphenylamine) (abbreviation: PVTPA) is also usable.

However, in addition to the above substances, any substance exhibiting a higher hole transportability than an electron transportability may be used. It should be noted that the layer containing the substance exhibiting a high hole transportability may be not only a single layer but also a laminate of two or more layers formed of the above substance(s).

When the hole transporting layer includes two or more layers, one of the layers with a larger energy gap is preferably provided closer to the emitting layer. An example of the material with a larger energy gap is HT2 used in later-described Examples.

### Electron Transporting Layer

The electron transporting layer is a layer containing a highly electron-transporting substance. For the electron transporting layer, 1) a metal complex such as an aluminum complex, beryllium complex, and zinc complex, 2) a hetero aromatic compound such as imidazole derivative, benzimidazole derivative, azine derivative, carbazole derivative, and phenanthroline derivative, and 3) a high polymer compound are usable. Specifically, as a low-molecule organic compound, a metal complex such as Alq, tris(4-methyl-8-quinolinato)aluminum (abbreviation: Almq₃), bis(10-hydroxybenzo[h]quinolinato)beryllium (abbreviation: BeBq₂), BAlq, Znq, ZnPBO and ZnBTZ is usable. In addition to the metal complex, a heteroaromatic compound such as 2-(4-biphenylyl)-5-(4-tert-butylphenyl)-1,3,4-oxadiazole (abbreviation: PBD), 1,3-bis[5-(ptert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzoxazole-2-yl)stilbene (abbreviation: BzOs) is usable. In the exemplary embodiment, a benzimidazole compound is preferably usable. The above-described substances mostly have an electron mobility of 10⁻⁶ cm²/(V·s) or more. It should be noted that any substance other than the above substance may be used for the electron transporting layer as long as the substance exhibits a higher electron transportability than the hole transportability. The electron transporting layer may be provided in the form of a single layer or a laminate of two or more layers of the above substance(s).

Moreover, a high polymer compound is usable for the electron transporting layer. For instance, poly[(9,9-dihexylfluorene-2,7-diyl)-co-(pyridine-3,5-diyl)] (abbreviation: PF-Py), poly[(9,9-dioctylfluorene-2,7-diyl)-co-(2,2′-bipyridine-6,6′-diyl)] (abbreviation: PF-BPy) and the like are usable.

### Electron Injecting Layer

The electron injecting layer is a layer containing a highly electron-injectable substance. Examples of a material for the electron injecting layer include an alkali metal, alkaline earth metal and a compound thereof, examples of which include lithium (Li), cesium (Cs), calcium (Ca), lithium fluoride (LiF), cesium fluoride (CsF), calcium fluoride (CaF₂), and lithium oxide (LiOx). In addition, the alkali metal, alkaline earth metal or the compound thereof may be added to the substance exhibiting the electron transportability in use. Specifically, for instance, magnesium (Mg) added to Alq may be used. In this case, the electrons can be more efficiently injected from the anode.

Alternatively, the electron injecting layer may be provided by a composite material in a form of a mixture of the organic compound and the electron donor. Such a composite material exhibits excellent electron injectability and electron transportability since electrons are generated in the organic compound by the electron donor. In this case, the organic compound is preferably a material excellent in transporting the generated electrons. Specifically, the above examples (e.g., the metal complex and the hetero aromatic compound) of the substance forming the electron transporting layer are usable. As the electron donor, any substance exhibiting electron donating property to the organic compound is usable. Specifically, the electron donor is preferably alkali metal, alkaline earth metal and rare earth metal such as lithium, cesium, magnesium, calcium, erbium and ytterbium. The electron donor is also preferably alkali metal oxide and alkaline earth metal oxide such as lithium oxide, calcium oxide, and barium oxide. Moreover, a Lewis base such as magnesium oxide is usable. Further, the organic compound such as tetrathiafulvalene (abbreviation: TTF) is usable.

### Layer Formation Method(s)

A method for forming each layer of the organic EL device in the third exemplary embodiment is subject to no limitation except for the above particular description. However, known methods of dry film-forming such as vacuum deposition, sputtering, plasma or ion plating and wet film-forming such as spin coating, dipping, flow coating or ink jet printing are applicable.

### Film Thickness

There is no restriction except for the above particular description for a film thickness of each of the organic layers of the organic EL device in the third exemplary embodiment. The thickness of each of the organic layers of the organic EL device is usually preferably in a range from several nanometers to 1 μm, because too small film thickness causes defects (e.g., pin holes) and too large film thickness requires application of high voltage, resulting in deterioration in the efficiency.

### Electronic Device

An electronic device according to the third exemplary embodiment is preferably installed with an organic EL device according to the third exemplary embodiment. Examples of the electronic device include a display device and a light-emitting unit.

Examples of the display device include a display component (e.g., an organic EL panel module), TV, mobile phone, tablet and personal computer. Examples of the light-emitting unit include an illuminator and a vehicle light.

According to the third exemplary embodiment, an organic electroluminescence device containing a compound capable of enhancing performance of the organic electroluminescence device and an electronic device including the organic electroluminescence device can be provided.

### Modification of Embodiment(s)

It should be noted that the invention is not limited to the above exemplary embodiments but may include any modification and improvement as long as such modification and improvement are compatible with the invention.

For instance, the emitting layer is not limited to a single layer, but may be provided by laminating a plurality of emitting layers. When the organic EL device has a plurality of emitting layers, it is only required that at least one of the emitting layers satisfies the conditions described in the above exemplary embodiment. For instance, the rest of the emitting layers may be a fluorescent emitting layer or a phosphorescent emitting layer with use of emission caused by electron transfer from the triplet excited state directly to the ground state.

When the organic EL device includes the plurality of emitting layers, the plurality of emitting layers may be adjacent to each other, or provide a so-called tandem-type organic EL device in which a plurality of emitting units are layered through an intermediate layer.

For instance, a blocking layer may be provided adjacent to at least one of a side near the anode and a side near the cathode of the emitting layer. The blocking layer is preferably provided in contact with the emitting layer to at least block holes, electrons or excitons.

For instance, when the blocking layer is provided in contact with the cathode-side of the emitting layer, the blocking layer permits transport of electrons, but blocks holes from reaching a layer provided near the cathode (e.g., the electron transporting layer) beyond the blocking layer. When the organic EL device includes the electron transporting layer, the organic EL device preferably includes the blocking layer between the emitting layer and the electron transporting layer.

When the blocking layer is provided in contact with the anode-side of the emitting layer, the blocking layer permits transport of holes, but blocks electrons from reaching a layer provided near the anode (e.g., the hole transporting layer) beyond the blocking layer. When the organic EL device includes the hole transporting layer, the organic EL device preferably includes the blocking layer between the emitting layer and the hole transporting layer.

Moreover, the blocking layer may abut on the emitting layer so that excited energy does not leak out from the emitting layer toward neighboring layer(s). The blocking layer blocks excitons generated in the emitting layer from being transferred to a layer(s) (e.g., the electron transporting layer and the hole transporting layer) closer to the electrode(s) beyond the blocking layer.

The emitting layer and the blocking layer are preferably bonded with each other.

Specific structure and shape of the components in the present invention may be designed in any manner as long as an object of the present invention can be achieved.

### EXAMPLES

Example(s) of the invention will be described below. However, the invention is not limited to Example(s).

### Compounds

Compounds represented by the formula (2), which are used in preparing organic EL devices according to Examples 1 to 6, are shown below.

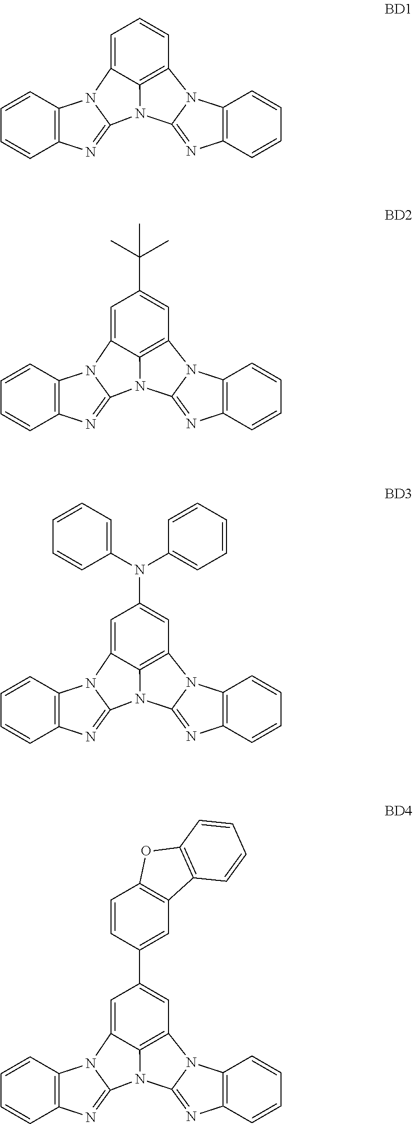 [SMILES: c1ccc2c(c1)nc1n2c2cccc3c2n1c1nc2ccccc2n31 ; CC(C)(C)c1cc2c3c(c1)n1c4ccccc4nc1n3c1nc3ccccc3n21 ; c1ccc(N(c2ccccc2)c2cc3c4c(c2)n2c5ccccc5nc2n4c2nc4ccccc4n32)cc1 ; c1ccc2c(c1)nc1n2c2cc(-c3ccc4oc5ccccc5c4c3)cc3c2n1c1nc2ccccc2n31]

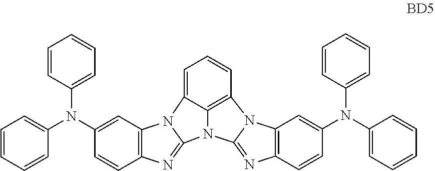 [SMILES: c1ccc(N(c2ccccc2)c2ccc3nc4n(c3c2)c2cccc3c2n4c2nc4ccc(N(c5ccccc5)c5ccccc5)cc4n32)cc1]

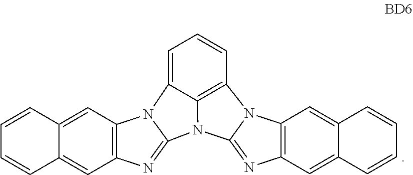 [SMILES: c1ccc2cc3c(cc2c1)nc1n3c2cccc3c2n1c1nc2cc4ccccc4cc2n31]

Compounds represented by the formula (10), which are used in preparing organic EL devices according to Examples 1 to 6, are shown below.

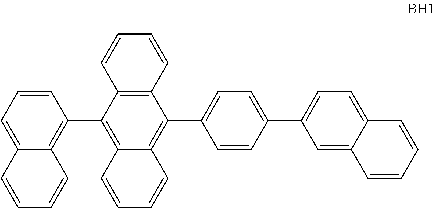 [SMILES: c1ccc2cc(-c3ccc(-c4c5ccccc5c(-c5cccc6ccccc56)c5ccccc45)cc3)ccc2c1]

The structures of other compounds used in preparing organic EL devices according to Examples 1 to 6 are shown below.

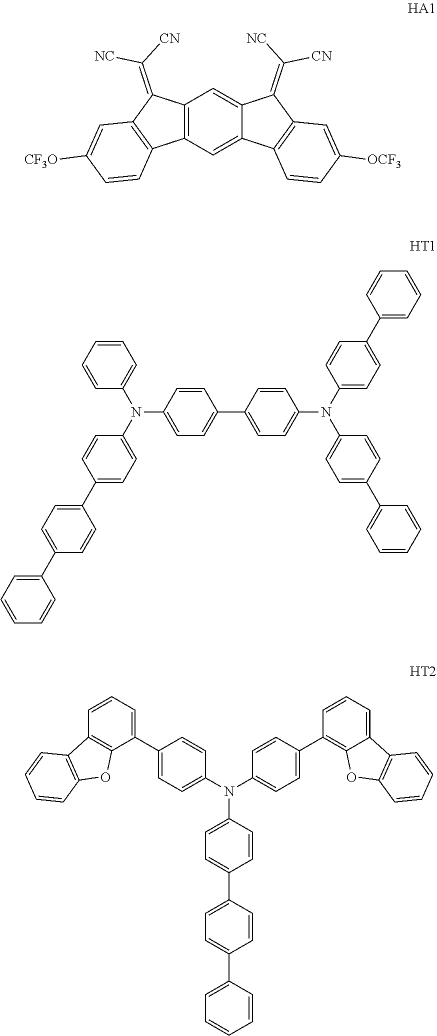

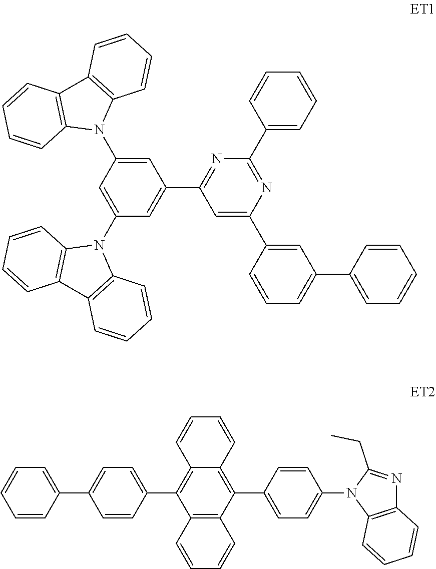 [SMILES: c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; CCc1nc2ccccc2n1-c1ccc(-c2c3ccccc3c(-c3ccc(-c4ccccc4)cc3)c3ccccc23)cc1]

### Preparation of Organic EL Device

The organic EL devices were prepared and evaluated as follows.

### Example 1

A glass substrate (size: 25 mm×75 mm×1.1 mm thick, manufactured by Geomatec Co., Ltd.) having an ITO transparent electrode (anode) was ultrasonic-cleaned in isopropyl alcohol for five minutes, and then UV/ozone-cleaned for 5 minutes. A film of ITO was 130 nm thick.

After the cleaned glass substrate having the transparent electrode was mounted on a substrate holder of a vacuum vapor-deposition apparatus, a compound HA1 was vapor-deposited on a surface of the glass substrate where the transparent electrode was provided in a manner to cover the transparent electrode, thereby forming a 5-nm-thick HA1 film. The HA1 film serves as a hole injecting layer.

Subsequently to the formation of the HA1 film, a compound HT1 was vapor-deposited to form an 80-nm-thick HT1 film on the HA1 film. The HT1 film serves as a hole transporting layer (first hole transporting layer).

Subsequently to the formation of the HT1 film, a compound HT2 was vapor-deposited to form a 10-nm-thick HT2 film on the HT1 film. The HT2 film serves as an electron blocking layer (second hole transporting layer).

A compound BH1 (host material) and a compound BD1 (dopant material) were co-vapor-deposited on the HT2 film so that the ratio of the compound BD1 accounted for 2 mass %, thereby forming a 25-nm-thick BH1:BD1 film. The BH1:BD1 film serves as an emitting layer.

The compound ET1 was vapor-deposited on the emitting layer to form a 10-nm-thick ET1 film. The ET1 film serves as a hole blocking layer.

A compound ET2 was vapor-deposited on the ET1 film to form a 15-nm-thick ET2 film. The ET2 film serves as an electron transporting layer. LiF was vapor-deposited on the ET2 film to form a 1-nm-thick LiF film. Metal Al was vapor-deposited on the LiF film to form an 80-nm-thick metal cathode to prepare an organic EL device.

A layer structure of the resultant organic EL device is as follows. ITO(130)/HA1(5)/HT1(80)/HT2(10)/BH1:BD1(25, 98 mass %:2 mass %)/ET1(10)/ET2(15)/LiF(1)/Al(80)

It should be noted that numerals in parentheses represent a film thickness (unit: nm). The numerals in the form of percentage in parentheses indicate ratios (mass %) of the compounds of the host and dopant materials in the emitting layer.

### Evaluation of Organic EL Device

Voltage was applied to the resultant organic EL device so that the current density became 10 mA/cm², and EL emission spectrum was measured using a spectroradiometer CS-1000 (manufactured by Konica Minolta, Inc.). EQE (External Quantum Efficiency) (%) was calculated based on the obtained spectral-radiance spectra. The results are shown in Table 1.

Emission color of the organic EL device was checked in measuring the EQE using the spectroradiometer CS-1000 (manufactured by Konica Minolta, Inc.).

### Examples 2 to 6

Organic EL devices were prepared and evaluated in the same manner as in Example 1 except that the host material (BH) and the dopant material (BD) were changed to compounds shown in Table 1. The results are shown in Table 1.

TABLE 1

| | Emitting Layer | | EQE | Emission |
|---|---|---|---|---|
| | Host Material | Dopant Material | [%] | Color |
| Ex. 1 | BH1 | BD1 | 5.2 | Blue |
| Ex. 2 | BH1 | BD2 | 5.4 | Blue |
| Ex. 3 | BH1 | BD3 | 5.6 | Blue |
| Ex. 4 | BH1 | BD4 | 5.4 | Blue |
| Ex. 5 | BH1 | BD5 | 5.6 | Blue |
| Ex. 6 | BH1 | BD6 | 5.3 | Blue |

The compound provided by the invention is usable as a blue emission material for an organic electroluminescence device.

### Synthesis Example

Synthesis examples of the compound represented by the formula (2) are described below. In the synthesis examples below, compounds 1 to 6 correspond to compounds BD1 to BD6 used in Examples 1 to 6, respectively.

### Synthesis Example 1

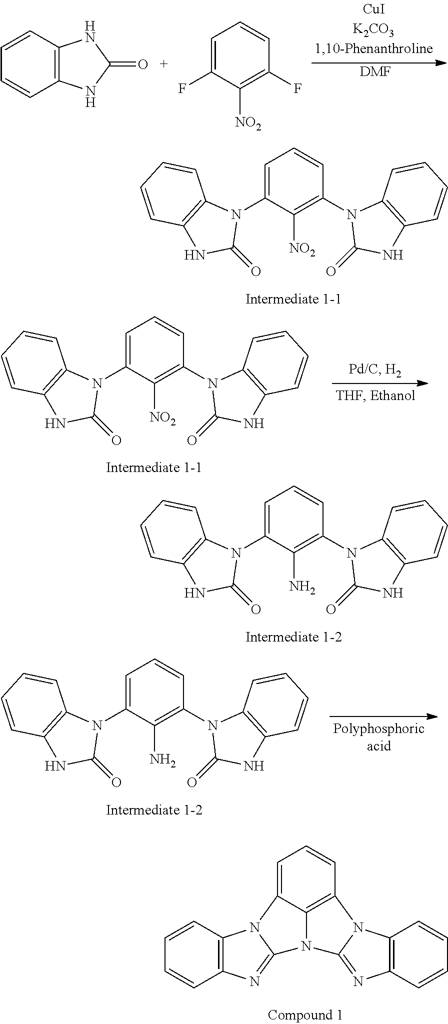

### Synthesis of Intermediate 1-1

Under argon atmosphere, a mixture of 2(3H)-benzimidazolone (8.0 g, 60.0 mmol), 2,6-difluoronitrobenzene (4.8 g, 30.0 mmol), copper iodide (CuI) (22.9 g, 120.0 mmol), potassium carbonate (K₂CO₃) (16.6 g, 120.0 mmol), 1,10-phenanthroline (0.54 g, 3.0 mmol), and dimethylformamide (DMF) (80 ml) was stirred at 130 degrees C. for 24 hours. After stirring, resultant reaction solution was cooled to a room temperature, added with water, and filtrated to obtain a precipitated solid. The obtained solid was purified through silica-gel column chromatography and recrystallization to obtain an intermediate 1-1 (4.6 g, yield rate 40%). The solid was identified as the intermediate 1-1 by FD-MS (Field Desorption Mass Spectrometry) analysis.

### Synthesis of Intermediate 1-2

Tetrahydrofuran and ethanol (1:1 (volume ratio), 125 ml) were added to the intermediate 1-1 (3.9 g, 10.0 mmol) and Pd/C (10%) (1 g) and the mixture was stirred under hydrogen atmosphere at 50 degrees C. for 24 hours. After the stirring, the resultant reaction solution was filtrated and filtrate was condensed. The obtained solid was purified through silica-gel column chromatography and recrystallization to obtain an intermediate 1-2 (3.2 g, yield rate 90%). The resultant substance was identified to be the intermediate 1-2 by FD-MS analysis.

### Synthesis of Compound 1

Under argon atmosphere, polyphosphoric acid (50 ml) was added to the intermediate 1-2 (3.0 g, 8.4 mmol) and the mixture was stirred at 240 degrees C. for 8 hours. After stirring, resultant reaction solution was cooled to a room temperature, added with water, and filtrated to obtain a precipitated solid. The obtained solid was purified through silica-gel column chromatography and recrystallization to obtain a compound 1 (0.70 g, yield rate 26%). The obtained solid was identified as the compound 1 by FD-MS analysis.

### Synthesis Example 2

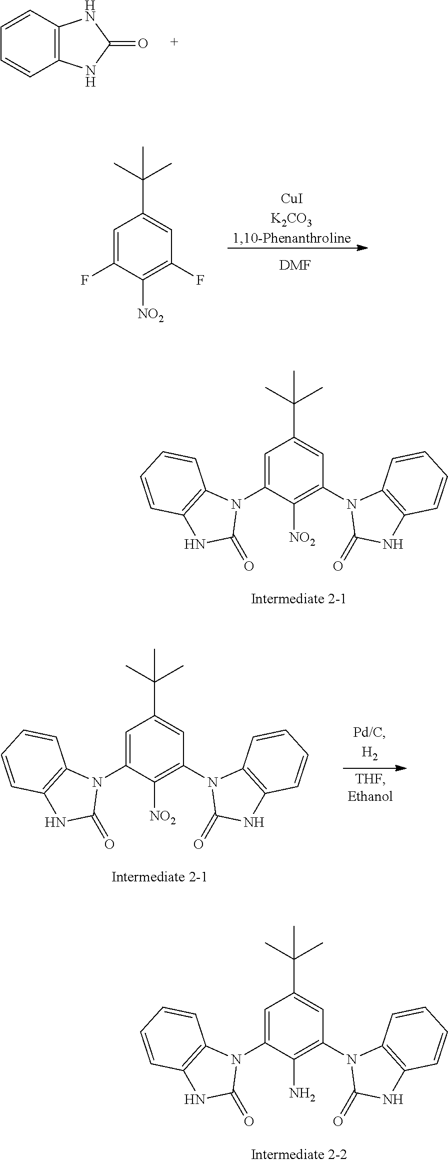

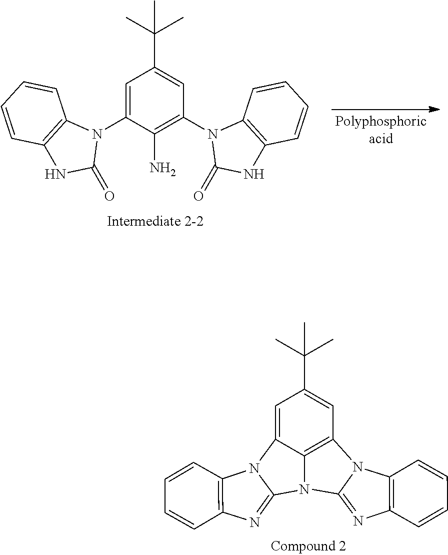 [SMILES: CC(C)(C)c1cc(-n2c(=O)[nH]c3ccccc32)c(N)c(-n2c(=O)[nH]c3ccccc32)c1 ; CC(C)(C)c1cc2c3c(c1)n1c4ccccc4nc1n3c1nc3ccccc3n21]

### Synthesis of Intermediate 2-1

Except for the use of 5-tert-buthyl-1,3-difluoro-2-nitrobenzene (6.5 g, 30.0 mmol) instead of 2,6-difluoronitrobenzene, the same reaction as in the synthesis example 1 (synthesis of intermediate 1-1) was performed to obtain an intermediate 2-1 (4.9 g, yield rate 37%). The resultant substance was identified to be the intermediate 2-1 by FD-MS analysis.

### Synthesis of Intermediate 2-2

Except for the use of the intermediate 2-1 (4.4 g, 10.0 mmol) instead of the intermediate 1-1, the same reaction as in the synthesis example 1 (synthesis of intermediate 1-2) was performed to obtain an intermediate 2-2 (3.8 g, yield rate 91%). The resultant substance was identified to be the intermediate 2-2 by FD-MS analysis.

### Synthesis of Compound 2

Except for the use of the intermediate 2-2 (3.5 g, 8.4 mmol) instead of the intermediate 1-2, the same reaction as in the synthesis example 1 (synthesis of compound 1) was performed to obtain a compound 2 (0.95 g, yield rate 30%). The substance was identified as the compound 2 by FD-MS analysis.

### Synthesis Example 3

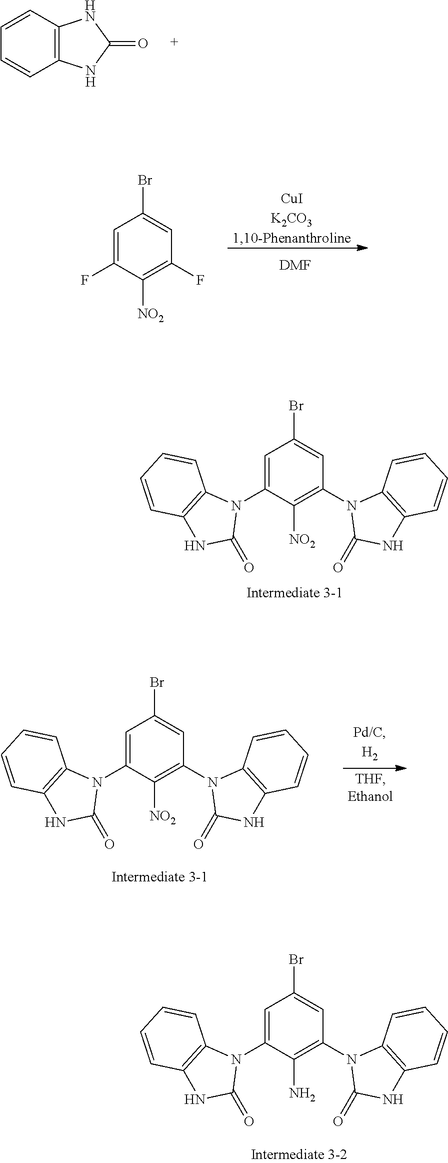

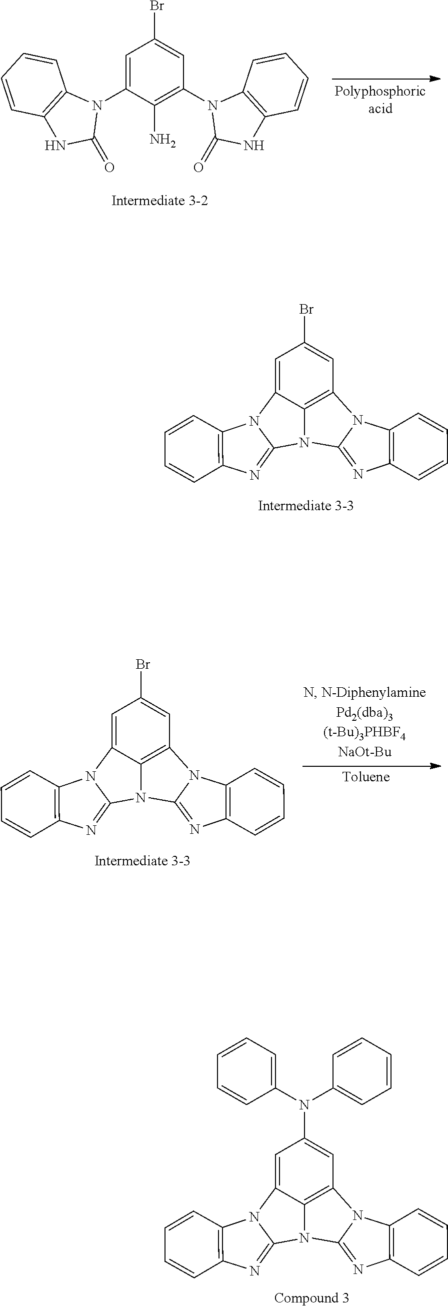 [SMILES: Nc1c(-n2c(=O)[nH]c3ccccc32)cc(Br)cc1-n1c(=O)[nH]c2ccccc21 ; Brc1cc2c3c(c1)n1c4ccccc4nc1n3c1nc3ccccc3n21 ; Brc1cc2c3c(c1)n1c4ccccc4nc1n3c1nc3ccccc3n21 ; c1ccc(N(c2ccccc2)c2cc3c4c(c2)n2c5ccccc5nc2n4c2nc4ccccc4n32)cc1]

### Synthesis of Intermediate 3-1

Except for the use of 5-bromo-1,3-difluoro-2-nitrobenzene (7.1 g, 30.0 mmol) instead of 2,6-difluoronitrobenzene, the same reaction as in the synthesis example 1 (synthesis of intermediate 1-1) was performed to obtain an intermediate 3-1 (5.5 g, yield rate 39%). The resultant substance was identified to be the intermediate 3-1 by FD-MS analysis.

### Synthesis of Intermediate 3-2

Except for the use of the intermediate 3-1 (4.6 g, 10.0 mmol) instead of the intermediate 1-1, the same reaction as in the synthesis example 1 (synthesis of intermediate 1-2) was performed to obtain an intermediate 3-2 (3.8 g, yield rate 87%). The resultant substance was identified to be the intermediate 3-2 by FD-MS analysis.

### Synthesis of Intermediate 3-3

Except for the use of the intermediate 3-2 (3.8 g, 8.7 mmol) instead of the intermediate 1-2, the same reaction as in the synthesis example 1 (synthesis of compound 1) was performed to obtain an intermediate 3-3 (1.1 g, yield rate 32%). The resultant substance was identified to be the intermediate 3-3 by FD-MS analysis.

### Synthesis of Compound 3

Under argon atmosphere, 10 ml anhydrous toluene was added to a mixture of the intermediate 3-3 (0.40 g, 1.0 mmol), N,N-diphenyl amine (0.17 g, 1.0 mmol), Pd₂(dba)₃ (14 mg, 0.015 mmol), P(tBu)₃HBF₄ (8.7 mg, 0.03 mmol), and sodium t-butoxy (1.9 g, 2.0 mmol), and the mixture was heated for reflux for eight hours.

After the reaction, the reaction solution was cooled to 50 degrees C. and was filtrated through celite/silica gel. Then the filtrate was condensed. The resultant condensed residue was purified through silica-gel column chromatography and recrystallization to obtain a compound 3 (0.28 g, yield rate 57%). The substance was identified as the compound 3 by FD-MS analysis.

### Synthesis Example 4

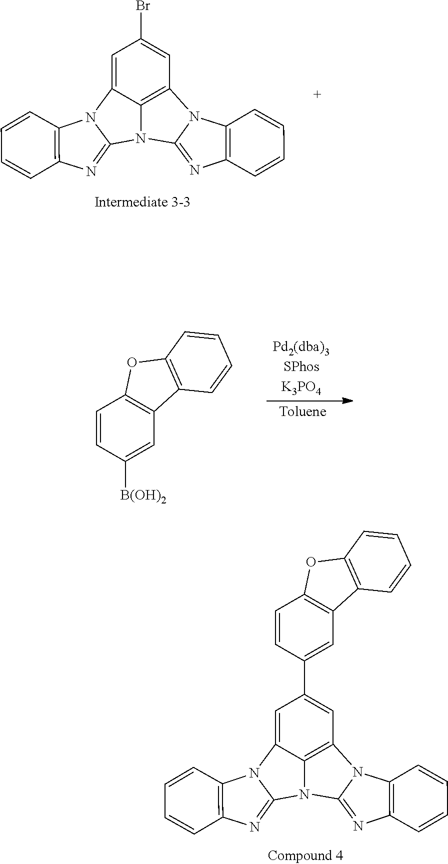 [SMILES: Brc1cc2c3c(c1)n1c4ccccc4nc1n3c1nc3ccccc3n21 ; OB(O)c1ccc2oc3ccccc3c2c1 ; c1ccc2c(c1)nc1n2c2cc(-c3ccc4oc5ccccc5c4c3)cc3c2n1c1nc2ccccc2n31]

### Synthesis of Compound 4

Under argon atmosphere, 5 mL toluene was added to a mixture of the intermediate 3-3 (0.2 g, 0.5 mmol), 2-dibenzofuran boronic acid (0.53 g, 2.5 mmol), tris(dibenzylideneacetone)dipalladium (19 mg, 0.02 mmol), SPhos (33 mg, 0.08 mmol), and potassium phosphate (1.1 g, 5.0 mmol) and the mixture was stirred at 160 degrees C. for three hours. After the reaction, the solution was filtrated to obtain a precipitated solid, which was washed with toluene and methanol to obtain a compound 4 (0.22 g, yield rate 90%). The substance was identified as the compound 4 by FD-MS analysis.

### Synthesis Example 5

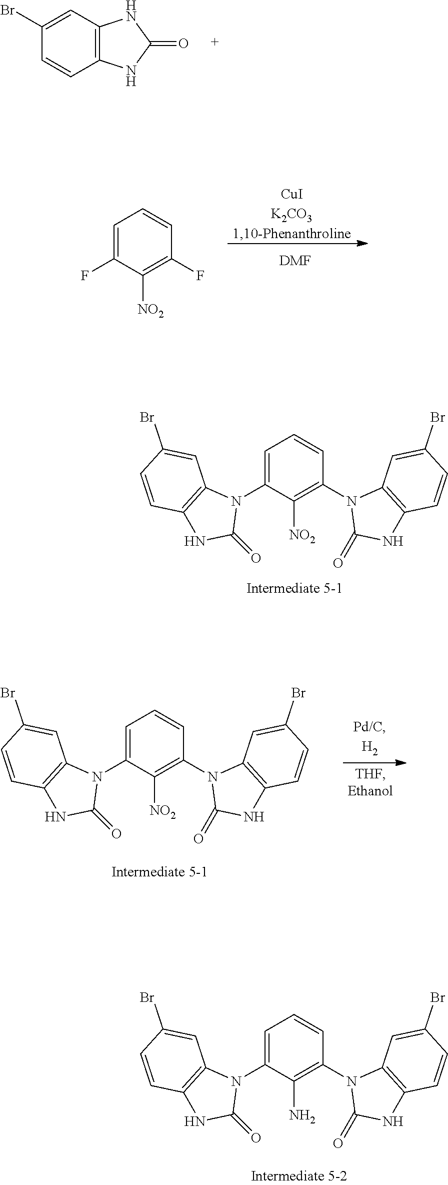

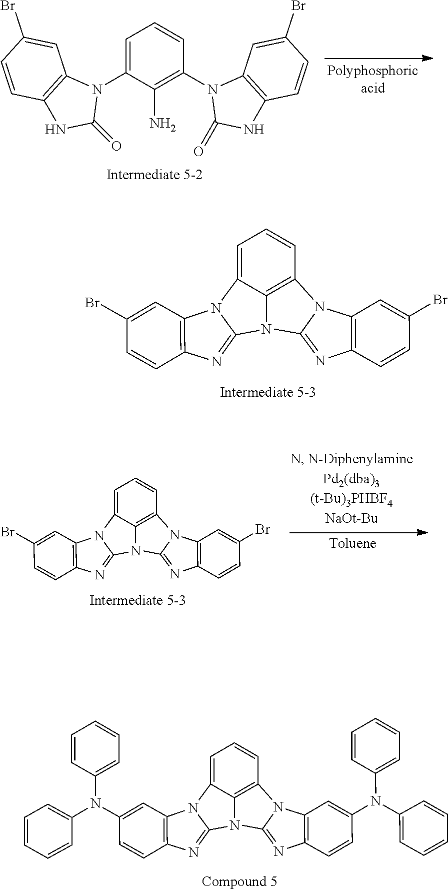 [SMILES: Nc1c(-n2c(=O)[nH]c3ccc(Br)cc32)cccc1-n1c(=O)[nH]c2ccc(Br)cc21 ; Brc1ccc2nc3n(c2c1)c1cccc2c1n3c1nc3ccc(Br)cc3n21 ; Brc1ccc2nc3n(c2c1)c1cccc2c1n3c1nc3ccc(Br)cc3n21 ; c1ccc(N(c2ccccc2)c2ccc3nc4n(c3c2)c2cccc3c2n4c2nc4ccc(N(c5ccccc5)c5ccccc5)cc4n32)cc1]

### Synthesis of Intermediate 5-1

Except for the use of 5-bromo-1,3-difluorobenzimidazole-2-one (12.8 g, 60.0 mmol) instead of 2(3H)-benzimidazolone, the same reaction as in the synthesis example 1 (synthesis of intermediate 1-1) was performed to obtain an intermediate 5-1 (4.1 g, yield rate 25%). The resultant substance was identified to be the intermediate 5-1 by FD-MS analysis.

### Synthesis of Intermediate 5-2

Except for the use of the intermediate 5-1 (4.1 g, 7.5 mmol) instead of the intermediate 1-1, the same reaction as in the synthesis example 1 (synthesis of intermediate 1-2) was performed to obtain an intermediate 5-2 (3.6 g, yield rate 92%). The resultant substance was identified to be the intermediate 5-2 by FD-MS analysis.

### Synthesis of Intermediate 5-3

Except for the use of the intermediate 5-2 (3.6 g, 6.9 mmol) instead of the intermediate 1-2, the same reaction as in the synthesis example 1 (synthesis of compound 1) was performed to obtain an intermediate 5-3 (0.99 g, yield rate 30%). The resultant substance was identified to be the intermediate 5-3 by FD-MS analysis.

### Synthesis of Compound 5

Under argon atmosphere, 10 ml anhydrous toluene was added to a mixture of the intermediate 5-3 (0.48 g, 1.0 mmol), N,N-diphenyl amine (0.37 g, 2.2 mmol), Pd₂(dba)₃ (14 mg, 0.015 mmol), P(tBu)₃HBF₄ (8.7 mg, 0.03 mmol), and sodium t-butoxy (1.9 g, 2.0 mmol), and the mixture was heated for reflux for eight hours.

After the reaction, the reaction solution was cooled to 50 degrees C. and was filtrated through celite/silica gel. Then the filtrate was condensed. The resultant condensed residue was purified through silica-gel column chromatography and recrystallization to obtain a compound 5 (0.34 g, yield rate 52%). The substance was identified as the compound 5 by FD-MS analysis.

### Synthesis Example 6

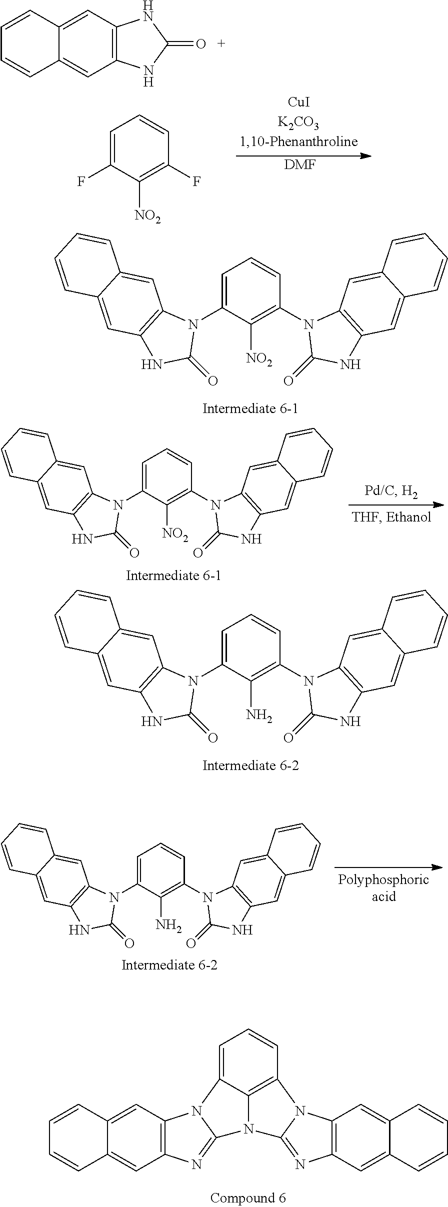

### Synthesis of Intermediate 6-1

Except for the use of 1H-naphtho[2,3-d]imidazole-2(3H)-one (11.1 g, 60.0 mmol) instead of 2(3H)-benzimidazolone, the same reaction as in the synthesis example 1 (synthesis of intermediate 1-1) was performed to obtain an intermediate 6-1 (3.5 g, yield rate 24%). The resultant substance was identified to be the intermediate 6-1 by FD-MS analysis.

### Synthesis of Intermediate 6-2

Except for the use of the intermediate 6-1 (3.5 g, 7.2 mmol) instead of the intermediate 1-1, the same reaction as in the synthesis example 1 (synthesis of intermediate 1-2) was performed to obtain an intermediate 6-2 (3.0 g, yield rate 90%). The resultant substance was identified to be the intermediate 6-2 by FD-MS analysis.

### Synthesis of Compound 6

Except for the use of the intermediate 6-2 (3.0 g, 6.5 mmol) instead of the intermediate 1-2, the same reaction as in the synthesis example 1 (synthesis of compound 1) was performed to obtain a compound 6 (0.49 g, yield rate 18%). The substance was identified as the compound 6 by FD-MS analysis.

What is claimed is:

1. A compound represented by a formula (2) below, 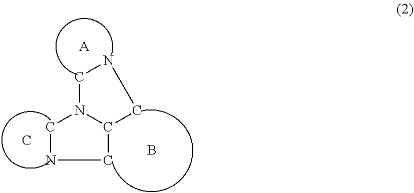
where, in the formula (2): rings A and C each independently represent a substituted or unsubstituted aromatic heterocycle having 5 to 50 ring atoms;
a ring B represents a substituted or unsubstituted aromatic heterocycle having 5 to 50 ring atoms, or a substituted or unsubstituted aromatic hydrocarbon ring having 6 to 50 ring carbon atoms;
substituents for the ring A having a substituent, the ring B having a substituent, and the ring C having a substituent are each independently a substituted or unsubstituted alkyl group having 1 to 50 carbon atoms, a substituted or unsubstituted fluoroalkyl group having 1 to 50 carbon atoms, a substituted or unsubstituted cycloalkyl group having 3 to 50 ring carbon atoms, a cyano group, a halogen atom, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms;
R₉₀₁, R₉₀₂, R₉₀₃, R₉₀₆, and R₉₀₇ are each independently a substituted or unsubstituted aryl group having 6 to 50 ring carbon atoms, a substituted or unsubstituted heterocyclic group having 5 to 50 ring atoms, a substituted or unsubstituted alkyl group having 1 to 50 carbon atoms, or a substituted or unsubstituted cycloalkyl group having 3 to 50 ring carbon atoms; and
R₉₀₄ and R₉₀₅ are each independently a substituted or unsubstituted aryl group having 6 to 50 ring carbon atoms, a substituted or unsubstituted heterocyclic group having 5 to 50 ring atoms, a substituted or unsubstituted alkyl group having 1 to 50 carbon atoms, a substituted or unsubstituted cycloalkyl group having 3 to 50 ring carbon atoms, or a substituted or unsubstituted fluoroalkyl group having 1 to 50 carbon atoms.

2. The compound according to claim 1, wherein the compound is represented by a formula (21) below, 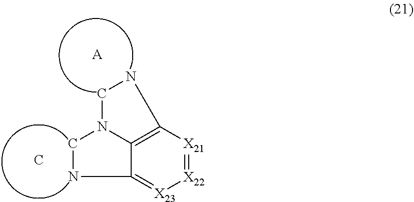
where, in the formula (21):
the ring A and the ring C represent the same as the ring A and the ring C in the formula (2);
X₂₁ is CR₂₁ or a nitrogen atom;
X₂₂ is CR₂₂ or a nitrogen atom;
X₂₃ is CR₂₃ or a nitrogen atom;
a combination of adjacent two or more of R₂₁, R₂₂ and R₂₃ are mutually bonded to form a substituted or unsubstituted saturated ring, mutually bonded to form a substituted or unsubstituted unsaturated ring, or not mutually bonded;
one or more of R₂₁, R₂₂ and R₂₃ not forming the saturated ring and not forming the unsaturated ring each independently represent a hydrogen atom, a substituted or unsubstituted alkyl group having 1 to 50 carbon atoms, a substituted or unsubstituted fluoroalkyl group having 1 to 50 carbon atoms, a substituted or unsubstituted cycloalkyl group having 3 to 50 ring carbon atoms, a cyano group, a halogen atom, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms; and
R₉₀₁, R₉₀₂, R₉₀₃, R₉₀₄, R₉₀₅, R₉₀₆ and R₉₀₇ represent the same as R₉₀₁, R₉₀₂, R₉₀₃, R₉₀₄, R₉₀₅, R₉₀₆ and R₉₀₇ in the formula (2).

3. The compound according to claim 1, wherein
the ring A is the same as the ring C.

4. The compound according to claim 1, wherein
the ring A is different from the ring C.

5. The compound according to claim 1, wherein the compound is represented by a formula (22) below, 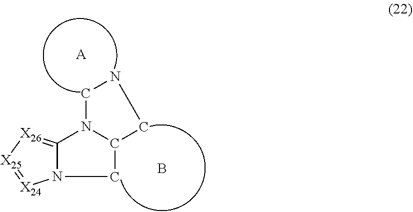
where, in the formula (22):
the ring A and the ring B represent the same as the ring A and the ring B in the formula (2);
X₂₄ is CR₂₄ or a nitrogen atom;
X₂₅ is CR₂₅ or a nitrogen atom;
X₂₆ is CR₂₆ or a nitrogen atom;
a combination of adjacent two or more of R₂₄, R₂₅ and R₂₆ are mutually bonded to form a substituted or unsubstituted saturated ring, mutually bonded to form a substituted or unsubstituted unsaturated ring, or not mutually bonded;
one or more of R₂₄, R₂₅ and R₂₆ not forming the saturated ring and not forming the unsaturated ring each independently represent a hydrogen atom, a substituted or unsubstituted alkyl group having 1 to 50 carbon atoms, a substituted or unsubstituted fluoroalkyl group having 1 to 50 carbon atoms, a substituted or unsubstituted cycloalkyl group having 3 to 50 ring carbon atoms, a cyano group, a halogen atom, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms; and
R₉₀₁, R₉₀₂, R₉₀₃, R₉₀₄, R₉₀₅, R₉₀₆ and R₉₀₇ represent the same as R₉₀₁, R₉₀₂, R₉₀₃, R₉₀₄, R₉₀₅, R₉₀₆ and R₉₀₇ in the formula (2).

6. The compound according to claim 1, wherein the compound is represented by a formula (23) below, 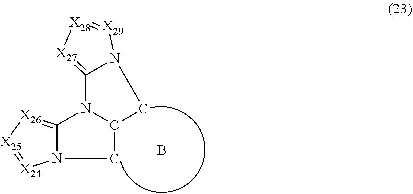
where, in the formula (23):
the ring B is the same as the ring B in the formula (2);
X₂₄ is CR₂₄ or a nitrogen atom;
X₂₅ is CR₂₅ or a nitrogen atom;
X₂₆ is CR₂₆ or a nitrogen atom;
X₂₇ is CR₂₇ or a nitrogen atom;
X₂₈ is CR₂₈ or a nitrogen atom;
X₂₉ is CR₂₉ or a nitrogen atom;
a combination of adjacent two or more of R₂₄, R₂₅ and R₂₆ are mutually bonded to form a substituted or unsubstituted saturated ring, mutually bonded to form a substituted or unsubstituted unsaturated ring, or not mutually bonded;
a combination of adjacent two or more of R₂₇, R₂₈ and R₂₉ are mutually bonded to form a substituted or unsubstituted saturated ring, mutually bonded to form a substituted or unsubstituted unsaturated ring, or not mutually bonded;
one or more of R₂₄, R₂₅, R₂₆, R₂₇, R₂₈, and R₂₉ not forming the saturated ring and not forming the unsaturated ring each independently represent a hydrogen atom, a substituted or unsubstituted alkyl group having 1 to 50 carbon atoms, a substituted or unsubstituted fluoroalkyl group having 1 to 50 carbon atoms, a substituted or unsubstituted cycloalkyl group having 3 to 50 ring carbon atoms, a cyano group, a halogen atom, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms; and
R₉₀₁, R₉₀₂, R₉₀₃, R₉₀₄, R₉₀₅, R₉₀₆ and R₉₀₇ represent the same as R₉₀₁, R₉₀₂, R₉₀₃, R₉₀₄, R₉₀₅, R₉₀₆ and R₉₀₇ in the formula (2).

7. The compound according to claim 6, wherein the compound is represented by a formula (23A) below, 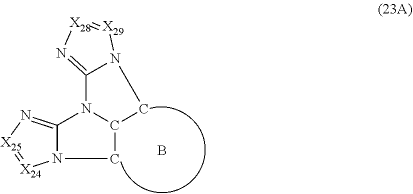
where, in the formula (23A):
the ring B is the same as the ring B in the formula (2);
X₂₄ is CR₂₄ or a nitrogen atom;
X₂₅ is CR₂₅ or a nitrogen atom;
X₂₈ is CR₂₈ or a nitrogen atom;
X₂₉ is CR₂₉ or a nitrogen atom;
R₂₄ and R₂₅ are mutually bonded to form a substituted or unsubstituted saturated ring, mutually bonded to form a substituted or unsubstituted unsaturated ring, or not mutually bonded;
R₂₈ and R₂₉ are mutually bonded to form a substituted or unsubstituted saturated ring, mutually bonded to form a substituted or unsubstituted unsaturated ring, or not mutually bonded;
one or more of R₂₄, R₂₅, R₂₈ and R₂₉ not forming the saturated ring and not forming the unsaturated ring each independently represent a hydrogen atom, a substituted or unsubstituted alkyl group having 1 to 50 carbon atoms, a substituted or unsubstituted fluoroalkyl group having 1 to 50 carbon atoms, a substituted or unsubstituted cycloalkyl group having 3 to 50 ring carbon atoms, a cyano group, a halogen atom, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms; and
R₉₀₁, R₉₀₂, R₉₀₃, R₉₀₄, R₉₀₅, R₉₀₆ and R₉₀₇ represent the same as R₉₀₁, R₉₀₂, R₉₀₃, R₉₀₄, R₉₀₅, R₉₀₆ and R₉₀₇ in the formula (2).

8. The compound according to claim 1, wherein the compound is represented by a formula (24) below, 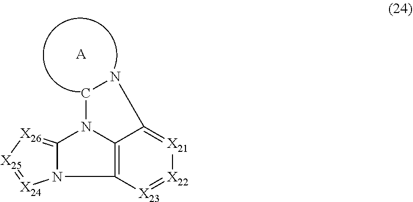
where, in the formula (24):
the ring A represents the same as the ring A in the formula (2);
X₂₁ is CR₂₁ or a nitrogen atom;
X₂₂ is CR₂₂ or a nitrogen atom;
X₂₃ is CR₂₃ or a nitrogen atom;
X₂₄ is CR₂₄ or a nitrogen atom;
X₂₅ is CR₂₅ or a nitrogen atom;
X₂₆ is CR₂₆ or a nitrogen atom;
a combination of adjacent two or more of R₂₁, R₂₂ and R₂₃ are mutually bonded to form a substituted or unsubstituted saturated ring, mutually bonded to form a substituted or unsubstituted unsaturated ring, or not mutually bonded;
a combination of adjacent two or more of R₂₄, R₂₅ and R₂₆ are mutually bonded to form a substituted or unsubstituted saturated ring, mutually bonded to form a substituted or unsubstituted unsaturated ring, or not mutually bonded;
one or more of R₂₁, R₂₂, R₂₃, R₂₄, R₂₅, and R₂₆ not forming the saturated ring and not forming the unsaturated ring each independently represent a hydrogen atom, a substituted or unsubstituted alkyl group having 1 to 50 carbon atoms, a substituted or unsubstituted fluoroalkyl group having 1 to 50 carbon atoms, a substituted or unsubstituted cycloalkyl group having 3 to 50 ring carbon atoms, a cyano group, a halogen atom, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms; and
R₉₀₁, R₉₀₂, R₉₀₃, R₉₀₄, R₉₀₅, R₉₀₆ and R₉₀₇ represent the same as R₉₀₁, R₉₀₂, R₉₀₃, R₉₀₄, R₉₀₅, R₉₀₆ and R₉₀₇ in the formula (2).

9. The compound according to claim 1, wherein the compound is represented by a formula (25) below, 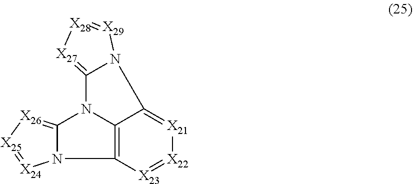
where, in the formula (25):
X₂₁ is CR₂₁ or a nitrogen atom;
X₂₂ is CR₂₂ or a nitrogen atom;
X₂₃ is CR₂₃ or a nitrogen atom;
X₂₄ is CR₂₄ or a nitrogen atom;
X₂₅ is CR₂₅ or a nitrogen atom;
X₂₆ is CR₂₆ or a nitrogen atom;
X₂₇ is CR₂₇ or a nitrogen atom;
X₂₈ is CR₂₈ or a nitrogen atom;
X₂₉ is CR₂₉ or a nitrogen atom;
a combination of adjacent two or more of R₂₁, R₂₂ and R₂₃ are mutually bonded to form a substituted or unsubstituted saturated ring, mutually bonded to form a substituted or unsubstituted unsaturated ring, or not mutually bonded;
a combination of adjacent two or more of R₂₄, R₂₅ and R₂₆ are mutually bonded to form a substituted or unsubstituted saturated ring, mutually bonded to form a substituted or unsubstituted unsaturated ring, or not mutually bonded;
a combination of adjacent two or more of R₂₇, R₂₈ and R₂₉ are mutually bonded to form a substituted or unsubstituted saturated ring, mutually bonded to form a substituted or unsubstituted unsaturated ring, or not mutually bonded;
one or more of R₂₁, R₂₂, R₂₃, R₂₄, R₂₅, R₂₆, R₂₇, R₂₈, and R₂₉ not forming the saturated ring and not forming the unsaturated ring each independently represent a hydrogen atom, a substituted or unsubstituted alkyl group having 1 to 50 carbon atoms, a substituted or unsubstituted fluoroalkyl group having 1 to 50 carbon atoms, a substituted or unsubstituted cycloalkyl group having 3 to 50 ring carbon atoms, a cyano group, a halogen atom, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms; and
R₉₀₁, R₉₀₂, R₉₀₃, R₉₀₄, R₉₀₅, R₉₀₆ and R₉₀₇ represent the same as R₉₀₁, R₉₀₂, R₉₀₃, R₉₀₄, R₉₀₅, R₉₀₆ and R₉₀₇ in the formula (2).

10. The compound according to claim 9, wherein the compound is represented by a formula (25A) below, 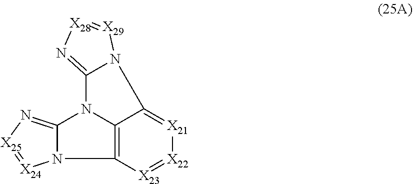
where, in the formula (25A):
X₂₁ is CR₂₁ or a nitrogen atom;
X₂₂ is CR₂₂ or a nitrogen atom;
X₂₃ is CR₂₃ or a nitrogen atom;
X₂₄ is CR₂₄ or a nitrogen atom;
X₂₅ is CR₂₅ or a nitrogen atom;
X₂₈ is CR₂₈ or a nitrogen atom;
X₂₉ is CR₂₉ or a nitrogen atom;
a combination of adjacent two or more of R₂₁, R₂₂ and R₂₃ are mutually bonded to form a substituted or unsubstituted saturated ring, mutually bonded to form a substituted or unsubstituted unsaturated ring, or not mutually bonded;
R₂₄ and R₂₅ are mutually bonded to form a substituted or unsubstituted saturated ring, mutually bonded to form a substituted or unsubstituted unsaturated ring, or not mutually bonded;
R₂₈ and R₂₉ are mutually bonded to form a substituted or unsubstituted saturated ring, mutually bonded to form a substituted or unsubstituted unsaturated ring, or not mutually bonded;
one or more of R₂₁, R₂₂, R₂₃, R₂₄, R₂₅, R₂₈ and R₂₉ not forming the saturated ring and not forming the unsaturated ring each independently represent a hydrogen atom, a substituted or unsubstituted alkyl group having 1 to 50 carbon atoms, a substituted or unsubstituted fluoroalkyl group having 1 to 50 carbon atoms, a substituted or unsubstituted cycloalkyl group having 3 to 50 ring carbon atoms, a cyano group, a halogen atom, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms; and
R₉₀₁, R₉₀₂, R₉₀₃, R₉₀₄, R₉₀₅, R₉₀₆ and R₉₀₇ represent the same as R₉₀₁, R₉₀₂, R₉₀₃, R₉₀₄, R₉₀₅, R₉₀₆ and R₉₀₇ in the formula (2).

11. The compound according to claim 10, wherein the compound is represented by a formula (25B) below, 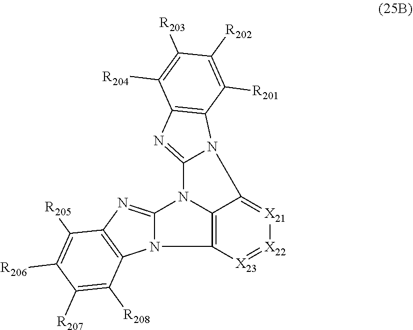
where, in the formula (25B):
X₂₁ is CR₂₁ or a nitrogen atom;
X₂₂ is CR₂₂ or a nitrogen atom;
X₂₃ is CR₂₃ or a nitrogen atom;
a combination of adjacent two or more of R₂₁, R₂₂ and R₂₃ are mutually bonded to form a substituted or unsubstituted saturated ring, mutually bonded to form a substituted or unsubstituted unsaturated ring, or not mutually bonded;
a combination of adjacent two or more of R₂₀₁, R₂₀₂, R₂₀₃ and R₂₀₄ are mutually bonded to form a substituted or unsubstituted saturated ring, mutually bonded to form a substituted or unsubstituted unsaturated ring, or not mutually bonded;
a combination of adjacent two or more of R₂₀₅, R₂₀₆, R₂₀₇ and R₂₀₈ are mutually bonded to form a substituted or unsubstituted saturated ring, mutually bonded to form a substituted or unsubstituted unsaturated ring, or not mutually bonded;
one or more of R₂₁, R₂₂, R₂₃, R₂₀₁, R₂₀₂, R₂₀₃, R₂₀₄, R₂₀₅, R₂₀₆, R₂₀₇, and R₂₀₈ not forming the saturated ring and not forming the unsaturated ring each independently represent a hydrogen atom, a substituted or unsubstituted alkyl group having 1 to 50 carbon atoms, a substituted or unsubstituted fluoroalkyl group having 1 to 50 carbon atoms, a substituted or unsubstituted cycloalkyl group having 3 to 50 ring carbon atoms, a cyano group, a halogen atom, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms; and
R₉₀₁, R₉₀₂, R₉₀₃, R₉₀₄, R₉₀₅, R₉₀₆ and R₉₀₇ represent the same as R₉₀₁, R₉₀₂, R₉₀₃, R₉₀₄, R₉₀₅, R₉₀₆ and R₉₀₇ in the formula (2).

12. The compound according to claim 10, wherein the compound is represented by a formula (25C) below, 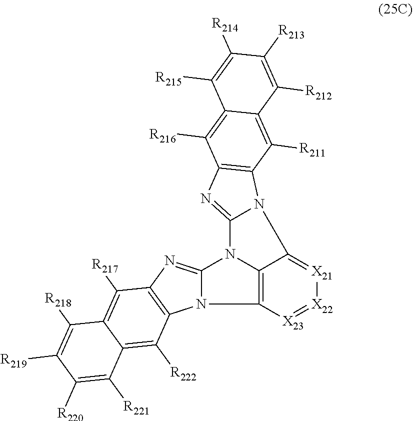
where, in the formula (25C):
X₂₁ is CR₂₁ or a nitrogen atom;
X₂₂ is CR₂₂ or a nitrogen atom;
X₂₃ is CR₂₃ or a nitrogen atom;
a combination of adjacent two or more of R₂₁, R₂₂ and R₂₃ are mutually bonded to form a substituted or unsubstituted saturated ring, mutually bonded to form a substituted or unsubstituted unsaturated ring, or not mutually bonded;
a combination of adjacent two or more of R₂₁₁, R₂₁₂, R₂₁₃, R₂₁₄, R₂₁₅ and R₂₁₆ are mutually bonded to form a substituted or unsubstituted saturated ring, mutually bonded to form a substituted or unsubstituted unsaturated ring, or not mutually bonded;
a combination of adjacent two or more of R₂₁₇, R₂₁₈, R₂₁₉, R₂₂₀, R₂₂₁ and R₂₂₂ are mutually bonded to form a substituted or unsubstituted saturated ring, mutually bonded to form a substituted or unsubstituted unsaturated ring, or not mutually bonded;
one or more of R₂₁, R₂₂, R₂₃, R₂₁₁, R₂₁₂, R₂₁₃, R₂₁₄, R₂₁₅, R₂₁₆, R₂₁₇, R₂₁₈, R₂₁₉, R₂₂₀, R₂₂₁ and R₂₂₂ not forming the saturated ring and not forming the unsaturated ring each independently represent a hydrogen atom, a substituted or unsubstituted alkyl group having 1 to 50 carbon atoms, a substituted or unsubstituted fluoroalkyl group having 1 to 50 carbon atoms, a substituted or unsubstituted cycloalkyl group having 3 to 50 ring carbon atoms, a cyano group, a halogen atom, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms; and
R₉₀₁, R₉₀₂, R₉₀₃, R₉₀₄, R₉₀₅, R₉₀₆ and R₉₀₇ represent the same as R₉₀₁, R₉₀₂, R₉₀₃, R₉₀₄, R₉₀₅, R₉₀₆ and R₉₀₇ in the formula (2).

13. The compound according to claim 10, wherein the compound is represented by a formula (251B), (252B), (253B) or (254B) below, 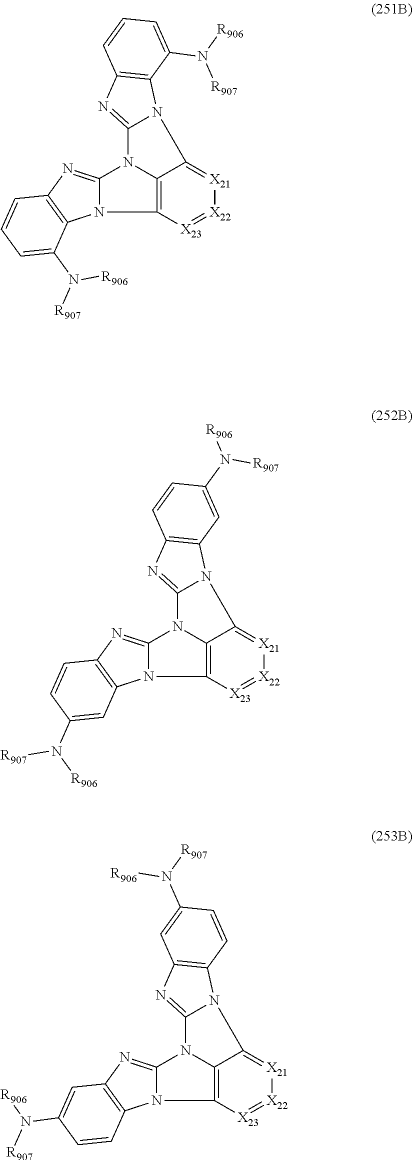 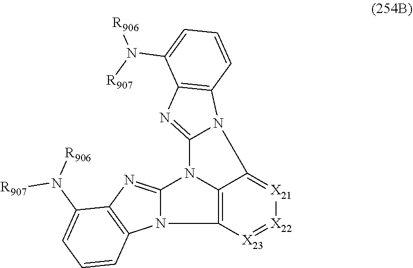
where, in the formula (251B), (252B), (253B) or (254B):
X₂₁ is CR₂₁ or a nitrogen atom;
X₂₂ is CR₂₂ or a nitrogen atom;
X₂₃ is CR₂₃ or a nitrogen atom;
a combination of adjacent two or more of R₂₁, R₂₂ and R₂₃ are mutually bonded to form a substituted or unsubstituted saturated ring, mutually bonded to form a substituted or unsubstituted unsaturated ring, or not mutually bonded;
one or more of R₂₁, R₂₂ and R₂₃ not forming the saturated ring and not forming the unsaturated ring each independently represent a hydrogen atom, a substituted or unsubstituted alkyl group having 1 to 50 carbon atoms, a substituted or unsubstituted fluoroalkyl group having 1 to 50 carbon atoms, a substituted or unsubstituted cycloalkyl group having 3 to 50 ring carbon atoms, a cyano group, a halogen atom, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms; and
R₉₀₁, R₉₀₂, R₉₀₃, R₉₀₄, R₉₀₅, R₉₀₆ and R₉₀₇ represent the same as R₉₀₁, R₉₀₂, R₉₀₃, R₉₀₄, R₉₀₅, R₉₀₆ and R₉₀₇ in the formula (2).

14. The compound according to claim 13, wherein R₉₀₆ and R₉₀₇ are each independently a substituted or unsubstituted aryl group having 6 to 50 ring carbon atoms.

15. The compound according to claim 2, wherein
X₂₁ is C R₂₁,
X₂₂ is C R₂₂,
X₂₃ is C R₂₃, and
R₂₁ and R₂₃ are each a hydrogen atom.

16. The compound according to claim 2, wherein
R₂₂ is a substituted or unsubstituted alkyl group having 1 to 50 carbon atoms, a substituted or unsubstituted fluoroalkyl group having 1 to 50 carbon atoms, a substituted or unsubstituted cycloalkyl group having 3 to 50 ring carbon atoms, a cyano group, a halogen atom, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms; and
R₉₀₁, R₉₀₂, R₉₀₃, R₉₀₄, R₉₀₅, R₉₀₆ and R₉₀₇ represent the same as R₉₀₁, R₉₀₂, R₉₀₃, R₉₀₄, R₉₀₅, R₉₀₆ and R₉₀₇ in the formula (2).

17. The compound according to claim 16, wherein
R₂₂ is a substituted or unsubstituted alkyl group having 1 to 6 carbon atoms, a substituted or unsubstituted fluoroalkyl group having 1 to 6 carbon atoms, a substituted or unsubstituted cycloalkyl group having 3 to 6 ring carbon atoms, a cyano group, a halogen atom, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 18 ring carbon atoms, or a substituted or unsubstituted heterocyclic group having 5 to 18 ring atoms;
R₉₀₁, R₉₀₂, R₉₀₃, R₉₀₆, and R₉₀₇ included in the group represented by R₂₂ are each independently a substituted or unsubstituted aryl group having 6 to 18 ring carbon atoms, a substituted or unsubstituted heterocyclic group having 5 to 18 ring atoms, a substituted or unsubstituted alkyl group having 1 to 6 carbon atoms, or a substituted or unsubstituted cycloalkyl group having 3 to 6 ring carbon atoms; and
R₉₀₄ and R₉₀₅ included in the group represented by R₂₂ are each independently a substituted or unsubstituted aryl group having 6 to 18 ring carbon atoms, a substituted or unsubstituted heterocyclic group having 5 to 18 ring atoms, a substituted or unsubstituted alkyl group having 1 to 6 carbon atoms, a substituted or unsubstituted cycloalkyl group having 3 to 6 ring carbon atoms, or a substituted or unsubstituted fluoroalkyl group having 1 to 6 carbon atoms.

18. An organic electroluminescence device comprising:
an anode;
a cathode; and
a first organic layer interposed between the anode and the cathode, wherein the first organic layer comprises the compound according to claim 1.

19. The organic electroluminescence device according to claim 18, wherein the first organic layer is an emitting layer.

20. The organic electroluminescence device according to claim 18, wherein
the first organic layer comprises a first compound and a second compound,
the first compound is represented by a formula (10) below, and
the second compound is the compound according to claim 1, 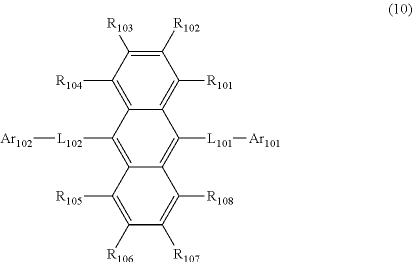
where, in the formula (10): R₁₀₁ to R₁₀₈ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₁₂₄, a group represented by —COOR₁₂₅, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by a formula (11) below,
-L₁₀₃-Ar₁₀₃ (11)
L₁₀₁, L₁₀₂, and L₁₀₃ are each independently a single bond, a substituted or unsubstituted arylene group having 6 to 50 ring carbon atoms, or a substituted or unsubstituted divalent heterocyclic group having 5 to 50 ring atoms;
Ar₁₀₂, and Ar₁₀₃ are each independently a substituted or unsubstituted aryl group having 6 to 50 ring carbon atoms, or a substituted or unsubstituted monovalent heterocyclic group having 5 to 50 ring atoms;
R₉₀₁, R₉₀₂, R₉₀₃, R₉₀₆, R₉₀₇, R₁₂₄ and R₁₂₅ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
R₉₀₄ and R₉₀₅ are each independently a substituted or unsubstituted alkyl group having 1 to 50 carbon atoms, or a substituted or unsubstituted aryl group having 6 to 50 ring carbon atoms;
a plurality of R₉₀₁ are mutually the same or different;
a plurality of R₉₀₂ are mutually the same or different;
a plurality of R₉₀₃ are mutually the same or different;
a plurality of R₉₀₄ are mutually the same or different;
a plurality of R₉₀₅ are mutually the same or different;
a plurality of R₉₀₆ are mutually the same or different;
a plurality of R₉₀₇ are mutually the same or different;
a plurality of R₁₂₄ are mutually the same or different;
a plurality of R₁₂₅ are mutually the same or different; and
a plurality of groups represented by the formula (11) are mutually the same or different.

21. The organic electroluminescence device according to claim 20, wherein a singlet energy S₁(H) of the first compound and a singlet energy S₁(D) of the second compound satisfy a relationship of Numerical Formula 1 below,
S₁(H)>S₁(D) (Numerical Formula 1).

22. The organic electroluminescence device according to claim 20, wherein the first compound is represented by a formula (13) below, 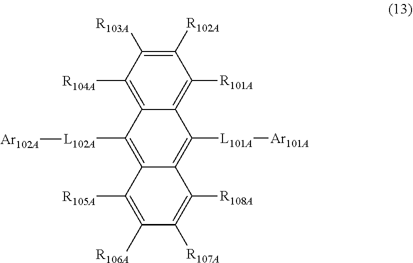
where, in the formula (13):
R_{101A} to R_{108A} each independently represent a hydrogen atom, or a substituted or unsubstituted aryl group having 6 to 50 ring carbon atoms;
L_{101A} and L_{102A} are each independently a single bond, or a substituted or unsubstituted arylene group having 6 to 30 ring carbon atoms; and
Ar_{101A} and Ar_{102A} are each independently a substituted or unsubstituted aryl group having 6 to 50 ring carbon atoms.

23. The organic electroluminescence device according to claim 20, wherein the first compound is represented by a formula (14) below, 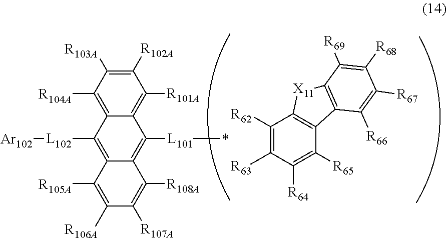
where, in the formula (14):
L₁₀₁, L₁₀₂ and Ar₁₀₂ represent the same as L₁₀₁, L₁₀₂ and Ar₁₀₂ in the formula (10);
R_{101A} to R_{108A} each independently represent a hydrogen atom, or a substituted or unsubstituted aryl group having 6 to 50 ring carbon atoms;
X₁₁ is an oxygen atom, sulfur atom, or N(R₆₁);
R₆₁ is a hydrogen atom, a substituted or unsubstituted alkyl group having 1 to 50 carbon atoms, or a substituted or unsubstituted aryl group having 6 to 50 ring carbon atoms;
one of R₆₂ to R₆₉ is a bond with L₁₀₁;
* represents a bonding position with L₁₀₁; and
at least one combination of adjacent two or more of R₆₂ to R₆₉ not bonded with L₁₀₁ are mutually bonded to form a substituted or unsubstituted saturated ring, mutually bonded to form a substituted or unsubstituted unsaturated ring, or not mutually bonded; and
one or more of R₆₂ to R₆₉ not bonded with L₁₀₁, not forming the saturated ring and not forming the unsaturated ring each independently represent a hydrogen atom, a substituted or unsubstituted alkyl group having 1 to 50 carbon atoms, or a substituted or unsubstituted aryl group having 6 to 50 ring carbon atoms.

24. The organic electroluminescence device according to claim 23, wherein one or more of R₆₂ to R₆₉ not bonded with L₁₀₁ are not mutually bonded.

25. The organic electroluminescence device according to claim 20, wherein the first compound is represented by a formula (15) below, 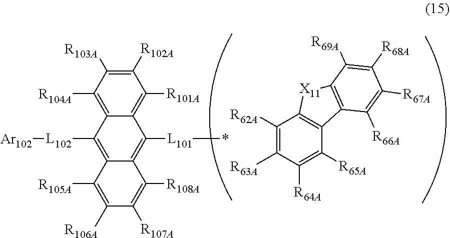
where, in the formula (15):
L₁₀₁, L₁₀₂ and Ar₁₀₂ represent the same as L₁₀₁, L₁₀₂ and Ar₁₀₂ in the formula (10);
R_{101A} to R_{108A} each independently represent a hydrogen atom, or a substituted or unsubstituted aryl group having 6 to 50 ring carbon atoms;
X₁₁ is an oxygen atom, sulfur atom, or N(R₆₁);
R₆₁ is a hydrogen atom, a substituted or unsubstituted alkyl group having 1 to 50 carbon atoms, or a substituted or unsubstituted aryl group having 6 to 50 ring carbon atoms;
at least one combination of adjacent two or more of R_{62A} to R_{69A} are mutually bonded to form a substituted or unsubstituted saturated ring, mutually bonded to form a substituted or unsubstituted unsaturated ring, or not mutually bonded;
the combination of adjacent two of R_{62A} to R_{69A} are mutually bonded to form a ring represented by a formula (15A) below;
one or more of R_{62A} to R_{69A} not forming the saturated ring and not forming the unsaturated ring each independently represent a hydrogen atom, a substituted or unsubstituted alkyl group having 1 to 50 carbon atoms, or a substituted or unsubstituted aryl group having 6 to 50 ring carbon atoms; 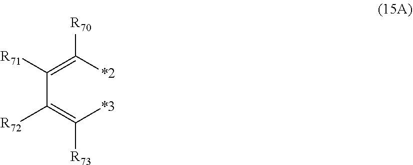
where, in the formula (15A):
*2 and *3 represent bonding portions with adjacent two of R_{62A} to R_{69A};
one of R_{62A} to R_{69A} and R₇₀ to R₇₃ is a bond with L₁₀₁;
* represents a bonding position with L₁₀₁; and
R₇₀ to R₇₃ not bonded with L₁₀₁ each independently represent a hydrogen atom, a substituted or unsubstituted alkyl group having 1 to 50 carbon atoms, or a substituted or unsubstituted aryl group having 6 to 50 ring carbon atoms.

26. The organic electroluminescence device according to claim 18, further comprising a hole transporting layer between the anode and the first organic layer.

27. The organic electroluminescence device according to claim 18, further comprising an electron transporting layer between the cathode and the first organic layer.

28. An electronic device comprising the organic electroluminescence device according to claim 18.
